(19) United States
(12) Patent Application Publication
(10) Pub. No.: US 2023/0057310 A1
(43) Pub. Date: Feb. 23, 2023

(54) TREATMENT METHODS

(71) Applicant: Genocea Biosciences, Inc., Cambridge, MA (US)

(72) Inventors: Jessica Baker Flechtner, Sudbury, MA (US); Marie Lossky-Elias, Cambridge, MA (US); Pamela M. Carroll, Boston, MA (US); Hubert Lam, Quincy, MA (US); Lisa K. McNeil, Watertown, MA (US); Wendy Jane Broom, Arlington, MA (US)

(21) Appl. No.: 17/280,594

(22) PCT Filed: Sep. 27, 2019
(86) PCT No.: PCT/US19/53672

§ 371 Date: Mar. 26, 2021

Related U.S. Application Data
(60) Provisional application No. 62/737,832, filed on Sep. 27, 2018.
(60) Provisional application No. 62/757,915, filed on Nov. 9, 2018.

(51) Int. Cl. A61K 39/00; A61K 39/39; A61P 35/00

(52) U.S. Cl. CPC A61K 39/001184; A61K 39/39; A61K 39/0011; A61K 2039/545; A61K 2039/55561; A61K 2039/55572; A61K 2039/55577; A61P 35/00

(57) ABSTRACT

Methods and compositions for identifying tumor antigens of human lymphocytes, and for treating subjects having cancer, are provided herein.

### CROSS REFERENCE TO RELATED APPLICATIONS

This application claims the benefit of U.S. Provisional Application No. 62/737,832, filed Sep. 27, 2018, and U.S. Provisional Application No. 62/757,915, filed Nov. 9, 2018, the contents of each of which are hereby incorporated by reference herein in their entirety.

### BACKGROUND

Cancer is characterized by proliferation of abnormal cells. Many treatments include costly and painful surgeries and chemotherapies. Although there is a growing interest in cancer therapies that target cancerous cells using a patient's own immune system, such therapies have had limited success.

### SUMMARY

The present invention features, inter alia, a method of inducing an immune response in a subject.

One aspect of the disclosure includes a method of inducing an immune response in a subject, comprising: administering to the subject (i) at least one inhibitory antigen (e.g., 1, 2, 3, 4, 5, 6, 7, 8, 9, 10, or more) and (ii) an effective amount of an agent or a combination of agents, thereby inducing an immune response in the subject. In some embodiments, administration of the inhibitory antigen to the subject, without an effective amount of the agent or the combination of agents, induces an immune response that impairs or reduces immune control of a tumor or cancer cell in the subject.

In some embodiments, the administering step induces an immune response that enhances immune control of the tumor or cancer. In some embodiments, administration of the effective amount of the agent or combination of agents redirects an immune response to the inhibitory antigen. In some embodiments, the immune response to the inhibitory antigen is redirected from an immune response that impairs or reduces immune control of the tumor or cancer to an immune response that does not alter, or that enhances immune control of the tumor or cancer.

In some embodiments, the agent or combination of agents comprises an adjuvant. In some embodiments, the adjuvant comprises a TLR agonist, an inflammasome activator, a NOD2 agonist, a RIG1 helicase inhibitor, or a STING agonist. In some embodiments, the agent or combination of agents comprises two or more adjuvants. In some embodiments, the two or more adjuvants comprise a TLR agonist, an inflammasome activator, a NOD2 agonist, a RIG1 helicase inhibitor, and/or a STING agonist. In some embodiments, the agent or combination of agents comprises a checkpoint inhibitor (e.g., a PD-1 inhibitor, a PD-L1 inhibitor, or a CTLA-4 inhibitor). In some embodiments, the combination of agents comprises a checkpoint inhibitor and an adjuvant. In some embodiments, the agent or combination of agents comprises a viral vector, a bacterial vector, an exosome, a liposome, DNA, mRNA, saRNA, a chemotherapeutic agent or an IDO inhibitor. In some embodiments, the agent or combination of agents comprises an agonist (e.g., a 4-1BB agonist, an OX40 agonist, or a GITR agonist).

In some embodiments, the inhibitory antigen is a tumor antigen (e.g., tumor specific antigen [TSA or neoantigen], tumor associated antigen [TAA], or cancer/testis antigen [CTA]). In some embodiments, the inhibitory antigen is a full-length polypeptide, or a fragment or peptide thereof.

In some embodiments, an immune response comprises a T cell-mediated immune response. In some embodiments, an immune response comprises an antigen presenting cell (APC)-mediated immune response. In some embodiments, an immune response comprises a B cell-mediated immune response. In some embodiments, an immune response comprises a response mediated by one or more cells of the innate immune system (e.g., an NK cell, an NKT cell, a macrophage, or a monocyte).

In some embodiments, an immune response that impairs or reduces immune control of a tumor or cancer cell comprises a deleterious or non-beneficial lymphocyte response. In some embodiments, the deleterious or non-beneficial lymphocyte response comprises a decrease or no measurable change, relative to a control, in the level of one or more co-stimulatory molecules or signals, one or more immune or cytokine signals, or one or more MHC molecules. In some embodiments, the deleterious or non-beneficial lymphocyte response comprises a decrease or no measurable change, relative to a control, in storage or secretion of immune lytic molecules (e.g., granzyme, or perforin), or other immune effector molecules. In some embodiments, the deleterious or non-beneficial lymphocyte response comprises a decrease or no measurable change, relative to a control, in cytotoxic CD8⁺ T cell and/or CD4⁺ Th1 activity. In some embodiments, the deleterious or non-beneficial lymphocyte response comprises a decrease or no measurable change, relative to a control, in recruitment of beneficial immune cell types. In some embodiments, the deleterious or non-beneficial lymphocyte response comprises an increase, relative to control, in storage or secretion of immunoregulatory cytokines (e.g., IL-10, or TGFβ).

In some embodiments, the deleterious or non-beneficial lymphocyte response comprises a reduction, relative to a control, in a level of an anti-tumor antibody. In some embodiments, the deleterious or non-beneficial lymphocyte response may include a reduction, relative to a control, in a level of antibody-dependent cell-mediated toxicity (ADCC) against a tumor. In some embodiments, the deleterious or non-beneficial lymphocyte response comprises a reduction, relative to a control, in a level of an antibody that binds the inhibitory antigen expressed by, or present on a surface of, the tumor.

In some embodiments, an immune response that enhances immune control of a tumor or cancer cell comprises a beneficial lymphocyte response. In some embodiments, the beneficial lymphocyte response comprises an increase, relative to a control, in the level of one or more immune co-stimulatory molecules or signals, one or more immune cytokines or cytokine signals, or one or more MHC molecules. In some embodiments, the beneficial lymphocyte response comprises an increase, relative to a control, in storage or secretion of immune lytic molecules (e.g., granzyme, or perforin), or other immune effector molecules. In some embodiments, the beneficial lymphocyte response comprises an increase, relative to a control, in cytotoxic CD8⁺ T cell activity. In some embodiments, the beneficial lymphocyte response comprises an increase, relative to a control, in CD4⁺ Th1 cell activity. In some embodiments, the beneficial lymphocyte response comprises an increase, relative to a control, in recruitment of beneficial immune cell types.

In some embodiments, the beneficial lymphocyte response comprises an increase, relative to a control, in a level of an anti-tumor antibody. In some embodiments, the beneficial lymphocyte response comprises an increase, relative to a control, in a level of antibody-dependent cell-mediated toxicity (ADCC) against a tumor. In some embodiments, the beneficial lymphocyte response comprises an increase, relative to a control, in a level of an antibody that binds the inhibitory antigen expressed by, or present on a surface of, the tumor.

In some embodiments, the inhibitory antigen and the agent or combination of agents are co-administered. In some embodiments, the inhibitory antigen and the agent or combination of agents are co-administered as a single composition. In some embodiments, the inhibitory antigen and the agent or combination of agents are co-administered as separate compositions.

In some embodiments, the inhibitory antigen is administered prior to the agent or combination of agents. In some embodiments, the inhibitory antigen is administered after the agent or combination of agents.

In some embodiments, an immune response that enhances immune control of the tumor or cancer comprises one or more beneficial clinical responses. In some embodiments, an immune response that enhances immune control of the tumor or cancer comprises clearance, or regression, or stabilization of the tumor or cancer, e.g., a level of one or more clinical measures associated with clearance, regression, or stabilization of a cancer. In some embodiments, immune control of the tumor or cancer comprises a complete response (CR), a partial response (PR), or stable disease (SD) using RECIST (Response Evaluation Criteria in Solid Tumors) criteria (including iRECIST and RECIST 1.1). In some embodiments, an immune response that enhances immune control of the tumor or cancer comprises an absence of relapse, recurrence, and/or metastasis of a cancer, e.g., over a defined period of time (e.g., at least 1, 2, 3, 4, 5, 6, 7, 8, 9, 10, 11, 12 weeks, or at least 1, 2, 3, 4, 5, 6, 7, 8, 9, 10, 11, 12 months, or at least 1, 2, 3, 4, 5, 6, 7, 8, 9, 10, 11, 12 years).

In some embodiments, an immune response that enhances immune control of the tumor or cancer comprises a positive cancer prognosis. In some embodiments, an immune response that enhances immune control of the tumor or cancer comprises an absence or reduction of one or more toxic responses and/or side effects (e.g., one or more measurable toxic responses and/or side effects) to a cancer therapy or combination of therapies.

In some embodiments, the method of inducing an immune response in a subject may further include administering to the subject a cancer therapy or combination of therapies.

In some embodiments, the method for inducing an immune response in a subject further comprises a module for identifying an inhibitory antigen and/or stimulatory antigen. The module may identify an inhibitory and/or stimulatory antigen through measuring secretion of one or more immune mediators associated with one or more deleterious or not beneficial responses to cancer.

In some embodiments, the method further comprises identifying one or more inhibitory antigens and/or one or more stimulatory antigens. In some embodiments, the method further comprises: a) obtaining, providing, or generating a library comprising bacterial cells or beads comprising a plurality of tumor antigens, wherein each bacterial cell or bead of the library comprises a different tumor antigen; b) contacting the bacterial cells or beads with antigen presenting cells (APCs) from a subject, wherein the APCs internalize the bacterial cells or beads; c) contacting the APCs with lymphocytes from the subject, under conditions suitable for activation of lymphocytes by a tumor antigen presented by one or more APCs; d) determining whether one or more lymphocytes are activated by, or not responsive to, one or more tumor antigens presented by one or more APCs, e.g., by assessing (e.g., detecting or measuring) a level (e.g., an increased or decreased level, relative to a control) of expression and/or secretion of one or more immune mediators or cell-surface molecules; e) identifying one or more tumor antigens that stimulate, inhibit and/or suppress, and/or have a minimal effect on a level of expression and/or secretion of one or more immune mediators; and (f) identifying as one or more inhibitory antigens one or more tumor antigens that increase expression or secretion of immune mediators associated with deleterious or not beneficial responses to cancer, and/or one or more tumor antigens that inhibit and/or suppress expression or secretion of immune mediators associated with beneficial responses to cancer; and/or (g) identifying as one or more stimulatory antigens (i) one or more tumor antigens that increase level of expression and/or secretion of one or more immune mediators associated with one or more beneficial responses to cancer, and/or (ii) one or more tumor antigens that inhibit and/or suppress level of expression and/or secretion of one or more immune mediators associated with one or more deleterious or not beneficial responses to cancer.

In some embodiments, the APCs are human APCs isolated from the subject; and/or the bacterial cells further comprise a cytolysin polypeptide; and/or the cytolysin polypeptide is listeriolysin O (LLO); and/or the APCs are provided in an array, and/or the APCs in each location of the array are contacted with a set of bacterial cells, each set comprising a different tumor antigen; and/or the APCs and lymphocytes are isolated from peripheral blood; and/or the APCs comprise immortalized cells; and/or the lymphocytes are derived from a cancer or tumor.

In some embodiments, the tumor antigens comprise full length polypeptides encoding mutations, splice variants, or translocations present in a cancer or tumor; and/or the tumor antigens comprise polypeptides that are fragments of full length polypeptides encoding mutations, splice variants, or translocations present in a cancer or tumor; and/or the tumor antigens comprise full length polypeptides encoded by a virus or other infectious agent present in a cancer or tumor; and/or the tumor antigens comprise polypeptides that are fragments of full length polypeptides encoded by a virus or other infectious agent present in a cancer or tumor; and/or the tumor antigens comprise full length polypeptides encoding autoantigens associated with a cancer or tumor; and/or the tumor antigens comprise polypeptides that are fragments of full length polypeptides encoding autoantigens associated with a cancer or tumor.

In some embodiments, the method comprises non-specifically stimulating T cell responses (e.g., with an anti-CD3 antibody, or a mitogen such as PHA, ConA, PMA and Ionomycin), then pulsing the T cells with peptides or protein antigens to detect a change in immune response relative to control.

In another aspect, the disclosure features compositions, e.g., immunogenic compositions. In some embodiments, an immunogenic composition comprises (i) at least one inhibitory antigen described herein and (ii) an effective amount of an agent or a combination of agents described herein. In some embodiments, the immunogenic composition further comprises a pharmaceutically acceptable carrier.

### BRIEF DESCRIPTION OF THE DRAWINGS

The present teachings described herein will be more fully understood from the following description of various illustrative embodiments, when read together with the accompanying drawings. It should be understood that the drawings described below are for illustration purposes only and are not intended to limit the scope of the present teachings in any way.

FIG. 1 is a graph showing normalized CD8⁺ T cell response levels, measured by production of either IFNγ (panel A) or TNFα (panel B), against different mutated tumor proteins.

FIG. 2 is a Venn diagram showing limited overlap between CD8⁺ T cell stimulatory and inhibitory antigens identified using methods of the disclosure compared to epitope prediction algorithms.

FIG. 3 shows a diagram of exemplary methods used to rank stimulatory and inhibitory antigens of the disclosure. Three screens were run measuring IFNγ and TNFα (panel A) and a ranked list was generated based on the three screens (panels B and C).

FIG. 4 is a graph showing the results of an IFNγ ELISPOT assay for determining the immunogenicity and level of T cell activation in response to immunization with the indicated pools of three or four antigens. Panel (A) shows the level of T cell activation in response to the indicated pools of three or four antigens administered with triple adjuvant A (CpG, 3D-PHAD, synthetic saponin). Panel (B) shows the level of T cell activation in response to the indicated pools of three or four antigens without adjuvant. Symbols represent responses from individual mice.

FIG. 5 is a graph showing mean tumor areas measured over time in mice immunized with the indicated pools of four antigens.

FIG. 6 shows multiple graphs of the tumor area (mm²) measured over time in individual mice of the indicated immunization groups. Panel (A) represents the tumor area in mice immunized with control PBS/DMSO only, panel (B) represents the tumor area in mice immunized with a pool of four stimulatory antigens, panel (C) represents the tumor area in mice immunized with a first pool of four inhibitory antigens, and panel (D) represents the tumor area in mice immunized with a second pool of four inhibitory antigens.

FIG. 7 is a graph showing mean tumor area measured over time in mice immunized with the indicated pools of three or four antigens and triple adjuvant A (CpG, 3D-PHAD, synthetic saponin).

FIG. 8 shows multiple graphs of the tumor area (mm²) measured over time in individual mice of the indicated immunization groups. Panel (A) represents the tumor area in control mice immunized with adjuvant only, panel (B) represents the tumor area in mice immunized with a pool of four stimulatory antigens and adjuvant, panel (C) represents the tumor area in mice immunized with a first pool of four inhibitory antigens and adjuvant, panel (D) represents the tumor area in mice immunized with a second pool of four stimulatory antigens and adjuvant, and panel (E) represents the tumor area in mice immunized with a pool of three previously known efficacious antigens (Published) and adjuvant. Adjuvant in all cases was triple adjuvant A (CpG, 3D-PHAD, synthetic saponin).

FIG. 9 shows multiple graphs of the percent survival of immunized mice over time. Panel (A) shows the percent survival of mice over time in experiments testing immunization with indicated pools of four antigens, or control PBS/DMSO only. Panel (B) shows the percent survival of mice over time in experiments testing immunization with indicated pools of three or four antigens plus triple adjuvant A (CpG, 3D-PHAD, synthetic saponin), or triple adjuvant A only.

FIG. 10 shows fluorescence scans of representative tumor sections from mice immunized with phosphate buffered saline (PBS) only, or a pool of inhibitory antigens only. Panel (A) shows a fluorescent CD8⁺ and DAPI stained section of a representative (average) tumor from a mouse immunized with PBS only. Panel (B) shows a fluorescent CD8⁺ and DAPI stained section of a hyper-progressive tumor from a mouse immunized with a pool of inhibitory antigens only.

FIG. 11 is a graph showing mean number of infiltrating CD8⁺ T cells in whole tumors (N=2) from mice immunized with phosphate buffered saline (PBS) only, or a pool of inhibitory antigens only.

FIG. 12 shows graphs of the mean tumor volume (mm³) measured over time in mice of the indicated immunization groups. Panel (A) represents the mean tumor volume for mice immunized with: (1) adjuvant only; (2) a pool comprising inhibitory antigen In21 and two previously known efficacious antigens with adjuvant (ln 21+Published); or (3) two previously known efficacious antigens only (Published). Panel (B) represents the mean tumor volume for mice immunized as in Panel A, and additionally for mice immunized with: (4) a pool comprising 4 inhibitory antigens and two previously known efficacious antigens with adjuvant (Inhib Pool+Published); or (5) a pool comprising inhibitory antigen In17 and two previously known efficacious antigens with adjuvant (ln 17+Published). Adjuvant in all cases was triple adjuvant B (CpG, 3D-PHAD, QS21).

FIG. 13 shows results of therapeutic immunization with a pool of 4 inhibitory antigens combined with triple adjuvant B (CpG, 3D-PHAD, QS21) compared to immunization with the adjuvant only. Results for Panels A-B are expressed as tumor volume in mm³ over time. Panel A shows mean curves for the two immunization groups. Panel B shows curves for individual mice in the two immunization groups. Panels C and D show the correlation between tumor volume in mm³ and IFNγ spot forming units per 200K cells, a measure of immunogenicity and T cell activation, using two different graphing conventions. In Panel C, square symbols represent IFNγ spot forming units per 200K cells. Circles represent tumor volume (mm³) on day 17, following injection with B16F10 cancer cells on day 0. Each symbol on the graphs represents the response of an individual mouse.

FIG. 14 shows results of IFNγ ELISPOT assays for determining the immunogenicity and level of T cell activation in peripheral blood cells of mice immunized with a pool of four inhibitory antigens in combination with the indicated adjuvant. Panel (A) shows T cell activation following immunization with inhibitory antigens and poly-IC adjuvant. Panel (B) shows T cell activation following immunization with inhibitory antigens and triple adjuvant B (Triple: CpG, 3D-PHAD, QS21). Panel (C) shows T cell activation following immunization with inhibitory antigens and incomplete Freund's adjuvant (IFA). Panel (D) shows T cell activation following immunization with inhibitory antigens and CpG adjuvant. Panel (E) shows T cell activation following immunization with inhibitory antigens and no adjuvant (Peptide only). Control mice were immunized with the indicated adjuvant only, or phosphate buffered saline (PBS). Peripheral blood cells of immunized mice were stimulated with overlapping peptides spanning the inhibitory antigens (Inhibitory Pool) or media only (Media), as indicated on the x-axis. Results are expressed as the number of IFNγ spot forming units per 200,000 cells. Each symbol on the graphs represents the response of an individual mouse.

FIG. 15 shows results of IFNγ ELISPOT assays for determining the immunogenicity and level of T cell activation in splenocytes of mice immunized with a pool of four inhibitory antigens in combination with the indicated adjuvant. Panel (A) shows T cell activation following immunization with inhibitory antigens and poly-IC adjuvant. Panel (B) shows T cell activation following immunization with inhibitory antigens and triple adjuvant B (Triple: CpG, 3D-PHAD, QS21). Panel (C) shows T cell activation following immunization with inhibitory antigens and incomplete Freund's adjuvant (IFA). Panel (D) shows T cell activation following immunization with inhibitory antigens and CpG adjuvant. Panel (E) shows T cell activation following immunization with inhibitory antigens and no adjuvant (Peptide Only). Control mice were immunized with the indicated adjuvant only, or phosphate buffered saline (PBS). Splenocytes of immunized mice were stimulated with overlapping peptides spanning the inhibitory antigens (Inhibitory Pool) or media only (Media), as indicated on the x-axis. Results are expressed as the number of IFNγ spot forming units per 400,000 cells. Each symbol on the graphs represents the response of an individual mouse.

FIG. 16 shows results of IFNγ ELISPOT assays for determining the immunogenicity and level of T cell activation in lymph node cells of mice immunized with a pool of four inhibitory antigens in combination with the indicated adjuvant. Panel (A) shows T cell activation following immunization with inhibitory antigens and poly-IC adjuvant. Panel (B) shows T cell activation following immunization with inhibitory antigens and triple adjuvant B (Triple: CpG, 3D-PHAD, QS21). Panel (C) shows T cell activation following immunization with inhibitory antigens and incomplete Freund's adjuvant. Panel (D) shows T cell activation following immunization with inhibitory antigens and CpG adjuvant. Panel (E) shows T cell activation following immunization with inhibitory antigens and no adjuvant. Control mice were immunized with the indicated adjuvant only, or phosphate buffered saline (PBS). Lymph node cells of immunized mice were stimulated with overlapping peptides spanning the inhibitory antigens (Inhibitory Pool) or media only (Media), as indicated on the x-axis. Results are expressed as the number of IFNγ spot forming units per 200,000 cells. Each symbol on the graphs represents the response of an individual mouse.

FIG. 17 shows the tumor volume measured in individual mice of the indicated immunization groups. Panel (A) represents the tumor volume over time (from day 0=injection with B16F10 cancer cells) in mice immunized with triple adjuvant B only (Triple: CpG, 3D-PHAD, QS21). Panel (B) represents the tumor volume (mm³) over time (from day 0=injection with B16F10 cancer cells) in mice immunized with a pool of four inhibitory antigens and triple adjuvant B (Triple: CpG, 3D-PHAD, QS21). Each line on the graphs represents the tumor volume (mm³) of an individual mouse.

FIG. 18 shows the fold-change in tumor volume measured over time in mice immunized with a pool of 4 inhibitory antigens and the indicated adjuvant, relative to control mice immunized with adjuvant only. Immunization groups indicated on the x axis comprised poly-IC adjuvant, triple adjuvant B (Triple: CpG, 3D-PHAD, QS21), incomplete Freund's adjuvant (IFA), CpG adjuvant, or phosphate-buffered saline (PBS). Panels (A), (B), (C), (D), and (E) represent the fold-change in tumor volume at days 7, 9, 11, 14 and 16, respectively, following injection with B16F10 cancer cells on day 0. Each bar on the graphs represents results for a group of immunized mice.

FIG. 19 shows the correlation between tumor volume and IFNγ spot forming units in peripheral blood cells, a measure of immunogenicity and T cell activation, for mice immunized with a pool of four inhibitory antigens in combination with triple adjuvant B (CpG, 3D-PHAD, QS21). Square symbols represent IFNγ spot forming units per 200K cells. Circles represent tumor volume (mm³) on day 17 (panel A) and day 22 (panel B), following injection with B16F10 cancer cells on day 0. Each symbol on the graphs represents results for an individual mouse. Lines connect results for an individual mouse. Black indicates correlation between low IFNγ (low immune response) and hyper-progressing tumor. Gray indicates correlation between higher IFNγ (higher immune response) and slower progressing tumor. White indicates no correlation.

### DEFINITIONS

Activate: As used herein, a peptide presented by an antigen presenting cell (APC) “activates” a lymphocyte if lymphocyte activity is detectably modulated after exposure to the peptide presented by the APC under conditions that permit antigen-specific recognition to occur. Any indicator of lymphocyte activity can be evaluated to determine whether a lymphocyte is activated, e.g., T cell proliferation, phosphorylation or dephosphorylation of a receptor, calcium flux, cytoskeletal rearrangement, increased or decreased expression and/or secretion of immune mediators such as cytokines or soluble mediators, increased or decreased expression of one or more cell surface markers.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be systemic or local. In some embodiments, administration may be enteral or parenteral. In some embodiments, administration may be by injection (e.g., intramuscular, intravenous, or subcutaneous injection). In some embodiments, injection may involve bolus injection, drip, perfusion, or infusion. In some embodiments administration may be topical. Those skilled in the art will be aware of appropriate administration routes for use with particular therapies described herein, for example from among those listed on www.fda.gov, which include auricular (otic), buccal, conjunctival, cutaneous, dental, endocervical, endosinusial, endotracheal, enteral, epidural, extra-amniotic, extracorporeal, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracorporus cavernosum, intradermal, intranodal, intradiscal, intraductal, intraduodenal, intradural, intraepidermal, intraesophageal, intragastic, intragingival, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravitreal, laryngeal, nasal, nasogastric, ophthalmic, oral, oropharyngeal, parenteral, percutaneous, periarticular, peridural, perineural, periodontal, rectal, respiratory (e.g., inhalation), retrobulbar, soft tissue, subarachnoid, subconjunctival, subcutaneous, sublingual, submucosal, topical, transdermal, transmucosal, transplacental, transtracheal, ureteral, urethral, or vaginal. In some embodiments, administration may involve electro-osmosis, hemodialysis, infiltration, iontophoresis, irrigation, and/or occlusive dressing. In some embodiments, administration may involve dosing that is intermittent (e.g., a plurality of doses separated in time) and/or periodic (e.g., individual doses separated by a common period of time) dosing. In some embodiments, administration may involve continuous dosing.

Antigen: The term “antigen”, as used herein, refers to a molecule (e.g., a polypeptide) that elicits a specific immune response. Antigen-specific immunological responses, also known as adaptive immune responses, are mediated by lymphocytes (e.g., T cells, B cells, NK cells) that express antigen receptors (e.g., T cell receptors, B cell receptors). In certain embodiments, an antigen is a T cell antigen, and elicits a cellular immune response. In certain embodiments, an antigen is a B cell antigen, and elicits a humoral (i.e., antibody) response. In certain embodiments, an antigen is both a T cell antigen and a B cell antigen. As used herein, the term “antigen” encompasses both a full-length polypeptide as well as a portion or immunogenic fragment of the polypeptide, and a peptide epitope within the polypeptides (e.g., a peptide epitope bound by a Major Histocompatibility Complex (MHC) molecule (e.g., MHC class I, or MH-RC class II)).

Antigen presenting cell: An “antigen presenting cell” or “APC” refers to a cell that presents peptides on MHC class I and/or MH-RC class II molecules for recognition by T cells. APC include both professional APC (e.g., dendritic cells, macrophages, B cells), which have the ability to stimulate naïve lymphocytes, and non-professional APC (e.g., fibroblasts, epithelial cells, endothelial cells, glial cells). In certain embodiments, APC are able to internalize (e.g., endocytose) members of a library (e.g., cells of a library of bacterial cells) that express heterologous polypeptides as candidate antigens.

Autolysin polypeptide: An “autolysin polypeptide” is a polypeptide that facilitates or mediates autolysis of a cell (e.g., a bacterial cell) that has been internalized by a eukaryotic cell. In some embodiments, an autolysin polypeptide is a bacterial autolysin polypeptide. Autolysin polypeptides include, and are not limited to, polypeptides whose sequences are disclosed in GenBank® under Acc. Nos. NP_388823.1, NP_266427.1, and P0AGC3.1.

Cancer: As used herein, the term “cancer” refers to a disease, disorder, or condition in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer may be characterized by one or more tumors. Those skilled in the art are aware of a variety of types of cancer including, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, myeloid leukemia), lymphoma (e.g., Burkitt lymphoma [non-Hodgkin lymphoma], cutaneous T cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

Cytolysin polypeptide: A “cytolysin polypeptide” is a polypeptide that has the ability to form pores in a membrane of a eukaryotic cell. A cytolysin polypeptide, when expressed in host cell (e.g., a bacterial cell) that has been internalized by a eukaryotic cell, facilitates release of host cell components (e.g., host cell macromolecules, such as host cell polypeptides) into the cytosol of the internalizing cell. In some embodiments, a cytolysin polypeptide is bacterial cytolysin polypeptide. In some embodiments, a cytolysin polypeptide is a cytoplasmic cytolysin polypeptide. Cytolysin polypeptides include, and are not limited to, polypeptides whose sequences are disclosed in U.S. Pat. No. 6,004,815, and in GenBank® under Acc. Nos. NP_463733.1, NP 979614, NP 834769, YP_084586, YP 895748, YP_694620, YP_012823, NP 346351, YP_597752, BAB41212.2, NP 561079.1, YP_001198769, and NP_359331.1.

Cytoplasmic cytolysin polypeptide: A “cytoplasmic cytolysin polypeptide” is a cytolysin polypeptide that has the ability to form pores in a membrane of a eukaryotic cell, and that is expressed as a cytoplasmic polypeptide in a bacterial cell. A cytoplasmic cytolysin polypeptide is not significantly secreted by a bacterial cell. Cytoplasmic cytolysin polypeptides can be provided by a variety of means. In some embodiments, a cytoplasmic cytolysin polypeptide is provided as a nucleic acid encoding the cytoplasmic ccytolysin polypeptide. In some embodiments, a cytoplasmic cytolysin polypeptide is provided attached to a bead. In some embodiments, a cytoplasmic cytolysin polypeptide has a sequence that is altered relative to the sequence of a secreted cytolysin polypeptide (e.g., altered by deletion or alteration of a signal sequence to render it nonfunctional). In some embodiments, a cytoplasmic cytolysin polypeptide is cytoplasmic because it is expressed in a secretion-incompetent cell. In some embodiments, a cytoplasmic cytolysin polypeptide is cytoplasmic because it is expressed in a cell that does not recognize and mediate secretion of a signal sequence linked to the cytolysin polypeptide. In some embodiments, a cytoplasmic cytolysin polypeptide is a bacterial cytolysin polypeptide.

Heterologous: The term “heterologous”, as used herein to refer to genes or polypeptides, refers to a gene or polypeptide that does not naturally occur in the organism in which it is present and/or being expressed, and/or that has been introduced into the organism by the hand of man. In some embodiments, a heterologous polypeptide is a tumor antigen described herein.

Immune mediator: As used herein, the term “immune mediator” refers to any molecule that affects the cells and processes involved in immune responses. Immune mediators include cytokines, chemokines, soluble proteins, and cell surface markers.

Improve, increase, inhibit, stimulate, suppress, or reduce: As used herein, the terms “improve”, “increase”, “inhibit”, “stimulate”, “suppress”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. The effect of a particular agent or treatment may be direct or indirect. In some embodiments, an appropriate reference measurement may be or may comprise a measurement in a comparable system known or expected to respond in a particular way, in presence of the relevant agent or treatment. In some embodiments, a peptide presented by an antigen presenting cell (APC) “stimulates” or is “stimulatory” to a lymphocyte if the lymphocyte is activated to a phenotype associated with beneficial responses, after exposure to the peptide presented by the APC under conditions that permit antigen-specific recognition to occur, as observed by, e.g., T cell proliferation, phosphorylation or dephosphorylation of a receptor, calcium flux, cytoskeletal rearrangement, increased or decreased expression and/or secretion of immune mediators such as cytokines or soluble mediators, increased or decreased expression of one or more cell surface markers, relative to a control. In some embodiments, a peptide presented by an antigen presenting cell “suppresses”, “inhibits” or is “inhibitory” to a lymphocyte if the lymphocyte is activated to a phenotype associated with deleterious or non-beneficial responses, after exposure to the peptide presented by the APC under conditions that permit antigen-specific recognition to occur, as observed by, e.g., phosphorylation or dephosphorylation of a receptor, calcium flux, cytoskeletal rearrangement, increased or decreased expression and/or secretion of immune mediators such as cytokines or soluble mediators, increased or decreased expression of one or more cell surface markers, relative to a control.

Inhibitory Antigen: An “inhibitory antigen” is an antigen that inhibits, suppresses, impairs and/or reduces immune control of a tumor or cancer. In some embodiments, an inhibitory antigen promotes tumor growth, enables tumor growth, ameliorates tumor growth, activates tumor growth, accelerates tumor growth, and/or increases and/or enables tumor metastasis. In some embodiments, an inhibitory antigen stimulates one or more lymphocyte responses that are deleterious or non-beneficial to a subject; and/or inhibits and/or suppresses one or more lymphocyte responses that are beneficial to a subject. In some embodiments, an inhibitory antigen is the target of one or more lymphocyte responses that are deleterious or non-beneficial to a subject; and/or inhibits and/or suppresses one or more lymphocyte responses that are beneficial to a subject.

Invasin polypeptide: An “invasin polypeptide” is a polypeptide that facilitates or mediates uptake of a cell (e.g., a bacterial cell) by a eukaryotic cell. Expression of an invasin polypeptide in a noninvasive bacterial cell confers on the cell the ability to enter a eukaryotic cell. In some embodiments, an invasin polypeptide is a bacterial invasin polypeptide. In some embodiments, an invasin polypeptide is a Yersinia invasin polypeptide (e.g., a Yersinia invasin polypeptide comprising a sequence disclosed in GenBank® under Acc. No. YP_070195.1).

Listeriolysin O (LLO): The terms “listeriolysin O” or “LLO” refer to a listeriolysin O polypeptide of Listeria monocytogenes and truncated forms thereof that retain pore-forming ability (e.g., cytoplasmic forms of LLO, including truncated forms lacking a signal sequence). In some embodiments, an LLO is a cytoplasmic LLO. Exemplary LLO sequences are shown in Table 1, below.

Polypeptide: The term “polypeptide”, as used herein, generally has its art-recognized meaning of a polymer of at least three amino acids. Those of ordinary skill in the art will appreciate, however, that the term “polypeptide” is intended to be sufficiently general as to encompass not only polypeptides having the complete sequence recited herein (or in a reference or database specifically mentioned herein), but also to encompass polypeptides that represent functional fragments (i.e., fragments retaining at least one activity) and immunogenic fragments of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides.

Primary cells: As used herein, “primary cells” refers to cells from an organism that have not been immortalized in vitro. In some embodiments, primary cells are cells taken directly from a subject (e.g., a human). In some embodiments, primary cells are progeny of cells taken from a subject (e.g., cells that have been passaged in vitro). Primary cells include cells that have been stimulated to proliferate in culture.

Re-educate: As used herein, in the context of the response of a lymphocyte, “re-educate” refers to alteration in one or more responses of a lymphocyte to a particular antigen. In certain embodiments, an antigen initially stimulates one or more lymphocyte responses that are deleterious or non-beneficial to a subject, and/or the antigen initially inhibits and/or suppresses one or more lymphocyte responses that are beneficial to a subject, and such lymphocyte is re-educated such that the antigen no longer stimulates one or more lymphocyte responses that are deleterious or non-beneficial to a subject, and/or the antigen no longer inhibits and/or suppresses one or more lymphocyte responses that are beneficial to a subject. In some such embodiments, such lymphocyte is re-educated such that the antigen stimulates one or more lymphocyte responses that are beneficial to a subject and/or the antigen inhibits and/or suppresses one or more lymphocyte response that are deleterious or non-beneficial to a subject.

Redirect: As used herein, in the context of an immune response, “redirect” refers to an alteration in one or more aspects of an immune response. In certain embodiments, an initial immune response (e.g., an initial immune response to an antigen) impairs or reduces immune control of a tumor or cancer, and such initial immune response is redirected such that the immune response (e.g., to the antigen) no longer impairs or reduces immune control of a tumor or cancer. In some such embodiments, such redirected immune response enhances immune control of a tumor.

Response: As used herein, in the context of a subject (a patient or experimental organism), “response”, “responsive”, or “responsiveness” refers to an alteration in a subject's condition that occurs as a result of, or correlates with, treatment. In certain embodiments, a response is a beneficial response. In certain embodiments, a beneficial response can include stabilization of a subject's condition (e.g., prevention or delay of deterioration expected or typically observed to occur absent the treatment), amelioration (e.g., reduction in frequency and/or intensity) of one or more symptoms of the condition, and/or improvement in the prospects for cure of the condition, etc. In certain embodiments, for a subject who has cancer, a beneficial response can include: the subject has a positive clinical response to cancer therapy or a combination of therapies; the subject has a spontaneous response to a cancer; the subject is in partial or complete remission from cancer; the subject has cleared a cancer; the subject has not had a relapse, recurrence or metastasis of a cancer; the subject has a positive cancer prognosis; the subject has not experienced toxic responses or side effects to a cancer therapy or combination of therapies. In certain embodiments, for a subject who had cancer, the beneficial responses occurred in the past, or are ongoing.

In certain embodiments, a response is a deleterious or non-beneficial response. In certain embodiments, a deleterious or non-beneficial response can include deterioration of a subject's condition, lack of amelioration (e.g., no reduction in frequency and/or intensity) of one or more symptoms of the condition, and/or degradation in the prospects for cure of the condition, etc. In certain embodiments, for a subject who has cancer, a deleterious or non-beneficial response can include: the subject has a negative clinical response to cancer therapy or a combination of therapies; the subject is not in remission from cancer; the subject has not cleared a cancer; the subject has had a relapse, recurrence or metastasis of a cancer; the subject has a negative cancer prognosis; the subject has experienced toxic responses or side effects to a cancer therapy or combination of therapies. In certain embodiments, for a subject who had cancer, the deleterious or non-beneficial responses occurred in the past, or are ongoing.

As used herein, in the context of a cell, organ, tissue, or cell component, e.g., a lymphocyte, “response”, “responsive”, or “responsiveness” refers to an alteration in cellular activity that occurs as a result of, or correlates with, administration of or exposure to an agent, e.g. a tumor antigen. In certain embodiments, a beneficial response can include increased expression and/or secretion of immune mediators associated with positive clinical responses or outcomes in a subject. In certain embodiments, a beneficial response can include decreased expression and/or secretion of immune mediators associated with negative clinical response or outcomes in a subject. In certain embodiments, a deleterious or non-beneficial response can include increased expression and/or secretion of immune mediators associated with negative clinical responses or outcomes in a subject. In certain embodiments, a deleterious or non-beneficial response can include decreased expression and/or secretion of immune mediators associated with positive clinical responses or outcomes in a subject. In certain embodiments, a response is a clinical response. In certain embodiments, a response is a cellular response. In certain embodiments, a response is a direct response. In certain embodiments, a response is an indirect response. In certain embodiments, “non-response”, “non-responsive”, or “non-responsiveness” mean minimal response or no detectable response. In certain embodiments, a “minimal response” includes no detectable response. In certain embodiments, presence, extent, and/or nature of response can be measured and/or characterized according to particular criteria. In certain embodiments, such criteria can include clinical criteria and/or objective criteria. In certain embodiments, techniques for assessing response can include, but are not limited to, clinical examination, positron emission tomography, chest X-ray, CT scan, MRI, ultrasound, endoscopy, laparoscopy, presence or level of a particular marker in a sample, cytology, and/or histology. Where a response of interest is a response of a tumor to a therapy, ones skilled in the art will be aware of a variety of established techniques for assessing such response, including, for example, for determining tumor burden, tumor size, tumor stage, etc. Methods and guidelines for assessing response to treatment are discussed in Therasse et al., J. Natl. Cancer Inst., 2000, 92(3):205-216; and Seymour et al., Lancet Oncol., 2017, 18:e143-52. The exact response criteria can be selected in any appropriate manner, provided that when comparing groups of tumors, patients or experimental organism, and/or cells, organs, tissues, or cell components, the groups to be compared are assessed based on the same or comparable criteria for determining response rate. One of ordinary skill in the art will be able to select appropriate criteria.

Stimulatory Antigen: A “stimulatory antigen” is an antigen that enhances, improves, increases and/or stimulates immune control of a tumor or cancer. In some embodiments, a stimulatory antigen is the target of an immune response that reduces, kills, shrinks, resorbs, and/or eradicates tumor growth; does not promote, enable, ameliorate, activate, and/or accelerate tumor growth; decreases tumor metastasis, and/or decelerates tumor growth. In some embodiments, a stimulatory antigen inhibits and/or suppresses one or more lymphocyte responses that are deleterious or non-beneficial to a subject; and/or stimulates one or more lymphocyte responses that are beneficial to a subject.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

### DETAILED DESCRIPTION

Recent advances in immune checkpoint inhibitor therapies such as ipilimumab, nivolumab, and pembrolizumab for cancer immunotherapy have resulted in dramatic efficacy in subjects suffering from NSCLC, among other indications. Nivolumab and pembroluzimab have been approved by the Food and Drug Administration (FDA) and European Medicines Agency (EMA) for use in patients with advanced NSCLC who have previously been treated with chemotherapy. They have solidified the importance of T cell responses in control of tumors. Neoantigens, potential cancer rejection antigens that are entirely absent from the normal human genome, are postulated to be relevant to tumor control; however, attempts to define them and their role in tumor clearance has been hindered by the paucity of available tools to define them in a biologically relevant and unbiased way (Schumacher and Schreiber, 2015 Science 348:69-74, Gilchuk et al., 2015 Curr Opin Immunol 34:43-51).

Taking non-small cell lung carcinoma (NSCLC) as an example, whole exome sequencing of NSCLC tumors from patients treated with pembrolizumab showed that higher non-synonymous mutation burden in tumors was associated with improved objective response, durable clinical benefit, and progression-free survival (Rizvi et al., (2015) Science 348(6230): 124-8). In this study, the median non-synonymous mutational burden of the discovery cohort was 209 and of the validation cohort was 200. However, simply because a mutation was identified by sequencing, does not mean that the epitope it creates can be recognized by a T cell or serves as a protective antigen for T cell responses (Gilchuk et al., 2015 Curr Opin Immunol 34:43-51), making the use of the word neoantigen somewhat of a misnomer. With 200 or more potential targets of T cells in NSCLC, it is not feasible to test every predicted epitope to determine which of the mutations serve as neoantigens, and which neoantigens are associated with clinical evidence of tumor control. Recently, a study by McGranahan et al., showed that clonal neoantigen burden and overall survival in primary lung adenocarcinomas are related. However, even enriching for clonal neoantigens results in potential antigen targets ranging from 50 to approximately 400 (McGranahan et al., 2016 Science 351:1463-69). Similar findings have been described for melanoma patients who have responded to ipilimumab therapy (Snyder et al., 2015 NEJM; Van Allen et al., 2015 Science) and in patients with mismatch-repair deficient colorectal cancer who were treated with pembrolizumab (Le et al., 2015 NEJM).

The present disclosure provides methods and systems for the rapid identification of tumor antigens (e.g., tumor specific antigens (TSAs, or neoantigens), tumor associated antigens (TAAs), or cancer/testis antigens (CTAs)) that elicit T cell responses and particularly that elicit human T cell responses, as well as polypeptides that are potential tumor antigens. For purposes of this disclosure, “tumor antigens” includes both tumor antigens and potential tumor antigens. As described herein, methods of the present disclosure identified stimulatory tumor antigens that were not identified by known algorithms. Further, methods of the present disclosure identified suppressive and/or inhibitory tumor antigens that are not identifiable by known algorithms. Methods of the present disclosure also identified polypeptides that are potential tumor antigens, i.e., polypeptides that activate T cells of non-cancerous subjects, but not T cells of subjects suffering from cancer. The present disclosure also provides methods of selecting tumor antigens and potential tumor antigens, methods of using the selected tumor antigens and potential tumor antigens, immunogenic compositions comprising the selected tumor antigens and potential tumor antigens, and methods of manufacturing immunogenic compositions.

In addition, the present disclosure provides methods of re-educating lymphocytes to alter one or more responses of lymphocytes to a particular antigen (e.g., an inhibitory antigen); methods of redirecting one or more immune responses (e.g., to an antigen, e.g., an inhibitory antigen); and methods of treating subjects (e.g., subjects having a tumor or cancer) by re-educating lymphocytes to alter one or more immune responses of lymphocytes to a particular antigen (e.g., an inhibitory antigen) and/or redirecting one or more immune responses (e.g., to an antigen, e.g., an inhibitory antigen).

### Library Generation

A library is a collection of members (e.g., cells or non-cellular particles, such as virus particles, liposomes, or beads (e.g., beads coated with polypeptides, such as in vitro translated polypeptides, e.g., affinity beads, e.g., antibody coated beads, or NTA-Ni beads bound to polypeptides of interest). According to the present disclosure, members of a library include (e.g., internally express or carry) polypeptides of interest described herein. In some embodiments, members of a library are cells that internally express polypeptides of interest described herein. In some embodiments, members of a library which are particles carry, and/or are bound to, polypeptides of interest. Use of a library in an assay system allows simultaneous evaluation in vitro of cellular responses to multiple candidate antigens. According to the present disclosure, a library is designed to be internalized by human antigen presenting cells so that peptides from library members, including peptides from internally expressed polypeptides of interest, are presented on MHC molecules of the antigen presenting cells for recognition by T cells.

Libraries can be used in assays that detect peptides presented by human MHC class I and MHC class II molecules. Polypeptides expressed by the internalized library members are digested in intracellular endocytic compartments (e.g., phagosomes, endosomes, lysosomes) of the human cells and presented on MHC class II molecules, which are recognized by human CD4⁺ T cells. In some embodiments, library members include a cytolysin polypeptide, in addition to a polypeptide of interest. In some embodiments, library members include an invasin polypeptide, in addition to the polypeptide of interest. In some embodiments, library members include an autolysin polypeptide, in addition to the polypeptide of interest. In some embodiments, library members are provided with cells that express a cytolysin polypeptide (i.e., the cytolysin and polypeptide of interest are not expressed in the same cell, and an antigen presenting cell is exposed to members that include the cytolysin and members that include the polypeptide of interest, such that the antigen presenting cell internalizes both, and such that the cytolysin facilitates delivery of polypeptides of interest to the MHC class I pathway of the antigen presenting cell). A cytolysin polypeptide can be constitutively expressed in a cell, or it can be under the control of an inducible expression system (e.g., an inducible promoter). In some embodiments, a cytolysin is expressed under the control of an inducible promoter to minimize cytotoxicity to the cell that expresses the cytolysin.

Once internalized by a human cell, a cytolysin polypeptide perforates intracellular compartments in the human cell, allowing polypeptides expressed by the library members to gain access to the cytosol of the human cell. Polypeptides released into the cytosol are presented on MHC class I molecules, which are recognized by CD8⁺ T cells.

A library can include any type of cell or particle that can be internalized by and deliver a polypeptide of interest (and a cytolysin polypeptide, in applications where a cytolysin polypeptide is desirable) to, antigen presenting cells for use in methods described herein. Although the term “cell” is used throughout the present specification to refer to a library member, it is understood that, in some embodiments, the library member is a non-cellular particle, such as a virus particle, liposome, or bead. In some embodiments, members of the library include polynucleotides that encode the polypeptide of interest (and cytolysin polypeptide), and can be induced to express the polypeptide of interest (and cytolysin polypeptide) prior to, and/or during internalization by antigen presenting cells.

In some embodiments, the cytolysin polypeptide is heterologous to the library cell in which it is expressed, and facilitates delivery of polypeptides expressed by the library cell into the cytosol of a human cell that has internalized the library cell. Cytolysin polypeptides include bacterial cytolysin polypeptides, such as listeriolysin O (LLO), streptolysin O (SLO), and perfringolysin O (PFO). Additional cytolysin polypeptides are described in U.S. Pat. No. 6,004,815. In certain embodiments, library members express LLO. In some embodiments, a cytolysin polypeptide is not significantly secreted by the library cell (e.g., less than 20%, 10%, 5%, or 1% of the cytolysin polypeptide produced by the cell is secreted). For example, the cytolysin polypeptide is a cytoplasmic cytolysin polypeptide, such as a cytoplasmic LLO polypeptide (e.g., a form of LLO which lacks the N-terminal signal sequence, as described in Higgins et al., Mol. Microbiol. 31(6):1631-1641, 1999). Exemplary cytolysin polypeptide sequences are shown in Table 1. The listeriolysin O (Δ3-25) sequence shown in the second row of Table 1 has a deletion of residues 3-25, relative to the LLO sequence in shown in the first row of Table 1, and is a cytoplasmic LLO polypeptide. In some embodiments, a cytolysin is expressed constitutively in a library host cell. In other embodiments, a cytolysin is expressed under the control of an inducible promoter. Cytolysin polypeptides can be expressed from the same vector, or from a different vector, as the polypeptide of interest in a library cell.

TABLE 1
Exemplary Cytolysin Polypeptides

| | Polypeptide | |
|---|---|---|
| Polypeptide Name | Accession No. | |
| (species) | GI No. | Polypeptide Sequence |
| listeriolysin O | NP_463733.1 | MKKIMLVFITLILVSLPIAQQTEAKDASAFNKENSISSMAPPASP |
| (Listeria | GI: 16802248 | PASPKTPIEKKHADEIDKYIQGLDYNKNNVLVYHGDAVINVPPRK |
| monocytogenes) | | GYKDGNEYIVVEKKKKSINQNNADIQVVNAISSLTYPGALVKANS |
| | | ELVENQPDVLPVKRDSLILSIDLPGMTNQDNKIVVKNATKSNVNN |
| | | AVNTLVERWNEKYAQAYPNVSAKIDYDDEMAYSESQLIAKFGTAF |
| | | KAVNNSLNVNFGAISEGKMQEEVISFKQIYYNVNVNEPTRPSRFF |
| | | GKAVIKEQLQALGVNAENPPAYISSVAYGRQVYLKLSINSHSTKV |
| | | KAAFDAAVSGKSVSGDVELTNIIKNSSFKAVIYGGSAKDEVQIID |
| | | GNLGDLRDILKKGATFNRETPGVPIAYTTNFLKDNELAVIKNNSE |
| | | YIETTSKAYTDGKINIDHSGGYVAQFNISWDEVNYDPEGNEIVQH |
| | | KNWSENNKSKLAHFISSIYLPGNARNINVYAKECTGLAWEWWRIV |
| | | IDDRNLPLVKNRNISIWGTTLYPKYSNKVDNPIE (SEQ ID |
| | | NO: 1) |
| listeriolysin O | | MKDASAFNKENSISSMAPPASPPASPKTPIEKKHADEIDKYIQGL |
| (Δ3-25) | | DYNKNNVLVYHGDAVTNVPPRKGYKDGNEYIVVEKKKKSINQNNA |
| | | DIQVVNAISSLTYPGALVKANSELVENQPDVLPVKRDSLILSIDL |
| | | PGMTNQDNKIVVKNATKSNVNNAVNTLVERWNEKYAQAYPNVSAK |
| | | IDYDDEMAYSESQLIAKEGTAFKAVNNSLNVNFGAISEGKMQEEV |
| | | ISFKQIYYNVNVNEPTRPSRFFGKAVIKEQLQALGVNAENPPAYI |
| | | SSVAYGRQVYLKLSTNSHSTKVKAAFDAAVSGKSVSGDVELTNII |
| | | KNSSFKAVIYGGSAKDEVQIIDGNLGDLRDILKKGATFNRETPGV |
| | | PIAYTINFLKDNELAVIKNNSEYIETTSKAYIDGKINIDHSGGYV |
| | | AQFNISWDEVNYDPEGNEIVQHKNWSENNKSKLAHFTSSIYLPGN |
| | | ARNINVYAKECTGLAWEWWRIVIDDRNLPLVKNRNISIWGITLYP |
| | | KYSNKVDNPIE(SEQ ID NO:2) |
| streptolysin O | BAB41212.2 | MSNKKTFKKYSRVAGLLTAALIIGNLVTANAESNKQNTASTETTT |
| (Streptococcus | GI: 71061060 | TSEQPKPESSELTIEKAGQKMDDMLNSNDMIKLAPKEMPLESAEK |
| pyogenes) | | EEKKSEDKKKSEEDHTEEINDKIYSLNYNELEVLAKNGETIENFV |
| | | PKEGVKKADKFIVIERKKKNINTTPVDISIIDSVTDRTYPAALQL |
| | | ANKGFTENKPDAVVIKRNPQKIHIDLPGMGDKATVEVNDPIYANV |
| | | STAIDNLVNQWHDNYSGGNILPARTQYTESMVYSKSQIEAALNVN |
| | | SKILDGILGIDFKSISKGEKKVMIAAYKQIFYIVSANLPNNPADV |
| | | FDKSVTFKDLQRKGVSNEAPPLFVSNVAYGRTVFVKLETSSKSND |
| | | VEAAFSAALKGTDVKTNGKYSDILENSSFTAVVLGGDAAEHNKVV |
| | | TKDFDVIRNVIKDNATFSRKNPAYPISYTSVFLKNNKIAGVNNRT |
| | | EYVETTSTEYTSGKINLSHQGAYVAQYEILWDEINYDDKGKEVIT |
| | | KRRWDNNWYSKTSPFSTVIPLGANSRNIRIMARECTGLAWEWWRK |
| | | VIDERDVKLSKEINVNISGSTLSPYGSITYK (SEQ ID NO:3) |
| perfringolysin O | NP_561079.1 | MIRFKKTKLIASIAMALCLFSQPVISFSKDITDKNQSIDSGISSL |
| (Clostridium | GI: 18309145 | SYNRNEVLASNGDKIESFVPKEGKKTGNKFIVVERQKRSLTTSPV |
| perfringens) | | DISIIDSVNDRTYPGALQLADKAFVENRPTILMVKRKPININIDL |
| | | PGLKGENSIKVDDPTYGKVSGAIDELVSKWNEKYSSTHTLPARTQ |
| | | YSESMVYSKSQISSALNVNAKVLENSLGVDFNAVANNEKKVMILA |
| | | YKQIFYTVSADLPKNPSDLFDDSVTFNDLKQKGVSNEAPPLMVSN |
| | | VAYGRTIYVKLETTSSSKDVQAAFKALIKNTDIKNSQQYKDIYEN |
| | | SSFTAVVLGGDAQEHNKVVTKDFDEIRKVIKDNATFSTKNPAYPI |
| | | SYTSVFLKDNSVAAVHNKTDYIETTSTEYSKGKINLDHSGAYVAQ |
| | | FEVAWDEVSYDKEGNEVLTHKTWDGNYQDKTAHYSTVIPLEANAR |
| | | NIRIKARECTGLAWEWWRDVISEYDVPLTNNINVSIWGTTLYPGS |
| | | SITYN (SEQ ID NO:4) |
| Pneumolysin | NP_359331.1 | MANKAVNDFILAMNYDKKKLLTHQGESIENRFIKEGNQLPDEFVV |
| (Streptococcus | GI: 933687 | IERKKRSLSTNTSDISVTATNDSRLYPGALLVVDETLLENNPTLL |
| pneumoniae) | | AVDRAPMTYSIDLPGLASSDSFLQVEDPSNSSVRGAVNDLLAKWH |
| | | QDYGQVNNVPARMQYEKITAHSMEQLKVKFGSDFEKTGNSLDIDF |
| | | NSVHSGEKQIQIVNFKQIYYTVSVDAVKNPGDVFQDTVTVEDLKQ |
| | | RGISAERPLVYISSVAYGRQVYLKLETTSKSDEVEAAFEALIKGV |
| | | KVAPQTEWKQILDNTEVKAVILGGDPSSGARVVTGKVDMVEDLIQ |
| | | EGSRFTADHPGLPISYTTSFLRDNVVATFQNSTDYVETKVTAYRN |
| | | GDLLLDHSGAYVAQYYITWDELSYDHQGKEVLTPKAWDRNGQDLT |
| | | AHFTTSIPLKGNVRNLSVKIRECTGLAWEWWRTVYEKTDLPLVRK |
| | | RTISIWGTTLYPQVEDKVEND (SEQ ID NO:5) |

In some embodiments, a library member (e.g., a library member which is a bacterial cell) includes an invasin that facilitates uptake by the antigen presenting cell. In some embodiments, a library member includes an autolysin that facilitates autolysis of the library member within the antigen presenting cell. In some embodiments, a library member includes both an invasin and an autolysin. In some embodiments, a library member which is an E. coli cell includes an invasin and/or an autolysin. In various embodiments, library cells that express an invasin and/or autolysin are used in methods that also employ non-professional antigen presenting cells or antigen presenting cells that are from cell lines. Isberg et al. (Cell, 1987, 50:769-778), Sizemore et al. (Science, 1995, 270:299-302) and Courvalin et al. (C.R. Acad. Sci. Paris, 1995, 318:1207-12) describe expression of an invasin to effect endocytosis of bacteria by target cells. Autolysins are described by Cao et al., Infect. Immun. 1998, 66(6): 2984-2986; Margot et al., J. Bacteriol. 1998, 180(3):749-752; Buist et al., Appl. Environ. Microbiol., 1997, 63(7):2722-2728; Yamanaka et al., FEMS Microbiol. Lett., 1997, 150(2): 269-275; Romero et al., FEMS Microbiol. Lett., 1993, 108(1):87-92; Betzner and Keck, Mol. Gen. Genet., 1989, 219(3): 489-491; Lubitz et al., J. Bacteriol., 1984, 159(1):385-387; and Tomasz et al., J. Bacteriol., 1988, 170(12): 5931-5934. In some embodiments, an autolysin has a feature that permits delayed lysis, e.g., the autolysin is temperature-sensitive or time-sensitive (see, e.g., Chang et al., 1995, J. Bact. 177, 3283-3294; Raab et al., 1985, J. Mol. Biol. 19, 95-105; Gerds et al., 1995, Mol. Microbiol. 17, 205-210). Useful cytolysins also include addiction (poison/antidote) autolysins, (see, e.g., Magnuson R, et al., 1996, J. Biol. Chem. 271(31), 18705-18710; Smith A S, et al., 1997, Mol. Microbiol. 26(5), 961-970).

In some embodiments, members of the library include bacterial cells. In certain embodiments, the library includes non-pathogenic, non-virulent bacterial cells. Examples of bacteria for use as library members include E. coli, mycobacteria, Listeria monocytogenes, Shigella flexneri, Bacillus subtilis, or Salmonella.

In some embodiments, members of the library include eukaryotic cells (e.g., yeast cells). In some embodiments, members of the library include viruses (e.g., bacteriophages). In some embodiments, members of the library include liposomes. Methods for preparing liposomes that include a cytolysin and other agents are described in Kyung-Dall et al., U.S. Pat. No. 5,643,599. In some embodiments, members of the library include beads. Methods for preparing libraries comprised of beads are described, e.g., in Lam et al., Nature 354: 82-84, 1991, U.S. Pat. Nos. 5,510,240 and 7,262,269, and references cited therein.

In certain embodiments, a library is constructed by cloning polynucleotides encoding polypeptides of interest, or portions thereof, into vectors that express the polypeptides of interest in cells of the library. The polynucleotides can be synthetically synthesized. The polynucleotides can be cloned by designing primers that amplify the polynucleotides. Primers can be designed using available software, such as Primer3Plus (available the following URL: bioinformatics.nl/cgi-bin/primer3plus/primer3plus.cgi; see Rozen and Skaletsky, In: Krawetz S, Misener S (eds) Bioinformatics Methods and Protocols: Methods in Molecular Biology. Humana Press, Totowa, N.J., pp. 365-386, 2000). Other methods for designing primers are known to those of skill in the art. In some embodiments, primers are constructed so as to produce polypeptides that are truncated, and/or lack hydrophobic regions (e.g., signal sequences or transmembrane regions) to promote efficient expression. The location of predicted signal sequences and predicted signal sequence cleavage sites in a given open reading frame (ORF) sequence can be determined using available software, see, e.g., Dyrlov et al., J. Mol. Biol., 340:783-795, 2004, and the following URL: cbs.dtu.dk/services/SignalP/). For example, if a signal sequence is predicted to occur at the N-terminal 20 amino acids of a given polypeptide sequence, a primer is designed to anneal to a coding sequence downstream of the nucleotides encoding the N-terminal 20 amino acids, such that the amplified sequence encodes a product lacking this signal sequence.

Primers can also be designed to include sequences that facilitate subsequent cloning steps. ORFs can be amplified directly from genomic DNA (e.g., genomic DNA of a tumor cell), or from polynucleotides produced by reverse transcription (RT-PCR) of mRNAs expressed by the tumor cell. RT-PCR of mRNA is useful, e.g., when the genomic sequence of interest contains intronic regions. PCR-amplified ORFs are cloned into an appropriate vector, and size, sequence, and expression of ORFs can be verified prior to use in immunological assays.

In some embodiments, a polynucleotide encoding a polypeptide of interest is linked to a sequence encoding a tag (e.g., an N-terminal or C-terminal epitope tag) or a reporter protein (e.g., a fluorescent protein). Epitope tags and reporter proteins facilitate purification of expressed polypeptides, and can allow one to verify that a given polypeptide is properly expressed in a library host cell, e.g., prior to using the cell in a screen. Useful epitope tags include, for example, a polyhistidine (His) tag, a V5 epitope tag from the P and V protein of paramyxovirus, a hemagglutinin (HA) tag, a myc tag, and others. In some embodiments, a polynucleotide encoding a polypeptide of interest is fused to a sequence encoding a tag which is a known antigenic epitope (e.g., an MHC class I- and/or MHC class II-restricted T cell epitope of a model antigen such as an ovalbumin), and which can be used to verify that a polypeptide of interest is expressed and that the polypeptide-tag fusion protein is processed and presented in antigen presentation assays. In some embodiments a tag includes a T cell epitope of a murine T cell (e.g., a murine T cell line). In some embodiments, a polynucleotide encoding a polypeptide of interest is linked to a tag that facilitates purification and a tag that is a known antigenic epitope. Useful reporter proteins include naturally occurring fluorescent proteins and their derivatives, for example, Green Fluorescent Protein (Aequorea Victoria) and Neon Green (Branchiostoma lanceolatum). Panels of synthetically derived fluorescent and chromogenic proteins are also available from commercial sources.

Polynucleotides encoding a polypeptide of interest are cloned into an expression vector for introduction into library host cells. Various vector systems are available to facilitate cloning and manipulation of polynucleotides, such as the Gateway® Cloning system (Invitrogen). As is known to those of skill in the art, expression vectors include elements that drive production of polypeptides of interest encoded by a polynucleotide in library host cells (e.g., promoter and other regulatory elements). In some embodiments, polypeptide expression is controlled by an inducible element (e.g., an inducible promoter, e.g., an IPTG- or arabinose-inducible promoter, or an IPTG-inducible phage T7 RNA polymerase system, a lactose (lac) promoter, a tryptophan (trp) promoter, a tac promoter, a trc promoter, a phage lambda promoter, an alkaline phosphatase (phoA) promoter, to give just a few examples; see Cantrell, Meth. in Mol. Biol., 235:257-276, Humana Press, Casali and Preston, Eds.). In some embodiments, polypeptides are expressed as cytoplasmic polypeptides. In some embodiments, the vector used for polypeptide expression is a vector that has a high copy number in a library host cell. In some embodiments, the vector used for expression has a copy number that is more than 25, 50, 75, 100, 150, 200, or 250 copies per cell. In some embodiments, the vector used for expression has a ColE1 origin of replication. Useful vectors for polypeptide expression in bacteria include pET vectors (Novagen), Gateway® pDEST vectors (Invitrogen), pGEX vectors (Amersham Biosciences), pPRO vectors (BD Biosciences), pBAD vectors (Invitrogen), pLEX vectors (Invitrogen), pMAL™ vectors (New England BioLabs), pGEMEX vectors (Promega), and pQE vectors (Qiagen). Vector systems for producing phage libraries are known and include Novagen T7Select® vectors, and New England Biolabs Ph.D.™ Peptide Display Cloning System.

In some embodiments, library host cells express (either constitutively, or when induced, depending on the selected expression system) a polypeptide of interest to at least 10%, 20%, 30%, 40%, 50%, 60%, or 70% of the total cellular protein. In some embodiments, the level a polypeptide available in or on a library member (e.g., cell, virus particle, liposome, bead) is such that antigen presenting cells exposed to a sufficient quantity of the library members are presented on MHC molecules polypeptide epitopes at a density that is comparable to the density presented by antigen presenting cells pulsed with purified peptides.

Methods for efficient, large-scale production of libraries are available. For example, site-specific recombinases or rare-cutting restriction enzymes can be used to transfer polynucleotides between expression vectors in the proper orientation and reading frame (Walhout et al., Meth. Enzymol. 328:575-592, 2000; Marsischky et al., Genome Res. 14:2020-202, 2004; Blommel et al., Protein Expr. Purif 47:562-570, 2006).

For production of liposome libraries, expressed polypeptides (e.g., purified or partially purified polypeptides) can be entrapped in liposomal membranes, e.g., as described in Wassef et al., U.S. Pat. No. 4,863,874; Wheatley et al., U.S. Pat. No. 4,921,757; Huang et al., U.S. Pat. No. 4,925,661; or Martin et al., U.S. Pat. No. 5,225,212.

A library can be designed to include full length polypeptides and/or portions of polypeptides. Expression of full length polypeptides maximizes epitopes available for presentation by a human antigen presenting cell, thereby increasing the likelihood of identifying an antigen. However, in some embodiments, it is useful to express portions of polypeptides, or polypeptides that are otherwise altered, to achieve efficient expression. For example, in some embodiments, polynucleotides encoding polypeptides that are large (e.g., greater than 1,000 amino acids), that have extended hydrophobic regions, signal peptides, transmembrane domains, or domains that cause cellular toxicity, are modified (e.g., by C-terminal truncation, N-terminal truncation, or internal deletion) to reduce cytotoxicity and permit efficient expression a library cell, which in turn facilitates presentation of the encoded polypeptides on human cells. Other types of modifications, such as point mutations or codon optimization, may also be used to enhance expression.

The number of polypeptides included in a library can be varied. For example, in some embodiments, a library can be designed to express polypeptides from at least 5%, 10%, 15%, 20%, 25%, 35%, 40%, 45%, 50%, 55%, 60%, 70%, 75%, 80%, 85%, 90%, 95%, 97%, 98%, 99%, or more, of ORFs in a target cell (e.g., tumor cell). In some embodiments, a library expresses at least 10, 15, 20, 25, 30, 40, 50, 75, 100, 150, 200, 250, 300, 350, 400, 450, 500, 550, 600, 650, 700, 750, 800, 850, 900, 950, 1000, 2500, 5000, 10,000, or more different polypeptides of interest, each of which may represent a polypeptide encoded by a single full length polynucleotide or portion thereof.

In some embodiments, assays may focus on identifying antigens that are secreted polypeptides, cell surface-expressed polypeptides, or virulence determinants, e.g., to identify antigens that are likely to be targets of both humoral and cell mediated immune responses.

In addition to polypeptides of interest, libraries can include tags or reporter proteins that allow one to easily purify, analyze, or evaluate MHC presentation, of the polypeptide of interest. In some embodiments, polypeptides expressed by a library include C-terminal tags that include both an MHC class I and an MHC class II-restricted T cell epitope from a model antigen, such as chicken ovalbumin (OVA). Library protein expression and MHC presentation is validated using these epitopes. In some embodiments, the epitopes are OVA₂₄₇₋₂₆₅ and OVA₂₅₈₋₂₆₅ respectfully, corresponding to positions in the amino acid sequence found in GenBank® under Acc. No. NP_990483. Expression and presentation of linked ORFs can be verified with antigen presentation assays using T cell hybridomas (e.g., B3Z T hybridoma cells, which are H2-K^{b} restricted, and KZO T hybridoma cells, which are H2-A^{k} restricted) that specifically recognize these epitopes.

Sets of library members (e.g., bacterial cells) can be provided on an array (e.g., on a solid support, such as a 96-well plate) and separated such that members in each location express a different polypeptide of interest, or a different set of polypeptides of interest.

Methods of using library members for identifying T cell antigens are described in detail below. In addition to these methods, library members also have utility in assays to identify B cell antigens. For example, lysate prepared from library members that include polypeptides of interest can be used to screen a sample comprising antibodies (e.g., a serum sample) from a subject (e.g., a subject who has been exposed to an infectious agent of interest, a subject who has cancer, and/or a control subject), to determine whether antibodies present in the subject react with the polypeptide of interest. Suitable methods for evaluating antibody reactivity are known and include, e.g., ELISA assays.

### Polypeptides of Interest

In some embodiments, methods and compositions described herein can be used to identify and/or detect immune responses to a polypeptide of interest. In some embodiments, a polypeptide of interest is encoded by an ORF from a target tumor cell, and members of a library include (e.g., internally express or carry) ORFs from a target tumor cell. In some such embodiments, a library can be used in methods described herein to assess immune responses to one or more polypeptides of interest encoded by one or more ORFs. In some embodiments, methods of the disclosure identify one or more polypeptides of interest as stimulatory antigens (e.g., that stimulate an immune response, e.g., a T cell response, e.g., expression and/or secretion of one or more immune mediators). In some embodiments, methods of the disclosure identify one or more polypeptides of interest as antigens or potential antigens that have minimal or no effect on an immune response (e.g., expression and/or secretion of one or more immune mediators). In some embodiments, methods of the disclosure identify one or more polypeptides of interest as inhibitory and/or suppressive antigens (e.g., that inhibit, suppress, down-regulate, impair, and/or prevent an immune response, e.g., a T cell response, e.g., expression and/or secretion of one or more immune mediators). In some embodiments, methods of the disclosure identify one or more polypeptides of interest as tumor antigens or potential tumor antigens, e.g., tumor specific antigens (TSAs, or neoantigens), tumor associated antigens (TAAs), or cancer/testis antigens (CTAs).

In some embodiments, a polypeptide of interest is a putative tumor antigen, and methods and compositions described herein can be used to identify and/or detect immune responses to one or more putative tumor antigens. For example, members of a library include (e.g., internally express or carry) putative tumor antigens (e.g., a polypeptide previously identified (e.g., by a third party) as a tumor antigen, e.g., identified as a tumor antigen using a method other than a method of the present disclosure). In some embodiments, a putative tumor antigen is a tumor antigen described herein. In some such embodiments, such libraries can be used to assess whether and/or the extent to which such putative tumor antigen mediates an immune response. In some embodiments, methods of the disclosure identify one or more putative tumor antigens as stimulatory antigens. In some embodiments, methods of the disclosure identify one or more putative tumor antigens as antigens that have minimal or no effect on an immune response. In some embodiments, methods of the disclosure identify one or more putative tumor antigens as inhibitory and/or suppressive antigens.

In some embodiments, a polypeptide of interest is a pre-selected tumor antigen, and methods and compositions described herein can be used to identify and/or detect immune responses to one or more pre-selected tumor antigens. For example, in some embodiments, members of a library include (e.g., internally express or carry) one or more polypeptides identified as tumor antigens using a method of the present disclosure and/or using a method other than a method of the present disclosure. In some such embodiments, such libraries can be used to assess whether and/or the extent to which such tumor antigens mediate an immune response by an immune cell from one or more subjects (e.g., a subject who has cancer and/or a control subject) to obtain one or more response profiles described herein. In some embodiments, methods of the disclosure identify one or more pre-selected tumor antigens as stimulatory antigens for one or more subjects. In some embodiments, methods of the disclosure identify one or more pre-selected tumor antigens as antigens that have minimal or no effect on an immune response for one or more subjects. In some embodiments, methods of the disclosure identify one or more pre-selected tumor antigens as inhibitory and/or suppressive antigens for one or more subjects.

In some embodiments, a polypeptide of interest is a known tumor antigen, and methods and compositions described herein can be used to identify and/or detect immune responses to one or more known tumor antigens. For example, in some embodiments, members of a library include (e.g., internally express or carry) one or more polypeptides identified as a tumor antigen using a method of the present disclosure and/or using a method other than a method of the present disclosure. In some such embodiments, such libraries can be used to assess whether and/or the extent to which such tumor antigens mediate an immune response by an immune cell from one or more subjects (e.g., a subject who has cancer and/or a control subject) to obtain one or more response profiles described herein. In some embodiments, methods of the disclosure identify one or more known tumor antigens as stimulatory antigens for one or more subjects. In some embodiments, methods of the disclosure identify one or more known tumor antigens as antigens that have minimal or no effect on an immune response for one or more subjects. In some embodiments, methods of the disclosure identify one or more known tumor antigens as inhibitory and/or suppressive antigens for one or more subjects.

In some embodiments, a polypeptide of interest is a potential tumor antigen, and methods and compositions described herein can be used to identify and/or detect immune responses to one or more potential tumor antigens. For example, in some embodiments, members of a library include (e.g., internally express or carry) one or more polypeptides identified as being of interest, e.g., encoding mutations associated with a tumor, using a method of the present disclosure and/or using a method other than a method of the present disclosure. In some such embodiments, such libraries can be used to assess whether and/or the extent to which such polypeptides mediate an immune response by an immune cell from one or more subjects (e.g., a subject who has cancer and/or a control subject) to obtain one or more response profiles described herein. In some embodiments, methods of the disclosure identify one or more polypeptides as stimulatory antigens for one or more subjects. In some embodiments, methods of the disclosure identify one or more polypeptides as antigens that have minimal or no effect on an immune response for one or more subjects. In some embodiments, methods of the disclosure identify one or more polypeptides as inhibitory and/or suppressive antigens for one or more subjects.

### Tumor Antigens

Polypeptides of interest used in methods and systems described herein include tumor antigens amd potential tumor antigens, e.g., tumor specific antigens (TSAs, or neoantigens), tumor associated antigens (TAAs), and/or cancer/testis antigens (CTAs). Exemplary tumor antigens include, e.g., MART-1/MelanA (MART-I or MLANA), gp100 (Pmel 17 or SILV), tyrosinase, TRP-1, TRP-2, MAGE-1, MAGE-3 (also known as HIP8), BAGE, GAGE-1, GAGE-2, p15, Calcitonin, Calretinin, Carcinoembryonic antigen (CEA), Chromogranin, Cytokeratin, Desmin, Epithelial membrane protein (EMA), Factor VIII, Glial fibrillary acidic protein (GFAP), Gross cystic disease fluid protein (GCDFP-15), HMB-45, Human chorionic gonadotropin (hCG), inhibin, lymphocyte marker, MART-1 (Melan-A), Myo Di, muscle-specific actin (MSA), neurofilament, neuron-specific enolase (NSE), placental alkaline phosphatase (PLAP), prostate-specific antigen, PTPRC (CD45), S100 protein, smooth muscle actin (SMA), synaptophysin, thyroglobulin, thyroid transcription factor-1, Tumor M2-PK, vimentin, p53, Ras, HER-2/neu, BCR-ABL, E2A-PRL, H4-RET, IGH-IGK, MYL-RAR, Epstein Barr virus antigens (e.g., EBNA1), human papillomavirus (HPV) antigen E6 or E7 (HPV_E6 or HPV_E7), TSP-180, MAGE-4, MAGE-5, MAGE-6, RAGE, NY-ESO-1 (also known as CTAGIB), erbB, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein (AFP), beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, MUC16, IL13Rα2, FRα, VEGFR2, Lewis Y, FAP, EphA2, CEACAMS, EGFR, CA6, CA9, GPNMB, EGP1, FOLR1, endothelial receptor, STEAPI, SLC44A4, Nectin-4, AGS-16, guanalyl cyclase C, MUC-1, CFC1B, integrin alpha 3 chain (of a3b1, a laminin receptor chain), TPS, CD19, CD20, CD22, CD30, CD31, CD72, CD180, CD171 (LlCAM), CD123, CD133, CD138, CD37, CD70, CD79a, CD79b, CD56, CD74, CD166, CD71, CD34, CD99, CD117, CD80, CD28, CD13, CD15, CD25, CD10, CLL-1/CLEC12A, ROR1, Glypican 3 (GPC3), Mesothelin, CD33/IL3Ra, c-Met, PSCA, PSMA, Glycolipid F77, EGFRvIII, BCMA, GD-2, PSAP, prostein (also known as P501S), PSMA, Survivin (also known as BIRC5), and MAGE-A3, MAGEA2, MAGEA4, MAGEA6, MAGEA9, MAGEA10, MAGEA12, BIRC5, CDH3, CEACAM3, CGB_isoform2, ELK4, ERBB2, HPSE1, HPSE2, KRAS_isoform1, KRAS_isoform2, MUC1, SMAD4, TER CGB_isoform1, IMPDH2, LCK, angiopoietin-1 (Ang1) (also known as ANGPT1), XIAP (also known as BIRC4), galectin-3 (also known as LGALS3), VEGF-A (also known as VEGF), ATP6S1 (also known as ATP6AP1), MAGE-A1, cIAP-1 (also known as BIRC2), macrophage migration inhibitory factor (MIF), galectin-9 (also known as LGALS9), progranulin PGRN (also known as granulin), OGFR, MLIAP (also known as BIRC7), TBX4 (also known as ICPPS, SPS or T-Box4), secretory leukocyte protein inhibitor (Slpi) (also known as antileukoproteinase), Ang2 (also known as ANGPT2), galectin-1 (also known as LGALS1), TRP-2 (also known as DCT), hTERT (telomerase reverse transcriptase) tyrosinase-related protein 1 (TRP-1, TYRP1), NOR-90/UBF-2 (also known as UBTF), LGMN, SPA17, PRTN3, TRRAP_1, TRRAP_2, TRRAP 3, TRRAP 4, MAGEC2, PRAME, SOX10, RAC1, HRAS, GAGE4, AR, CYP1B1, MMP8, TYR, PDGFRB, KLK3, PAX3, PAX5, ST3GAL5, PLAC1, RhoC, MYCN, REG3A, CSAG2, CTAG2-1a, CTAG2-1b, PAGE4, BRAF, GRM3, ERBB4, KIT, MAPK1, MFI2, SART3, ST8SIA1, WDR46, AKAP-4, RGS5, FOSL1, PRM2, ACRBP, CTCFL, CSPG4, CCNB1, MSLN, WT1, SSX2, KDR, ANKRD30A, MAGED1, MAP3K9, XAGE1B, PREX2, CD276, TEK, AIM1, ALK, FOLH1, GRIN2A MAP3K5 and one or more isoforms of any preceding tumor antigens. Exemplary tumor antigens are provided in the accompanying list of sequences. In some embodiments, a tumor antigen comprises a variant of an amino acid sequence provided in the accompanying list of sequences (e.g., a sequence that is at least about 85%, 90%, 95%, 96%, 97% 98%, 99% identical to an amino acid sequence provided in the accompanying list of sequences and/or a sequence that includes a mutation, deletion, and/or insertion of at least one amino acid (e.g., 1, 2, 3, 4, 5, 6, 7, 8, 9, 10, or more amino acids) relative to an amino acid sequence provided in the accompanying list of sequences).

Tumor specific antigens (TSAs, or neoantigens) are tumor antigens that are not encoded in normal host genome (see, e.g., Yarchoan et al., Nat. Rev. Cancer. 2017 Feb. 24. doi: 10.1038/nrc.2016.154; Gubin et al., J. Clin. Invest. 125:3413-3421 (2015)). In some embodiments, TSAs arise from somatic mutations and/or other genetic alterations. In some embodiments, TSAs arise from missense or in-frame mutations. In some embodiments, TSAs arise from frame-shift mutations or loss-of-stop-codon mutations. In some embodiments, TSAs arise from insertion or deletion mutations. In some embodiments, TSAs arise from duplication or repeat expansion mutations. In some embodiments, TSAs arise from splice variants or improper splicing. In some embodiments, TSAs arise from gene fusions. In some embodiments, TSAs arise from translocations. In some embodiments, TSAs include oncogenic viral proteins. For example, as with Merkel cell carcinoma (MCC) associated with the Merkel cell polyomavirus (MCPyV) and cancers of the cervix, oropharynx and other sites associated with the human papillomavirus (HPV), TSAs include proteins encoded by viral open reading frames. For purposes of this disclosure, the terms “mutation” and “mutations” encompass all mutations and genetic alterations that may give rise to an antigen encoded in the genome of a cancer or tumor cell of a subject, but not in a normal or non-cancerous cell of the same subject. In some embodiments, TSAs are specific (personal) to a subject. In some embodiments, TSAs are shared by more than one subject, e.g., less than 1%, 1-3%, 1-5%, 1-10%, or more of subjects suffering from a cancer. In some embodiments, TSAs shared by more than one subject may be known or pre-selected.

In some embodiments, a TSA is encoded by an open reading frame from a virus. For example, a library can be designed to express polypeptides from one of the following viruses: an immunodeficiency virus (e.g., a human immunodeficiency virus (HIV), e.g., HIV-1, HIV-2), a hepatitis virus (e.g., hepatitis B virus (HBV), hepatitis C virus (HCV), hepatitis A virus, non-A and non-B hepatitis virus), a herpes virus (e.g., herpes simplex virus type I (HSV-1), HSV-2, Varicella-zoster virus, Epstein Barr virus, human cytomegalovirus, human herpesvirus 6 (HHV-6), HHV-7, HHV-8), a poxvirus (e.g., variola, vaccinia, monkeypox, Molluscum contagiosum virus), an influenza virus, a human papilloma virus, adenovirus, rhinovirus, coronavirus, respiratory syncytial virus, rabies virus, coxsackie virus, human T cell leukemia virus (types I, II and III), parainfluenza virus, paramyxovirus, poliovirus, rotavirus, rhinovirus, rubella virus, measles virus, mumps virus, adenovirus, yellow fever virus, Norwalk virus, West Nile virus, a Dengue virus, Severe Acute Respiratory Syndrome Coronavirus (SARS-CoV), bunyavirus, Ebola virus, Marburg virus, Eastern equine encephalitis virus, Venezuelan equine encephalitis virus, Japanese encephalitis virus, St. Louis encephalitis virus, Junin virus, Lassa virus, and Lymphocytic choriomeningitis virus. Libraries for other viruses can also be produced and used according to methods described herein.

Tumor specific antigens are known in the art, any of which can be used in methods described herein. In some embodiments, gene sequences encoding polypeptides that are potential or putative neoantigens are determined by sequencing the genome and/or exome of tumor tissue and healthy tissue from a subject having cancer using next generation sequencing technologies. In some embodiments, genes that are selected based on their frequency of mutation and ability to encode a potential or putative neoantigen are sequenced using next-generation sequencing technology. Next-generation sequencing applies to genome sequencing, genome resequencing, transcriptome profiling (RNA-Seq), DNA-protein interactions (ChIP-sequencing), and epigenome characterization (de Magalhaes et al. (2010) Ageing Research Reviews 9 (3): 315-323; Hall N (2007) J. Exp. Biol. 209 (Pt 9): 1518-1525; Church (2006) Sci. Am. 294 (1): 46-54; ten Bosch et al. (2008) Journal of Molecular Diagnostics 10 (6): 484-492; Tucker T et al. (2009) The American Journal of Human Genetics 85 (2): 142-154). Next-generation sequencing can be used to rapidly reveal the presence of discrete mutations such as coding mutations in individual tumors, e.g., single amino acid changes (e.g., missense mutations, in-frame mutations) or novel stretches of amino acids generated by frame-shift insertions, deletions, gene fusions, read-through mutations in stop codons, duplication or repeat expansion mutations, and translation of splice variants or improperly spliced introns, and translocations (e.g., “neoORFs”).

Another method for identifying potential or putative neoantigens is direct protein sequencing. Protein sequencing of enzymatic digests using multidimensional MS techniques (MSn) including tandem mass spectrometry (MS/MS)) can also be used to identify neoantigens. Such proteomic approaches can be used for rapid, highly automated analysis (see, e.g., Gevaert et al., Electrophoresis 21:1145-1154 (2000)). High-throughput methods for de novo sequencing of unknown proteins can also be used to analyze the proteome of a subject's tumor to identify expressed potential or putative neoantigens. For example, meta shotgun protein sequencing may be used to identify expressed potential or putative neoantigens (see e.g., Guthals et al. (2012) Molecular and Cellular Proteomics 11(10):1084-96).

Potential or putative neoantigens may also be identified using MHC multimers to identify neoantigen-specific T cell responses. For example, high-throughput analysis of neoantigen-specific T cell responses in patient samples may be performed using MHC tetramer-based screening techniques (see e.g., Hombrink et al. (2011) PLoS One; 6(8): e22523; Hadrup et al. (2009) Nature Methods, 6(7):520-26; van Rooij et al. (2013) Journal of Clinical Oncology, 31:1-4; and Heemskerk et al. (2013) EMBO Journal, 32(2):194-203).

In some embodiments, one or more known or pre-selected tumor specific antigens, or one or more potential or putative tumor specific antigens identified using one of these methods, can be included in a library described herein.

Tumor associated antigens (TAAs) include proteins encoded in a normal genome (see, e.g., Ward et al., Adv. Immunol. 130:25-74 (2016)). In some embodiments, TAAs are either normal differentiation antigens or aberrantly expressed normal proteins. Overexpressed normal proteins that possess growth/survival-promoting functions, such as Wilms tumor 1 (WT1) (Ohminami et al., Blood 95:286-293 (2000)) or Her2/neu (Kawashima et al., Cancer Res. 59:431-435 (1999)), are TAAs that directly participate in the oncogenic process. Post-translational modifications, such as phosphorylation, of proteins may also lead to formation of TAAs (Doyle, J. Biol. Chem. 281:32676-32683 (2006); Cobbold, Sci. Transl. Med. 5:203ra125 (2013)). TAAs are generally shared by more than one subject, e.g., less than 1%, 1-3%, 1-5%, 1-10%, 1-20%, or more of subjects suffering from a cancer. In some embodiments, TAAs are known or pre-selected tumor antigens. In some embodiments, with respect to an individual subject, TAAs are potential or putative tumor antigens.Cancer/testis antigens (CTAs) are expressed by various tumor types and by reproductive tissues (for example, testes, fetal ovaries and trophoblasts) but have limited or no detectable expression in other normal tissues in the adult and are generally not presented on normal reproductive cells, because these tissues do not express MHC class I molecules (see, e.g., Coulie et al., Nat. Rev. Cancer 14:135-146 (2014); Simpson et al., Nat. Rev. Cancer 5:615-625 (2005); Scanlan et al., Immunol. Rev. 188:22-32 (2002)). Library Screens

### Human Cells for Antigen Presentation

The present disclosure provides, inter alia, compositions and methods for identifying tumor antigens recognized by human immune cells. Human antigen presenting cells express ligands for antigen receptors and other immune activation molecules on human lymphocytes. Given differences in MHC peptide binding specificities and antigen processing enzymes between species, antigens processed and presented by human cells are more likely to be physiologically relevant human antigens in vivo than antigens identified in non-human systems. Accordingly, methods of identifying these antigens employ human cells to present candidate tumor antigen polypeptides. Any human cell that internalizes library members and presents polypeptides expressed by the library members on MHC molecules can be used as an antigen presenting cell according to the present disclosure. In some embodiments, human cells used for antigen presentation are primary human cells. The cells can include peripheral blood mononuclear cells (PBMC) of a human. In some embodiments, peripheral blood cells are separated into subsets (e.g., subsets comprising dendritic cells, macrophages, monocytes, B cells, or combinations thereof) prior to use in an antigen presentation assay. In some embodiments, a subset of cells that expresses MHC class II is selected from peripheral blood. In one example, a cell population including dendritic cells is isolated from peripheral blood. In some embodiments, a subset of dendritic cells is isolated (e.g., plasmacytoid, myeloid, or a subset thereof). Human dendritic cell markers include CD1c, CD1a, CD303, CD304, CD141, and CD209. Cells can be selected based on expression of one or more of these markers (e.g., cells that express CD303, CD1c, and CD141).

Dendritic cells can be isolated by positive selection from peripheral blood using commercially available kits (e.g., from Miltenyi Biotec Inc.). In some embodiments, the dendritic cells are expanded ex vivo prior to use in an assay. Dendritic cells can also be produced by culturing peripheral blood cells under conditions that promote differentiation of monocyte precursors into dendritic cells in vitro. These conditions typically include culturing the cells in the presence of cytokines such as GM-CSF and IL-4 (see, e.g., Inaba et al., Isolation of dendritic cells, Curr. Protoc. Immunol. May; Chapter 3: Unit 3.7, 2001). Procedures for in vitro expansion of hematopoietic stem and progenitor cells (e.g., taken from bone marrow or peripheral blood), and differentiation of these cells into dendritic cells in vitro, is described in U.S. Pat. No. 5,199,942, and U.S. Pat. Pub. 20030077263. Briefly, CD34⁺ hematopoietic stem and progenitor cells are isolated from peripheral blood or bone marrow and expanded in vitro in culture conditions that include one or more of Flt3-L, IL-1, IL-3, and c-kit ligand.

In some embodiments, immortalized cells that express human MHC molecules (e.g., human cells, or non-human cells that are engineered to express human MHC molecules) are used for antigen presentation. For example, assays can employ COS cells transfected with human MHC molecules or HeLa cells.

In some embodiments, both the antigen presenting cells and immune cells used in the method are derived from the same subject (e.g., autologous T cells and APC are used). In these embodiments, it can be advantageous to sequentially isolate subsets of cells from peripheral blood of the subject, to maximize the yield of cells available for assays. For example, one can first isolate CD4⁺ and CD8⁺ T cell subsets from the peripheral blood. Next, dendritic cells (DC) are isolated from the T cell-depleted cell population. The remaining T- and DC-depleted cells are used to supplement the DC in assays, or are used alone as antigen presenting cells. In some embodiments, DC are used with T- and DC-depleted cells in an assay, at a ratio of 1:2, 1:3, 1:4, or 1:5. In some embodiments, the antigen presenting cells and immune cells used in the method are derived from different subjects (e.g., heterologous T cells and APC are used).

Antigen presenting cells can be isolated from sources other than peripheral blood. For example, antigen presenting cells can be taken from a mucosal tissue (e.g., nose, mouth, bronchial tissue, tracheal tissue, the gastrointestinal tract, the genital tract (e.g., vaginal tissue), or associated lymphoid tissue), peritoneal cavity, lymph nodes, spleen, bone marrow, thymus, lung, liver, kidney, neuronal tissue, endocrine tissue, or other tissue, for use in screening assays. In some embodiments, cells are taken from a tissue that is the site of an active immune response (e.g., an ulcer, sore, or abscess). Cells may be isolated from tissue removed surgically, via lavage, or other means.

Antigen presenting cells useful in methods described herein are not limited to “professional” antigen presenting cells. In some embodiments, non-professional antigen presenting cells can be utilized effectively in the practice of methods of the present disclosure. Non-professional antigen presenting cells include fibroblasts, epithelial cells, endothelial cells, neuronal/glial cells, lymphoid or myeloid cells that are not professional antigen presenting cells (e.g., T cells, neutrophils), muscle cells, liver cells, and other types of cells.

Antigen presenting cells are cultured with library members that express a polypeptide of interest (and, if desired, a cytolysin polypeptide) under conditions in which the antigen presenting cells internalize, process and present polypeptides expressed by the library members on MHC molecules. In some embodiments, library members are killed or inactivated prior to culture with the antigen presenting cells. Cells or viruses can be inactivated by any appropriate agent (e.g., fixation with organic solvents, irradiation, freezing). In some embodiments, the library members are cells that express ORFs linked to a tag (e.g., a tag which comprises one or more known T cell epitopes) or reporter protein, expression of which has been verified prior to the culturing.

In some embodiments, antigen presenting cells are incubated with library members at 37° C. for between 30 minutes and 5 hours (e.g., for 45 min. to 1.5 hours). After the incubation, the antigen presenting cells can be washed to remove library members that have not been internalized. In certain embodiments, the antigen presenting cells are non-adherent, and washing requires centrifugation of the cells. The washed antigen presenting cells can be incubated at 37° C. for an additional period of time (e.g., 30 min. to 2 hours) prior to exposure to lymphocytes, to allow antigen processing. In some embodiments, it is desirable to fix and kill the antigen presenting cells prior to exposure to lymphocytes (e.g., by treating the cells with 1% paraformaldehyde).

The antigen presenting cell and library member numbers can be varied, so long as the library members provide quantities of polypeptides of interest sufficient for presentation on MHC molecules. In some embodiments, antigen presenting cells are provided in an array, and are contacted with sets of library cells, each set expressing a different polypeptide of interest. In certain embodiments, each location in the array includes 1×10³-1×10⁶ antigen presenting cells, and the cells are contacted with 1×10³-1×10⁸ library cells which are bacterial cells.

In any of the embodiments described herein, antigen presenting cells can be freshly isolated, maintained in culture, and/or thawed from frozen storage prior to incubation with library cells, or after incubation with library cells.

### Human Lymphocytes

In methods of the present disclosure, human lymphocytes are tested for antigen-specific reactivity to antigen presenting cells, e.g., antigen presenting cells that have been incubated with libraries expressing polypeptides of interest as described above. The methods of the present disclosure permit rapid identification of human antigens using pools of lymphocytes isolated from an individual, or progeny of the cells. The detection of antigen-specific responses does not rely on laborious procedures to isolate individual T cell clones. In some embodiments, the human lymphocytes are primary lymphocytes. In some embodiments, human lymphocytes are NKT cells, gamma-delta T cells, or NK cells. Just as antigen presenting cells may be separated into subsets prior to use in antigen presentation assays, a population of lymphocytes having a specific marker or other feature can be used. In some embodiments, a population of T lymphocytes is isolated. In some embodiments, a population of CD4⁺ T cells is isolated. In some embodiments, a population of CD8⁺ T cells is isolated. CD8⁺ T cells recognize peptide antigens presented in the context of MHC class I molecules. Thus, in some embodiments, the CD8⁺ T cells are used with antigen presenting cells that have been exposed to library host cells that co-express a cytolysin polypeptide, in addition to a polypeptide of interest. T cell subsets that express other cell surface markers may also be isolated, e.g., to provide cells having a particular phenotype. These include CLA (for skin-homing T cells), CD25, CD30, CD69, CD154 (for activated T cells), CD45RO (for memory T cells), CD294 (for Th2 cells), γ/δ TCR-expressing cells, CD3 and CD56 (for NK T cells). Other subsets can also be selected.

Lymphocytes can be isolated, and separated, by any means known in the art (e.g., using antibody-based methods such as those that employ magnetic bead separation, panning, or flow cytometry). Reagents to identify and isolate human lymphocytes and subsets thereof are well known and commercially available.

Lymphocytes for use in methods described herein can be isolated from peripheral blood mononuclear cells, or from other tissues in a human. In some embodiments, lymphocytes are taken from tumors, lymph nodes, a mucosal tissue (e.g., nose, mouth, bronchial tissue, tracheal tissue, the gastrointestinal tract, the genital tract (e.g., vaginal tissue), or associated lymphoid tissue), peritoneal cavity, spleen, thymus, lung, liver, kidney, neuronal tissue, endocrine tissue, peritoneal cavity, bone marrow, or other tissues. In some embodiments, cells are taken from a tissue that is the site of an active immune response (e.g., an ulcer, sore, or abscess). Cells may be isolated from tissue removed surgically, via lavage, or other means.

Lymphocytes taken from an individual can be maintained in culture or frozen until use in antigen presentation assays. In some embodiments, freshly isolated lymphocytes can be stimulated in vitro by antigen presenting cells exposed to library cells as described above. In some embodiments, these lymphocytes exhibit detectable stimulation without the need for prior non-antigen specific expansion. However, primary lymphocytes also elicit detectable antigen-specific responses when first stimulated non-specifically in vitro. Thus, in some embodiments, lymphocytes are stimulated to proliferate in vitro in a non-antigen specific manner, prior to use in an antigen presentation assay. Lymphocytes can also be stimulated in an antigen-specific manner prior to use in an antigen presentation assay. In some embodiments, cells are stimulated to proliferate by a library (e.g., prior to use in an antigen presentation assay that employs the library). Expanding cells in vitro provides greater numbers of cells for use in assays. Primary T cells can be stimulated to expand, e.g., by exposure to a polyclonal T cell mitogen, such as phytohemagglutinin or concanavalin, by treatment with antibodies that stimulate proliferation, or by treatment with particles coated with the antibodies. In some embodiments, T cells are expanded by treatment with anti-CD2, anti-CD3, and anti-CD28 antibodies. In some embodiments, T cells are expanded by treatment with interleukin-2 (IL-2). In some embodiments, lymphocytes are thawed from frozen storage and expanded (e.g., stimulated to proliferate, e.g., in a non-antigen specific manner or in an antigen-specific manner) prior to contacting with antigen presenting cells. In some embodiments, lymphocytes are thawed from frozen storage and are not expanded prior to contacting with antigen presenting cells. In some embodiments, lymphocytes are freshly isolated and expanded (e.g., stimulated to proliferate, e.g., in a non-antigen specific manner or in an antigen-specific manner) prior to contacting with antigen presenting cells.

### Antigen Presentation Assays

In antigen presentation assays, T cells are cultured with antigen presenting cells prepared according to the methods described above, under conditions that permit T cell recognition of peptides presented by MHC molecules on the antigen presenting cells. In some embodiments, T cells are incubated with antigen presenting cells at 37° C. for between 12-48 hours (e.g., for 24 hours). In some embodiments, T cells are incubated with antigen presenting cells at 37° C. for 3, 4, 5, 6, 7, or 8 days. Numbers of antigen presenting cells and T cells can be varied. In some embodiments, the ratio of T cells to antigen presenting cells in a given assay is 1:10, 1:5, 1:2, 1:1, 2:1, 5:1, 10:1, 20:1, 25:1, 30:1, 32:1, 35:1 or 40:1. In some embodiments, antigen presenting cells are provided in an array (e.g., in a 96-well plate), wherein cells in each location of the array have been contacted with sets of library cells, each set including a different polypeptide of interest. In certain embodiments, each location in the array includes 1×10³-1×10⁶ antigen presenting cells, and the cells are contacted with 1×10³-1×10⁶ T cells.

After T cells have been incubated with antigen presenting cells, cultures are assayed for activation. Lymphocyte activation can be detected by any means known in the art, e.g., T cell proliferation, phosphorylation or dephosphorylation of a receptor, calcium flux, cytoskeletal rearrangement, increased or decreased expression and/or secretion of immune mediators such as cytokines or soluble mediators, increased or decreased expression of one or more cell surface markers. In some embodiments, culture supernatants are harvested and assayed for increased and/or decreased expression and/or secretion of one or more polypeptides associated with activation, e.g., a cytokine, soluble mediator, cell surface marker, or other immune mediator. In some embodiments, the one or more cytokines are selected from TRAIL, IFN-gamma, IL-12p70, IL-2, TNF-alpha, MIP1-alpha, MIP1-beta, CXCL9, CXCL10, MCP1, RANTES, IL-1 beta, IL-4, IL-6, IL-8, IL-9, IL-10, IL-13, IL-15, CXCL11, IL-3, IL-5, IL-17, IL-18, IL-21, IL-22, IL-23A, IL-24, IL-27, IL-31, IL-32, TGF-beta, CSF, GM-CSF, TRANCE (also known as RANK L), MIP3-alpha, and fractalkine. In some embodiments, the one or more soluble mediators are selected from granzyme A, granzyme B, sFas, sFasL, perforin, and granulysin. In some embodiments, the one or more cell surface markers are selected from CD107a, CD107b, CD25, CD69, CD45RA, CD45RO, CD137 (4-1BB), CD44, CD62L, CD27, CCR7, CD154 (CD40L), KLRG-1, CD71, HLA-DR, CD122 (IL-2RB), CD28, IL7Ra (CD127), CD38, CD26, CD134 (OX-40), CTLA-4 (CD152), LAG-3, TIM-3 (CD366), CD39, PD1 (CD279), FoxP3, TIGIT, CD160, BTLA, 2B4 (CD244), and KLRG1. Cytokine secretion in culture supernatants can be detected, e.g., by ELISA, bead array, e.g., with a Luminex® analyzer. Cytokine production can also be assayed by RT-PCR of mRNA isolated from the T cells, or by ELISPOT analysis of cytokines released by the T cells. In some embodiments, proliferation of T cells in the cultures is determined (e.g., by detecting ³H thymidine incorporation). In some embodiments, target cell lysis is determined (e.g., by detecting T cell dependent lysis of antigen presenting cells labeled with Na₂⁵¹CrO₄). Target cell lysis assays are typically performed with CD8⁺ T cells. Protocols for these detection methods are known. See, e.g., Current Protocols In Immunology, John E. Coligan et al. (eds), Wiley and Sons, New York, N.Y., 2007. One of skill in the art understands that appropriate controls are used in these detection methods, e.g., to adjust for non-antigen specific background activation, to confirm the presenting capacity of antigen presenting cells, and to confirm the viability of lymphocytes.

In some embodiments, antigen presenting cells and lymphocytes used in the method are from the same individual. In some embodiments, antigen presenting cells and lymphocytes used in the method are from different individuals.

In some embodiments, antigen presentation assays are repeated using lymphocytes from the same individual that have undergone one or more previous rounds of exposure to antigen presenting cells, e.g., to enhance detection of responses, or to enhance weak initial responses. In some embodiments, antigen presentation assays are repeated using antigen presenting cells from the same individual that have undergone one or more previous rounds of exposure to a library, e.g., to enhance detection of responses, or to enhance weak initial responses. In some embodiments, antigen presentation assays are repeated using lymphocytes from the same individual that have undergone one or more previous rounds of exposure to antigen presenting cells, and antigen presenting cells from the same individual that have undergone one or more previous rounds of exposure to a library, e.g., to enhance detection of responses, or to enhance weak initial responses. In some embodiments, antigen presentation assays are repeated using antigen presenting cells and lymphocytes from different individuals, e.g., to identify antigens recognized by multiple individuals, or compare reactivities that differ between individuals.

### Methods of Identifying Tumor Antigens

One advantage of methods described herein is their ability to identify clinically relevant human antigens. Humans that have cancer may have lymphocytes that specifically recognize tumor antigens, which are the product of an adaptive immune response arising from prior exposure. In some embodiments, these cells are present at a higher frequency than cells from an individual who does not have cancer, and/or the cells are readily reactivated when re-exposed to the proper antigenic stimulus (e.g., the cells are “memory” cells). Thus, humans that have or have had cancer are particularly useful donors of cells for identifying antigens in vitro. The individual may be one who has recovered from cancer. In some embodiments, the individual has been recently diagnosed with cancer (e.g., the individual was diagnosed less than one year, three months, two months, one month, or two weeks, prior to isolation of lymphocytes and/or antigen presenting cells from the individual). In some embodiments, the individual was first diagnosed with cancer more than three months, six months, or one year prior to isolation of lymphocytes and/or antigen presenting cells.

In some embodiments, lymphocytes are screened against antigen presenting cells that have been contacted with a library of cells whose members express or carry polypeptides of interest, and the lymphocytes are from an individual who has not been diagnosed with cancer. In some embodiments, such lymphocytes are used to determine background (i.e., non-antigen-specific) reactivities. In some embodiments, such lymphocytes are used to identify antigens, reactivity to which exists in non-cancer individuals.

Cells from multiple donors (e.g., multiple subjects who have cancer) can be collected and assayed in methods described herein. In some embodiments, cells from multiple donors are assayed in order to determine if a given tumor antigen is reactive in a broad portion of the population, or to identify multiple tumor antigens that can be later combined to produce an immunogenic composition that will be effective in a broad portion of the population.

Antigen presentation assays are useful in the context of both infectious and non-infectious diseases. The methods described herein are applicable to any context in which a rapid evaluation of human cellular immunity is beneficial. In some embodiments, antigenic reactivity to polypeptides that are differentially expressed by neoplastic cells (e.g., tumor cells) is evaluated. Sets of nucleic acids differentially expressed by neoplastic cells have been identified using established techniques such as subtractive hybridization. Methods described herein can be used to identify antigens that were functional in a subject in which an anti-tumor immune response occurred. In other embodiments, methods are used to evaluate whether a subject has lymphocytes that react to a tumor antigen or set of tumor antigens.

In some embodiments, antigen presentation assays are used to examine reactivity to autoantigens in cells of an individual, e.g., an individual predisposed to, or suffering from, an autoimmune condition. Such methods can be used to provide diagnostic or prognostic indicators of the individual's disease state, or to identify autoantigens. For these assays, in some embodiments, libraries that include an array of human polypeptides are prepared. In some embodiments, libraries that include polypeptides from infectious agents which are suspected of eliciting cross-reactive responses to autoantigens are prepared. For examples of antigens from infectious agents thought to elicit cross-reactive autoimmune responses, see Barzilai et al., Curr Opin Rheumatol., 19(6):636-43, 2007; Ayada et al., Ann NY Acad Sci., 1108:594-602, 2007; Drouin et al., Mol Immunol., 45(1):180-9, 2008; and Bach, J Autoimmun., 25 Suppl:74-80, 2005.

As discussed, the present disclosure includes methods in which polypeptides of interest are included in a library (e.g., expressed in library cells or carried in or on particles or beads). After members of the library are internalized by antigen presenting cells, the polypeptides of interest are proteolytically processed within the antigen presenting cells, and peptide fragments of the polypeptides are presented on MHC molecules expressed in the antigen presenting cells. The identity of the polypeptide that stimulates a human lymphocyte in an assay described herein can be determined from examination of the set of library cells that were provided to the antigen presenting cells that produced the stimulation. In some embodiments, it is useful to map the epitope within the polypeptide that is bound by MHC molecules to produce the observed stimulation. This epitope, or the longer polypeptide from which it is derived (both of which are referred to as an “antigen” herein) can form the basis for an immunogenic composition, or for an antigenic stimulus in future antigen presentation assays.

Methods for identifying peptides bound by MHC molecules are known. In some embodiments, epitopes are identified by generating deletion mutants of the polypeptide of interest and testing these for the ability to stimulate lymphocytes. Deletions that lose the ability to stimulate lymphocytes, when processed and presented by antigen presenting cells, have lost the peptide epitope. In some embodiments, epitopes are identified by synthesizing peptides corresponding to portions of the polypeptide of interest and testing the peptides for the ability to stimulate lymphocytes (e.g., in antigen presentation assays in which antigen presenting cells are pulsed with the peptides). Other methods for identifying MHC bound peptides involve lysis of the antigen presenting cells that include the antigenic peptide, affinity purification of the MHC molecules from cell lysates, and subsequent elution and analysis of peptides from the MHC (Falk, K. et al. Nature 351:290, 1991, and U.S. Pat. No. 5,989,565).

In other embodiments, it is useful to identify the clonal T cell receptors that have been expanded in response to the antigen. Clonal T cell receptors are identified by DNA sequencing of the T cell receptor repertoire (Howie et al, 2015 Sci Trans Med 7:301). By identifying TCR specificity and function, TCRs can be transfected into other cell types and used in functional studies or for novel immunotherapies.

In other embodiments, it is useful to identify and isolate T cells responsive to a tumor antigen in a subject. The isolated T cells can be expanded ex vivo and administered to a subject for cancer therapy or prophylaxis.

### Methods of Identifying Immune Responses of a Subject

The disclosure provides methods of identifying one or more immune responses of a subject. In some embodiments, one or more immune responses of a subject are determined by a) providing a library described herein that includes a panel of tumor antigens (e.g., known tumor antigens, tumor antigens described herein, or tumor antigens, potential tumor antigens, and/or other polypeptides of interest identified using a method described herein); b) contacting the library with antigen presenting cells from the subject; c) contacting the antigen presenting cells with lymphocytes from the subject; and d) determining whether one or more lymphocytes are stimulated by, inhibited and/or suppressed by, activated by, or non-responsive to one or more tumor antigens presented by one or more antigen presenting cells. In some embodiments, the library includes about 1, 3, 5, 10, 15, 20, 25, 30, 40, 50, 60, 70, 80, 90, 100, or more tumor antigens.

In some embodiments, lymphocyte stimulation, non-stimulation, inhibition and/or suppression, activation, and/or non-responsiveness is determined by assessing levels of one or more expressed or secreted cytokines or other immune mediators described herein. In some embodiments, levels of one or more expressed or secreted cytokines that is at least 20%, 40%, 60%, 80%, 100%, 120%, 140%, 160%, 180%, 200% or more, higher than a control level indicates lymphocyte stimulation. In some embodiments, a level of one or more expressed or secreted cytokines that is at least 1, 2, 3, 4 or 5 standard deviations greater than the mean of a control level indicates lymphocyte stimulation. In some embodiments, a level of one or more expressed or secreted cytokines that is at least 1, 2, 3, 4 or 5 median absolute deviations (MADs) greater than a median response level to a control indicates lymphocyte stimulation. In some embodiments, a control is a negative control, for example, a clone expressing Neon Green (NG). In some embodiments, a level of one or more expressed or secreted cytokines that is at least 20%, 40%, 60%, 80%, 100%, 120%, 140%, 160%, 180%, 200% or more, lower than a control level indicates lymphocyte inhibition and/or suppression. In some embodiments, a level of one or more expressed or secreted cytokines that is at least 1, 2, 3, 4 or 5 standard deviations lower than the mean of a control level indicates lymphocyte inhibition and/or suppression. In some embodiments, a level of one or more expressed or secreted cytokines that is at least 1, 2, 3, 4 or 5 median absolute deviations (MADs) lower than a median response level to a control indicates lymphocyte inhibition and/or suppression. In some embodiments, a control is a negative control, for example, a clone expressing Neon Green (NG). In some embodiments, levels of one or more expressed or secreted cytokines that is at least 20%, 40%, 60%, 80%, 100%, 120%, 140%, 160%, 180%, 200% or more, higher or lower than a control level indicates lymphocyte activation. In some embodiments, a level of one or more expressed or secreted cytokines that is at least 1, 2, 3, 4 or 5 standard deviations greater or lower than the mean of a control level indicates lymphocyte activation. In some embodiments, a level of one or more expressed or secreted cytokines that is at least 1, 2, 3, 4 or 5 median absolute deviations (MADs) greater or lower than a median response level to a control indicates lymphocyte activation. In some embodiments, a control is a negative control, for example, a clone expressing Neon Green (NG). In some embodiments, a level of one or more expressed or secreted cytokines that is within about 20%, 15%, 10%, 5%, or less, of a control level indicates lymphocyte non-responsiveness or non-stimulation. In some embodiments, a level of one or more expressed or secreted cytokines that is less than 1 or 2 standard deviations higher or lower than the mean of a control level indicates lymphocyte non-responsiveness or non-stimulation. In some embodiments, a level of one or more expressed or secreted cytokines that is less than 1 or 2 median absolute deviations (MADs) higher or lower than a median response level to a control indicates lymphocyte non-responsiveness or non-stimulation. In some embodiments, a subject response profile can include a quantification, identification, and/or representation of a panel of different cytokines (e.g., 1, 2, 3, 4, 5, 6, 7, 8, 9, 10, 12, 14, 16, 18, 20, or more cytokines) and of the total number of tumor antigens (e.g., of all or a portion of different tumor antigens from the library) that stimulate, do not stimulate, inhibit and/or suppress, activate, or have no or minimal effect on production, expression or secretion of each member of the panel of cytokines.

### Methods of Selecting Tumor Antigens and Methods of Inducing or Inhibiting an Immune Response in a Subject

In general, immune responses can be usefully defined in terms of their integrated, functional end-effects. Dhabar et al. (2014) have proposed that immune responses can be categorized as being immunoprotective, immunopathological, and immunoregulatory/inhibitory. While these categories provide useful constructs with which to organize ideas, an overall in vivo immune response is likely to consist of several types of responses with varying amounts of dominance from each category. Immunoprotective or beneficial responses are defined as responses that promote efficient wound healing, eliminate infections and cancer, and mediate vaccine-induced immunological memory. These responses are associated with cytokines and mediators such as IFN-gamma, IL-12, IL-2, Granzyme B, CD107, etc. Immunopathological or deleterious responses are defined as those that are directed against self (autoimmune disease like multiple sclerosis, arthritis, lupus) or innocuous antigens (asthma, allergies) and responses involving chronic, non-resolving inflammation. These responses can also be associated with molecules that are implicated in immunoprotective responses, but also include immune mediators such as TNF-alpha, IL-10, IL-13, IL-17, IL-4, IgE, histamine, etc. Immunoregulatory responses are defined as those that involve immune cells and factors that regulate (mostly down-regulate) the function of other immune cells. Recent studies suggest that there is an arm of the immune system that functions to inhibit immune responses. For example, regulatory CD4⁺CD25⁺FoxP3⁺ T cells, IL-10, and TGF-beta, among others have been shown to have immunoregulatory/inhibitory functions. The physiological function of these factors is to keep pro-inflammatory, allergic, and autoimmune responses in check, but they may also suppress anti-tumor immunity and be indicative of negative prognosis for cancer. In the context of tumors, the expression of co-stimulatory molecules often decreases, and the expression of co-inhibitory ligands increases. MHC molecules are often down-regulated on tumor cells, favoring their escape. The tumor micro-environment, including stromal cells, tumor associated immune cells, and other cell types, produce many inhibitory factors, such as, IL-10, TGF-β, and IDO. Inhibitory immune cells, including T regs, Tr cells, immature DCs (iDCs), pDCs, and MDSC can be found in the tumor micro-environment. (Y Li UT GSBS Thesis 2016). Examples of mediators and their immune effects are shown in Table 2.

TABLE 2
Immune Mediators

| | | | Beneficial Outcomes | | | Deleterious Outcomes | | |
|---|---|---|---|---|---|---|---|---|
| Cytokine | Function | Secreted by | Cancer | ID | AI | Cancer | ID | AI |
| TRAIL | Induces apoptosis of | Most cells | X | X | ? | X | ? | ? |
| | tumor cells, induces | | | | | | | |
| | immune suppressor | | | | | | | |
| | cells | | | | | | | |
| IFN- | Critical for innate | T cells, | X | X | ? | X | ? | X |
| gamma | and adaptive immunity | NK cells, | | | | | | |
| | to pathogens, inhibits | NKT cells | | | | | | |
| | viral replication, | | | | | | | |
| | increases MHC Class | | | | | | | |
| | I expression | | | | | | | |
| IL-12 | Th1 differentiation; | DCs, | X | X | ? | X | ? | X |
| | stimulates T cell | macrophages, | | | | | | |
| | growth, induces | neutronphils | | | | | | |
| | IFN-gamma/TNF-alpha | | | | | | | |
| | secretion from T cells, | | | | | | | |
| | enhances CTLs | | | | | | | |
| IL-2 | T cell proliferation, | T cells, APCs | X | X | X | ? | ? | ? |
| | differentiation into | | | | | | | |
| | effector and memory | | | | | | | |
| | T cells and | | | | | | | |
| | regulatory T cells | | | | | | | |
| TNF- | Induces fevers, | Macrophages, | X | X | ? | X | ? | X |
| alpha | apoptosis, | APCs | | | | | | |
| | inflammation, | | | | | | | |
| | inhibits viral | | | | | | | |
| | replication | | | | | | | |
| MIP-1 | Chemotactic/pro- | Macrophages, | X | X | ? | ? | ? | X |
| alpha | inflammatory | DCs, T cells | | | | | | |
| | effects, activates | | | | | | | |
| | granulocytes, | | | | | | | |
| | induces secretion of | | | | | | | |
| | IL-1/IL6/TNF-alpha | | | | | | | |
| MIP-1 | Chemotactic/pro- | Macrophages, | X | X | ? | ? | ? | X |
| beta | inflammatory | DCs, T cells | | | | | | |
| | effects, activates | | | | | | | |
| | granulocytes, induces | | | | | | | |
| | secretion of | | | | | | | |
| | IL-1/IL6/TNF-alpha | | | | | | | |
| CXCL9 | T cell | APCs | X | X | ? | X | ? | X |
| | chemoattractant, | | | | | | | |
| | induced by IFN-gamma | | | | | | | |
| CXCL10 | Chemoattractant for | APCs | X | X | ? | ? | ? | X |
| | T cells, macrophages, | | | | | | | |
| | NK and DCs, promotes | | | | | | | |
| | T cell adhesion to | | | | | | | |
| | endothelial cells | | | | | | | |
| MCP-1 | Recruits monocytes, | most cells | X | X | ? | X | ? | X |
| | memory T cells and | | | | | | | |
| | DCS | | | | | | | |
| RANTES | Recruits T cells, | T cells | X | X | ? | ? | ? | X |
| | eosinophils, | | | | | | | |
| | basophils, induces | | | | | | | |
| | proliferation/ | | | | | | | |
| | activation of NK | | | | | | | |
| | cells, T cell | | | | | | | |
| | activation marker | | | | | | | |
| CXCL11 | Chemoattractant for | APCs | X | X | ? | ? | ? | X |
| | activated T cells | | | | | | | |
| IL-3 | Stimulates | T cells, APCs | X | X | ? | ? | ? | ? |
| | proliferation of | | | | | | | |
| | myeloid cells, | | | | | | | |
| | induces growth of | | | | | | | |
| | T cells | | | | | | | |
| IL-17 | Produced by Th17 | T cells | X | X | ? | X | ? | X |
| I | cells, induces | | | | | | | |
| | production of IL6, | | | | | | | |
| | GCSF, GMCSF, IL1b, | | | | | | | |
| | TGF-beta, TNF-alpha, | | | | | | | |
| | chemokines | | | | | | | |
| IL-18 | Pro-inflammatory, | Macrophages | X | X | ? | X | ? | X |
| | induces cell-mediated | | | | | | | |
| | immunity, production | | | | | | | |
| | of IFN-gamma | | | | | | | |
| IL-21 | Induces proliferation, | CD4 T cells | X | X | X | X | ? | ? |
| | upregulated in | | | | | | | |
| | Th2/Th17 TFh | | | | | | | |
| IL-22 | Cell-mediated | NK cells, | X | X | ? | X | ? | X |
| | immunity, pro- | T cells | | | | | | |
| | inflammatory | | | | | | | |
| IL-23 | Pro-inflammatory | APCs | X | X | ? | X | ? | X |
| IL-24 | Controls survival | Monocytes | X | X | ? | ? | ? | X |
| | and proliferation | macrophages, | | | | | | |
| | | Th2 cells | | | | | | |
| IL-27 | Induces differentiation | APCs, T cells | X | X | X | X | ? | X |
| | of T cells, upregulates | | | | | | | |
| | IL-10, can be pro-or | | | | | | | |
| | anti-inflammatory; | | | | | | | |
| | promotes Th1/Tr1, | | | | | | | |
| | inhibits Th2/Th17/ | | | | | | | |
| | regulatory T cells | | | | | | | |
| IL-32 | Pro-inflammatory, | T cells, | X | X | ? | X | ? | X |
| | increases secretion | NK cells | | | | | | |
| | of inflammatory | | | | | | | |
| | cytokines and | | | | | | | |
| | chemokines | | | | | | | |
| CSF | Induces myeloid cells | APCs | X | X | X | ? | ? | ? |
| | to proliferate and | | | | | | | |
| | differentiate | | | | | | | |
| GM-CSF | Promotes macrophage | T cells, | X | X | ? | ? | ? | X |
| | and Eosinophil | macrophages | | | | | | |
| | proliferation and | | | | | | | |
| | maturation, growth | | | | | | | |
| | factor | | | | | | | |
| TRANCE | Helps DC maturation/ | T cells | ? | X | ? | X | ? | ? |
| | survival, T cell | | | | | | | |
| | activation marker, | | | | | | | |
| | anti-apoptotic, | | | | | | | |
| | stimulates osteoclast | | | | | | | |
| | activity | | | | | | | |
| MIP-3 | Chemotactic for T | | X | X | ? | ? | ? | X |
| alpha | cells, DCs | | | | | | | |
| fractalkine | Chemotactic for T | Endothelial | X | X | ? | ? | ? | X |
| | cells and monocytes | cells | | | | | | |
| IL-4 | Stimulates B cells, | Th2 cells, | ? | X | ? | X | X | X |
| | Th2 proliferation, | basophils | | | | | | |
| | plasma cell | | | | | | | |
| | differentiation, IgE, | | | | | | | |
| | upregulates MHC | | | | | | | |
| | Class II expression, | | | | | | | |
| | decreases IFN- | | | | | | | |
| | gamma production | | | | | | | |
| IL-10 | Downregulates Th1 | Monocytes | X | ? | X | X | X | X |
| | cytokines/MHC Class | Th2 cells, | | | | | | |
| | II expression/Co- | regulatory | | | | | | |
| | stimulatory molecule | T cells | | | | | | |
| | expression | | | | | | | |
| IL-5 | Stimulates B cells, | Th2 cells, | ? | X | ? | X | X | X |
| | Ig secretion, eosinophil | mast cells | | | | | | |
| | activation | | | | | | | |
| IL-13 | Similar to IL4, induces | Th2 cells, | ? | X | ? | X | X | X |
| | IgE production, Th2 | NK cells, | | | | | | |
| | cytokine | mast cells, | | | | | | |
| | | eosinophils, | | | | | | |
| | | basophils | | | | | | |
| TGF-beta | Inhibits T cell | regulatory | ? | ? | X | X | X | ? |
| | proliferation, | T cells | | | | | | |
| | activity, function; | | | | | | | |
| | blocks effects of | | | | | | | |
| | pro-inflammatory | | | | | | | |
| | cytokines | | | | | | | |
| IL-1 beta | Induces fevers, pro- | Macrophages | X | X | ? | X | ? | X |
| | inflammatory | | | | | | | |
| IL-6 | Pro-inflammatory, | T cells, | ? | X | ? | X | X | X |
| | drives osteoclast | macrophages | | | | | | |
| | formation, drives | | | | | | | |
| | Th17 | | | | | | | |
| IL-8 | Recruits neutrophils | Macrophages, | ? | X | ? | X | ? | X |
| | to site of infection | epithelial | | | | | | |
| | | cells | | | | | | |
| IL-31 | Cell-mediated immunity, | Th2 cells, | X | X | ? | X | ? | X |
| | pro-inflammatory | macrophages, | | | | | | |
| | | DCs | | | | | | |
| IL-15 | T cell proliferation | T cells, | X | X | X | ? | ? | ? |
| | and survival | NK cells | | | | | | |
| IL-9 | Th2 proliferation, | T cells, | ? | ? | X | X | X | ? |
| | cytokine secretion | neutrophils, | | | | | | |
| | | mast cells | | | | | | |

ID = Infectious disease
IA = Autoimmune disease

The disclosure provides methods and systems for identifying and selecting (or deselecting) tumor antigens (e.g., stimulatory and/or inhibitory antigens). In some embodiments, a stimulatory antigen is a tumor antigen (e.g., a tumor antigen described herein) that stimulates one or more lymphocyte responses that are beneficial to the subject. In some embodiments, a stimulatory antigen is a tumor antigen (e.g., a tumor antigen described herein) that inhibits and/or suppresses one or more lymphocyte responses that are deleterious or non-beneficial to the subject. Examples of immune responses that may lead to beneficial anti-tumor responses (e.g., that may enhance immune control of a tumor) include but are not limited to 1) cytotoxic CD8⁺ T cells which can effectively kill cancer cells and release the mediators performn and/or granzymes to drive tumor cell death; and 2) CD4⁺ Th1 T cells which play an important role in host defense and can secrete IL-2, IFN-gamma and TNF-alpha. These are induced by IL-12, IL-2, and IFN gamma among other cytokines.

In some embodiments, an inhibitory antigen is a tumor antigen (e.g., a tumor antigen described herein) that stimulates one or more lymphocyte responses that are deleterious or non-beneficial to the subject. In some embodiments, an inhibitory antigen is a tumor antigen (e.g., a tumor antigen described herein) that inhibits and/or suppresses one or more lymphocyte responses that are beneficial to the subject. Examples of immune responses that may lead to deleterious or non-beneficial anti-tumor responses (e.g., that may impair or reduce control of a tumor) include but are not limited to 1) T regulatory cells which are a population of T cells that can suppress an immune response and secrete immunosuppressive cytokines such as TGF-beta and IL-10 and express the molecules CD25 and FoxP3; and 2) Th2 cells which target responses against allergens but are not productive against cancer. These are induced by increased IL-4 and IL-10 and can secrete IL-4, IL-5, IL-6, IL-9 and IL-13.

Additionally or alternatively, tumor antigens may be identified and/or selected (or de-selected) based on association with desirable or beneficial responses, e.g., clinical responses. Additionally or alternatively, tumor antigens may be identified and/or selected (or de-selected) based on association with undesirable, deleterious or non-beneficial responses, e.g., clinical responses. Tumor antigens may be identified and/or selected (or de-selected) based on a combination of the preceding methods, applied in any order.

Responses whereby tumor antigens or immunogenic fragments thereof (i) stimulate lymphocyte responses that are beneficial to the subject, (ii) stimulate expression of cytokines that are beneficial to the subject, (iii) inhibit and/or suppress lymphocyte responses that are deleterious or non-beneficial to the subject, or (iv) inhibit and/or suppress expression of cytokines that are deleterious or non-beneficial to the subject, are termed “beneficial responses”.

In some embodiments, a selected tumor antigen stimulates one or more lymphocyte responses that are beneficial to the subject. In some embodiments, a selected tumor antigen inhibits and/or suppresses one or more lymphocyte responses that are deleterious or non-beneficial to the subject.

In some embodiments, a selected tumor antigen increases expression and/or secretion of cytokines that are beneficial to the subject. In some embodiments, a selected tumor antigen inhibits and/or suppresses expression of cytokines that are deleterious or non-beneficial to the subject.

In some embodiments, administration of one or more selected tumor antigens to the subject elicits an immune response of the subject. In some embodiments, administration of one or more selected tumor antigens to the subject elicits a beneficial immune response of the subject. In some embodiments, administration of one or more selected tumor antigens to the subject elicits a beneficial response of the subject. In some embodiments, administration of one or more selected tumor antigens to the subject improves clinical response of the subject to a cancer therapy.

Responses whereby tumor antigens or immunogenic fragments thereof (i) stimulate lymphocyte responses that are deleterious or not beneficial to the subject, (ii) stimulate expression of cytokines that are deleterious or not beneficial to the subject, (iii) inhibit and/or suppress lymphocyte responses that are beneficial to the subject, or (iv) inhibit and/or suppress expression of cytokines that are beneficial to the subject, are termed “deleterious or non-beneficial responses”.

In some embodiments, one or more tumor antigens are selected (or de-selected) based on association with desirable or beneficial immune responses. In some embodiments, one or more tumor antigens are selected (or de-selected) based on association with undesirable, deleterious, or non-beneficial immune responses.

In some embodiments, a selected tumor antigen stimulates one or more lymphocyte responses that are deleterious or non-beneficial to the subject. In some embodiments, a selected tumor antigen inhibits and/or suppresses one or more lymphocyte responses that are beneficial to the subject.

In some embodiments, a selected tumor antigen increases expression and/or secretion of cytokines that are deleterious or non-beneficial to the subject. In some embodiments, a selected tumor antigen inhibits and/or suppresses expression of cytokines that are beneficial to the subject.

In some embodiments, the one or more tumor antigens are de-selected by the methods herein.

In some embodiments, the one or more selected tumor antigens are excluded from administration to a subject.

### Methods of Selecting Potential Tumor Antigens

In well-established tumors, activation of endogenous anti-tumor T cell responses is often insufficient to result in complete tumor regression. Moreover, T cells that have been educated in the context of the tumor micro-environment sometimes are sub-optimally activated, have low avidity, and ultimately fail to recognize the tumor cells that express antigen. In addition, tumors are complex and comprise numerous cell types with varying degrees of expression of mutated genes, making it difficult to generate polyclonal T cell responses that are adequate to control tumor growth. As a result, researchers in the field have proposed that it is important in cancer subjects to identify the mutations that are “potential tumor antigens” in addition to those that are confirmed in the cancer subject to be recognized by their T cells.

There are currently no reliable methods of identifying potential tumor antigens in a comprehensive way. Computational methods have been developed in an attempt to predict what is an antigen, however there are many limitations to these approaches. First, modeling epitope prediction and presentation needs to take into account the greater than 12,000 HLA alleles encoding MHC molecules, with each subject expressing as many as 14 of them, all with different epitope affinities. Second, the vast majority of predicted epitopes fail to be found presented by tumors when they are evaluated using mass spectrometry. Third, the predictive algorithms do not take into account T cell recognition of the antigen, and the majority of predicted epitopes are incapable of eliciting T cell responses even when they are present. Finally, the second arm of cellular immunity, the CD4⁺ T cell subset, is often overlooked; the majority of in silico tools focus on MHC class I binders. The tools for predicting MHC class II epitopes are under-developed and more variable.

The present disclosure provides methods to a) identify polypeptides that are potential tumor antigens in antigen presentation assays of the disclosure, and b) select polypeptides on the basis of their antigenic potential. The methods are performed without making predictions about what could be a target of T cell responses or presented by MHC, and without the need for deconvolution. The methods can be expanded to explore antigenic potential in healthy subjects who share the same MHC alleles as a subject, to identify those potential tumor antigens that would be most suitable to include in an immunogenic composition or vaccine formulation. The methods ensure that the potential tumor antigen is processed and presented in the context of subject MHC molecules, and that T cells can respond to the potential tumor antigen if they are exposed to the potential tumor antigen under the right conditions (e.g., in the context of a vaccine with a strong danger signal from an adjuvant or delivery system).

The preceding methods for selection of tumor antigens may be applied to selection of potential tumor antigens, that is, polypeptides encoding one or more mutations present or expressed in a cancer or tumor cell of a subject.

Methods of Redirecting Immune Responses and/or Re-Educating Lymphocytes

As discussed herein, the disclosure provides methods of redirecting one or more immune responses (e.g., one or more immune responses described herein), e.g., by re-educating one or more lymphocytes. In some embodiments, methods include administering to a subject (i) an inhibitory antigen described herein and (ii) an effective amount of an agent or a combination of agents, thereby inducing an immune response in the subject. In some embodiments, administration of the inhibitory antigen to the subject, without an effective amount of the agent or the combination of agents, induces an immune response that impairs or reduces immune control of a tumor or cancer cell in the subject. In certain embodiments, an inhibitory antigen and an agent or a combination of agents are formulated as a pharmaceutical composition, e.g., a vaccine composition described herein.

As discussed herein, in some embodiments, the present disclosure provides methods and systems related to redirecting one or more immune responses in a subject. In some embodiments, an initial immune response in a subject impairs or reduces immune control of a tumor or cancer cell in the subject (e.g., the subject has a clinically negative response, or is clinically non-responsive). In some embodiments, an initial immune response in a subject that impairs or reduces immune control of a tumor or cancer cell in the subject is redirected (e.g., using methods of the disclosure) such that the immune response in a subject enhances immune control of a tumor or cancer cell in the subject (e.g., the subject has a clinically positive response).

Whether an immune response impairs or enhances immune control of a tumor or cancer cell can be measured and/or characterized according to particular criteria. In certain embodiments, such criteria can include clinical criteria and/or objective criteria. In certain embodiments, techniques for assessing response can include, but are not limited to, clinical examination, positron emission tomography, chest X-ray, CT scan, MRI, ultrasound, endoscopy, laparoscopy, presence or level of a particular marker in a sample, cytology, and/or histology. A positive response, a negative response, and/or no response, of a tumor can be assessed by ones skilled in the art using a variety of established techniques for assessing such response, including, for example, for determining one or more of tumor burden, tumor size, tumor stage, etc. Methods and guidelines for assessing response to treatment are discussed in Therasse et al., J. Natl. Cancer Inst., 2000, 92(3):205-216; and Seymour et al., Lancet Oncol., 2017, 18:e143-52.

In some embodiments, enhanced immune control of a tumor or cancer results in a measured decrease in tumor burden, tumor size, and/or tumor stage. In some embodiments, impaired immune control of a tumor or cancer does not result in a measured decrease in tumor burden, tumor size, or tumor stage. In some embodiments, impaired immune control of a tumor or cancer results in a measured increase in tumor burden, tumor size, or tumor stage.

Exemplary agents that can be used to re-educate a T cell and/or to redirect an immune response include adjuvants, cytokines, immune checkpoint blockade therapies (e.g., described herein), viral vectors, bacterial vectors, exosomes, liposomes, DNAs, mRNAs, saRNAs, chemotherapeutic agents, and IDO inhibitors.

Adjuvants

Adjuvants can be broadly separated into two classes, based on their principal mechanisms of action: vaccine delivery systems and immunostimulatory adjuvants (see, e.g., Singh et al., Curr. HIV Res. 1:309-20, 2003). Vaccine delivery systems are often particulate formulations, e.g., emulsions, microparticles, immune-stimulating complexes (ISCOMs), which may be, for example, particles and/or matrices, and liposomes. In contrast, immunostimulatory adjuvants are sometimes derived from pathogens and can represent pathogen associated molecular patterns (PAMP), e.g., lipopolysaccharides (LPS), monophosphoryl lipid (MPL), or CpG-containing DNA, which activate cells of the innate immune system.

Alternatively, adjuvants may be classified as organic and inorganic. Inorganic adjuvants include alum salts such as aluminum phosphate, amorphous aluminum hydroxyphosphate sulfate, and aluminum hydroxide, which are commonly used in human vaccines. Organic adjuvants comprise organic molecules including macromolecules. An example of an organic adjuvant is cholera toxin.

Adjuvants may also be classified by the response they induce, and adjuvants can activate more than one type of response. In some embodiments, the adjuvant induces the activation of CD4⁺ T cells. The adjuvant may induce activation of TH1 cells and/or activation of TH17 cells and/or activation of TH2 cells. Alternately, the adjuvant may induce activation of TH1 cells and/or TH17 cells but not activation of TH2 cells, or vice versa. In some embodiments, the adjuvant induces activation of CD8⁺ T cells. In further embodiments, the adjuvant may induce activation of Natural Killer T (NKT) cells. In some embodiments, the adjuvant induces the activation of TH1 cells or TH17 cells or TH2 cells. In other embodiments, the adjuvant induces the activation of B cells. In yet other embodiments, the adjuvant induces the activation of antigen-presenting cells. These categories are not mutually exclusive; in some cases, an adjuvant activates more than one type of cell.

In certain embodiments, an adjuvant is a substance that increases the numbers or activity of antigen presenting cells such as dendritic cells. In certain embodiments, an adjuvant promotes the maturation of antigen presenting cells such as dendritic cells. In some embodiments, an adjuvant is an inflammasome activator. In some embodiments the inflammasome activator is aluminum potassium sulfate, a RIG-I agonist such as Poly(dA:dT), a TLR5 agonist such as flagellin, or a dectin-1 antagonist such as Curdlan. In some embodiments, the adjuvant is or comprises a saponin. Typically, the saponin is a triterpene glycoside, such as those isolated from the bark of the Quillaja saponaria tree. A saponin extract from a biological source can be further fractionated (e.g., by chromatography) to isolate the portions of the extract with the best adjuvant activity and with acceptable toxicity. Typical fractions of extract from Quillaja saponaria tree used as adjuvants are known as fractions A and C. An exemplary saponin adjuvant is QS-21, which is available from Antigenics. QS-21 is an oligosaccharide-conjugated small molecule. Optionally, QS-21 may be admixed with a lipid such as 3D-MPL or cholesterol.

A particular form of saponins that may be used in vaccine formulations described herein is immunostimulating complexes (ISCOMs). ISCOMs are an art-recognized class of adjuvants, that generally comprise Quillaja saponin fractions and lipids (e.g., cholesterol and phospholipids such as phosphatidyl choline). In certain embodiments, an ISCOM is assembled together with a polypeptide or nucleic acid of interest. However, different saponin fractions may be used in different ratios. In addition, the different saponin fractions may either exist together in the same particles or have substantially only one fraction per particle (such that the indicated ratio of fractions A and C are generated by mixing together particles with the different fractions). In this context, “substantially” refers to less than 20%, 15%, 10%, 5%, 4%, 3%, 2% or even 1%. Such adjuvants may comprise fraction A and fraction C mixed into a ratio of 70-95 A:30-5 C, such as 70 A:30 C to 75 A:25 C, 75 A:25 C to 80 A:20 C, 80 A:20 C to 85 A:15 C, 85 A:15 C to 90 A:10 C, 90 A:10 C to 95 A:5 C, or 95 A:5 C to 99 A:1 C. ISCOMatrix, produced by CSL, and AbISCO 100 and 300, produced by Isconova, are ISCOM matrices comprising saponin, cholesterol and phospholipid (lipids from cell membranes), which form cage-like structures typically 40-50 nm in diameter. Posintro, produced by Nordic Vaccines, is an ISCOM matrix where the immunogen is bound to the particle by a multitude of different mechanisms, e.g. electrostatic interaction by charge modification, incorporation of chelating groups or direct binding.

In some embodiments, the adjuvant is a TLR agonist, a STING agonist, or a molecule that triggers the inflammasome. In some embodiments, the TLR agonist is a TLR2 agonist such as Pam3CSK4. In some embodiments, the TLR agonist is a TLR3 agonist such as Poly-IC or Poly-ICLC (Hiltonol). In some embodiments, the TLR agonist is a TLR4 agonist such as 3D-PHAD. In some embodiments the TLR agonist is a TLR7 agonist such as imiquimod or R848. In some embodiments, the TLR agonist is a TLR5 agonist such as flagellin. In some embodiments, the TLR agonist is a TLR9 agonist such as CpG.

In some embodiments, the adjuvant is a nanoemulsion that is a high-energy, oil-in-water emulsion with a size of 150-400 nanometers, and includes surfactants to provide stability.

Adjuvants may be covalently bound to antigens (e.g., the polypeptides described above). In some embodiments, the adjuvant may be a protein which induces inflammatory responses through activation of antigen-presenting cells (APCs). In some embodiments, one or more of these proteins can be recombinantly fused with an antigen of choice, such that the resultant fusion molecule promotes dendritic cell maturation, activates dendritic cells to produce cytokines and chemokines, and ultimately, enhances presentation of the antigen to T cells and initiation of T cell responses (see Wu et al., Cancer Res 2005; 65(11), pp 4947-4954). Other exemplary adjuvants that may be covalently bound to antigens comprise polysaccharides, small molecules, synthetic peptides, lipopeptides, and nucleic acids.

The adjuvant can be used alone or in combination of two or more kinds. Adjuvants may be directly conjugated to antigens. Adjuvants may also be combined to increase the magnitude of the immune response to the antigen. Typically, the same adjuvant or mixture of adjuvants is administered a teach stimulation event (e.g., vaccination, prime injection, or boost injection). Optionally, however, an adjuvant may be administered at the first stimulation but not subsequent stimulations. Alternatively, a strong adjuvant may be administered at initial stimulation, and a weaker adjuvant or lower dose of the strong adjuvant may be administered at subsequent re=stimulations. The adjuvant can be administered before the antigen, concurrent with the antigen or after administration of the antigen to a subject (sometimes within 1, 2, 6, or 12 hours; sometimes within 1, 2, or 5 days; sometimes within 1, 2, or 3 months; sometimes within 6, 12, or 18 months; sometimes within 2, 3, 4, 5, 10, or 15 years). In some embodiments, an adjuvant may be directly combined or formulated with an antigen to make a vaccine composition. In certain embodiments, an adjuvant may be administered separately from an antigen. An adjuvant may be administered separately but concurrently with an antigen, or may be administered separately in between doses of an antigen.

Chemotherapeutic Agents

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Nonlimiting examples of chemotherapeutic agents include erlotinib (TARCEVA®, Genentech/OSI Pharm.), docetaxel (TAXOTER®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine,dichloroplatinum(II), CAS No. 15663-27-1), carboplatin (CAS No. 41575-94-4), paclitaxel (TAXOL®, Bristol-Myers Squibb Oncology, Princeton, N.J.), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethyl-ethanamine, NOLVADEX®, ISTUBAL®, VALODEX®), and doxorubicin (ADRIAMYCIN®), Akti-1/2, HPPD, and rapamycin.

Additional examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (MEK inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, calicheamicin gammalI, calicheamicin omegaIl (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside (Ara-C); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

In some embodiments, an antibody may be used as an agent to bind to tumor cells expressing the inhibitory antigen to stimulate an antibody-dependent cell-mediated cytotoxicity (ADCC) against the tumor cells. Antibodies may bind to tumor cells expressing the inhibitory antigen and prevent activation of the antigen-specific inhibitory T cell (ie “cap” the inhibitory antigen).

### Methods of Measuring Change in Lymphocyte Response

The redirection of an immune response or re-education of a lymphocyte may be determined by measuring the change in lymphocyte response to one or more antigens.

In some embodiments, lymphocyte response may be measured at a cellular level. In some embodiments, lymphocyte response may be measured by performing assays to measure the level of certain immune mediators. Assays may include, but are not limited to the antigen presentation assays described previously. Immune mediators measured may be known immune mediators and immune mediators described herein, for example, cytokines. An exemplary assay to measure lymphocyte response may be an assay that uses an enzyme-linked immunosorbent assay (ELISA) technique, such as an ELISPOT assay. Assays may also include analysis of upregulation of cell surface molecules such as co-stimulatory molecules (i.e. CD28, LFA-1, CD137 [4-1BB], CD154 [CD40L]), effector memory markers (i.e. CD45RO, CD62L), or HLA molecules by flow cytometry. Assays may also include evaluation of beneficial genes via gene chip analyses.

At a cellular level, redirection of immune responses or re-education of lymphocytes may be determined by the percent change in cytokine secretion in response to an identified antigen compared to a control level where the antigen is not presented for example, by more than 5%, 6%, 7%, 8%, 9%, 10%, or 20%. A control level may be without presentation of an antigen or without the addition of a composition to induce redirection of an immune response or re-education, such as an adjuvant. Redirection of an immune response or re-education may be determined by a change in levels of immune mediators in response to an antigen presented alone compared to an antigen presented in combination with an adjuvant. Redirection of an immune response or re-education may be determined by a change in levels of one or more immune mediators over time, for example, by more than 5%, 6%, 7%, 8%, 9%, 10%, or 20%. In some embodiments, redirection of an immune response or re-education may be determined by a change in the levels of different immune mediators produced by a lymphocyte, or the change in the predominant type of immune mediator produced by a lymphocyte in response to the presentation of an antigen. For example, the change in expression and/or secretion of IL-10 to IFN-gamma may indicate redirection or re-education from an immunosuppressive response to an immunostimulatory response.

At the tissue level, an immune response may be measured by the pathology of a tissue in a subject. In some embodiments, RECIST criteria (http://recist.eortc.org/publications/) can be used to determine if the tumors shrink, grow, or stay the same. In some embodiments, pathologies characterizing tumors as may be used to characterize an immune response over time and can include tumor size, altered expression of genetic markers, invasion of adjacent organs and/or lymph nodes by tumor cells. In some embodiments, immune response may be evidenced by the size of a tumor, using a metric such as tumor area and/or volume. Tumor area and/or volume may be measured over time and immune response may be indicated by the change in size and/or growth kinetics of the tumor. In some embodiments, a change in tumor size or rate of growth in a subject immunized with an immunogenic composition may be compared to the change in tumor size or rate of growth in an un-immunized control subject. In some embodiments, infiltration of the tumors with immune cells can be monitored with multi-parameter immunohistochemistry, T cell receptor sequencing, or evaluation of enriched tumor infiltrating lymphocytes using conventional immunoassays. Redirection of immune response or re-education of lymphocytes can be determined by an increase in tumor infiltration by T cells.

Redirection of immune responses or re-education of lymphocytes at a tissue level may be determined by a change in the growth of a tumor over time in a subject immunized with antigen compared to a control, for example, by more than 5%, 6%, 7%, 8%, 9%, 10%, or 20%. Redirection of immune responses or re-education of lymphocytes at a tissue level may be demonstrated by a difference in tumor area or volume in a subject immunized with antigen compared to a control, for example, by more than 5%, 6%, 7%, 8%, 9%, 10%, or 20%. A control level may be without presentation of an antigen or without the addition of a composition to induce redirection of an immune response or re-education, such as an adjuvant.

Immunogenic Compositions and Uses thereof

The present disclosure provides compositions that include a tumor antigen or tumor antigens described herein and/or identified or selected by methods described herein, nucleic acids encoding the tumor antigens, and methods of using the compositions. In some embodiments, a composition includes tumor antigens that are peptides 8-40 amino acids, 8-60 amino acids, 8-100. 8-150, or 8-200 amino acids in length (e.g., MHC binding peptides, e.g., peptides 23-29, 24-28, 25-27, 8-30, 8-29, 8-28, 8-27, 8-26, 8-25, 8-24, 8-23, 8-22, 8-21, 8-20, 8-15, 8-12 amino acids in length). In some embodiments, a composition includes one or more tumor antigens that are about 70%, 75%, 80%, 85%, 90%, 95%, 96%, 97%, 98%, 99% or 100% of the length of the full-length polypeptides. In some embodiments, a composition includes one or more tumor antigens that are truncated by about 1, 2, 3, 4, 5, 10, 15, 20, 25, 30, 35, 40, 45, 50, or more amino acids, relative to the full-length polypeptides. The compositions can include tumor antigens that are, or that comprise, MHC class I-binding peptides, MHC class II-binding peptides, or both MHC class I and MHC class II-binding peptides. Compositions can include a single tumor antigen, or multiple tumor antigens. In some embodiments, a composition includes a set of two, three, four, five, six, seven, eight, nine, ten, or more tumor antigens. In some embodiments, a composition includes ten, fifteen, twenty, twenty-five, thirty, or more tumor antigens. In some embodiments, the tumor antigens or peptides are provided as one or more fusion proteins. In some embodiments, a composition comprises nucleic acids encoding the tumor antigens or peptides. In some embodiments, the nucleic acids encoding the tumor antigens or peptides are provided as one or more fusion constructs.

The disclosure also provides nucleic acids encoding the tumor antigens. The nucleic acids can be used to produce expression vectors, e.g., for recombinant production of the tumor antigens, or for nucleic acid-based administration in vivo (e.g., DNA vaccination).

In some embodiments, tumor antigens are used in diagnostic assays. For these assays, compositions including the tumor antigens can be provided in kits, e.g., for detecting antibody reactivity, or cellular reactivity, in a sample from an individual.

In some embodiments, tumor antigen compositions are used to induce an immune response in a subject. In some embodiments, the subject is a human. In some embodiments, the subject is a non-human animal. The tumor antigen compositions can be used to raise antibodies (e.g., in a non-human animal, such as a mouse, rat, hamster, or goat), e.g., for use in diagnostic assays, and for therapeutic applications. In some embodiments, a tumor antigen discovered by a method described herein may be a potent B cell antigen. Preparations of antibodies may be produced by immunizing a subject with the tumor antigen and isolating antiserum from the subject. Methods for eliciting high titers of high affinity, antigen-specific antibodies, and for isolating the tumor antigen-specific antibodies from antisera, are known in the art. In some embodiments, the tumor antigen compositions are used to raise monoclonal antibodies, e.g., human monoclonal antibodies. In some embodiments, the tumor antigen compositions may induce a T cell response. In some embodiments, the tumor antigen compositions may induce a T cell response and a B cell response.

In some embodiments, a tumor antigen composition is used to induce an immune response in a human subject to provide a therapeutic response. In some embodiments, a tumor antigen composition is used to induce an immune response in a human subject that redirects an undesirable immune response. In some embodiments, a tumor antigen composition elicits an immune response that causes the subject to have a positive clinical response described herein, e.g., as compared to a subject who has not been administered the tumor antigen composition. In some embodiments, a tumor antigen composition elicits an immune response that causes the subject to have an improved clinical response, e.g., as compared to a subject who has not been administered the tumor antigen composition. In some embodiments, a tumor antigen composition is used to induce an immune response in a human subject for palliative effect. The immune response can result in complete or partial therapy.

In some embodiments, a tumor antigen composition is used to induce an immune response in a human subject to provide a prophylactic response. The immune response can result in complete or partial protection.

In some embodiments, the composition includes a pharmaceutically acceptable carrier or excipient in order to alter, redirect, or re-educate the immune response of a subject or a lymphocyte. An immunogenic composition may also include an adjuvant for enhancing the immunogenicity of the formulation, (e.g., oil in water, incomplete Freund's adjuvant, aluminum phosphate, aluminum hydroxide, saponin adjuvants, toll-like receptor agonists, or muramyl dipeptides) or any of the adjuvants previously described.

In some embodiments, immunogenicity of a tumor antigen is evaluated in vivo. In some embodiments, humoral responses to a tumor antigen are evaluated (e.g., by detecting antibody titers to the administered tumor antigen). In some embodiments, cellular immune responses to a tumor antigen are evaluated, e.g., by detecting the frequency of antigen-specific cells in a sample from the subject (e.g., by staining T cells from the subject with MHC/peptide tetramers containing the antigenic peptide, to detect antigen-specific T cells, or by detecting antigen-specific cells using an antigen presentation assay such as an assay described herein). In some embodiments, the ability of a tumor antigen or antigens to elicit protective or therapeutic immunity is evaluated in an animal model. In some embodiments, the ability of a tumor antigen or antigens to stimulate or to suppress and/or inhibit immunity is evaluated in an animal model.

In some embodiments, an immunogenic composition includes a tumor antigen linked to a carrier protein. Examples of carrier proteins include, e.g., toxins and toxoids (chemical or genetic), which may or may not be mutant, such as anthrax toxin, PA and DNI (PharmAthene, Inc.), diphtheria toxoid (Massachusetts State Biological Labs; Serum Institute of India, Ltd.) or CRM 197, tetanus toxin, tetanus toxoid (Massachusetts State Biological Labs; Serum Institute of India, Ltd.), tetanus toxin fragment Z, exotoxin A or mutants of exotoxin A of Pseudomonas aeruginosa, bacterial flagellin, pneumolysin, an outer membrane protein of Neisseria meningitidis (strain available from the ATCC (American Type Culture Collection, Manassas, Va.)), Pseudomonas aeruginosa Hcp1 protein, E. coli heat labile enterotoxin, shiga-like toxin, human LTB protein, a protein extract from whole bacterial cells, and any other protein that can be cross-linked by a linker. Other useful carrier proteins include high density lipoprotein (HDL), bovine serum albumin (BSA), P40, and chicken riboflavin. Many carrier proteins are commercially available (e.g., from Sigma Aldrich).

In some embodiments, an immunogenic composition including a tumor antigen identified by a method described herein is used in conjunction with an available vaccine. For example, an antigen identified as described herein can be used as a supplemental component of a vaccine formulation, or as a boosting antigen in a vaccination protocol.

In some embodiments, an immunogenic composition is in a volume of about 0.5 mL for subcutaneous injection, 0.1 mL for intradermal injection, or 0.002-0.02 mL for percutaneous administration. A 0.5 ml dose of the composition may contain approximately 2-500 μg of the tumor antigen.

In some embodiments an immunogenic composition is administered parenterally (for instance, by subcutaneous, intramuscular, intravenous, or intradermal injection). In some embodiments, delivery by a means that physically penetrates the dermal layer is used (e.g., a needle, airgun, or abrasion).

In some embodiments, an immunogenic composition is administered to a subject, e.g., by intramuscular injection, intradermal injection, or transcutaneous immunization with appropriate immune adjuvants. Compositions can be administered, one or more times, often including a second administration designed to boost an immune response in a subject. The frequency and quantity of dosage of the composition can vary depending on the specific activity of the composition and clinical response of the subject, and can be determined by routine experimentation.

The formulations of immunogenic compositions can be provided in unit-dose or multi-dose containers, for example, sealed ampoules and vials and may be stored in a freeze-dried (lyophilized) condition requiring only the addition of the sterile liquid carrier immediately prior to use.

### Production of Tumor Antigens

A tumor antigen (e.g., a tumor antigen described herein) suitable for use in any method or composition of the disclosure may be produced by any available means, such as recombinantly or synthetically (see, e.g., Jaradat Amino Acids 50:39-68 (2018); Behrendt et al., J. Pept. Sci. 22:4-27 (2016)). For example, a tumor antigen may be recombinantly produced by utilizing a host cell system engineered to express a tumor antigen-encoding nucleic acid. Alternatively or additionally, a tumor antigen may be produced by activating endogenous genes.

Where proteins are recombinantly produced, any expression system can be used. To give but a few examples, known expression systems include, for example, E. coli, egg, baculovirus, plant, yeast, or mammalian cells.

In some embodiments, recombinant tumor antigen suitable for the present invention are produced in mammalian cells. Non-limiting examples of mammalian cells that may be used in accordance with the present invention include BALB/c mouse myeloma line (NSO/l, ECACC No: 85110503); human retinoblasts (PER.C6, CruCell, Leiden, The Netherlands); monkey kidney CV1 line transformed by SV40 (COS-7, ATCC CRL 1651); human embryonic kidney line (HEK293 or 293 cells subcloned for growth in suspension culture, Graham et al., J. Gen Virol., 36:59, 1977); human fibrosarcoma cell line (e.g., HT1080); baby hamster kidney cells (BHK21, ATCC CCL 10); Chinese hamster ovary cells +/−DHFR (CHO, Urlaub and Chasin, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68, 1982); MRC 5 cells; FS4 cells; and a human hepatoma line (Hep G2).

In some embodiments, the present invention provides recombinant tumor antigen produced from human cells. In some embodiments, the present invention provides recombinant tumor antigen produced from CHO cells or HT1080 cells.

Typically, cells that are engineered to express a recombinant tumor antigen may comprise a transgene that encodes a recombinant tumor antigen described herein. It should be appreciated that the nucleic acids encoding recombinant tumor antigen may contain regulatory sequences, gene control sequences, promoters, non-coding sequences and/or other appropriate sequences for expressing the recombinant tumor antigen. Typically, the coding region is operably linked with one or more of these nucleic acid components.

The coding region of a transgene may include one or more silent mutations to optimize codon usage for a particular cell type. For example, the codons of a tumor antigen transgene may be optimized for expression in a vertebrate cell. In some embodiments, the codons of a tumor antigen transgene may be optimized for expression in a mammalian cell. In some embodiments, the codons of a tumor antigen transgene may be optimized for expression in a human cell.

Alternatively or additionally, a tumor antigen may be partially or fully prepared by chemical synthesis. These methods may include chemical synthesis such as solid phase and/or solution phase polypeptide synthesis. See for example, the methodology as described in Bruckdorfer, T. et al. (Curr. Pharm. Biotechnol. 5, 29-43 (2004)).

### Cancer and Cancer Therapy

The present disclosure provides methods and systems related to subjects having or diagnosed with cancer, such as a tumor. In some embodiments, the subject has (or had) a positive clinical response to a cancer therapy or combination of therapies. In some embodiments, the subject had a spontaneous response to a cancer. In some embodiments, the subject is in partial or complete remission from cancer. In some embodiments, the subject has cleared a cancer. In some embodiments, the subject has not had a relapse, recurrence or metastasis of a cancer. In some embodiments, the subject has a positive cancer prognosis. In some embodiments, the subject has not experienced toxic responses or side effects to a cancer therapy or combination of therapies. In some embodiments, the subject has (or had) a negative clinical response to a cancer therapy or combination of therapies. In some embodiments, the subject has not cleared a cancer. In some embodiments, the subject has had a relapse, recurrence or metastasis of a cancer. In some embodiments, the subject has a negative cancer prognosis. In some embodiments, the subject has experienced toxic responses or side effects to a cancer therapy or combination of therapies.

In some embodiments, after treatment (e.g., immunization) with an immunogenic composition described herein, one or more immune responses of the subject adapts. For example, successful cancer therapy leads to a reduced level of one or more tumor antigens to which an immune response is raised.

In some embodiments, a tumor is or comprises a hematologic malignancy, including but not limited to, acute lymphoblastic leukemia, acute myeloid leukemia, chronic lymphocytic leukemia, chronic myelogenous leukemia, hairy cell leukemia, AIDS-related lymphoma, Hodgkin lymphoma, non-Hodgkin lymphoma, Langerhans cell histiocytosis, multiple myeloma, or myeloproliferative neoplasms.

In some embodiments, a tumor is or comprises a solid tumor, including but not limited to breast carcinoma, a squamous cell carcinoma, a colon cancer, a head and neck cancer, ovarian cancer, a lung cancer, mesothelioma, a genitourinary cancer, a rectal cancer, a gastric cancer, or an esophageal cancer.

In some particular embodiments, a tumor is or comprises an advanced tumor, and/or a refractory tumor. In some embodiments, a tumor is characterized as advanced when certain pathologies are observed in a tumor (e.g., in a tissue sample, such as a biopsy sample, obtained from a tumor) and/or when cancer patients with such tumors are typically considered not to be candidates for conventional chemotherapy. In some embodiments, pathologies characterizing tumors as advanced can include tumor size, altered expression of genetic markers, invasion of adjacent organs and/or lymph nodes by tumor cells. In some embodiments, a tumor is characterized as refractory when patients having such a tumor are resistant to one or more known therapeutic modalities (e.g., one or more conventional chemotherapy regimens) and/or when a particular patient has demonstrated resistance (e.g., lack of responsiveness) to one or more such known therapeutic modalities.

In some embodiments, compositions comprising an inhibitory antigen and an agent described herein can be administered in combination with a cancer therapy. The present disclosure is not limited to any specific cancer therapy, and any known or developed cancer therapy is encompassed by the present disclosure. Known cancer therapies include, e.g., administration of chemotherapeutic agents, radiation therapy, surgical excision, chemotherapy following surgical excision of tumor, adjuvant therapy, localized hypothermia or hyperthermia, anti-tumor antibodies, and anti-angiogenic agents. In some embodiments, cancer and/or adjuvant therapy includes a TLR agonist (e.g., CpG, Poly I:C, etc., see, e.g., Wittig et al., Crit. Rev. Oncol. Hematol. 94:31-44 (2015); Huen et al., Curr. Opin. Oncol. 26:237-44 (2014); Kaczanowska et al., J. Leukoc. Biol. 93:847-863 (2013)), a STING agonist (see, e.g., US20160362441; US20140329889; Fu et al., Sci. Transl. Med. 7:283ra52 (2015); and WO2014189805), a non-specific stimulus of innate immunity, and/or dendritic cells, or administration of GM-CSF, Interleukin-12, Interleukin-7, Flt-3, or other cytokines. In some embodiments, the cancer therapy is or comprises oncolytic virus therapy, e.g., talimogene leherparepvec. (see, e.g., Fukuhara et al., Cancer Sci. 107:1373-1379 (2016)). In some embodiments, the cancer therapy is or comprises bi-specific antibody therapy (e.g., Choi et al., 2011 Expert Opin Biol Ther; Huehls et al., 2015, Immunol and Cell Biol). In some embodiments, the cancer therapy is or comprises cellular therapy such as chimeric antigen receptor T (CAR-T) cells, TCR-transduced T cells, dendritic cells, tumor infiltrating lymphocytes (TIL), or natural killer (NK) cells (e.g., as reviewed in Sharpe and Mount, 2015, Dis Model Mech 8:337-50).

Anti-tumor antibody therapies (i.e., therapeutic regimens that involve administration of one or more anti-tumor antibody agents) are rapidly becoming the standard of care for treatment of many tumors. Antibody agents have been designed or selected to bind to tumor antigens, particularly those expressed on tumor cell surfaces. Various review articles have been published that describe useful anti-tumor antibody agents (see, for example, Adler et al., Hematol. Oncol. Clin. North Am. 26:447-81 (2012); Li et al., Drug Discov. Ther. 7:178-84 (2013); Scott et al., Cancer Immun. 12:14 (2012); and Sliwkowski et al., Science 341:1192-1198 (2013)). The below Table 8 presents a non-comprehensive list of certain human antigens targeted by known, available antibody agents, and notes certain cancer indications for which the antibody agents have been proposed to be useful:

TABLE 8

| | Antibody (commercial or | |
|---|---|---|
| Human Antigen | scientific name) | Cancer indication |
| CD2 | Siplizumab | Non-Hodgkin's Lymphoma |
| CD3 | UCHT1 | Peripheral or Cutaneous T-cell Lymphoma |
| CD4 | HuMax-CD4 | |
| CD19 | SAR3419, MEDI-551 | Diffuse Large B-cell Lymphoma |
| CD19 and CD3 or | Bispecific antibodies such as | Non-Hodgkin's Lymphoma |
| CD22 | Blinatumomab, DT2219ARL | |
| CD20 | Rituximab, Veltuzumab, | B cell malignancies (Non-Hodgkin's |
| | Tositumomab, Ofatumumab, | lymphoma, Chronic lymphocytic leukemia) |
| | Ibritumomab, Obinutuzumab, | |
| CD22 (SIGLEC2) | Inotuzumab, tetraxetan, CAT- | Chemotherapy-resistant hairy cell leukemia, |
| | 8015, DCDT2980S, Bectumomab | Hodgkin's lymphoma |
| CD30 | Brentuximab vedotin | |
| CD33 | Gemtuzumab ozogamicin | Acute myeloid leukemia |
| | (Mylotarg) | |
| CD37 | 16 | Chronic lymphocytic leukemia |
| CD38 | mumab | Multiple myeloma, hematological tumors |
| CD40 | mumab | Non-Hodgkin's lymphoma |
| CD52 | Alemtuzumab (Campath) | Chronic lymphocytic leukemia |
| CD56 (NCAM1) | Lorvotuzumab | Small Cell Lung Cancer |
| CD66e (CEA) | Labetuzumab | Breast, colon and lung tumors |
| CD70 | SGN-75 | Non-Hodgkin's lymphoma |
| CD74 | Milatuzumab | Non-Hodgkin's lymphoma |
| CD138 (SYND1) | BT062 | Multiple Myeloma |
| CD152 (CTLA-4) | Ipilimumab | Metastatic melanoma |
| CD221 (IGF1R) | AVE1642, IMC-A12, MK-0646, | Glioma, lung, breast, head and neck, |
| | R150, CP 751871 | prostate and thyroid cancer |
| CD254 (RANKL) | Denosumab | Breast and prostate carcinoma |
| CD261 (TRAILR1) | Mapatumumab | Colon, lung and pancreas tumors and |
| CD262 (TRAILR2) | HGS-ETR2, CS-1008 | haematological malignancies |
| CD326 (Epcam) | Edrecolomab, 17-1A, IGN101, | Colon and rectal cancer, malignant ascites, |
| | Catumaxomab, Adecatumumab | epithelial tumors (breast, colon, lung) |
| CD309 (VEGFR2) | IM-2C6, CDP791 | Epithelium-derived solid tumors |
| CD319 (SLAMF7) | HuLuc63 | Multiple myeloma |
| CD340 (HER2) | Trastuzumab, Pertuzumab, Ado- | Breast cancer |
| | trastuzumab emtansine | |
| CAIX (CA9) | cG250 | Renal cell carcinoma |
| EGFR (c-erbB) | Cetuximab, Panitumumab, | Solid tumors including glioma, lung, breast, |
| | nimotuzumab and 806 | colon, and head and neck tumors |
| EPHA3 (HEK) | KB004, IIIA4 | Lung, kidney and colon tumors, melanoma, |
| | | glioma and haematological malignancies |
| Episialin | Epitumomab | Epithelial ovarian tumors |
| FAP | Sibrotuzumab and F19 | Colon, breast, lung, pancreas, and head and |
| | | neck tumors |
| HLA-DR beta | Apolizumab | Chronic lymphocytic leukemia, non- |
| | | Hodkin's lymphoma |
| FOLR-1 | Farletuzumab | Ovarian tumors |
| 5T4 | Anatumomab | Non-small cell lung cancer |
| GD3/GD2 | 3F8, ch14.18, KW-2871 | Neuroectodermal and epithelial tumors |
| gpA33 | huA33 | Colorectal carcinoma |
| GPNMB | Glembatumumab | Breast cancer |
| HER3 (ERBB3) | MM-121 | Breast, colon, lung, ovarian, and prostate |
| | | tumors |
| Integrin αVβ3 | Etaracizumab | Tumor vasculature |
| Integrin α5β1 | Volociximab | Tumor vasculature |
| Lewis-Y antigen | hu3S193, IgN311 | Breast, colon, lung and prostate tumors |
| MET (HGFR) | AMG 102, METMAB, SCH900105 | Breast, ovary and lung tumors |
| Mucin-1/CanAg | Pemtumomab, oregovomab, | Breast, colon, lung and ovarian tumors |
| | Cantuzumab | |
| PSMA | ADC, J591 | Prostate Cancer |
| Phosphatidylserine | Bavituximab | Solid tumors |
| TAG-72 | Minretumomab | Breast, colon and lung tumors |
| Tenascin | 81C6 | Glioma, breast and prostate tumours |
| VEGF | Bevacizumab | Tumour vasculature |
| PD-L1 | Avelumab | Non-small cell lung cancer, MCC |
| CD274 | Durvalumab | Non-small cell lung cancer |
| IDO enzyme | IDO inhibitors | Multiple |

In some embodiments, a cancer therapy is or comprises immune checkpoint blockade therapy (see, e.g., Martin-Liberal et al., Cancer Treat. Rev. 54:74-86 (2017); Menon et al., Cancers (Basel) 8:106 (2016)), or immune suppression blockade therapy. Certain cancer cells thrive by taking advantage of immune checkpoint pathways as a major mechanism of immune resistance, particularly with respect to T cells that are specific for tumor antigens. For example, certain cancer cells may overexpress one or more immune checkpoint proteins responsible for inhibiting a cytotoxic T cell response. Thus, immune checkpoint blockade therapy may be administered to overcome the inhibitory signals and permit and/or augment an immune attack against cancer cells. Immune checkpoint blockade therapy may facilitate immune cell responses against cancer cells by decreasing, inhibiting, or abrogating signaling by negative immune response regulators (e.g., CTLA-4). In some embodiments, a cancer therapy or may stimulate or enhance signaling of positive regulators of immune response (e.g., CD28).

Examples of immune checkpoint blockade and immune suppression blockade therapy include agents targeting one or more of A2AR, B7-H4, BTLA, CTLA-4, CD28, CD40, CD137, GITR, IDO, KIR, LAG-3, PD-1, PD-L1, OX40, TIM-3, and VISTA. Specific examples of immune checkpoint blockade agents include the following monoclonal antibodies: ipilimumab (targets CTLA-4); tremelimumab (targets CTLA-4); atezolizumab (targets PD-L1); pembrolizumab (targets PD-1); nivolumab (targets PD-1); avelumab; durvalumab; and cemiplimab.

Specific examples of immune suppression blockade agents include: Vista (B7-H5, v-domain Ig suppressor of T cell activation) inhibitors; Lag-3 (lymphocyte-activation gene 3, CD223) inhibitors; IDO (indolemamine-pyrrole-2,3,-dioxygenase-1,2) inhibitors; KIR receptor family (killer cell immunoglobulin-like receptor) inhibitors; CD47 inhibitors; and Tigit (T cell immunoreceptor with Ig and ITIM domain) inhibitors.

In some embodiments, a cancer therapy is or comprises immune activation therapy. Specific examples of immune activators include: CD40 agonists; GITR (glucocorticoid-induced TNF-R-related protein, CD357) agonists; OX40 (CD134) agonists; 4-1BB (CD137) agonists; ICOS (inducible T cell stimulator); CD278 agonists; IL-2 (interleukin 2) agonists; and interferon agonists.

In some embodiments, cancer therapy is or comprises a combination of one or more immune checkpoint blockade agents, immune suppression blockade agents, and/or immune activators, or a combination of one or more immune checkpoint blockade agents, immune suppression blockade agents, and/or immune activators, and other cancer therapies.

Methods described herein can include preparing and/or providing a report, such as in electronic, web-based, or paper form. The report can include one or more outputs from a method described herein, e.g., a subject response described herein. In some embodiments, a report is generated, such as in paper or electronic form, which identifies the presence or absence of one or more tumor antigens (e.g., one or more stimulatory and/or inhibitory and/or suppressive tumor antigens, or tumor antigens to which lymphocytes are not responsive, described herein) for a cancer patient, and optionally, a recommended course of cancer therapy. In some embodiments, the report includes an identifier for the cancer patient. In one embodiment, the report is in web-based form.

In some embodiments, additionally or alternatively, a report includes information on prognosis, resistance, or potential or suggested therapeutic options. The report can include information on the likely effectiveness of a therapeutic option, the acceptability of a therapeutic option, or the advisability of applying the therapeutic option to a cancer patient, e.g., identified in the report. For example, the report can include information, or a recommendation, on the administration of a cancer therapy, e.g., the administration of a pre-selected dosage or in a pre-selected treatment regimen, e.g., in combination with one or more alternative cancer therapies, to the patient. The report can be delivered, e.g., to an entity described herein, within 7, 14, 21, 30, or 45 days from performing a method described herein. In some embodiments, the report is a personalized cancer treatment report.

In some embodiments, a report is generated to memorialize each time a cancer subject is tested using a method described herein. The cancer subject can be reevaluated at intervals, such as every month, every two months, every six months or every year, or more or less frequently, to monitor the subject for responsiveness to a cancer therapy and/or for an improvement in one or more cancer symptoms, e.g., described herein. In some embodiments, the report can record at least the treatment history of the cancer subject.

In one embodiment, the method further includes providing a report to another party. The other party can be, for example, the cancer subject, a caregiver, a physician, an oncologist, a hospital, clinic, third-party payor, insurance company or a government office.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

Methods for identifying antigens that stimulate and inhibit the immune response in a tumor environment are detailed below. In addition to identification of stimulatory or inhibitory antigens, methods of redirecting immune responses and/or re-educating T cells by administration of one or more adjuvants or other immune modulating agents are also demonstrated.

A melanoma model was employed to identify murine stimulatory and inhibitory antigens using ATLAS. Mice were implanted subcutaneously with B16F10 tumors, which were subsequently resected for whole exome sequencing and assessed for non-synonymous mutations. ATLAS libraries individually expressing each mutation were constructed and used to screen splenic T cells from tumor-bearing mice to identify stimulatory or inhibitory antigens. Candidate antigens were manufactured as synthetic long peptides and delivered subcutaneously to C57BL/6 mice with or without adjuvant to elucidate the ability of vaccines comprising stimulatory or inhibitory antigens to impact tumor growth.

### Example 1. Identification of Stimulatory and Inhibitory Antigens Using mATLAS Screens

Methods

A cohort of C57BL/6J mice bearing B16F10 tumors were euthanized and their tumors and spleens harvested. DNA obtained from pooled tumors was sequenced and analyzed for non-synonymous mutations. Over 1600 such mutations were identified, and these were synthesized as 399 bp DNA fragments centered upon the base pair change and transformed individually into E. coli bacteria expressing cLLO to build a candidate neoantigen library. Splenocytes frozen from pooled spleens of the tumor-bearing mice were thawed, and CD8⁺ T cells were sorted using a negative selection bead kit. These were subsequently expanded with CD3/CD28 beads and IL-2 for 7 days followed by 1 day of rest after removal of beads and cytokine. Mouse APCs (RAW309 Cr.1 macrophage cell line) were cultured overnight, washed with PBS, then co-cultured with the bacterial library for 2 hours, washed with PBS, and then cultured with the non-specifically expanded and rested CD8⁺ T cells overnight. Harvested supernatant from the co-culture was tested for IFNγ and TNFα by a custom mouse 384-well Meso Scale Discovery (MSD) electrochemiluminescence assay.

Results

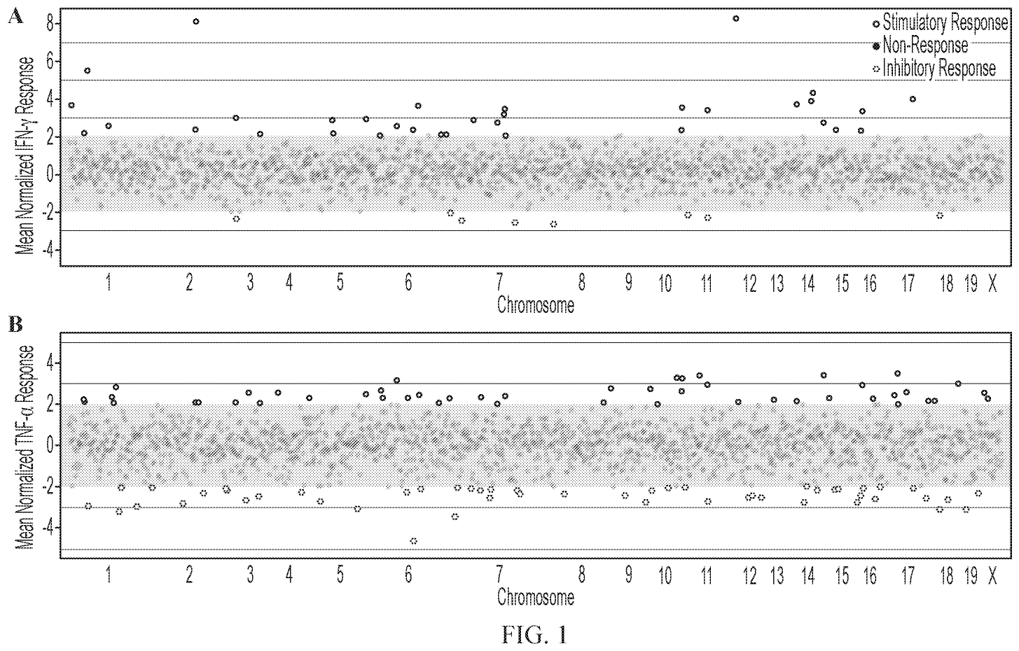

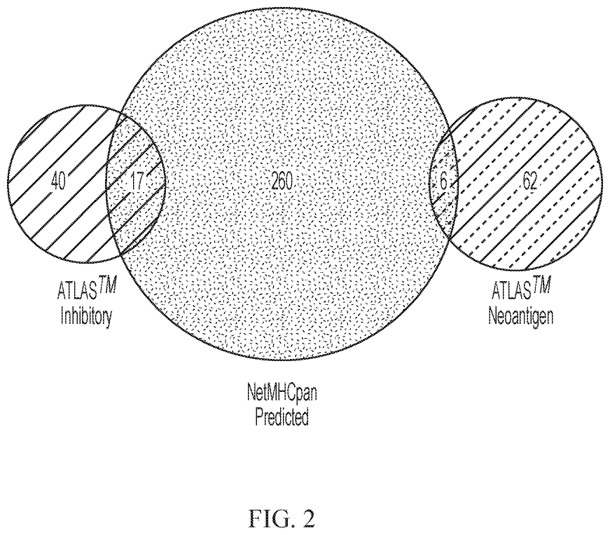

Sixty-eight antigens were identified as stimulatory (exceeding a statistical threshold above the negative control, a 399 bp fragment of the mouse actin gene) and 57 antigens were identified as inhibitory (reduced beyond a statistical threshold below the negative control), for either IFNγ, TNFα, or both (FIG. 1). Only 2% (6 of 283) of NetMHCpan (Nielsen et al., PLoS One. 2007 Aug. 29; 2(8):e796) predicted binding antigens were empirically identified by mATLAS as stimulatory antigens. 6% (17 of 283) of NetMHCpan predicted antigens were identified by mATLAS as inhibitory antigens (FIG. 2).

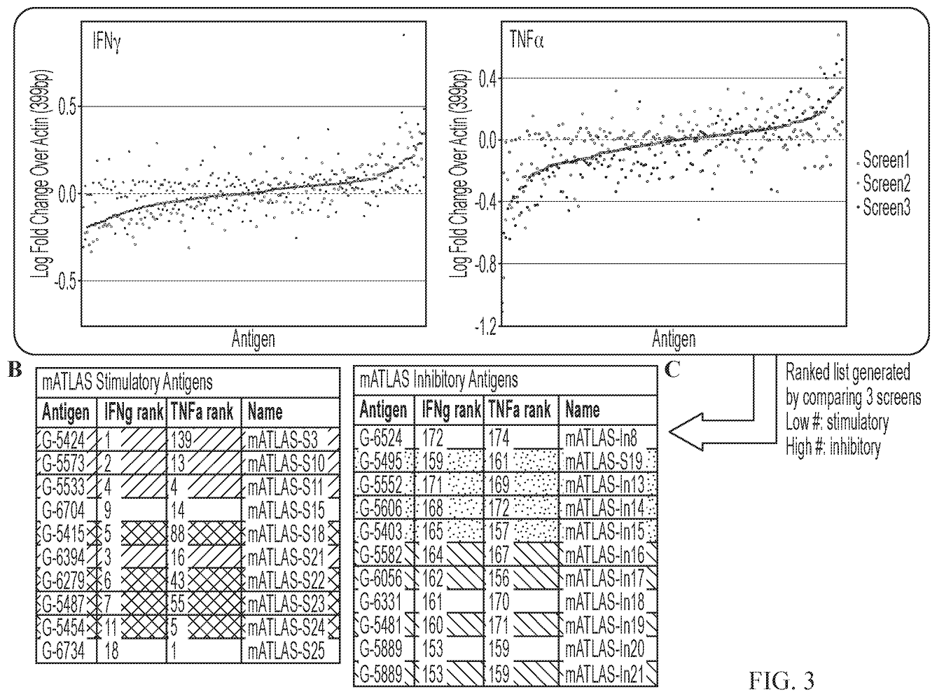

The top 50 stimulatory and 50 inhibitory antigens, and approximately 50 antigens closest to the negative control (non-responses), were used in two additional repeat mATLAS screens with increased replicates. Each antigen was ranked by its IFNγ signal across all 3 screens, as well as a separate rank for its TNFα signal across all 3 screens. The top 10 ranked antigens (stimulatory) and 8 of the bottom 10 ranked antigens (inhibitory) were each synthesized as 27mer synthetic long peptides (SLPs) for use in mouse vaccination, as well as four 15mer overlapping peptides (OLPs) for use in ex vivo assays (FIG. 3 panels A-C).

### Example 2. Mouse Cancer Vaccine Study (Therapeutic Vaccination)

Methods

The top 8 stimulatory and top 8 inhibitory antigens identified and synthesized in Example 1 were divided into 2 groups of 4 stimulatory antigens and 2 groups of 4 inhibitory antigens, respectively. Individual lyophilized synthetic long peptides (SLPs), 27 amino acids in length, were reconstituted in 50% ACN in H2O and pre-mixed, then frozen and lyophilized for 21h and subsequently frozen again as lyophilized pools. The pools of 4 antigens are denoted Stim 1, Stim 2, Inhib 1, and Inhib 2. These were reconstituted on the day of immunization in either PBS/DMSO or PBS/adjuvants/DMSO (final DMSO concentration: 4%).

The pools of 4 stimulatory or inhibitory antigens were used to immunize B16F10 tumor-bearing mice with or without a triple adjuvant combination (CpG, 3D-PHAD, synthetic saponin), denoted triple adjuvant A, on the following schedule: cancer cells were injected subcutaneously on the right flank on day 0 (ATCC-passage 7, 100K cells in 100 □l of 20% Matrigel), vaccine formulations were administered subcutaneously in the tail base on day 3, day 10, and day 17. For SLP-only vaccines, the control group was injected with PBS/DMSO; for adjuvanted vaccines, the control group was injected with triple adjuvant A. A positive control group was injected with 3 published B16F10 antigens: M27 (CD8+ neoantigen), M30 (CD4+ neoantigen), and Trp2 (CD8+ tumor-associated antigen, TAA), previously shown to have both immunogenicity and efficacy in treating the B16F10 tumor model (Castle J C, Kreiter S et al (2012). Exploiting the Mutanome for Tumor Vaccination. Cancer Research 72(5); Kreiter S et al (2015). Mutant MHC class II epitopes drive therapeutic immune responses to cancer. Nature 520(7549)). SLPs dosage was 50 □g per SLP/mouse/day.

Heparinized whole blood was collected on day 17 of the study (i.e., 6 days after vaccine injection #2), red blood cells were lysed, and remaining cells resuspended in OpTmizer media. Cells were counted by a Guava instrument, normalized to one cell concentration, and seeded into an IFNγ ELISPOT plate with overlapping peptides (OLPs; 15mers overlapping by 11aa) for overnight culture. Cells from each individual mouse sample were split into 2 wells: well 1 contained media alone, well 2 contained pooled OLPs (1 μg/ml) specific to the vaccine that the mouse received. For example, for a mouse immunized with peptide antigens 1-4, the cells were stimulated with OLPs 1a-d, 2a-d, 3a-d and 4a-d (16 individual 15mers overlapping by 11 aa total).

Tumor size was measured 3× per week and subsequently on a daily basis, after reaching a specified size threshold. Mice were euthanized when tumors reached maximum size, or became ulcerated and did not heal within 24 hours. No mice in this study were euthanized for other health reasons.

Results

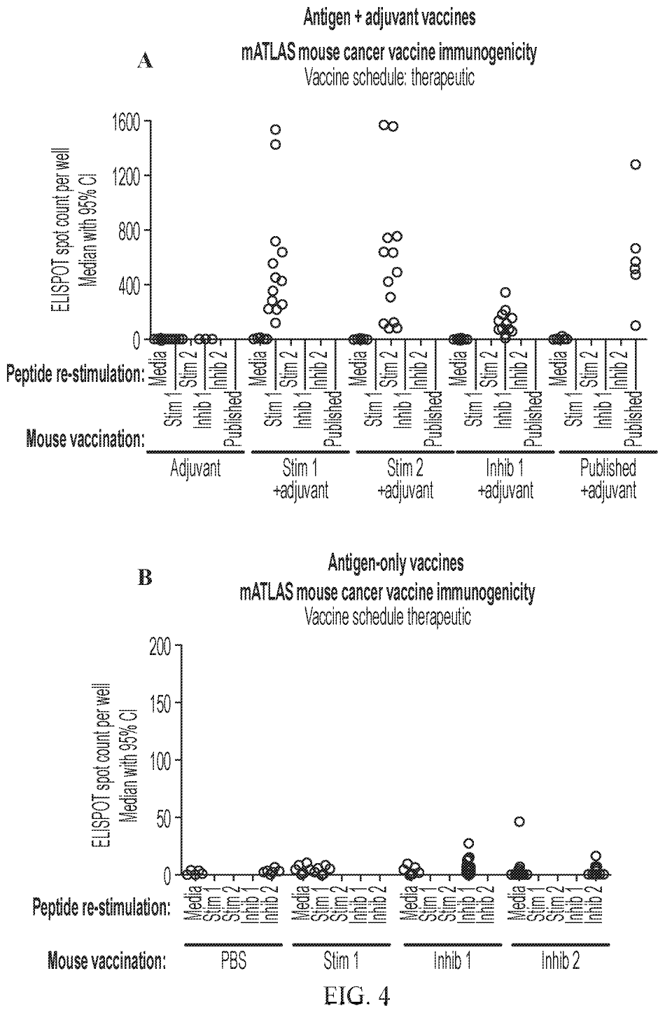

As shown in FIG. 4B, mice that were vaccinated with pools of 4 stimulatory or inhibitory antigens (without adjuvant), generally did not secrete IFNγ above the PBS/DMSO control level upon re-stimulation. However, as seen in FIG. 4A, mice that were vaccinated with 2 different pools of stimulatory antigens (Stim 1 and Stim 2) combined with triple adjuvant A had vigorous T cell responses to antigen re-stimulation, with responses that were comparable to the positive control (Published). Mice vaccinated with a pool of inhibitory antigens (Inhib 1) combined with triple adjuvant A showed weak IFNγ responses in the ELISPOT assay.

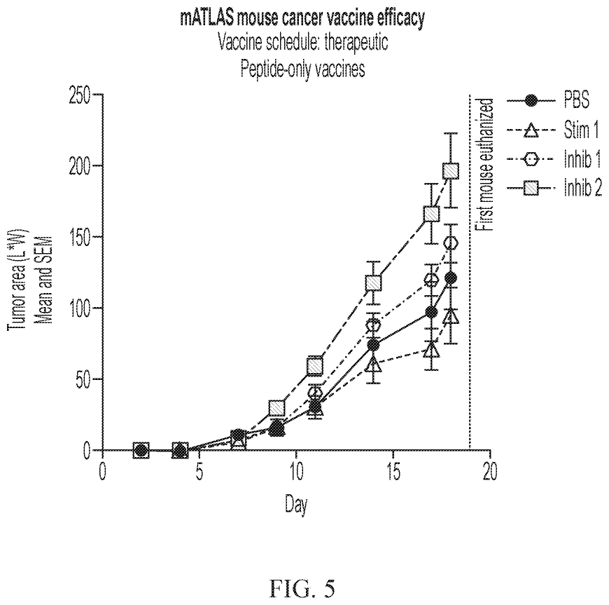

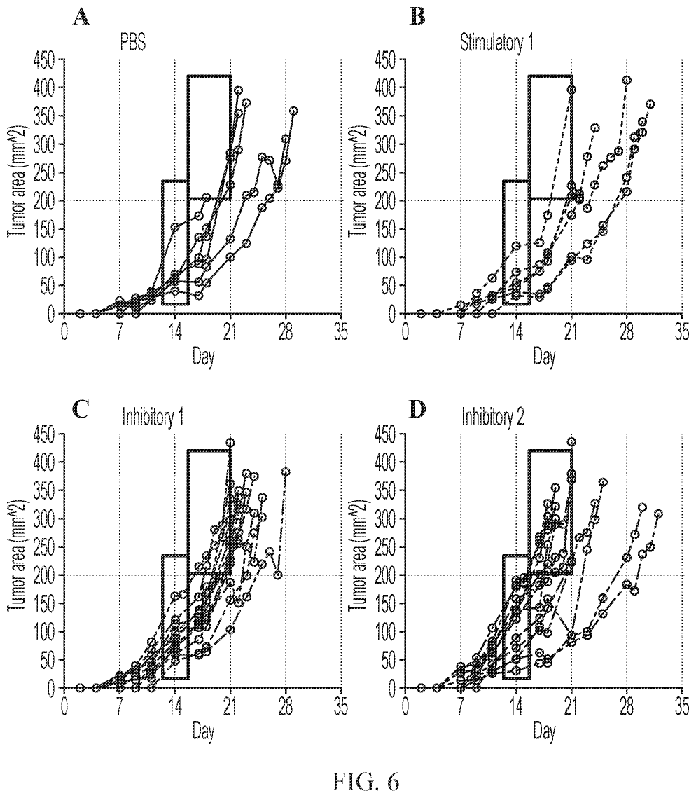

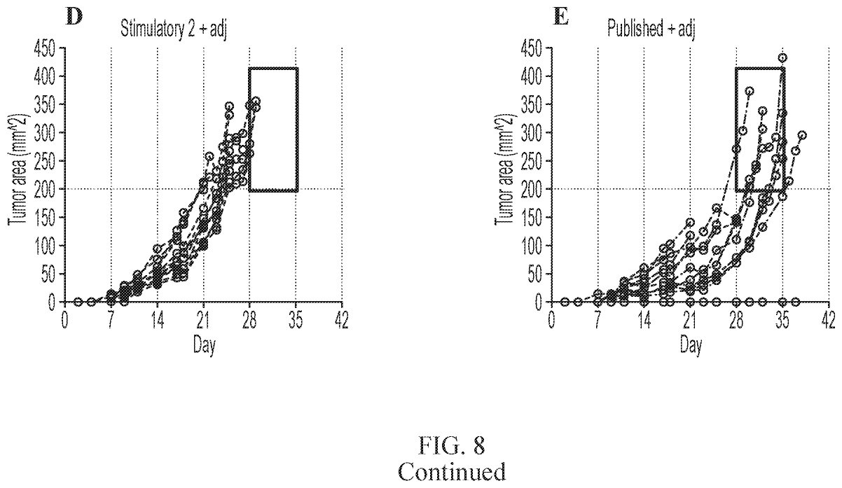

Therapeutic immunization with 2 different pools of inhibitory antigens in the absence of adjuvant led to a marked and significant increase in tumor growth kinetics (FIG. 5, Inhib 1 and Inhib 2). On day 14, individual mice that had been immunized with pools of inhibitory antigens (Inhib 1 and Inhib 2) had larger tumors than mice immunized with PBS/DMSO or a pool of stimulatory antigens (lower boxes, FIGS. 6C and 6D vs. 6A and 6B). By day 21, more than half or the mice in the Inhib 2 group had to be euthanized due to the size of their tumors (upper box, FIG. 6D), which resulted in the decreased survival rates depicted in FIG. 9A.

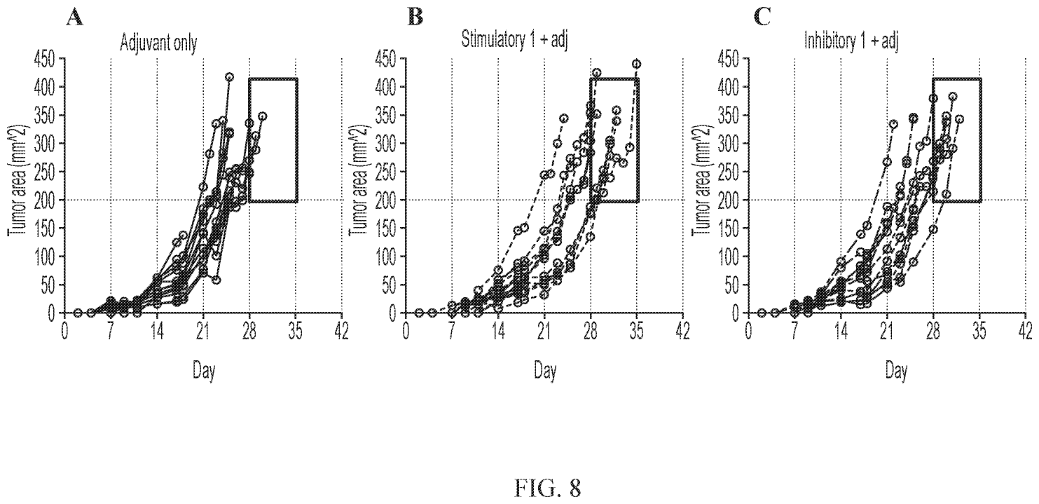

Surprisingly, therapeutic immunization with a pool of inhibitory antigens (Inhib 1) combined with triple adjuvant A led to a slight delay in tumor growth kinetics, most evident after Day 28 relative to adjuvant only (boxes, FIG. 8C compared to FIG. 8A). A modest increase in survival rates relative to adjuvant only was also observed (FIG. 9B, Inhib 1+adj compared to Adjuvant only). These effects were not discernible in Days 1-18 of the experiment.

Therapeutic immunization with a pool of stimulatory antigens (Stim 1) combined with triple adjuvant A also led to a delay in tumor growth kinetics relative to adjuvant only (boxes, FIG. 8B compared to FIG. 8A). These mice had better survival relative to adjuvant only (FIG. 9B, Stim 1+adj compared to Adjuvant only).

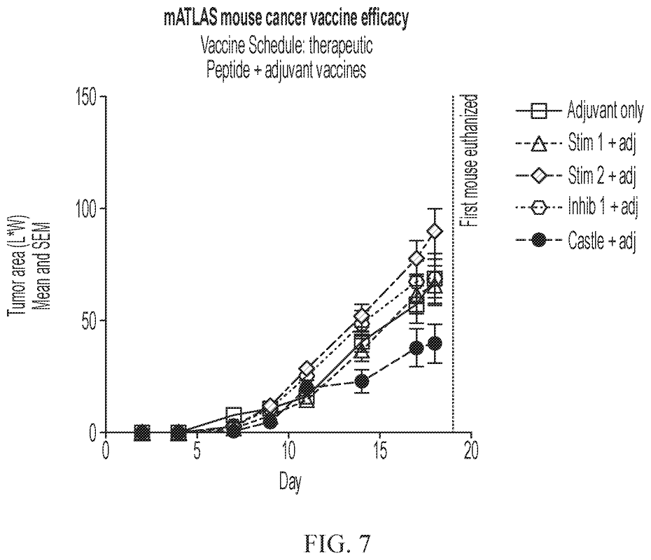

FIG. 7 shows mean tumor area for the groups of mice immunized with pools of stimulatory antigens or inhibitory antigens combined with triple adjuvant A (Stim 1+adj, Stim 2+adj, Inhib 1+adj), the positive control pool of 3 previously known efficacious B16F10 antigens combined with triple adjuvant A (Castle+adj), or triple adjuvant A only.

### Example 3. Mouse Cancer Vaccine Study: Deconvolution of a Pool of 4 Inhibitory Antigens (Therapeutic Vaccination)

Methods

The top 8 stimulatory and inhibitory antigens identified and synthesized in Example 1 are each divided into 2 groups of 4 antigens. Individual lyophilized SLPs are reconstituted in 50% ACN in H2O and pre-mixed, then frozen and lyophilized for 21h and subsequently frozen again as lyophilized pools. These are reconstituted on the day of immunization in either PBS/DMSO or PBS/adjuvants/DMSO (final DMSO concentration: 4%).

A pool of 4 stimulatory antigens, a pool of 4 inhibitory antigens, or 4 individual inhibitory antigens (without adjuvant) are used to vaccinate B16F10 tumor-bearing mice on the following schedule: cancer cells are injected subcutaneously on the right flank on d0 (ATCC-passage 7, 100K cells in 100 μl of 20% Matrigel), vaccine formulations are injected subcutaneously at the tail base on d3, d10, d17. The control group is injected with PBS/DMSO. SLPs dosage is 50 μg per SLP/mouse/day.

Heparinized whole blood is collected on d16 of the study (i.e., 6 days after vaccine injection #2), red blood cells are lysed, and remaining cells resuspended in OpTmizer media. Cells are normalized to one cell concentration and seeded into an IL10 ELISPOT plate with stimulants for overnight culture. Cells from each individual mouse sample is split into 2 wells: well 1 contains media alone, well 2 contains pooled OLPs (1 μg/ml) specific to the vaccine that the mouse receives. For example, for a mouse immunized with peptides 1-4, the cells are stimulated with OLPs 1a-d, 2a-d, 3a-d and 4a-d (16 individual 15mers overlapping by 11 aa total).

Tumor size is measured 3×/week and subsequently on a daily basis after reaching a specified size threshold. Mice are euthanized when tumors reach maximum size, or tumors became ulcerated and do not heal within 24 hours.

### Example 4. Mouse Cancer Vaccine Study with Adjuvant Poly-ICLC (Therapeutic Vaccination)

Methods

The top 8 inhibitory antigens identified and synthesized in Example 1 are each divided into 2 groups of 4 antigens. Individual lyophilized SLPs are reconstituted in 50% ACN in H2O and pre-mixed, then frozen and lyophilized for 21h and subsequently frozen again as lyophilized pools. These are reconstituted on the day of immunization in either PBS/DMSO or PBS/adjuvants/DMSO (final DMSO concentration: 4%).

A pool of 4 inhibitory antigens, with and without adjuvant poly-ICLC, are used to vaccinate B16F10 tumor-bearing mice on the following schedule: cancer cells are injected subcutaneously on the right flank on d0 (ATCC-passage 7, 100K cells in 100 μl of 20% Matrigel), vaccine formulations are injected subcutaneously at the tail base on d3, d10, d17. The control group is injected with PBS/DMSO. SLPs dosage is 50 μg per SLP/mouse/day.

Heparinized whole blood is collected on d16 of the study (i.e., 6 days after vaccine injection #2), red blood cells are lysed, and remaining cells resuspended in OpTmizer media. Cells are normalized to one cell concentration and seeded into an IFNγ ELISPOT plate with stimulants for overnight culture. Cells from each individual mouse sample is split into 2 wells: well 1 contains media alone, well 2 contains pooled OLPs (1 μg/ml) specific to the vaccine that the mouse receives. For example, for a mouse immunized with peptides 1-4, the cells are stimulated with OLPs 1a-d, 2a-d, 3a-d and 4a-d (16 individual 15mers overlapping by 11 aa total).

Tumor size is measured 3×/week and subsequently on a daily basis after reaching a specified size threshold. Mice are euthanized when tumors reach maximum size, or tumors became ulcerated and do not heal within 24 hours.

### Example 5. Mouse Cancer Vaccine Study: Antigen Competition (Therapeutic Vaccination)

This therapeutic vaccination study examines whether inhibitory antigens can compete with previously known stimulatory antigens. Two types of competition vaccines are assessed: systemic (where a pool of 3 previously known stimulatory antigens is injected with adjuvant into one site, and a pool of 4 stimulatory or inhibitory antigens is injected without adjuvant into another site), or pooled (where a pool of 3 previously known stimulatory antigens plus a single stimulatory or inhibitory antigen is injected, with adjuvant, into one site).

Methods

The top 8 stimulatory and inhibitory antigens identified and synthesized according to Example 1 are each divided into 2 groups of 4 antigens. Individual lyophilized SLPs are reconstituted in 50% ACN in H2O and pre-mixed, then frozen and lyophilized for 21h and subsequently frozen again as lyophilized pools. These are reconstituted on the day of immunization in either PBS/DMSO or PBS/adjuvants/DMSO (final DMSO concentration: 4%).

B16F10 tumor-bearing mice are vaccinated on the following schedule: cancer cells are injected subcutaneously on the right flank on d0 (ATCC-passage 7, 100K cells in 100 μl of 20% Matrigel), vaccine is injected subcutaneously either at the tail base or scuff of the neck on d3, d10, d17. The experimental groups are injected with: 1) a pool of 3 previously known stimulatory B16F10 antigens: M27 (CD8 neoantigen), M30 (CD4 neoantigen), and Trp2 (CD8 tumor-associated antigen, TAA) plus adjuvant; 2) the same pool of 3 known stimulatory antigens plus adjuvant at one site, and a pool of 4 stimulatory antigens at a second site; 3) the same pool of 3 known stimulatory antigens plus adjuvant at one site, and a pool of 4 inhibitory antigens at a second site; 4) the same pool of 3 known stimulatory antigens plus 1 stimulatory antigen plus adjuvant at one site; or 5) the same pool of 3 known stimulatory antigens plus 1 inhibitory antigen plus adjuvant at one site. The control groups are injected with PBS/DMSO, adjuvant alone, a pool of 4 stimulatory antigens, or a pool of 4 inhibitory antigens. SLPs dosage is 50 μg per SLP/mouse/day.

Heparinized whole blood is collected on d16 of the study (i.e., 6 days after vaccine injection #2), red blood cells are lysed, and remaining cells resuspended in OpTmizer media. Cells are normalized to one cell concentration and seeded into an IFNγ ELISPOT plate with stimulants for overnight culture. Cells from each individual mouse sample is split into 2 wells: well 1 contains media alone, well 2 contains pooled OLPs (1 μg/ml) specific to the vaccine that the mouse receives. For example, for a mouse immunized with peptides 1-4, the cells are stimulated with OLPs 1a-d, 2a-d, 3a-d and 4a-d (16 individual 15mers overlapping by 11 aa total).

Tumor size is measured 3×/week and subsequently on a daily basis after reaching a specified size threshold. Mice are euthanized when tumors reach maximum size, or tumors became ulcerated and do not heal within 24 hours.

### Example 6. Mouse Cancer Vaccine Study: Combination of Vaccine and Checkpoint Inhibitor (Therapeutic Vaccination)

This therapeutic vaccination study includes study arms with and without adjuvant, and with and without checkpoint inhibition (CPI, anti-PD1). The effect of CPI alone or CPI with adjuvant in conjunction with vaccination with pools of stimulatory, inhibitory, and previously known stimulatory antigens is assessed. CPI is administered 1 and 4 days following each of the 3 vaccinations, and then every 3 days for an additional 3 treatments (ending on d30).

Methods

The top 8 stimulatory and inhibitory antigens identified and synthesized according to Example 1 are each divided into 2 groups of 4 antigens. Individual lyophilized SLPs are reconstituted in 50% ACN in H2O and pre-mixed, then frozen and lyophilized for 21h and subsequently frozen again as lyophilized pools. These are reconstituted on the day of immunization in either PBS/DMSO or PBS/adjuvants/DMSO (final DMSO concentration: 4%).

B16F10 tumor-bearing mice are vaccinated on the following schedule: cancer cells are injected subcutaneously on the right flank on d0 (ATCC-passage 7, 100K cells in 100 μl of 20% Matrigel), vaccine is injected subcutaneously at the tail base on d3, d10, d17. CPI is administered 1 and 4 days following each of the 3 vaccinations, and then every 3 days for an additional 3 treatments (ending on d30). The experimental groups are injected subcutaneously at the tail base with: 1) a pool of 4 stimulatory antigens; 2) a pool of 4 inhibitory antigens; 3) a pool of 4 stimulatory antigens plus adjuvant; 4) a pool of 4 inhibitory antigens plus adjuvant; 5) a pool of 3 known stimulatory B16F10 antigens: M27 (CD8 neoantigen), M30 (CD4 neoantigen), and Trp2 (CD8 tumor-associated antigen, TAA) plus adjuvant; 6) a pool of 4 stimulatory antigens plus adjuvant; 7) a pool of 4 inhibitory antigens plus adjuvant; or 8) a pool of 3 known stimulatory antigens plus adjuvant. Each formulation is administered in absence or presence of CPI, as described. The control groups are injected with PBS/DMSO, adjuvant alone, CPI alone, adjuvant and CPI, a pool of 4 stimulatory antigens, or a pool of 4 inhibitory antigens. SLPs dosage is 50 μg per SLP/mouse/day.

Heparinized whole blood is collected on d16 of the study (i.e., 6 days after vaccine injection #2), red blood cells are lysed, and remaining cells resuspended in OpTmizer media. Cells are normalized to one cell concentration and seeded into an IFNγ ELISPOT plate with stimulants for overnight culture. Cells from each individual mouse sample is split into 2 wells: well 1 contains media alone, well 2 contains pooled OLPs (1 μg/ml) specific to the vaccine that the mouse receives. For example, for a mouse immunized with peptides 1-4, the cells are stimulated with OLPs 1a-d, 2a-d, 3a-d and 4a-d (16 individual 15mers overlapping by 11 aa total).

Tumor size is measured 3×/week and subsequently on a daily basis after reaching a specified size threshold. Mice are euthanized when tumors reach maximum size, or tumors became ulcerated and do not heal within 24 hours.

### Example 7. Mouse Tumor Histology

Methods

Tumors were harvested from the euthanized mice of Example 2. Briefly, the top 8 stimulatory and top 8 inhibitory antigens identified and synthesized in Example 1 were divided into 2 groups of 4 stimulatory antigens and 2 groups of 4 inhibitory antigens, respectively. The pools of antigens were used to vaccinate B16F10 tumor-bearing mice with or without triple adjuvant A (CpG, 3D-PHAD, synthetic saponin) on the following schedule: cancer cells were injected on day 0, vaccine was injected on day 3, day 10, and day 17. For SLP-only vaccines, the control group was injected with PBS/DMSO; for adjuvanted vaccines, the control group was injected with triple adjuvant A.

Tumor size was measured 3× per week and subsequently on a daily basis, after reaching a specified size threshold. Mice were euthanized when tumors reached maximum size, or became ulcerated and did not heal within 24 hours. No mice in this study were euthanized for other health reasons.

Harvested tumors were fixed with formalin and stained were stained by fluorescent immunohistochemistry for CD8+(red) with DAPI (blue) as a nuclear counterstain. Tumors were imaged by whole slide scanning and CD8+ T cells were counted by ImageJ software using fluorescent thresholding and size criteria. Graph indicates cell counts from whole tumors.

Results

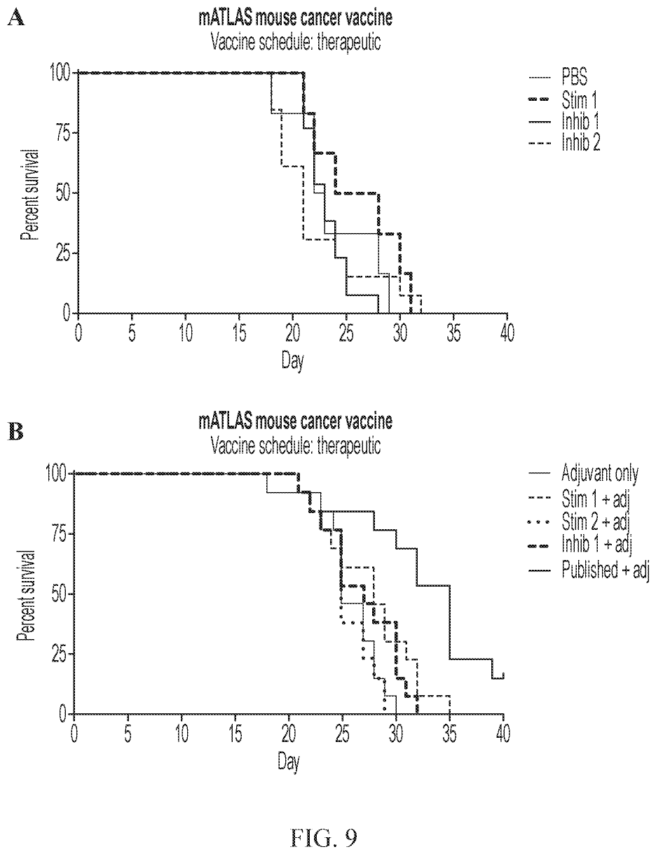

FIG. 10 shows fluorescence scans of representative tumor sections from mice immunized with PBS or a pool of inhibitory antigens. Panel (A) shows a fluorescent CD8+ and DAPI stained section of a representative (average) tumor from a mouse immunized with PBS only. Panel (B) shows a fluorescent CD8+ and DAPI stained section of a representative hyper-progressive tumor from a mouse immunized with a pool of inhibitory antigens only. White arrows point to infiltrating CD8+ T cells (red dots). As can be seen from comparison of Panels A and B, the representative hyper-progressive tumor from mice immunized with inhibitory antigens only contains fewer infiltrating CD8+ T cells than the representative tumor from mice immunized with PBS only.

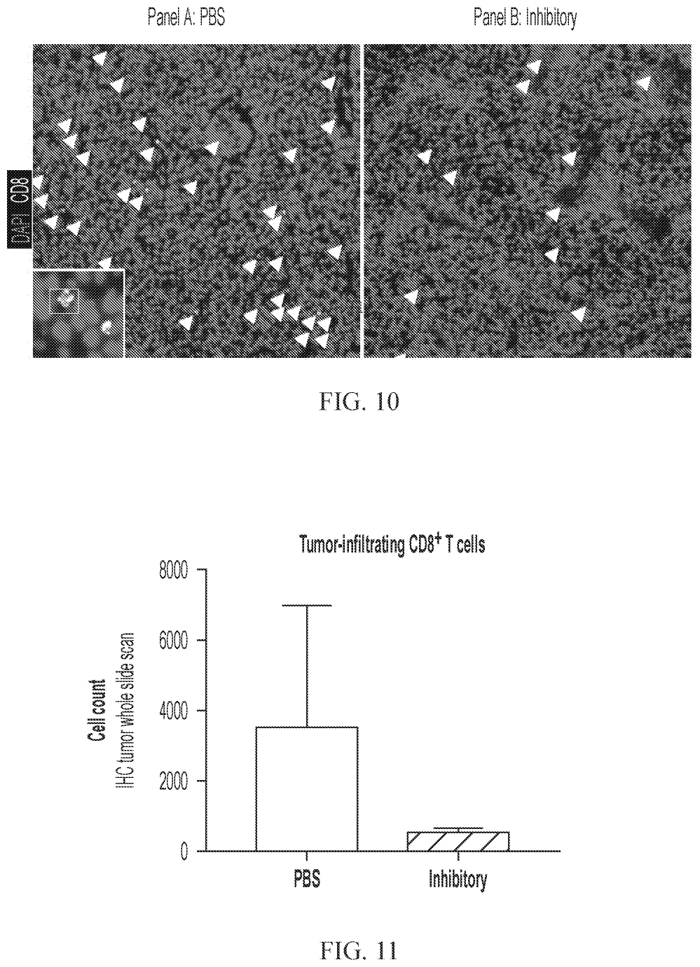

FIG. 11 is a graph showing mean number of infiltrating CD8+ T cells in whole tumors (N=2) from mice treated with PBS only, or a pool of inhibitory antigens only. As in FIG. 10, hyper-progressive tumors from mice immunized with inhibitory antigens contain substantially fewer infiltrating CD8+ T cells than tumors from mice immunized with PBS only.

CD8+ T cell infiltration is considered an indication of anti-tumor immunity and correlates to improved prognosis. Reduced CD8+ T cell infiltration may be a contributing factor to observed hyper-progression of tumors.

### Example 8. Mouse Cancer Vaccine Study: Antigen Competition (Therapeutic Vaccination)

To assess whether inhibitory antigens can compete with known efficacious antigens and decrease protection against tumors, pools of previously published antigens plus single inhibitory antigens identified in Example 1, combined with a triple adjuvant combination of CpG, 3D-PHAD, and QS-21 (denoted triple adjuvant B or Triple), were used to immunize mice.

Methods

The 4 inhibitory antigen constituents of the pool denoted Inhib 2, from Example 1, were re-synthesized. Individual lyophilized SLPs were reconstituted in 50% ACN in H2O and a portion pre-mixed, then frozen and lyophilized for 48h and subsequently frozen again as individual peptides and lyophilized pools. These were reconstituted on the day of immunization in either PBS/DMSO or PBS/adjuvants/DMSO (final DMSO concentration: 4%). The known efficacious antigens were as noted in Example 2: M27 (CD8⁺ neoantigen), M30 (CD4⁺ neoantigen) and Trp2 (CD8⁺ tumor-associated antigen, TAA), shown to have both immunogenicity and efficacy in treating the B16F10 tumor model (Castle J C, Kreiter S et al (2012). Exploiting the Mutanome for Tumor Vaccination. Cancer Research 72(5); Kreiter S et al (2015). Mutant MHC class II epitopes drive therapeutic immune responses to cancer. Nature 520(7549))

B16F10 tumor-bearing mice were vaccinated on the following schedule: cancer cells were injected subcutaneously on the right flank on day 0 (ATCC-passage 6, 100K cells in 100 μl of 20% Matrigel), vaccine was injected subcutaneously at the tail base on day 3, day 10, and day 17. The experimental groups were injected with: 1) a pool of 2 previously known efficacious B16F10 antigens, denoted Published: M30 (CD4⁺ neoantigen) and Trp2 (CD8⁺ tumor-associated antigen, TAA), with triple adjuvant B; 2) the same pool as 1) plus all 4 inhibitory antigens of the Inhib 2 pool (described in Example 1), with triple adjuvant B; 3-4) the same pool as 1) plus one each of two of the 4 inhibitory antigen constituents of the Inhib 2 pool (In21, In17), with triple adjuvant B. The control group was injected with triple adjuvant B only. SLPs dosage was 50 μg per SLP/mouse/day.

Tumor size was measured 3× per week and subsequently on a daily basis, after reaching a specified size threshold. Mice are euthanized when tumors reached maximum size, or became ulcerated and did not heal within 24 hours.

Results

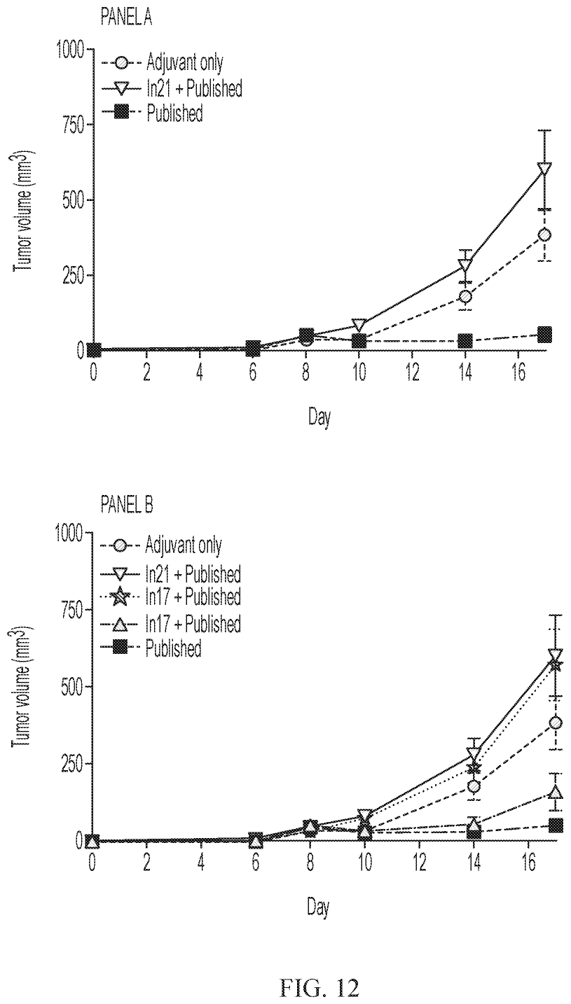

FIG. 12 shows that addition of an inhibitory antigen can significantly abrogate protective effects of known efficacious antigens. In Panel A, immunization with a pool comprising inhibitory antigen In21 and known efficacious antigens reversed the protection from tumor growth observed with the pool of known efficacious antigens alone (Published), to a greater degree even than the adjuvant-only negative control. Panel B shows variability in the deleterious effects of inhibitory antigens. Immunization with a pool comprising inhibitory antigen In17 and known efficacious antigens resulted in slight reduction of protection.

### Example 9. Mouse Cancer Vaccine Study H (Therapeutic Vaccination)

Methods

The 4 inhibitory antigen constituents of the pool denoted Inhib 2, from Example 1, were re-synthesized. Individual lyophilized SLPs were reconstituted in 50% ACN in H2O and pre-mixed, then frozen and lyophilized for 48h and subsequently frozen again as lyophilized pools. These were reconstituted on the day of immunization in either PBS/DMSO or PBS/adjuvants/DMSO (final DMSO concentration: 4%).

The Inhib 2 pool of 4 inhibitory antigens was combined with triple adjuvant B (CpG, 3D-PHAD, QS21) and used to immunize B16F10 tumor-bearing mice on the following schedule: cancer cells were injected subcutaneously on the right flank on day 0 (ATCC-passage 6, 100K cells in 100 ul of 20% Matrigel), vaccine formulations were administered subcutaneously in the tail base on day 3, day 10, and day 17. The control group was injected with triple adjuvant B only. SLPs dosage was 50 ug per SLP/mouse/day. Triple adjuvant B dosage was CpG (5 ug/mouse), 3D-PHAD (5 ug/mouse), and QS21 (25 ug prime, 12.5 ug boost/mouse).

Heparinized whole blood was collected on day 17 of the study (i.e., 6 days after vaccine injection #2), red blood cells were lysed, and remaining cells resuspended in OpTmizer media. Cells were counted by a Guava instrument, normalized to one cell concentration, and seeded into an IFNγ ELISPOT plate with stimulants for overnight culture. Cells from each individual mouse sample were split into 2 wells: well 1 contained media alone, well 2 contained pooled OLPs (1 μg/ml) specific to the vaccine that the mouse received, i.e., for a mouse immunized with peptide antigens 5-8 (Inhib 2 pool), the cells were stimulated with OLPs 5a-d, 6a-d, 7a-d and 8a-d (16 individual 15mers overlapping by 1 laa total).

Tumor size was measured 3× per week and subsequently on a daily basis, after reaching a specified size threshold. Mice were euthanized when tumors reached maximum size, or became ulcerated and did not heal within 24 hours. No mice in this study were euthanized for other health reasons.

Results

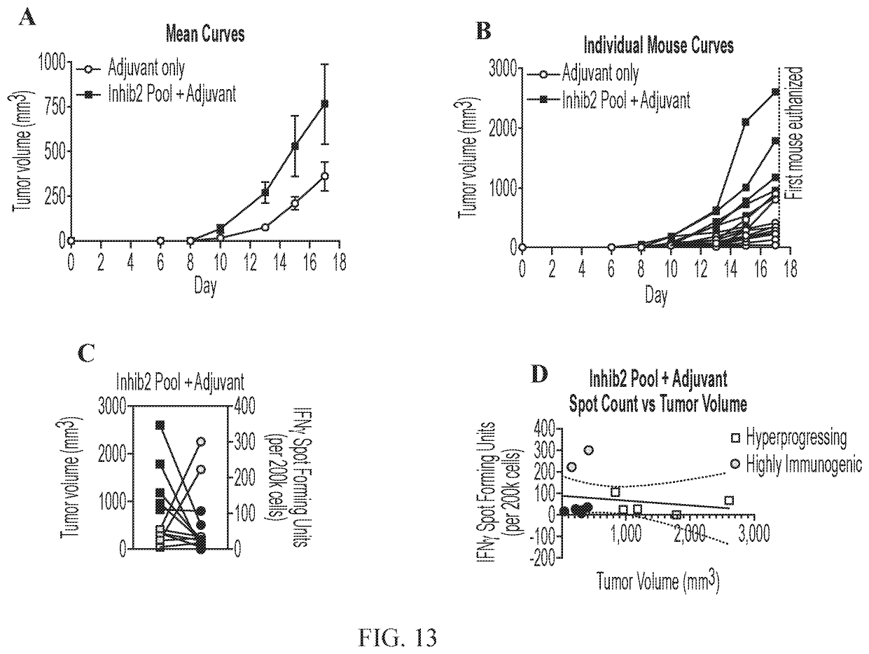

FIG. 13 shows results of therapeutic immunization with the Inhib 2 pool of 4 inhibitory antigens combined with triple adjuvant B. Approximately half of the immunized mice had a marked and significant increase in tumor growth kinetics (hyper-progression), as compared to control immunization with triple adjuvant B only. Hyper-progression correlated with lower IFNγ secretion, i.e., lower immune response. Results for Panels A-B are expressed as tumor volume in mm³ over time. Panel A shows mean curves for the two immunization groups. Panel B shows curves for individual mice in the two immunization groups. Panels C and D show the correlation between tumor volume in mm³ and IFNγ spot forming units per 200K cells. These results contrast with results obtained for the Inhib 1 pool combined with triple adjuvant A (CpG, 3D-PHAD, synthetic saponin) shown in FIG. 8, suggesting that hyper-progression may be adjuvant-dependent, antigen-dependent, or both.

### Example 10. Differential Impact of Adjuvanted Inhibitory Antigens on Tumor Growth in Mice

Methods

The 4 inhibitory antigen constituents of the pool denoted Inhib 2, from Example 1, were re-synthesized. Individual lyophilized SLPs were reconstituted in 50% ACN in H2O and pre-mixed, then frozen and lyophilized for 48h and subsequently frozen again as lyophilized pools. These were reconstituted on the day of immunization in either PBS/DMSO or PBS/adjuvants/DMSO (final DMSO concentration: 4%).

Pools of 4 inhibitory antigens were used to vaccinate B16F10 tumor-bearing mice with or without the following adjuvants: 1) incomplete Freund's adjuvant (IFA); 2) CpG; 3) poly-IC; or 4) triple adjuvant B (CpG, 3D-PHAD, QS-21). The following schedule was employed: cancer cells were injected on day 0 (ATCC-passage 6, 100K cells in 100 μl of 20% Matrigel, subcutaneously on the right flank). Vaccine was injected on day 3, day 10, and day 17. Control groups were injected with PBS or each adjuvant alone, in the absence of antigens. Fifteen mice per group were evaluated. SLP dosage was 50 μg per SLP/mouse/day. Adjuvant dosage per mouse per day was: IFA=1:1 emulsion with antigens; CpG (5 μg); poly-IC (5 μg); triple adjuvant B=prime: QS-21 (25 μg), 3D-PHAD (5 μg), CpG (5 μg) and boost: QS-21 (12.5 μg), 3D-PHAD (5 μg), CpG (5 μg). The final formulated vaccines were injected by subcutaneous tail base injection (50 μl on each side of the tail base for a total of 100 μl).

Blood was drawn by retro-orbital bleed on day 17 of the study (i.e., 6 days after vaccine injection #2), red blood cells were lysed, and remaining cells resuspended in OpTmizer media. In addition, in a subset of mice, spleens and draining lymph nodes were collected between days 20-35. Cells were counted by a Guava instrument, normalized to one cell concentration, and seeded onto an IFNγ ELISPOT plate with stimulants for overnight culture. Each individual mouse blood or cell sample was split into 2 wells, and stimulated with media only or with overlapping peptides (OLPs; 15mers overlapping by 11 aa) spanning each of the vaccine antigens. Each OLP was used at 1 μg/ml in the overnight ELISPOT culture plate.

Tumor size was measured 3× per week and subsequently on a daily basis after reaching a specified size threshold (2000 mm³). Mice were euthanized when tumors reached maximum size, or became ulcerated and did not heal within 24 hours. No mice in this study were euthanized for other health reasons.

Results

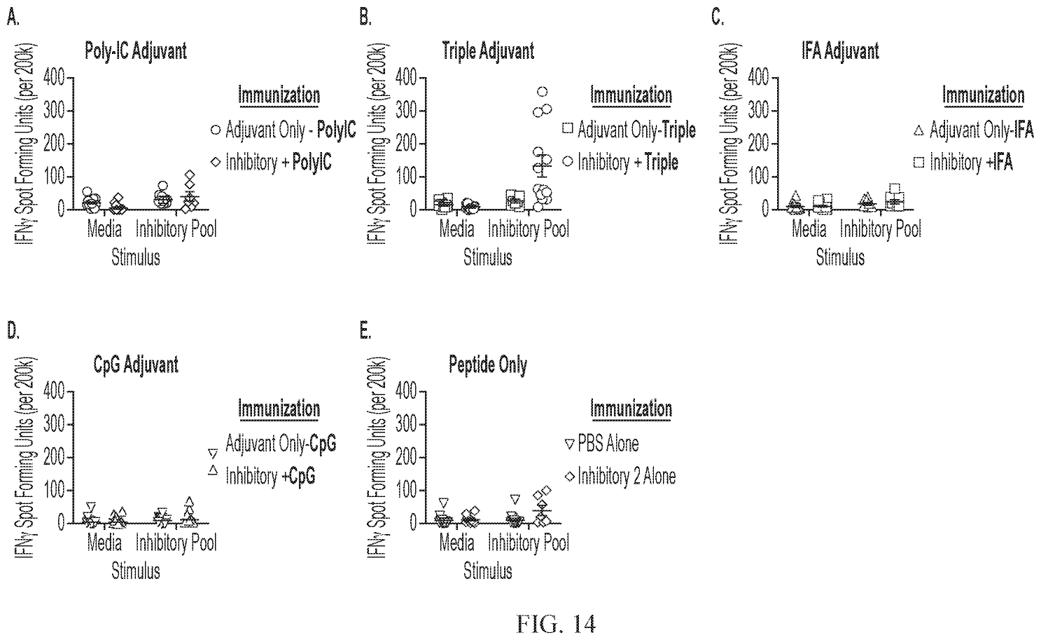

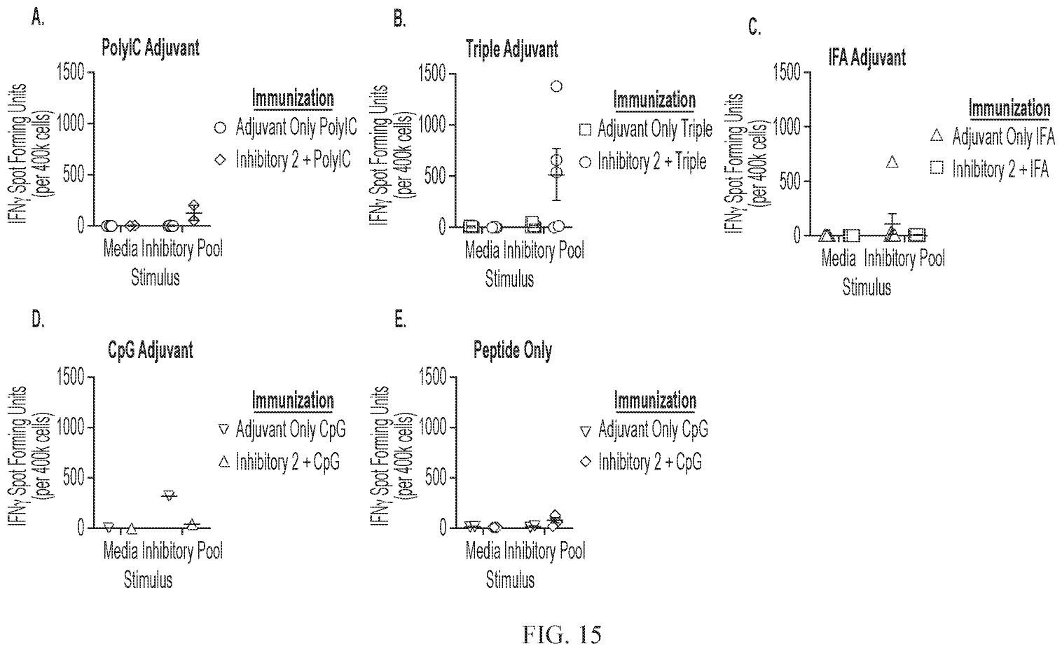

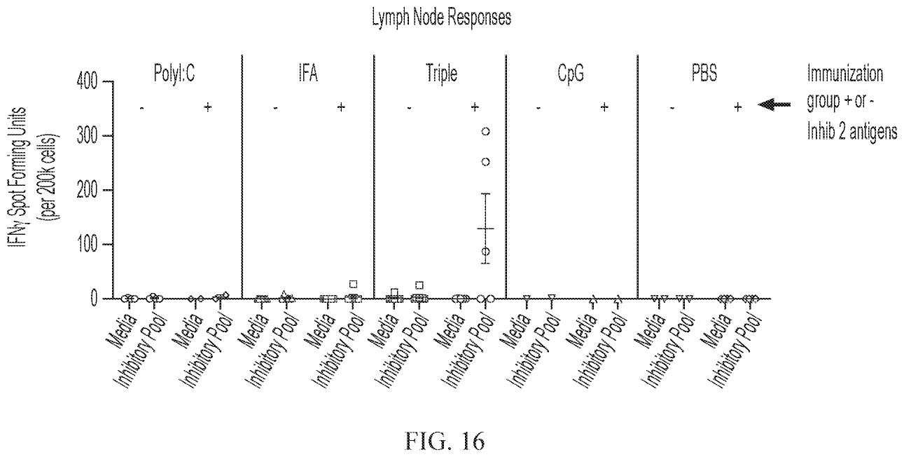

As shown in FIG. 14, mice that were vaccinated with pools of 4 inhibitory antigens with or without adjuvant generally did not secrete IFNγ above the adjuvant-only control level upon stimulation. The one exception was mice that were vaccinated with antigens combined with triple adjuvant B, where there was a statistically significant increase in cytokine secretion from peripheral blood T cells in response to vaccination. The effect was observed in approximately half of the mice, i.e., half responded, and half failed to respond. The same was true for splenocytes (FIG. 15) and lymph node cells (FIG. 16) evaluated from a subset of mice in the study; there was a large increase in the proportion of cells secreting IFNγ in about half of the mice evaluated in the group immunized with inhibitory antigens and triple adjuvant B. None of the other adjuvants induced stimulatory T cell responses in splenocytes or lymph node cells of the immunized tumor-bearing mice.

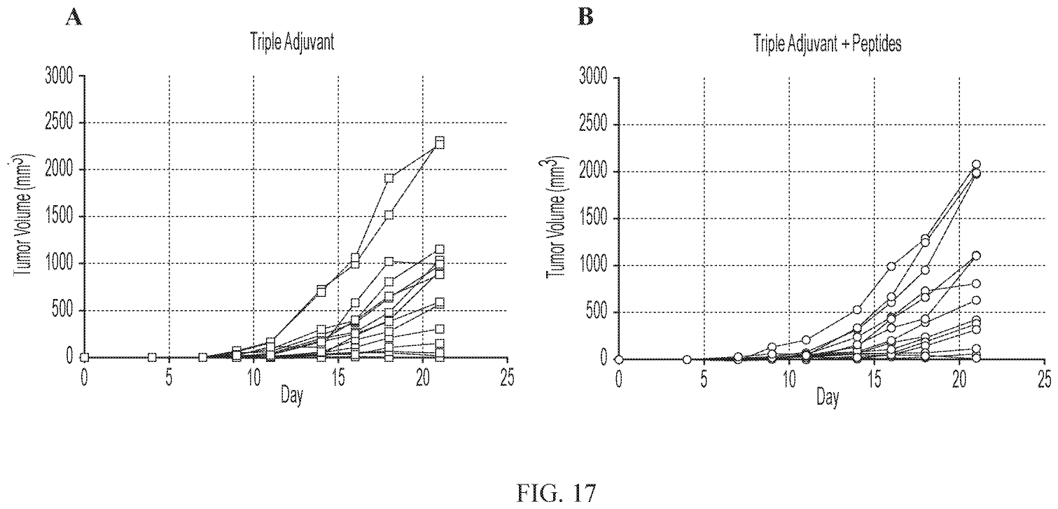

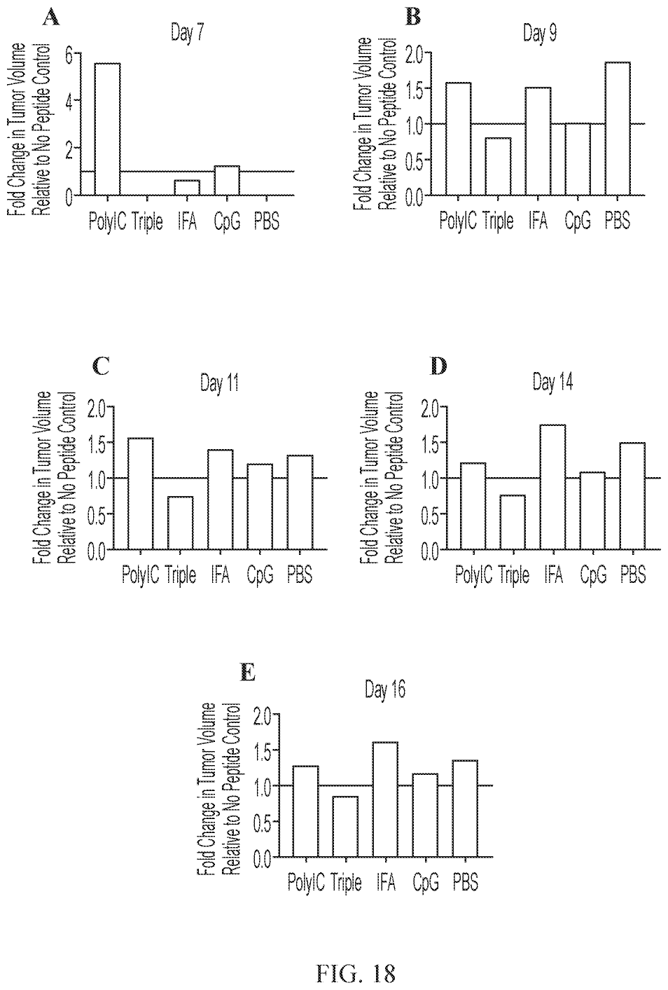

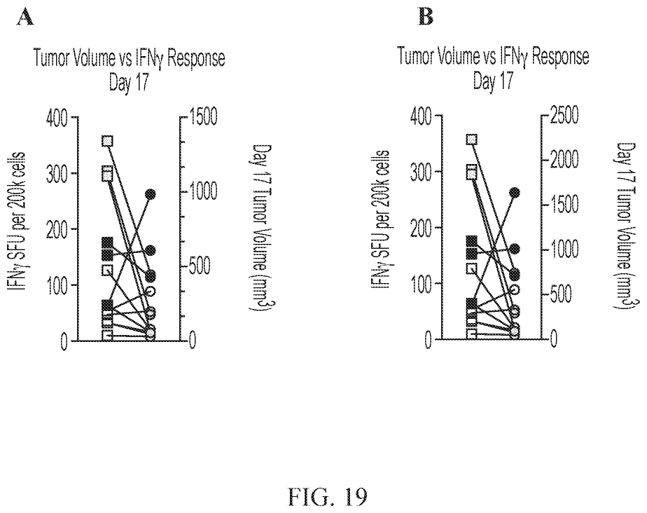

Strikingly, therapeutic immunization with different adjuvants led to different kinetics of tumor growth. Consistent with the immunogenicity data shown in FIG. 14-16, mice that received inhibitory antigens with triple adjuvant B showed slightly reduced tumor growth kinetics compared to mice that received triple adjuvant B only. The growth curves in FIG. 17 show a delay of tumor growth in mice with tumors exceeding 500 mm² (day 14 for adjuvant only and day 17 for adjuvant plus antigens), as well as no mice reaching tumor sizes exceeding 1500 mm² by day 18, and fewer mice reaching 1000 mm² or exceeding 1500 mm² by day 21 in the antigen-containing group. In contrast, as shown in FIG. 18, on day 7, mice vaccinated with inhibitory antigens adjuvanted with poly-IC had marked increase in tumor size relative to mice who received poly-IC only (or any of the other groups). This effect was maintained throughout the time-course, although the fold-change decreased with time. Similarly, mice that received unadjuvanted inhibitory antigens or inhibitory antigens adjuvanted with IFA had larger tumor sizes relative to mice that received PBS or IFA only, respectively. By day 17 of the study, mice that received inhibitory antigens adjuvanted with IFA maintained tumor sizes that were 1.5-fold higher than their IFA only counterparts. In contrast, there was essentially no difference in tumor growth between mice that received CpG with inhibitory antigens and those that received CpG alone. FIG. 19 shows the correlation between tumor volume in mm³ and IFNγ spot forming units per 200K cells for the Inhib 2+triple adjuvant B immunization group. As in Example 9, hyper-progression correlated with lower IFNγ secretion, i.e., lower immune response.

Taken together, these results demonstrate that immune responses and control of tumor growth in response to vaccination with inhibitory antigens are malleable. With the appropriate adjuvant, responses to inhibitory antigens that impair or reduce immune control of tumors can be abrogated.

| LISTING OF SEQUENCES | |
|---|---|
| Heparanase isoform 1, preproprotein, NP_001092010.1, NP_006656.2 | |
| (SEQ ID NO: 6) | |
| 1 | mllrskpalp pplmllllgp lgplspgalp rpaqaqdvvd ldfftqeplh lvspsflsvt |
| 61 | idanlatdpr flillgspkl rtlarglspa ylrfggtktd flifdpkkes tfeersywqs |
| 121 | qvnqdickyg sippdveekl rlewpyqeql llrehyqkkf knstysrssv dvlytfancs |
| 181 | gldlifglna llrtadlqwn ssnaqllldy csskgynisw elgnepnsfl kkadifings |
| 241 | qlgedfiqlh kllrkstfkn aklygpdvgq prrktakmlk sflkaggevi dsvtwhhyyl |
| 301 | ngrtatkedf lnpdvldifi ssvqkvfqvv estrpgkkvw lgetssaygg gapllsdtfa |
| 361 | agfmwldklg lsarmgievv mrqvffgagn yhlvdenfdp lpdywlsllf kklvgtkvlm |
| 421 | asvqgskrrk lrvylhctnt dnprykegdl tlyainlhnv tkylrlpypf snkqvdkyll |
| 481 | rplgphglls ksvqlngltl kmvddqtlpp lmekplrpgs slglpafsys ffvirnakva |
| 541 | aci |
| Heparanase isoform 2, preproprotein, NP_001159970.1 | |
| (SEQ ID NO: 7) | |
| 1 | mllrskpalp pplmllllgp lgplspgalp rpaqaqdvvd ldfftqeplh lvspsflsvt |
| 61 | idanlatdpr flillgspkl rtlarglspa ylrfggtktd flifdpkkes tfeersywqs |
| 121 | qvnqdickyg sippdveekl rlewpygeql llrehyqkkf knstysrssv dvlytfancs |
| 181 | gldlifglna llrtadlqwn ssnaqllldy csskgynisw elgnepnsfl kkadifings |
| 241 | qlgedfiqlh kllrkstfkn aklygpdvgq prrktakmlk sflkaggevi dsvtwhhyyl |
| 301 | ngrtatkedf lnpdvldifi ssvqkvfqdy wlsllfkklv gtkvlmasvq gskrrklrvy |
| 361 | lhctntdnpr ykegdltlya inlhnvtkyl rlpypfsnkq vdkyllrplg phgllsksvq |
| 421 | lngltlkmvd dqtlpplmek plrpgsslgl pafsysffvi rnakvaaci |
| SMAD family member 4, mothers against decapentaplegic homolog 4, | |
| NP_005350.1 | |
| (SEQ ID NO: 8) | |
| 1 | mdnmsitntp tsndaclsiv hslmchrqgg esetfakrai eslvkklkek kdeldslita |
| 61 | ittngahpsk cvtiqrtldg rlqvagrkgf phviyarlwr wpdlhknelk hvkycqyafd |
| 121 | lkcdsvcvnp yhyervvspg idlsgltlqs napssmmvkd eyvhdfegqp slsteghsiq |
| 181 | tiqhppsnra stetystpal lapsesnats tanfpnipva stsqpasilg gshsegllqi |
| 241 | asgpqpgqqq ngftgqpaty hhnstttwtg srtapytpnl phhqnghlqh hppmpphpgh |
| 301 | ywpvhnelaf qppisnhpap eywcsiayfe mdvqvgetfk vpsscpivtv dgyvdpsggd |
| 361 | rfclgqlsnv hrteaierar lhigkgvqle ckgegdvwvr clsdhavfvq syyldreagr |
| 421 | apgdavhkiy psayikvfdl rqchrqmqqq aataqaaaaa qaaavagnip gpgsvggiap |
| 481 | aislsaaagi gvddlrrlci lrmsfvkgwg pdyprqsike tpcwieihlh ralqlldevl |
| 541 | htmpiadpqp ld |
| Cadherin 3, isoform 1 preproprotein, NP_001784.2 | |
| 1 | mglprgplas llllqvcwlq caaseperav freaevtlea ggaeqepgqa lgkvfmgcpg |
| 61 | qepalfstdn ddftvrnget vqerrslker nplkifpskr ilrrhkrdwv vapisvpeng |
| 121 | kgpfpqrlnq lksnkdrdtk ifysitgpga dsppegvfav eketgwllln kpldreeiak |
| 181 | yelfghavse ngasvedpmn isiivtdqnd hkpkftqdtf rgsvlegvlp gtsvmqvtat |
| 241 | deddaiytyn gvvaysihsq epkdphdlmf tihrstgtis vissgldrek vpeytltiqa |
| 301 | tdmdgdgstt tavavveild andnapmfdp qkyeahvpen avghevqrlt vtdldapnsp |
| 361 | awratylimg gddgdhftit thpesnqgil ttrkgldfea knqhtlyvev tneapfvlkl |
| 421 | ptstativvh vedvneapvf vppskvvevq egiptgepvc vytaedpdke nqkisyrilr |
| 481 | dpagwlamdp dsgqvtavgt ldredeqfvr nniyevmvla mdngsppttg tgtllltlid |
| 541 | vndhgpvpep rqiticngsp vrqvlnitdk dlsphtspfq aqltddsdiy wtaevneegd |
| 601 | tvvlslkkfl kqdtydvhls lsdhgnkeql tviratvcdc hghvetcpgp wkggfilpvl |
| 661 | gavlallfll lvllllvrkk rkikeplllp eddtrdnvfy ygeegggeed qdyditqlhr |
| 721 | glearpevvl rndvaptiip tpmyrprpan pdeignfiie nlkaantdpt appydtllvf |
| 781 | dyegsgsdaa slssltssas dqdqdydyln ewgsrfkkla dmygggedd |
| Cadherin 3, isoform 2 precursor, NP_001304124.1 | |
| 1 | mglprgplas llllqvcwlq caasepcrav freaevtlea ggaeqepgqa lgkvfmgcpg |
| 61 | qepalfstdn ddftvrnget vqerrslker nplkifpskr ilrrhkrdwv vapisvpeng |
| 121 | kgpfpqrlnq lksnkdrdtk ifysitgpga dsppegvfav eketgwllln kpldreeiak |
| 181 | yelfghavse ngasvedpmn isiivtdqnd hkpkftqdtf rgsvlegvlp gtsvmqvtat |
| 241 | deddaiytyn gvvaysihsq epkdphdlmf tihrstgtis vissgldrek vpeytltiqa |
| 301 | tdmdgdgstt tavavveild andnapmfdp qkyeahvpen avghevqrlt vtdldapnsp |
| 361 | awratylimg gddgdhftit thpesnqgil ttrkgldfea knqhtlyvev tneapfvlkl |
| 421 | ptstativvh vedvneapvf vppskvvevq egiptgepvc vytaedpdke nqkisyrilr |
| 481 | dpagwlamdp dsgqvtavgt ldredeqfvr nniyevmvla mdngsppttg tgtllltlid |
| 541 | vndhgpvpep rqiticnqsp vrqvlnitdk dlsphtspfq aqltddsdiy wtaevneegd |
| 601 | tvvlslkkfl kqdtydvhls lsdhgnkeql tviratvcdc hghvetcpgp wkggfilpvl |
| 661 | gavlallfll lvllllvrkk rkikeplllp eddtrdnvfy ygeegggeed qdyditqlhr |
| 721 | glearpevvl rndvaptiip tpmyrprpan pdeignfiie grgergsqrg ngglqlargr |
| 781 | trrs |
| Cadherin 3, isoform 3, NP_001304125.1 | |
| 1 | mgcpgqepal fstdnddftv rngetvqerr slkernplki fpskrilrrh krdwvvapis |
| 61 | vpengkgpfp qrlnqlksnk drdtkifysi tgpgadsppe gvfaveketg wlllnkpldr |
| 121 | eeiakyelfg havsengasv edpmnisiiv tdqndhkpkf tqdtfrgsvl egvlpgtsvm |
| 181 | qvtatdedda iytyngvvay sihsqepkdp hdlmftihrs tgtisvissg ldrekvpeyt |
| 241 | ltiqatdmdg dgstttavav veildandna pmfdpqkyea hvpenavghe vqrltvtdld |
| 301 | apnspawrat ylimggddgd hftitthpes nqgilttrkg ldfeaknqht lyvevtneap |
| 361 | fvlklptsta tivvhvedvn eapvfvppsk vvevqegipt gepvcvytae dpdkenqkis |
| 421 | yrilrdpagw lamdpdsgqv tavgtldred eqfvrnniye vmvlamdngs ppttgtgtll |
| 481 | ltlidvndhg pvpeprqiti cnqspvrqvl nitdkdlsph tspfqaqltd dsdiywtaev |
| 541 | neegdtvvls lkkflkqdty dvhlslsdhg nkeqltvira tvcdchghve tcpgpwkggf |
| 601 | ilpvlgavla llflllvlll lvrkkrkike plllpeddtr dnvfyygeeg ggeedqdydi |
| 661 | tqlhrglear pevvlrndva ptiiptpmyr prpanpdeig nfiienlkaa ntdptappyd |
| 721 | tllvfdyegs gsdaaslssl tssasdqdqd ydylnewgsr fkkladmygg gedd |
| Chorionic gonadotropin beta subunit 3, precursor, NP_000728.1 | |
| 1 | memfggllll lllsmggtwa skeplrprcr pinatlavek egcpvcitvn tticagycpt |
| 61 | mtrvlqgvlp alpqvvcnyr dvrfesirlp gcprgvnpvv syavalscqc alcrrsttdc |
| 121 | ggpkdhpltc ddprfqdsss skapppslps psrlpgpsdt pilpq |
| Chorionic gonadotropin beta subunit 5, precursor, NP_149032.1 | |
| 1 | memfqgllll lllsmggtwa skeplrprcr pinatlavek egcpvcitvn tticagycpt |
| 61 | mtrvlqgvlp alpqvvcnyr dvrfesirlp gcprgvnpvv syavalscqc alcrrsttdc |
| 121 | ggpkdhpltc ddprfqdsss skapppslps psrlpgpsdt pilpq |
| Cytochrome c oxidase assembly factor 1 homolog, isoform a, | |
| NP_001308126.1, NP_001308127.1, NP_001308128.1, NP_001308129.1, | |
| NP_001337853.1, NP_001337854.1, NP_001337855.1, NP_001337856.1, | |
| NP_060694.2 | |
| 1 | mmwqkyagsr rsmplgaril fhgvfyaggf aivyyliqkf hsralyykla veqlqshpea |
| 61 | qealgpplni hylklidren fvdivdaklk ipvsgskseg llyvhssrgg pfqrwhldev |
| 121 | flelkdgqqi pvfklsgeng devkke |
| Cytochrome c oxidase assembly factor 1 homolog, isoform b, | |
| NP_001308130.1 | |
| 1 | mplgarilfh gvfyaggfai vyyliqkfhs ralyyklave qlqshpeage algpplnihy |
| 61 | lklidrenfv divdaklkip vsgsksegll yvhssrggpf qrwhldevfl elkdgqqipv |
| 121 | fklsgengde vkke |
| Cytochrome c oxidase assembly factor 1 homolog, isoform c, | |
| NP_001308131.1, NP_001308132.1, NP_001308133.1, NP_001308134.1 | |
| 1 | mmwqkyagsr rsmplgaril fhgvfyaggf aivyyliqsk ypasrlrpdl llacscssir |
| 61 | gnt |
| Cytochrome c oxidase assembly factor 1 homolog, isoform d, | |
| NP_001337857.1 | |
| 1 | mqeaggqclw eqgsfstvcs mpgalplcit sfkfhsraly yklaveqlqs hpeaqealgp |
| 61 | plnihylkli drenfvdivd aklkipvsgs ksegllyvhs srggpfqrwh ldevflelkd |
| 121 | gqqipvfkls gengdevkke |
| Estrogen receptor binding site associated, antigen, 9, NP_001265867.1, | |
| NP_004206.1, NP_936056.1, NP_001308129.1, | |
| 1 | maitqfrlfk fctclatvfs flkrlicrsg rgrklsgdqi tlpttvdyss vpkqtdveew |
| 61 | tswdedapts vkieggngnv atqqnsleql epdyfkdmtp tirktqkivi kkreplnfgi |
| 121 | pdgstgfssr laatqdlpfi hqsselgdld twqentnawe eeedaawqae evlrqqklad |
| 181 | rekraaeqqr kkmekeaqrl mkkeqnkigv kls |
| ETS transcription factor, isoform a, NP_001964.2 | |
| 1 | mdsaitlwqf llqllqkpqn khmicwtsnd gqfkllqaee varlwgirkn kpnmnydkls |
| 61 | ralryyyvkn iikkvngqkf vykfvsypei lnmdpmtvgr iegdceslnf sevsssskdv |
| 121 | enggkdkppq pgaktssrnd yihsglyssf tlnslnssnv klfklikten paeklaekks |
| 181 | pqeptpsvik fvttpskkpp vepvaatisi gpsispssee tiqaletlvs pklpsleapt |
| 241 | sasnvmtafa ttppissipp lqepprtpsp plsshpdidt didsvasqpm elpenlslep |
| 301 | kdqdsvllek dkvnnssrsk kpkglelapt lvitssdpsp lgilspslpt asltpaffsq |
| 361 | tpiiltpspl lssihfwstl spvaplspar lqgantlfqf psvlnshgpf tlsgldgpst |
| 421 | pgpfspdlqk t |
| ETS transcription factor, isoform b, NP_068567.1 | |
| 1 | mdsaitlwqf llqllqkpqn khmicwtsnd gqfkllqaee varlwgirkn kpnmnydkls |
| 61 | ralryyyvkn iikkvngqkf vykfvsypei lnmdpmtvgr iegdceslnf sevsssskdv |
| 121 | enggkdkppq pgaktssrnd yihsglyssf tlnslnssnv klfklikten paeklaekks |
| 181 | pqeptpsvik fvttpskkpp vepvaatisi gpsispssee tiqaletlvs pklpsleapt |
| 241 | sasnvmtafa ttppissipp lqepprtpsp plsshpdidt didsvasqpm elpenlslep |
| 301 | kdqdsvllek dkvnnssrsk kpkglelapt lvitssdpsp lgilspslpt asltpaffsq |
| 361 | vacslfmvsp llsficpfkg ignlytqvcf lllrfvlerl cvtvm |
| Receptor tyrosine-protein kinase erbB-2, isoform a precursor, | |
| NP_004439.2 | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytmrrl lqetelvepl tpsgampnqa qmrilketel |
| 721 | rkvkvlgsga fgtvykgiwi pdgenvkipv aikvlrents pkankeilde ayvmagvgsp |
| 781 | yvsrllgicl tstvqlvtql mpygclldhv renrgrlgsq dllnwcmqia kgmsyledvr |
| 841 | lvhrdlaarn vlvkspnhvk itdfglarll dideteyhad ggkvpikwma lesilrrrft |
| 901 | hqsdvwsygv tvwelmtfga kpydgipare ipdllekger lpqppictid vymimvkcwm |
| 961 | idsecrprfr elvsefsrma rdpqrfvviq nedlgpaspl dstfyrslle dddmgdlvda |
| 1021 | eeylvpqqgf fcpdpapgag gmvhhrhrss strsgggdlt lglepseeea prsplapseg |
| 1081 | agsdvfdgdl gmgaakglqs lpthdpsplq rysedptvpl psetdgyvap ltcspqpeyv |
| 1141 | nqpdvrpqpp spregplpaa rpagatlerp ktlspgkngv vkdvfafgga venpeyltpq |
| 1201 | ggaapqphpp pafspafdnl yywdqdpper gappstfkgt ptaenpeylg ldvpv |
| Receptor tyrosine-protein kinase erbB-2, isoform b, NP_001005862.1 | |
| 1 | mklrlpaspe thldmlrhly qgcqvvqgnl eltylptnas lsflqdiqev qgyvliahnq |
| 61 | vrqvplqrlr ivrgtqlfed nyalavldng dplnnttpvt gaspgglrel qlrslteilk |
| 121 | ggvliqrnpq lcyqdtilwk difhknnqla ltlidtnrsr achpcspmck gsrcwgesse |
| 181 | dcqsltrtvc aggcarckgp lptdccheqc aagctgpkhs dclaclhfnh sgicelhcpa |
| 241 | lvtyntdtfe smpnpegryt fgascvtacp ynylstdvgs ctlvcplhnq evtaedgtqr |
| 301 | cekcskpcar vcyglgmehl revravtsan iqefagckki fgslaflpes fdgdpasnta |
| 361 | plqpeqlqvf etleeitgyl yisawpdslp dlsvfqnlqv irgrilhnga ysltlqglgi |
| 421 | swlglrslre lgsglalihh nthlcfvhtv pwdqlfrnph qallhtanrp edecvgegla |
| 481 | chqlcarghc wgpgptqcvn csqflrggec veecrvlqgl preyvnarhc lpchpecqpq |
| 541 | ngsvtcfgpe adqcvacahy kdppfcvarc psgvkpdlsy mpiwkfpdee gacqpcpinc |
| 601 | thscvdlddk gcpaegrasp ltsiisavvg illvvvlgvv fgilikrrqq kirkytmrrl |
| 661 | lqetelvepl tpsgampnqa qmrilketel rkvkvlgsga fgtvykgiwi pdgenvkipv |
| 721 | aikvlrents pkankeilde ayvmagvgsp yvsrllgicl tstvqlvtql mpygclldhv |
| 781 | renrgrlgsq dllnwcmqia kgmsyledvr lvhrdlaarn vlvkspnhvk itdfglarll |
| 841 | dideteyhad ggkvpikwma lesilrrrft hqsdvwsygv tvwelmtfga kpydgipare |
| 901 | ipdllekger lpqppictid vymimvkcwm idsecrprfr elvsefsrma rdpqrfvviq |
| 961 | nedlgpaspl dstfyrslle dddmgdlvda eeylvpqqgf fcpdpapgag gmvhhrhrss |
| 1021 | strsgggdlt lglepseeea prsplapseg agsdvfdgdl gmgaakglqs lpthdpsplq |
| 1081 | rysedptvpl psetdgyvap ltcspqpeyv nqpdvrpqpp spregplpaa rpagatlerp |
| 1141 | ktlspgkngv vkdvfafgga venpeyltpq ggaapqphpp pafspafdnl yywdqdpper |
| 1201 | gappstfkgt ptaenpeylg ldvpv |
| Receptor tyrosine-protein kinase erbB-2, isoform c, NP_001276865.1 | |
| 1 | mprgswkpqv ctgtdmklrl paspethldm lrhlyqgcqv vqgnleltyl ptnaslsflq |
| 61 | diqevqgyvl iahnqvrqvp lqrlrivrgt qlfednyala vldngdplnn ttpvtgaspg |
| 121 | glrelqlrsl teilkggvli grnpqlcyqd tilwkdifhk nnqlaltlid tnrsrachpc |
| 181 | spmckgsrcw gessedcqsl trtvcaggca rckgplptdc cheqcaagct gpkhsdclac |
| 241 | lhfnhsgice lhcpalvtyn tdtfesmpnp egrytfgasc vtacpynyls tdvgsctlvc |
| 301 | plhnqevtae dgtqrcekcs kpcarvcygl gmehlrevra vtsaniqefa gckkifgsla |
| 361 | flpesfdgdp asntaplqpe qlqvfetlee itgylyisaw pdslpdlsvf qnlqvirgri |
| 421 | lhngaysltl qglgiswlgl rslrelgsgl alihhnthlc fvhtvpwdql frnphqallh |
| 481 | tanrpedecv geglachqlc arghcwgpgp tqcvncsqfl rgqecveecr vlqglpreyv |
| 541 | narhclpchp ecqpqngsvt cfgpeadqcv acahykdppf cvarcpsgvk pdlsympiwk |
| 601 | fpdeegacqp cpincthscv dlddkgcpae qraspltsii savvgillvv vlgvvfgili |
| 661 | krrqqkirky tmrrllqete lvepltpsga mpnqaqmril ketelrkvkv lgsgafgtvy |
| 721 | kgiwipdgen vkipvaikvl rentspkank eildeayvma gvgspyvsrl lgicltstvq |
| 781 | lvtqlmpygc lldhvrenrg rlgsqdllnw cmqiakgmsy ledvrlvhrd laarnvlvks |
| 841 | pnhvkitdfg larlldidet eyhadggkvp ikwmalesil rrrfthqsdv wsygvtvwel |
| 901 | mtfgakpydg ipareipdll ekgerlpqpp ictidvymim vkcwmidsec rprfrelvse |
| 961 | fsrmardpqr fvviqnedlg paspldstfy rslledddmg dlvdaeeylv pqqgffcpdp |
| 1021 | apgaggmvhh rhrssstrsg ggdltlglep seeeaprspl apsegagsdv fdgdlgmgaa |
| 1081 | kglqslpthd psplqrysed ptvplpsetd gyvapltcsp qpeyvnqpdv rpqppspreg |
| 1141 | plpaarpaga tlerpktlsp gkngvvkdvf afggavenpe yltpqggaap qphpppafsp |
| 1201 | afdnlyywdq dppergapps tfkgtptaen peylgldvpv |
| Receptor tyrosine-protein kinase erbB-2, isoform d precursor, | |
| NP_001276866.1 | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaegrasp ltsiisavvg |
| 661 | illvvvlgvv fgilikrrqq kirkytmrrl lqetelvepl tpsgampnqa qmrilketel |
| 721 | rkvkvlgsga fgtvykgiwi pdgenvkipv aikvlrents pkankeilde ayvmagvgsp |
| 781 | yvsrllgicl tstvglvtql mpygclldhv renrgrlgsq dllnwcmqia kgmsyledvr |
| 841 | lvhrdlaarn vlvkspnhvk itdfglarll dideteyhad ggkvpikwma lesilrrrft |
| 901 | hqsdvwsygv tvwelmtfga kpydgipare ipdllekger lpqppictid vymimvkcwm |
| 961 | idsecrprfr elvsefsrma rdpqrfvviq nedlgpaspl dstfyrslle dddmgdlvda |
| 1021 | eeylvpqqgf fcpdpapgag gmvhhrhrss strnm |
| Receptor tyrosine-protein kinase erbB-2, isoform e, NP_001276867.1 | |
| 1 | mklrlpaspe thldmlrhly qgcqvvqgnl eltylptnas lsflqdiqev qgyvliahnq |
| 61 | vrqvplqrlr ivrgtqlfed nyalavldng dplnnttpvt gaspgglrel qlrslteilk |
| 121 | ggvliqrnpq lcyqdtilwk difhknnqla ltlidtnrsr achpcspmck gsrcwgesse |
| 181 | dcqsltrtvc aggcarckgp lptdccheqc aagctgpkhs dclaclhfnh sgicelhcpa |
| 241 | lvtyntdtfe smpnpegryt fgascvtacp ynylstdvgs ctlvcplhnq evtaedgtqr |
| 301 | cekcskpcar vcyglgmehl revravtsan iqefagckki fgslaflpes fdgdpasnta |
| 361 | plqpeqlqvf etleeitgyl yisawpdslp dlsvfqnlqv irgrilhnga ysltlqglgi |
| 421 | swlglrslre lgsglalihh nthlcfvhtv pwdqlfrnph qallhtanrp edecvgegla |
| 481 | chqlcarghc wgpgptqcvn csqflrgqec veecrvlqgl preyvnarhc lpchpecqpq |
| 541 | ngsvtcfgpe adqcvacahy kdppfcvarc psgvkpdlsy mpiwkfpdee gacqpcpinc |
| 601 | ths |
| Inosine monophosphate dehydrogenase 2, NP_000875.2 | |
| 1 | madylisggt syvpddglta qqlfncgdgl tyndflilpg yidftadqvd ltsaltkkit |
| 61 | lktplvsspm dtvteagmai amaltggigf ihhnctpefq anevrkvkky eqgfitdpvv |
| 121 | lspkdrvrdv feakarhgfc gipitdtgrm gsrlvgiiss rdidflkeee hdcfleeimt |
| 181 | kredlvvapa gitlkeanei lqrskkgklp ivneddelva iiartdlkkn rdyplaskda |
| 241 | kkqllcgaai gtheddkyrl dllaqagvdv vvldssqgns ifqinmikyi kdkypnlqvi |
| 301 | ggnvvtaaqa knlidagvda lrvgmgsgsi citqevlacg rpqatavykv seyarrfgvp |
| 361 | viadggiqnv ghiakalalg astvmmgsll aatteapgey ffsdgirlkk yrgmgsldam |
| 421 | dkhlssqnry fseadkikva qgvsgavqdk gsihkfvpyl iagiqhscqd igaksltqvr |
| 481 | ammysgelkf ekrtssaqve ggvhslhsye krlf |
| KRAS proto-oncogene, GTPase, isoform a, NP_203524.1 | |
| 1 | mteyklvvvg aggvgksalt iqliqnhfvd eydptiedsy rkqvvidget clldildtag |
| 61 | geeysamrdq ymrtgegflc vfainntksf edihhyreqi krvkdsedvp mvlvgnkcdl |
| 121 | psrtvdtkqa qdlarsygip fietsaktrq rvedafytlv reirqyrlkk iskeektpgc |
| 181 | vkikkciim |
| KRAS proto-oncogene, GTPase, isoform b, NP_004976.2 | |
| 1 | mteyklvvvg aggvgksalt iqliqnhfvd eydptiedsy rkqvvidget clldildtag |
| 61 | qeeysamrdq ymrtgegflc vfainntksf edihhyreqi krvkdsedvp mvlvgnkcdl |
| 121 | psrtvdtkqa qdlarsygip fietsaktrq gvddafytlv reirkhkekm skdgkkkkkk |
| 181 | sktkcvim |
| Transforming growth factor beta receptor 2, isoform A precursor, | |
| NP_001020018.1 | |
| 1 | mgrgllrglw plhivlwtri astipphvqk sdvemeaqkd eiicpscnrt ahplrhinnd |
| 61 | mivtdnngav kfpqlckfcd vrfstcdnqk scmsncsits icekpqevcv avwrkndeni |
| 121 | tletvchdpk lpyhdfiled aaspkcimke kkkpgetffm cscssdecnd niifseeynt |
| 181 | snpdlllvif qvtgisllpp lgvaisviii fycyrvnrqq klsstwetgk trklmefseh |
| 241 | caiileddrs disstcanni nhntellpie ldtlvgkgrf aevykaklkq ntseqfetva |
| 301 | vkifpyeeya swktekdifs dinlkhenil qfltaeerkt elgkqywlit afhakgnlqe |
| 361 | yltrhviswe dlrklgssla rgiahlhsdh tpcgrpkmpi vhrdlkssni lvkndltccl |
| 421 | cdfglslrld ptlsvddlan sgqvgtarym apevlesrmn lenvesfkqt dvysmalvlw |
| 481 | emtsrcnavg evkdyeppfg skvrehpcve smkdnvlrdr grpeipsfwl nhqgiqmvce |
| 541 | tltecwdhdp earltaqcva erfselehld rlsgrscsee kipedgslnt tk |
| Transforming growth factor beta receptor 2, isoform B precursor, | |
| NP_003233.4 | |
| 1 | mgrgllrglw plhivlwtri astipphvqk svnndmivtd nngavkfpql ckfcdvrfst |
| 61 | cdnqkscmsn csitsicekp qevcvavwrk ndenitletv chdpklpyhd filedaaspk |
| 121 | cimkekkkpg etffmcscss decndniifs eeyntsnpdl llvifqvtgi sllpplgvai |
| 181 | sviiifycyr vnrqqklsst wetgktrklm efsehcaiil eddrsdisst canninhnte |
| 241 | llpieldtlv gkgrfaevyk aklkqntseq fetvavkifp yeeyaswkte kdifsdinlk |
| 301 | henilqflta eerktelgkq ywlitafhak gnlqeyltrh viswedlrkl gsslargiah |
| 361 | lhsdhtpcgr pkmpivhrdl kssnilvknd ltcclcdfgl slrldptlsv ddlansgqvg |
| 421 | tarymapevl esrmnlenve sfkqtdvysm alvlwemtsr cnavgevkdy eppfgskvre |
| 481 | hpcvesmkdn vlrdrgrpei psfwlnhqgi qmvcetltec wdhdpearlt aqcvaerfse |
| 541 | lehldrlsgr scseekiped gslnttk |
| Actinin alpha 4, isoform 1, NP_004915.2 | |
| 1 | mvdyhaanqs yqygpssagn gaggggsmgd ymaqeddwdr dllldpawek qqrktftawc |
| 61 | nshlrkagtq ienidedfrd glklmlllev isgerlpkpe rgkmrvhkin nvnkaldfia |
| 121 | skgvklvsig aeeivdgnak mtlgmiwtii lrfaiqdisv eetsakegll lwcqrktapy |
| 181 | knvnvqnfhi swkdglafna lihrhrpeli eydklrkddp vtnlnnafev aekyldipkm |
| 241 | ldaedivnta rpdekaimty vssfyhafsg aqkaetaanr ickvlavnqe nehlmedyek |
| 301 | lasdllewir rtipwledrv pqktiqemqq kledfrdyrr vhkppkvqek cqleinfntl |
| 361 | qtklrlsnrp afmpsegkmv sdinngwqhl eqaekgyeew llneirrler ldhlaekfrq |
| 421 | kasiheawtd gkeamlkhrd yetatlsdik alirkheafe sdlaahqdrv eqiaaiaqel |
| 481 | neldyydshn vntrcqkicd qwdalgslth srrealekte kqleaidqlh leyakraapf |
| 541 | nnwmesamed lqdmfivhti eeieglisah dqfkstlpda drereailai hkeaqriaes |
| 601 | nhiklsgsnp yttvtpqiin skwekvqqlv pkrdhallee qskqqsnehl rrqfasqanv |
| 661 | vgpwiqtkme eigrisiemn gtledqlshl kqyersivdy kpnldlleqq hqliqealif |
| 721 | dnkhtnytme hirvgweqll ttiartinev enqiltrdak gisqeqmqef rasfnhfdkd |
| 781 | hggalgpeef kaclislgyd vendrqgeae fnrimslvdp nhsglvtfqa fidfmsrett |
| 841 | dtdtadqvia sfkvlagdkn fitaeelrre lppdqaeyci armapyqgpd avpgaldyks |
| 901 | fstalygesd l |
| Actinin alpha 4, isoform 2, NP_001308962.1 | |
| 1 | mvdyhaanqs yqygpssagn gaggggsmgd ymaqeddwdr dllldpawek qqrktftawc |
| 61 | nshlrkagtq ienidedfrd glklmlllev isgerlpkpe rgkmrvhkin nvnkaldfia |
| 121 | skgvklvsig aeeivdgnak mtlgmiwtii lrfaiqdisv eetsakegll lwcqrktapy |
| 181 | knvnvqnfhi swkdglafna lihrhrpeli eydklrkddp vtnlnnafev aekyldipkm |
| 241 | ldaedivgtl rpdekaimty vscfyhafsg aqkaetaanr ickvlavnqe nehlmedyek |
| 301 | lasdllewir rtipwledrv pqktiqemqq kledfrdyrr vhkppkvqek cgleinfntl |
| 361 | qtklrlsnrp afmpsegkmv sdinngwqhl eqaekgyeew llneirrler ldhlaekfrq |
| 421 | kasiheawtd gkeamlkhrd yetatlsdik alirkheafe sdlaahqdrv eqiaaiaqel |
| 481 | neldyydshn vntrcqkicd qwdalgslth srrealekte kqleaidqlh leyakraapf |
| 541 | nnwmesamed lqdmfivhti eeieglisah dqfkstlpda drereailai hkeaqriaes |
| 601 | nhiklsgsnp yttvtpqiin skwekvqqlv pkrdhallee qskqqsnehl rrgfasganv |
| 661 | vgpwiqtkme eigrisiemn gtledqlshl kqyersivdy kpnldlleqq hqliqealif |
| 721 | dnkhtnytme hirvgweqll ttiartinev enqiltrdak gisqeqmqef rasfnhfdkk |
| 781 | qtgsmdsddf rallistgys lgeaefnrim slvdpnhsgl vtfqafidfm srettdtdta |
| 841 | dqviasfkvl agdknfitae elrrelppdq aeyciarmap yqgpdavpga ldyksfstal |
| 901 | ygesdl |
| Activin A receptor type 1, NP_001096.1, NP_001104537.1, | |
| NP_001334592.1, NP_001334593.1, NP_001334594.1, NP_001334595.1, | |
| NP_001334596.1 | |
| 1 | mvdgvmilpv limialpsps medekpkvnp klymcvcegl scgnedhceg qqcfsslsin |
| 61 | dgfhvyqkgc fqvyeqgkmt cktppspgqa veccqgdwcn rnitaqlptk gksfpgtqnf |
| 121 | hlevgliils vvfavcllac llgvalrkfk rrngerlnpr dveygtiegl ittnvgdstl |
| 181 | adlldhscts gsgsglpflv qrtvarqitl lecvgkgryg evwrgswqge nvavkifssr |
| 241 | dekswfrete lyntvmlrhe nilgfiasdm tsrhsstqlw lithyhemgs lydylqlttl |
| 301 | dtvsclrivl siasglahlh ieifgtqgkp aiahrdlksk nilvkkngqc ciadlglavm |
| 361 | hsqstnqldv gnnprvgtkr ymapevldet iqvdcfdsyk rvdiwafglv lwevarrmvs |
| 421 | ngivedykpp fydvvpndps fedmrkvvcv dqqrpnipnr wfsdptltsl aklmkecwyq |
| 481 | npsarltalr ikktltkidn sldklktdc |
| Alcohol dehydrogenase 1C (class I), gamma polypeptide, NP_000660.1 | |
| 1 | mstagkvikc kaavlwelkk pfsieeveva ppkahevrik mvaagicrsd ehvvsgnlvt |
| 61 | plpvilghea agivesvgeg vttvkpgdkv iplftpqcgk cricknpesn yclkndlgnp |
| 121 | rgtlqdgtrr ftcsgkpihh fvgvstfsqy tvvdenavak idaasplekv cligcgfstg |
| 181 | ygsavkvakv tpgstcavfg lggvglsvvm gckaagaari iavdinkdkf akakelgate |
| 241 | cinpqdykkp iqevlkemtd ggvdfsfevi grldtmmasl lccheacgts vivgvppdsq |
| 301 | nlsinpmlll tgrtwkgaif ggfkskesvp klvadfmakk fsldalitni lpfekinegf |
| 361 | dllrsgksir tvltf |
| Adenosine A2a receptor, NP_000666.2, NP_001265426.1, NP_001265427.1, | |
| NP_001265428.1, NP_001265429.1 | |
| 1 | mpimgssvyi tvelaiavla ilgnvlvcwa vwlnsnlqnv tnyfvvslaa adiavgvlai |
| 61 | pfaitistgf caachgclfi acfvlvltqs sifsllaiai dryiairipl rynglvtgtr |
| 121 | akgiiaicwv lsfaigltpm lgwnncgqpk egknhsqgcg egqvaclfed vvpmnymvyf |
| 181 | nffacvlvpl llmlgvylri flaarrqlkq mesqplpger arstlqkevh aakslaiivg |
| 241 | lfalcwlplh iincftffcp dcshaplwlm ylaivlshtn svvnpfiyay rirefrqtfr |
| 301 | kiirshvlrq qepfkaagts arvlaahgsd geqvslrlng hppgvwangs aphperrpng |
| 361 | yalglvsggs aqesqgntgl pdvellshel kgvcpeppgl ddplaqdgag vs |
| Rho guanine nucleotide exchange factor 16, NP_055263.2 | |
| 1 | maqrhsdssl eekllghrfh selrldaggn pasglpmvrg sprvrddaaf qpqvpappqp |
| 61 | rppgheepwp ivlstespaa lklgtqqlip kslavaskak tparhqsfga avlsreaarr |
| 121 | dpkllpapsf slddmdvdkd pggmlrrnlr nqsyraamkg lgkpggqgda iqlspklqal |
| 181 | aeepsqphtr spaknkktlg rkrghkgsfk ddpqlyqeiq erglntsqes dddildesss |
| 241 | pegtqkvdat ivvksyrpaq vtwsqlpevv elgildqlst eerkrqeamf eiltsefsyq |
| 301 | hslsilveef lqskelratv tqmehhhlfs nildvlgasq rffedleqrh kaqvlvedis |
| 361 | dileehaekh fhpyiaycsn evyqqrtlqk lissnaafre alreierrpa cgglpmlsfl |
| 421 | ilpmqrvtrl pllmdtlclk tqghseryka asralkaisk lvrqcnegah rmermeqmyt |
| 481 | lhtqldfskv kslplisasr wllkrgelfl veetglfrki asrptcylfl fndvlvvtkk |
| 541 | kseesymvqd yagmnhiqve kiepselplp gggnrsssvp hpfqvtllrn segrgeqlll |
| 601 | ssdsasdrar wivalthser qwqglsskgd lpqveitkaf fakqadevtl qqadvvlvlq |
| 661 | qedgwlyger lrdgetgwfp edfarfitsr vavegnvrrm erlrvetdv |
| B-cell linker, isoform 1, NP_037446.1 | |
| 1 | mdklnkitvp asqklrqlqk mvhdiknneg gimnkikklk vkappsvprr dyasespade |
| 61 | eeqwsddfds dyenpdehsd semyvmpaee naddsyeppp veqetrpvhp alpfargeyi |
| 121 | dnrssqrhsp pfsktlpskp swpsekarlt stlpaltalq kpqvppkpkg lledeadyvv |
| 181 | pvedndenyi hptesssppp ekapmvnrst kpnsstpasp pgtasgrnsg awetkspppa |
| 241 | apsplpragk kpttplkttp vasqqnassv ceekpipaer hrgsshrqea vqspvfppaq |
| 301 | kqihqkpipl prfteggnpt vdgplpsfss nstiseqeag vlckpwyaga cdrksaeeal |
| 361 | hrsnkdgsfl irkssghdsk qpytlvvffn krvynipvrf ieatkqyalg rkkngeeyfg |
| 421 | svaeiirnhq hsplvlidsq nntkdstrlk yavkvs |
| B-cell linker, isoform 2, NP_001107566.1 | |
| 1 | mdklnkitvp asqklrqlqk mvhdiknneg gimnkikklk vkappsvprr dyasespade |
| 61 | eeqwsddfds dyenpdehsd semyvmpaee naddsyeppp vegetrpvhp alpfargeyi |
| 121 | dnrssqrhsp pfsktlpskp swpsekarlt stlpaltalq kpqvppkpkg lledeadyvv |
| 181 | pvedndenyi hptesssppp ekgrnsgawe tkspppaaps plpragkkpt tplkttpvas |
| 241 | qqnassvcee kpipaerhrg sshrqeavqs pvfppaqkqi hqkpiplprf teggnptvdg |
| 301 | plpsfssnst iseqeagvlc kpwyagacdr ksaeealhrs nkdgsflirk ssghdskqpy |
| 361 | tlvvffnkrv ynipvrfiea tkqyalgrkk ngeeyfgsva eiirnhqhsp lvlidsqnnt |
| 421 | kdstrlkyav kvs |
| B-cell linker, isoform 3, NP_001245369.1 | |
| 1 | mdklnkitvp asqklrqlqk mvhdiknneg gimnkikklk vkappsvprr dyasespade |
| 61 | eeqwsddfds dyenpdehsd semyvmpaee naddsyeppp vegetrpvhp alpfargeyi |
| 121 | dnrssqrhsp pfsktlpskp swpsekarlt stlpaltalq kpqvppkpkg lledeadyvv |
| 181 | pvedndenyi hptesssppp ekapmvnrst kpnsstpasp pgtasgrnsg awetkspppa |
| 241 | apsplpragk kpttplkttp vasqqnassv ceekpipaer hrgsshrqea vqspvfppaq |
| 301 | kqihqkpipl prfteggnpt vdgplpsfss nstiseqeag vlckpwyaga cdrksaeeal |
| 361 | hrsnkyfgsv aeiirnhqhs plvlidsqnn tkdstrlkya vkvs |
| B-cell linker, isoform 4, NP_001245370.1 | |
| 1 | mdklnkitvp asqklrqlqk mvhdiknneg gimnkikklk vkappsvprr dyasespade |
| 61 | eeqwsddfds dyenpdehsd semyvmpaee naddsyeppp vegetrpvhp alpfargeyi |
| 121 | dnrssqrhsp pfsktlpskp swpsekarlt stlpaltalq kpqvppkpkg lledeadyvv |
| 181 | pvedndenyi hptesssppp ekgrnsgawe tkspppaaps plpragkkpt tplkttpvas |
| 241 | qqnassvcee kpipaerhrg sshrqeavqs pvfppaqkqi hqkpiplprf teggnptvdg |
| 301 | plpsfssnst iseqeagvlc kpwyagacdr ksaeealhrs nkyfgsvaei irnhqhsplv |
| 361 | lidsqnntkd strlkyavkv s |
| B-cell linker, isoform 5, NP_001245371.1 | |
| 1 | mdklnkitvp asqklrqlqk mvhdiknneg gimnkikklk vkappsvprr dyasespade |
| 61 | eeqwsddfds dyenpdehsd semyvmpaee naddsyeppp vegetrpvhp alpfargtas |
| 121 | grnsgawetk spppaapspl pragkkpttp lkttpvasqq nassvceekp ipaerhrgss |
| 181 | hrqeavqspv fppaqkqihq kpiplprfte ggnptvdgpl psfssnstis eqeagvlckp |
| 241 | wyagacdrks aeealhrsnk yfgsvaeiir nhqhsplvli dsqnntkdst rlkyavkvs |
| Basonuclin 1, isoform a, NP_001708.3 | |
| 1 | mrrrppsrgg rgaararetr rqprhrsgrr maeaisctln cscqsfkpgk inhrqcdqck |
| 61 | hgwvahalsk lrippmypts qveivqsnvv fdisslmlyg tqaipvrlki lldrlfsvlk |
| 121 | qdevlqilha ldwtlqdyir gyvlqdasgk vldhwsimts eeevatlqqf lrfgetksiv |
| 181 | elmaiqekee qsiiippsta nvdirafies cshrssslpt pvdkgnpssi hpfenlisnm |
| 241 | tfmlpfqffn plppaligsl peqymleqgh dqsqdpkqev hgpfpdssfl tssstpfqve |
| 301 | kdqclncpda itkkedsthl sdsssynivt kfertqlspe akvkpernsl gtkkgrvfct |
| 361 | acektfydkg tlkihynavh lkikhkctie gcnmvfsslr srnrhsanpn prlhmpmnrn |
| 421 | nrdkdlrnsl nlassenykc pgftvtspdc rpppsypgsg edskgqpafp nigqngvlfp |
| 481 | nlktvqpvlp fyrspatpae vantpgilps lpllsssipe qlisnempfd alpkkksrks |
| 541 | smpikiekea veianekrhn lssdedmplq vvsedeqeac spqshrvsee qhvqsgglgk |
| 601 | pfpegerpch resviessga isqtpeqath nsereteqtp alimvpreve dgghehyftp |
| 661 | gmepqvpfsd ymelqqrlla gglfsalsnr gmafpcleds kelehvgqha larqieenrf |
| 721 | qcdickktfk nacsvkihhk nmhvkemhtc tvegcnatfp srrsrdrhss nlnlhqkals |
| 781 | qealessedh fraayllkdv akeayqdvaf tqqasqtsvi fkgtsrmgsl vypitqvhsa |
| 841 | slesynsgpl segtildlst tssmksesss hsswdsdgvs eegtvlmeds dgncegsslv |
| 901 | pgedeypicv lmekadqsla slpsglpitc hlcqktysnk gtfrahyktv hlrqlhkckv |
| 961 | pgcntmfssv rsrnrhsqnp nlhkslassp shlq |
| Basonuclin 1, isoform b, NP_001288135.1 | |
| 1 | mrcrnmffsf kaslcgcgaa tapsltaisc tlncscqsfk pgkinhrqcd qckhgwvaha |
| 61 | lsklrippmy ptsqveivqs nvvfdisslm lygtqaipvr lkilldrlfs vlkqdevlqi |
| 121 | lhaldwtlqd yirgyvlqda sgkvldhwsi mtseeevatl qqflrfgetk sivelmaiqe |
| 181 | keeqsiiipp stanvdiraf iescshrsss lptpvdkgnp ssihpfenli snmtfmlpfq |
| 241 | ffnplppali gslpeqymle qghdqsqdpk qevhgpfpds sfltssstpf qvekdqclnc |
| 301 | pdaitkkeds thlsdsssyn ivtkfertql speakvkper nslgtkkgrv fctacektfy |
| 361 | dkgtlkihyn avhlkikhkc tiegcnmvfs slrsrnrhsa npnprlhmpm nrnnrdkdlr |
| 421 | nslnlassen ykcpgftvts pdcrpppsyp gsgedskgqp afpnigqngv lfpnlktvqp |
| 481 | vlpfyrspat paevantpgi lpslpllsss ipeqlisnem pfdalpkkks rkssmpikie |
| 541 | keaveianek rhnlssdedm plqvvsedeq eacspqshrv seeqhvgsgg lgkpfpeger |
| 601 | pchresvies sgaisqtpeq athnserete qtpalimvpr evedgghehy ftpgmepqvp |
| 661 | fsdymelqqr llagglfsal snrgmafpcl edskelehvg qhalarqiee nrfqcdickk |
| 721 | tfknacsvki hhknmhvkem htctvegcna tfpsrrsrdr hssnlnlhqk alsqealess |
| 781 | edhfraayll kdvakeayqd vaftqqasqt svifkgtsrm gslvypitqv hsaslesyns |
| 841 | gplsegtild lsttssmkse ssshsswdsd gvseegtvlm edsdgncegs slvpgedeyp |
| 901 | icvlmekadq slaslpsglp itchlcqkty snkgtfrahy ktvhlrqlhk ckvpgcntmf |
| 961 | ssvrsrnrhs qnpnlhksla sspshlq |
| BPI fold containing family A member 1, precursor, NP_001230122.1, | |
| NP_057667.1, NP_570913.1 | |
| 1 | mfqtgglivf ygllaqtmaq fgglpvpldq tlplnvnpal plsptglags ltnalsngll |
| 61 | sggllgilen lplldilkpg ggtsggllgg llgkvtsvip glnniidikv tdpqllelgl |
| 121 | vqspdghrly vtiplgiklq vntplvgasl lrlavkldit aeilavrdkq erihlvlgdc |
| 181 | thspgslqis lldglgplpi qglldsltgi lnkvlpelvq gnvcplvnev lrglditlvh |
| 241 | divnmlihgl qfvikv |
| Calcium voltage-gated channel auxiliary subunit beta 3, isoform 1, | |
| NP_000716.2 | |
| 1 | myddsyvpgf edseagsads ytsrpsldsd vsleedresa rrevesqaqq qlerakhkpv |
| 61 | afavrtnvsy cgvldeecpv qgsgvnfeak dflhikekys ndwwigrlvk eggdiafips |
| 121 | pqrlesirlk qeqkarrsgn psslsdignr rspppslakq kqkqaehvpp ydvvpsmrpv |
| 181 | vlvgpslkgy evtdmmqkal fdflkhrfdg risitrvtad lslakrsvln npgkrtiier |
| 241 | ssarssiaev qseierifel akslqlvvld adtinhpaql aktslapiiv fvkvsspkvl |
| 301 | qrlirsrgks qmkhltvqmm aydklvqcpp esfdvilden qledacehla eylevywrat |
| 361 | hhpapgpgll gppsaipglq nqqllgerge ehsplerdsl mpsdeasess rqawtgssqr |
| 421 | ssrhleedya dayqdlyqph rqhtsglpsa nghdpqdrll aqdsehnhsd rnwqrnrpwp |
| 481 | kdsy |
| Calcium voltage-gated channel auxiliary subunit beta 3, isoform 2, | |
| NP_001193844.1 | |
| 1 | myddsyvpgf edseagsads ytsrpsldsd vsleedresa rrevesqaqq qlerakkysn |
| 61 | dwwigrlvke ggdiafipsp grlesirlkg eqkarrsgnp sslsdignrr spppslakqk |
| 121 | qkqaehvppy dvvpsmrpvv lvgpslkgye vtdmmqkalf dflkhrfdgr isitrvtadl |
| 181 | slakrsvlnn pgkrtiiers sarssiaevq seierifela kslqlvvlda dtinhpaqla |
| 241 | ktslapiivf vkvsspkvlq rlirsrgksq mkhltvqmma ydklvqcppe sfdvildenq |
| 301 | ledacehlae ylevywrath hpapgpgllg ppsaipglqn qqllgergee hsplerdslm |
| 361 | psdeasessr qawtgssqrs srhleedyad ayqdlyqphr qhtsglpsan ghdpqdrlla |
| 421 | qdsehnhsdr nwqrnrpwpk dsy |
| Calcium voltage-gated channel auxiliary subunit beta 3, isoform 3, | |
| NP_001193845.1 | |
| 1 | msfsdssatf llnegsadsy tsrpsldsdv sleedresar revesqaqqq lerakhkpva |
| 61 | favrtnvsyc gvldeecpvq gsgvnfeakd flhikekysn dwwigrlvke ggdiafipsp |
| 121 | qrlesirlkq eqkarrsgnp sslsdignrr spppslakqk qkqaehvppy dvvpsmrpvv |
| 181 | lvgpslkgye vtdmmqkalf dflkhrfdgr isitrvtadl slakrsvlnn pgkrtiiers |
| 241 | sarssiaevq seierifela kslqlvvlda dtinhpaqla ktslapiivf vkvsspkvlq |
| 301 | rlirsrgksq mkhltvqmma ydklvqcppe sfdvildenq ledacehlae ylevywrath |
| 361 | hpapgpgllg ppsaipglqn qqllgergee hsplerdslm psdeasessr qawtgssqrs |
| 421 | srhleedyad ayqdlyqphr qhtsglpsan ghdpqdrlla qdsehnhsdr nwqrnrpwpk |
| 481 | dsy |
| Calcium voltage-gated channel auxiliary subunit beta 3, isoform 4, | |
| NP_001193846.1 | |
| 1 | megsadsyts rpsldsdvsl eedresarre vesqaqqqle rakhkpvafa vrtnvsycgv |
| 61 | ldeecpvqgs gvnfeakdfl hikekysndw wigrlvkegg diafipspqr lesirlkqeq |
| 121 | karrsgnpss lsdignrrsp ppslakqkqk qaehvppydv vpsmrpvvlv gpslkgyevt |
| 181 | dmmqkalfdf lkhrfdgris itrvtadlsl akrsvlnnpg krtiierssa rssiaevqse |
| 241 | ierifelaks lqlvvldadt inhpaqlakt slapiivfvk vsspkvlqrl irsrgksqmk |
| 301 | hltvqmmayd klvqcppesf dvildenqle dacehlaeyl evywrathhp apgpgllgpp |
| 361 | saipglqnqq llgergeehs plerdslmps deasessrqa wtgssqrssr hleedyaday |
| 421 | qdlyqphrqh tsglpsangh dpqdrllaqd sehnhsdrnw grnrpwpkds y |
| Caspase 3, preproprotein, NP_001341706.1, NP_001341707.1, NP_004346.3, | |
| NP_116786.1 | |
| 1 | mentensvds ksiknlepki ihgsesmdsg isldnsykmd ypemglciii nnknfhkstg |
| 61 | mtsrsgtdvd aanlretfrn lkyevrnknd ltreeivelm rdvskedhsk rssfvcvlls |
| 121 | hgeegiifgt ngpvdlkkit nffrgdrcrs ltgkpklfii qacrgteldc gietdsgvdd |
| 181 | dmachkipve adflyaysta pgyyswrnsk dgswfiqslc amlkqyadkl efmhiltrvn |
| 241 | rkvatefesf sfdatfhakk qipcivsmlt kelyfyh |
| Caspase 3, isoform b, NP_001341708.1, NP001341709.1 | |
| 1 | mdsgisldns ykmdypemgl ciiinnknfh kstgmtsrsg tdvdaanlre tfrnlkyevr |
| 61 | nkndltreei velmrdvske dhskrssfvc vllshgeegi ifgtngpvdl kkitnffrgd |
| 121 | rcrsltgkpk lfiiqacrgt eldcgietds gvdddmachk ipveadflya ystapgyysw |
| 181 | rnskdgswfi qslcamlkqy adklefmhil trvnrkvate fesfsfdatf hakkqipciv |
| 241 | smltkelyfy h |
| Caspase 3, isoform c, NP_001341710.1, NP001341711.1 | |
| 1 | mentensvds ksiknlepki ihgsesmdsg isldnsykmd ypemglciii nnknfhkstg |
| 61 | mtsrsgtdvd aanlretfrn lkyevrnknd ltreeivelm rdvskedhsk rssfvcvlls |
| 121 | hgeegiifgt ngpvdlkkit nffrgdrcrs ltgkpklfii qviilgeiqr mapgsssrfv |
| 181 | pc |
| Caspase 3, isoform d, NP_001341712.1 | |
| 1 | msdalikvsm entensvdsk siknlepkii hgsesmdsgi sldnsykmdy pemglciiin |
| 61 | nknfhkstgm tsrsgtdvda anlretfrnl kyevrnkndl treeivelmr dvskedhskr |
| 121 | ssfvcvllsh geegiifgtn gpvdlkkitn ffrgdrcrsl tgkpklfiiq viilgeiqrm |
| 181 | apgsssrfvp c |
| Caspase 3, isoform e, NP_001341713.1 | |
| 1 | mdsgisldns ykmdypemgl ciiinnknfh kstgmtsrsg tdvdaanlre tfrnlkyevr |
| 61 | nkndltreei velmrdvske dhskrssfvc vllshgeegi ifgtngpvdl kkitnffrgd |
| 121 | rcrsltgkpk lfiiqviilg eiqrmapgss srfvpc |
| Caveolin 1, isoform alpha, NP_001744.2 | |
| 1 | msggkyvdse ghlytvpire qgniykpnnk amadelsekq vydahtkeid lvnrdpkhln |
| 61 | ddvvkidfed viaepegths fdgiwkasft tftvtkywfy rllsalfgip maliwgiyfa |
| 121 | ilsflhiwav vpciksflie iqcisrvysi yvhtvcdplf eavgkifsnv rinlqkei |
| Caveolin 1, isoform beta, NP_001166366.1, NP_001166367.1, | |
| NP_001166368.1 | |
| 1 | madelsekqv ydahtkeidl vnrdpkhlnd dvvkidfedv iaepegthsf dgiwkasftt |
| 61 | ftvtkywfyr llsalfgipm aliwgiyfai lsflhiwavv pciksfliei qcisrvysiy |
| 121 | vhtvcdplfe avgkifsnvr inlqkei |
| Cadherin 1, isoform 1 preproprotein, NP_004351.1 | |
| 1 | mgpwsrslsa lllllqvssw lcqepepchp gfdaesytft vprrhlergr vlgrvnfedc |
| 61 | tgrqrtayfs ldtrfkvgtd gvitvkrplr fhnpqihflv yawdstyrkf stkvtlntvg |
| 121 | hhhrppphqa svsgiqaell tfpnsspglr rqkrdwvipp iscpenekgp fpknlvqiks |
| 181 | nkdkegkvfy sitgqgadtp pvgvfiiere tgwlkvtepl dreriatytl fshavssngn |
| 241 | avedpmeili tvtdqndnkp eftqevfkgs vmegalpgts vmevtatdad ddvntynaai |
| 301 | aytilsqdpe lpdknmftin rntgvisvvt tgldresfpt ytlvvqaadl qgeglsttat |
| 361 | avitvtdtnd nppifnptty kgqvpenean vvittlkvtd adapntpawe avytilnddg |
| 421 | gqfvvttnpv nndgilktak gldfeakqqy ilhvavtnvv pfevslttst atvtvdvldv |
| 481 | neapifvppe krvevsedfg vgqeitsyta qepdtfmeqk ityriwrdta nwleinpdtg |
| 541 | aistraeldr edfehvknst ytaliiatdn gspvatgtgt lllilsdvnd napipeprti |
| 601 | ffcernpkpq viniidadlp pntspftael thgasanwti qyndptqesi ilkpkmalev |
| 661 | gdykinlklm dnqnkdqvtt levsvcdceg aagvcrkaqp veaglqipai lgilggilal |
| 721 | lililllllf lrrravvkep llppeddtrd nvyyydeegg geedqdfdls qlhrgldarp |
| 781 | evtrndvapt lmsvprylpr panpdeignf idenlkaadt dptappydsl lvfdyegsgs |
| 841 | eaaslsslns sesdkdqdyd ylnewgnrfk kladmyggge dd |
| Cadherin 1, isoform 2 precursor, NP_001304113.1 | |
| 1 | mgpwsrslsa lllllqvssw lcqepepchp gfdaesytft vprrhlergr vlgrvnfedc |
| 61 | tgrqrtayfs ldtrfkvgtd gvitvkrplr fhnpqihflv yawdstyrkf stkvtlntvg |
| 121 | hhhrppphqa sysgiqaell tfpnsspglr rqkrdwvipp iscpenekgp fpknlvqiks |
| 181 | nkdkegkvfy sitgqgadtp pvgvfiiere tgwlkvtepl dreriatytl fshayssngn |
| 241 | avedpmeili tvtdqndnkp eftqevfkgs vmegalpgts vmevtatdad ddvntynaai |
| 301 | aytilsqdpe lpdknmftin rntgvisvvt tgldresfpt ytlvvqaadl qgeglsttat |
| 361 | avitvtdtnd nppifnpttg ldfeakqqyi lhvavtnvvp fevslttsta tvtvdvldvn |
| 421 | eapifvppek rvevsedfgv gqeitsytaq epdtfmeqki tyriwrdtan wleinpdtga |
| 481 | istraeldre dfehvknsty taliiatdng spvatgtgtl llilsdvndn apipeprtif |
| 541 | fcernpkpqv iniidadlpp ntspftaelt hgasanwtiq yndptqesii lkpkmalevg |
| 601 | dykinlklmd nqnkdqvttl evsvcdcega agvcrkaqpv eaglqipail gilggilall |
| 661 | ililllllfl rrravvkepl lppeddtrdn vyyydeeggg eedqdfdlsq lhrgldarpe |
| 721 | vtrndvaptl msvprylprp anpdeignfi denlkaadtd ptappydsll vfdyegsgse |
| 781 | aaslsslnss esdkdqdydy lnewgnrfkk ladmyggged d |
| Cadherin 1, isoform 3, NP_001304114.1 | |
| 1 | meqkityriw rdtanwlein pdtgaistra eldredfehv knstytalii atdngspvat |
| 61 | gtgtlllils dvndnapipe prtiffcern pkpqviniid adlppntspf taelthgasa |
| 121 | nwtiqyndpt qesiilkpkm alevgdykin lklmdnqnkd qvttlevsvc dcegaagvcr |
| 181 | kaqpveaglq ipailgilgg ilallilill lllflrrrav vkepllpped dtrdnvyyyd |
| 241 | eegggeedqd fdlsqlhrgl darpevtrnd vaptlmsvpr ylprpanpde ignfidenlk |
| 301 | aadtdptapp ydsllvfdye gsgseaasls slnssesdkd qdydylnewg nrfkkladmy |
| 361 | gggedd |
| Cadherin 1, isoform 4, NP_001304115.1 | |
| 1 | malevgdyki nlklmdnqnk dqvttlevsv cdcegaagvc rkaqpveagl qipailgilg |
| 61 | gilallilil llllflrrra vvkepllppe ddtrdnvyyy deegggeedq dfdlsqlhrg |
| 121 | ldarpevtrn dvaptlmsvp rylprpanpd eignfidenl kaadtdptap pydsllvfdy |
| 181 | egsgseaasl sslnssesdk dqdydylnew gnrfkkladm ygggedd |
| Cytochrome c oxidase subunit 8C, NP_892016.1 | |
| 1 | mpllrgrcpa rrhyrrlall glqpaprfah sgpprqrpls aaemavglvv ffttfltpaa |
| 61 | yvlgnlkqfr rn |
| Carnitine palmitoyltransferase 1A, isoform 1, NP_001867.2 | |
| 1 | maeahqavaf qftvtpdgid lrlshealrq iylsglhswk kkfirfkngi itgvypasps |
| 61 | swlivvvgvm ttmyakidps lgiiakinrt letancmssq tknvvsgvlf gtglwvaliv |
| 121 | tmryslkvll syhgwmfteh gkmsratkiw mgmvkifsgr kpmlysfqts lprlpvpavk |
| 181 | dtvnrylqsv rplmkeedfk rmtalaqdfa vglgprlqwy lklkswwatn yvsdwweeyi |
| 241 | ylrgrgplmv nsnyyamdll yilpthiqaa ragnaihail lyrrkldree ikpirllgst |
| 301 | iplcsaqwer mfntsripge etdtiqhmrd skhivvyhrg ryfkvwlyhd grllkpreme |
| 361 | qqmqrildnt sepqpgearl aaltagdrvp warcrqayfg rgknkqslda vekaaffvtl |
| 421 | deteegyrse dpdtsmdsya ksllhgrcyd rwfdksftfv vfkngkmgln aehswadapi |
| 481 | vahlweyvms idslqlgyae dghckgdinp nipyptrlqw dipgecqevi etslntanll |
| 541 | andvdfhsfp fvafgkgiik kcrtspdafv qlalqlahyk dmgkfcltye asmtrlfreg |
| 601 | rtetvrsctt escdfvramv dpaqtveqrl klfklasekh qhmyrlamtg sgidrhlfcl |
| 661 | yvvskylave spflkevlse pwrlstsqtp qqqvelfdle nnpeyvssgg gfgpvaddgy |
| 721 | gvsyilvgen linfhisskf scpetdshrf grhlkeamtd iitlfglssn skk |
| Carnitine palmitoyltransferase 1A, isoform 2, NP_001027017.1 | |
| 1 | maeahqavaf qftvtpdgid lrlshealrq iylsglhswk kkfirfkngi itgvypasps |
| 61 | swlivvvgvm ttmyakidps lgiiakinrt letancmssq tknvvsgvlf gtglwvaliv |
| 121 | tmryslkvll syhgwmfteh gkmsratkiw mgmvkifsgr kpmlysfqts lprlpvpavk |
| 181 | dtvnrylqsv rplmkeedfk rmtalaqdfa vglgprlqwy lklkswwatn yvsdwweeyi |
| 241 | ylrgrgplmv nsnyyamdll yilpthiqaa ragnaihail lyrrkldree ikpirllgst |
| 301 | iplcsaqwer mfntsripge etdtiqhmrd skhivvyhrg ryfkvwlyhd grllkpreme |
| 361 | qqmqrildnt sepqpgearl aaltagdrvp warcrqayfg rgknkgslda vekaaffvtl |
| 421 | deteegyrse dpdtsmdsya ksllhgrcyd rwfdksftfv vfkngkmgln aehswadapi |
| 481 | vahlweyvms idslqlgyae dghckgdinp nipyptrlqw dipgecqevi etslntanll |
| 541 | andvdfhsfp fvafgkgiik kcrtspdafv qlalqlahyk dmgkfcltye asmtrlfreg |
| 601 | rtetvrsctt escdfvramv dpaqtveqrl klfklasekh qhmyrlamtg sgidrhlfcl |
| 661 | yvvskylave spflkevlse pwrlstsqtp qqqvelfdle nnpeyvssgg gfgpvaddgy |
| 721 | gvsyilvgen linfhisskf scpetgiisq gpssdt |
| Cancer/testis antigen 1A, NP_640343.1 | |
| 1 | mqaegrgtgg stgdadgpgg pgipdgpggn aggpgeagat ggrgprgaga arasgpggga |
| 61 | prgphggaas glngccrcga rgpesrllef ylampfatpm eaelarrsla qdapplpvpg |
| 121 | vllkeftvsg niltirltaa dhrqlqlsis sclqqlsllm witqcflpvf laqppsgqrr |
| C-X-C motif chemokine ligand 13, NP_006410.1 | |
| 1 | mkfistslll mllvsslspv qgvlevyyts lrcrcvqess vfiprrfidr iqilprgngc |
| 61 | prkeiivwkk nksivcvdpq aewiqrmmev lrkrssstlp vpvfkrkip |
| Diacylglycerol kinase eta, isoform 1, NP_001191433.1, NP_690874.2 | |
| 1 | magaggqhhp pgaaggaaag agaavtsaaa sagpgedssd seaeqegpqk lirkvstsgq |
| 61 | irtktsikeg qllkqtssfq rwkkryfklr grtlyyakds kslifdevdl sdasvaeast |
| 121 | knannsftii tpfrrlmlca enrkemedwi sslksvqtre pyevaqfnve hfsgmhnwya |
| 181 | csharptfcn vcreslsgvt shglscevck fkahkrcavr atnnckwttl asigkdiied |
| 241 | edgvamphqw legnlpvsak cavcdktcgs vlrlqdwkcl wcktmvhtac kdlyhpicpl |
| 301 | gqckvsiipp ialnstdsdg fcratfsfcv spllvfvnsk sgdnqgvkfl rrfkqllnpa |
| 361 | qvfdlmnggp hlglrlfqkf dnfrilvcgg dgsvgwvlse idklnlnkqc qlgvlplgtg |
| 421 | ndlarvlgwg gsydddtqlp qilekleras tkmldrwsim tyelklppka sllpgppeas |
| 481 | eefymtiyed svathltkil nsdehavvis saktlcetvk dfvakvekty dktlenavva |
| 541 | davaskcsvl nekleqllqa lhtdsqaapv lpglsplive edavesssee slgeskeqlg |
| 601 | ddvtkpssqk avkpreimlr anslkkavrq vieeagkvmd dptvhpcepa nqssdydste |
| 661 | tdeskeeakd dgakesitvk taprspdara syghsqtdsv pgpavaaske nlpvintrii |
| 721 | cpglraglaa siagssiink mllanidpfg atpfidpdld svdgysekcv mnnyfgigld |
| 781 | akislefnnk reehpekcrs rtknlmwygv lgtrellqrs yknleqrvql ecdgqyiplp |
| 841 | slqgiavlni psyaggtnfw ggtkeddifa apsfddkile vvaifdsmqm avsrviklqh |
| 901 | hriaqcrtvk itifgdegvp vqvdgeawvq ppgiikivhk nraqmltrdr afestlkswe |
| 961 | dkqkcdsgkp vlrthlyihh aidlateevs qmqlcsqaae elitricdaa tihclleqel |
| 1021 | ahavnacsha lnkanprcpe sltrdtatei ainvkalyne tesllvgrvp lqlespheer |
| 1081 | vsnalhsvev elqklteipw lyyilhpned eeppmdctkr nnrstvfriv pkfkkekvqk |
| 1141 | qktssqpgsg dtesgscean spgn |
| Diacylglycerol kinase eta, isoform 2, NP_821077.1 | |
| 1 | magaggqhhp pgaaggaaag agaavtsaaa sagpgedssd seaeqegpqk lirkvstsgq |
| 61 | irtktsikeg qllkqtssfq rwkkryfklr grtlyyakds kslifdevdl sdasvaeast |
| 121 | knannsftii tpfrrlmlca enrkemedwi sslksvqtre pyevaqfnve hfsgmhnwya |
| 181 | csharptfcn vcreslsgvt shglscevck fkahkrcavr atnnckwttl asigkdiied |
| 241 | edgvamphqw legnlpvsak cavcdktcgs vlrlqdwkcl wcktmvhtac kdlyhpicpl |
| 301 | gqckvsiipp ialnstdsdg fcratfsfcv spllvfvnsk sgdnqgvkfl rrfkqllnpa |
| 361 | qvfdlmnggp hlglrlfqkf dnfrilvcgg dgsvgwvlse idklnlnkqc qlgvlplgtg |
| 421 | ndlarvlgwg gsydddtqlp qilekleras tkmldrwsim tyelklppka sllpgppeas |
| 481 | eefymtiyed svathltkil nsdehavvis saktlcetvk dfvakvekty dktlenavva |
| 541 | davaskcsvl nekleqllqa lhtdsqaapv lpglsplive edavesssee slgeskeqlg |
| 601 | ddvtkpssqk avkpreimlr anslkkavrq vieeagkvmd dptvhpcepa nqssdydste |
| 661 | tdeskeeakd dgakesitvk taprspdara syghsqtdsv pgpavaaske nlpvlntrii |
| 721 | cpglraglaa siagssiink mllanidpfg atpfidpdld svdgysekcv mnnyfgigld |
| 781 | akislefnnk reehpekcrs rtknlmwygv lgtrellqrs yknlegrvql ecdgqyiplp |
| 841 | slqgiavlni psyaggtnfw ggtkeddifa apsfddkile vvaifdsmqm aysrviklqh |
| 901 | hriaqcrtvk itifgdegvp vqvdgeawvq ppgiikivhk nraqmltrdr afestlkswe |
| 961 | dkqkcdsgkp vlrthlyihh aidlateevs qmqlcsqaae elitricdaa tihclleqel |
| 1021 | ahavnacsha lnkanprcpe sltrdtatei ainvkalyne tesllvgrvp lqlespheer |
| 1081 | vsnalhsvev elqklteipw lyyilhpned eeppmdctkr nnrstvfriv pkfkkekvqk |
| 1141 | qktssqpvqk wgteevaawl dllnlgeykd ifirhdirga ellhlerrdl kdlgipkvgh |
| 1201 | vkrilqgike lgrstpqsev |
| Diacylglycerol kinase eta, isoform 3, NP_001191434.1 | |
| 1 | mlcaenrkem edwisslksv qtrepyevaq fnvehfsgmh nwyacsharp tfcnvcresl |
| 61 | sgvtshglsc evckfkahkr cavratnnck wttlasigkd iiededgvam phqwlegnlp |
| 121 | vsakcavcdk tcgsvlrlqd wkclwcktmv htackdlyhp icplgqckvs iippialnst |
| 181 | dsdgfcratf sfcvspllvf vnsksgdnqg vkflrrfkql lnpaqvfdlm nggphlglrl |
| 241 | fqkfdnfril vcggdgsvgw vlseidklnl nkqcqlgvlp lgtgndlarv lgwggsyddd |
| 301 | tqlpqilekl erastkmldr wsimtyelkl ppkasllpgp peaseefymt iyedsvathl |
| 361 | tkilnsdeha vvissaktlc etvkdfvakv ektydktlen avvadavask csvlnekleq |
| 421 | llqalhtdsq aapvlpglsp liveedaves sseeslgesk eqlgddvtkp ssqkavkpre |
| 481 | imlranslkk avrqvieeag kvmddptvhp cepanqssdy dstetdeske eakddgakes |
| 541 | itvktaprsp darasyghsq tdsvpgpava askenlpvln triicpglra glaasiagss |
| 601 | iinkmllani dpfgatpfid pdldsvdgys ekcvmnnyfg igldakisle fnnkreehpe |
| 661 | kcrsrtknlm wygvlgtrel lqrsyknleq rvqlecdgqy iplpslqgia vlnipsyagg |
| 721 | tnfwggtked difaapsfdd kilevvaifd smqmavsrvi klqhhriaqc rtvkitifgd |
| 781 | egvpvqvdge awvqppgiik ivhknragml trdrafestl kswedkqkcd sgkpvlrthl |
| 841 | yihhaidlat eevsqmqlcs qaaeelitri cdaatihcll eqelahavna cshalnkanp |
| 901 | rcpesltrdt ateiainvka lynetesllv grvplqlesp heervsnalh svevelqklt |
| 961 | eipwlyyilh pnedeeppmd ctkrnnrstv frivpkfkke kvqkqktssq pvqkwgteev |
| 1021 | aawldllnlg eykdifirhd irgaellhle rrdlkntvge krdtkengkh mdlgipkvgh |
| 1081 | vkrilqgike lgrstpqsev |
| Diacylglycerol kinase eta, isoform 4, NP_001191435.1 | |
| 1 | mlcaenrkem edwisslksv qtrepyevaq fnvehfsgmh nwyacsharp tfcnvcresl |
| 61 | sgvtshglsc evckfkahkr cavratnnck wttlasigkd iiededgvam phqwlegnlp |
| 121 | vsakcavcdk tcgsvlrlqd wkclwcktmv htackdlyhp icplgqckvs iippialnst |
| 181 | dsdgfcratf sfcvspllvf vnsksgdnqg vkflrrfkql lnpaqvfdlm nggphlglrl |
| 241 | fqkfdnfril vcggdgsvgw vlseidklnl nkqcqlgvlp lgtgndlarv lgwggsyddd |
| 301 | tqlpqilekl erastkmldr wsimtyelkl ppkasllpgp peaseefymt iyedsvathl |
| 361 | tkilnsdeha vvissaktlc etvkdfvakv ektydktlen avvadavask csvlnekleq |
| 421 | llqalhtdsq aapvlpglsp liveedaves sseeslgesk eqlgddvtkp ssqkavkpre |
| 481 | imlranslkk avrqvieeag kvmddptvhp cepanqssdy dstetdeske eakddgakes |
| 541 | itvktaprsp darasyghsq tdsvpgpava askenlpvln triicpglra glaasiagss |
| 601 | iinkmllani dpfgatpfid pdldsvdgys ekcvmnnyfg igldakisle fnnkreehpe |
| 661 | kcrsrtknlm wygvlgtrel lqrsyknleq rvqlecdgqy iplpslqgia vlnipsyagg |
| 721 | tnfwggtked difaapsfdd kilevvaifd smqmavsrvi klqhhriaqc rtvkitifgd |
| 781 | egvpvqvdge awvqppgiik ivhknraqml trdrafestl kswedkqkcd sgkpvlrthl |
| 841 | yihhaidlat eevsqmqlcs qaaeelitri cdaatihcll eqelahavna cshalnkanp |
| 901 | rcpesltrdt ateiainvka lynetesllv grvplqlesp heervsnalh svevelqklt |
| 961 | eipwlyyilh pnedeeppmd ctkrnnrstv frivpkfkke kvqkqktssq pvqkwgteev |
| 1021 | aawldllnlg eykdifirhd irgaellhle rrdlkdlgip kvghvkrilq gikelgrstp |
| 1081 | qsev |
| Diacylglycerol kinase eta, isoform 5, NP_001284358.1 | |
| 1 | mwnisqgctt gtpaptpdpp svtcaervfl esppmacpak vhtackdlyh picplgqckv |
| 61 | siippialns tdsdgfcrat fsfcvspllv fvnsksgdnq gvkflrrfkq llnpaqvfdl |
| 121 | mnggphlglr lfqkfdnfri lvcggdgsvg wvlseidkln lnkqcqlgvl plgtgndlar |
| 181 | vlgwggsydd dtqlpqilek lerastkmld rwsimtyelk lppkasllpg ppeaseefym |
| 241 | tiyedsvath ltkilnsdeh avvissaktl cetvkdfvak vektydktle navvadavas |
| 301 | kcsvlnekle qllqalhtds qaapvlpgls pliveedave ssseeslges keqlgddvtk |
| 361 | pssqkavkpr eimlranslk kavrqvieea gkvmddptvh pcepanqssd ydstetdesk |
| 421 | eeakddgake sitvktaprs pdarasyghs qtdsvpgpav aaskenlpvl ntriicpglr |
| 481 | aglaasiags siinkmllan idpfgatpfi dpdldsvdgy sekcvmnnyf gigldakisl |
| 541 | efnnkreehp ekcrsrtknl mwygvlgtre llqrsyknle qrvqlecdgq yiplpslqgi |
| 601 | avlnipsyag gtnfwggtke ddifaapsfd dkilevvaif dsmqmavsrv iklqhhriaq |
| 661 | crtvkitifg degvpvqvdg eawvqppgii kivhknraqm ltrdrafest lkswedkqkc |
| 721 | dsgkpvlrth lyihhaidla teevsgmqlc sqaaeelitr icdaatihcl leqelahavn |
| 781 | acshalnkan prcpesltrd tateiainvk alynetesll vgrvplqles pheervsnal |
| 841 | hsvevelqkl teipwlyyil hpnedeeppm dctkrnnrst vfrivpkfkk ekvqkqktss |
| 901 | qpgsgdtesg sceanspgn |
| Eukaryotic translation elongation factor 2, NP_001952.1 | |
| 1 | mvnftvdqir aimdkkanir nmsviahvdh gkstltdslv ckagiiasar agetrftdtr |
| 61 | kdeqerciti kstaislfye lsendlnfik qskdgagfli nlidspghvd fssevtaalr |
| 121 | vtdgalvvvd cvsgvcvqte tvlrqaiaer ikpvlmmnkm drallelqle peelyqtfqr |
| 181 | ivenvnviis tygegesgpm gnimidpvlg tvgfgsglhg waftlkqfae myvakfaakg |
| 241 | egqlgpaera kkvedmmkkl wgdryfdpan gkfsksatsp egkklprtfc qlildpifkv |
| 301 | fdaimnfkke etakliekld ikldsedkdk egkpllkavm rrwlpagdal lqmitihlps |
| 361 | pvtaqkyrce llyegppdde aamgikscdp kgplmmyisk mvptsdkgrf yafgrvfsgl |
| 421 | vstglkvrim gpnytpgkke dlylkpiqrt ilmmgryvep iedvpcgniv glvgvdqflv |
| 481 | ktgtittfeh ahnmrvmkfs vspvvrvave aknpadlpkl veglkrlaks dpmvqciiee |
| 541 | sgehiiagag elhleiclkd leedhacipi kksdpvvsyr etvseesnvl clskspnkhn |
| 601 | rlymkarpfp dglaedidkg evsarqelkq rarylaekye wdvaearkiw cfgpdgtgpn |
| 661 | iltditkgvq ylneikdsvv agfgwatkeg alceenmrgv rfdvhdvtlh adaihrgggq |
| 721 | iiptarrcly asvltaqprl mepiylveiq cpeqvvggiy gvlnrkrghv feesqvagtp |
| 781 | mfvvkaylpv nesfgftadl rsntggqafp qcvfdhwqil pgdpfdnssr psqvvaetrk |
| 841 | rkglkegipa ldnfldkl |
| Eukaryotic translation initiation factor 5A, isoform A, NP_001137232.1 | |
| 1 | mcgtggtdsk trrpphrasf lkrleskplk maddldfetg dagasatfpm qcsalrkngf |
| 61 | vvlkgrpcki vemstsktgk hghakvhlvg idiftgkkye dicpsthnmd vpnikrndfq |
| 121 | ligiqdgyls llqdsgevre dlrlpegdlg keieqkydcg eeilitvlsa mteeaavaik |
| 181 | amak |
| Eukaryotic translation initiation factor 5A, isoform B, | |
| NP_001137233.1, NP_001137234.1, NP_001961.1 | |
| 1 | maddldfetg dagasatfpm qcsalrkngf vvlkgrpcki vemstsktgk hghakvhlvg |
| 61 | idiftgkkye dicpsthnmd vpnikrndfq ligiqdgyls llqdsgevre dlrlpegdlg |
| 121 | keieqkydcg eeilitvlsa mteeaavaik amak |
| Fibronectin 1, isoform 1 precursor, NP_997647.1 | |
| 1 | mlrgpgpgll llavqclgta vpstgasksk rqaqqmvqpq spvaysgskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipevpql tdlsfvditd ssiglrwtpl nsstiigyri tvvaagegip ifedfvdssv |
| 1321 | gyytvtglep gidydisvit linggesapt tltqqtavpp ptdlrftnig pdtmrvtwap |
| 1381 | ppsidltnfl vryspvknee dvaelsisps dnavvltnll pgteyvvsvs svyeqhestp |
| 1441 | lrgrqktgld sptgidfsdi tansftvhwi apratitgyr irhhpehfsg rpredrvphs |
| 1501 | rnsitltnlt pgteyvvsiv alngreespl ligqqstvsd vprdlevvaa tptslliswd |
| 1561 | apavtvryyr itygetggns pvqeftvpgs kstatisglk pgvdytitvy avtgrgdspa |
| 1621 | sskpisinyr teidkpsqmq vtdvqdnsis vkwlpssspv tgyrvtttpk ngpgptktkt |
| 1681 | agpdqtemti eglqptveyv vsvyaqnpsg esqplvqtav tnidrpkgla ftdvdvdsik |
| 1741 | iawespqgqv sryrvtyssp edgihelfpa pdgeedtael qglrpgseyt vsvvalhddm |
| 1801 | esqpligtqs taipaptdlk ftqvtptsls aqwtppnvql tgyrvrvtpk ektgpmkein |
| 1861 | lapdsssvvv sglmvatkye vsvyalkdtl tsrpaqgvvt tlenvspprr arvtdatett |
| 1921 | itiswrtkte titgfqvdav pangqtpiqr tikpdvrsyt itglqpgtdy kiylytlndn |
| 1981 | arsspvvida staidapsnl rflattpnsl lvswqpprar itgyiikyek pgspprevvp |
| 2041 | rprpgvteat itglepgtey tiyvialknn qksepligrk ktdelpqlvt lphpnlhgpe |
| 2101 | ildvpstvqk tpfvthpgyd tgngiqlpgt sgqqpsvgqq mifeehgfrr ttppttatpi |
| 2161 | rhrprpyppn vgeeiqighi predvdyhly phgpglnpna stgqealsqt tiswapfqdt |
| 2221 | seyiischpv gtdeeplqfr vpgtstsatl tgltrgatyn iivealkdqq rhkvreevvt |
| 2281 | vgnsvnegln qptddscfdp ytvshyavgd ewermsesgf kllcqclgfg sghfrcdssr |
| 2341 | wchdngvnyk igekwdrqge ngqmmsctcl gngkgefkcd pheatcyddg ktyhvgeqwq |
| 2401 | keylgaicsc tcfggqrgwr cdncrrpgge pspegttgqs ynqysqryhq rtntnvncpi |
| 2461 | ecfmpldvqa dredsre |
| Fibronectin 1, isoform 3 precursor, NP_002017.1 | |
| 1 | mlrgpgpgll llavqclgta vpstgasksk rqaqqmvqpq spvavsqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveengest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipavppp tdlrftnigp dtmrvtwapp psidltnflv ryspvkneed vaelsispsd |
| 1321 | navvltnllp gteyvvsvss vyeqhestpl rgrqktglds ptgidfsdit ansftvhwia |
| 1381 | pratitgyri rhhpehfsgr predrvphsr nsitltnltp gteyvvsiva lngreespll |
| 1441 | igqqstvsdv prdlevvaat ptslliswda pavtvryyri tygetggnsp vqeftvpgsk |
| 1501 | statisglkp gvdytitvya vtgrgdspas skpisinyrt eidkpsqmqv tdvqdnsisv |
| 1561 | kwlpssspvt gyrvtttpkn gpgptktkta gpdqtemtie glqptveyvv svyaqnpsge |
| 1621 | sqplvqtavt nidrpkglaf tdvdvdsiki awespqgqvs ryrvtysspe dgihelfpap |
| 1681 | dgeedtaelq glrpgseytv svvalhddme sqpligtqst aipaptdlkf tqvtptslsa |
| 1741 | qwtppnvqlt gyrvrvtpke ktgpmkeinl apdsssvvvs glmvatkyev svyalkdtlt |
| 1801 | srpaqgvvtt lenvspprra rvtdatetti tiswrtktet itgfqvdavp angqtpiqrt |
| 1861 | ikpdvrsyti tglqpgtdyk iylytlndna rsspvvidas taidapsnlr flattpnsll |
| 1921 | vswqpprari tgyiikyekp gspprevvpr prpgvteati tglepgteyt iyvialknnq |
| 1981 | ksepligrkk tdelpqlvtl phpnlhgpei ldvpstvqkt pfvthpgydt gngiqlpgts |
| 2041 | gqqpsvgqqm ifeehgfrrt tppttatpir hrprpyppnv gqealsqtti swapfqdtse |
| 2101 | yiischpvgt deeplqfrvp gtstsatltg ltrgatynii vealkdqqrh kvreevvtvg |
| 2161 | nsvneglnqp tddscfdpyt vshyavgdew ermsesgfkl lcgclgfgsg hfrcdssrwc |
| 2221 | hdngvnykig ekwdrqgeng qmmsctclgn gkgefkcdph eatcyddgkt yhvgeqwqke |
| 2281 | ylgaicsctc fggqrgwrcd ncrrpggeps pegttgqsyn qysqryhqrt ntnvncpiec |
| 2341 | fmpldvqadr edsre |
| Fibronectin 1, isoform 4 precursor, NP_997643.1 | |
| 1 | mlrgpgpgll llavqclgta vpstgasksk rqaqqmvqpq spvavsqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipavppp tdlrftnigp dtmrvtwapp psidltnflv ryspvkneed vaelsispsd |
| 1321 | navvltnllp gteyvvsvss vyeqhestpl rgrqktglds ptgidfsdit ansftvhwia |
| 1381 | pratitgyri rhhpehfsgr predrvphsr nsitltnltp gteyvvsiva lngreespll |
| 1441 | igqqstvsdv prdlevvaat ptslliswda pavtvryyri tygetggnsp vqeftvpgsk |
| 1501 | statisglkp gvdytitvya vtgrgdspas skpisinyrt eidkpsqmqv tdvqdnsisv |
| 1561 | kwlpssspvt gyrvtttpkn gpgptktkta gpdqtemtie glqptveyvv svyaqnpsge |
| 1621 | sqplvqtavt nidrpkglaf tdvdvdsiki awespqgqvs ryrvtysspe dgihelfpap |
| 1681 | dgeedtaelq glrpgseytv svvalhddme sqpligtqst aipaptdlkf tqvtptslsa |
| 1741 | qwtppnvqlt gyrvrvtpke ktgpmkeinl apdsssvvvs glmvatkyev svyalkdtlt |
| 1801 | srpaqgvvtt lenvspprra rvtdatetti tiswrtktet itgfqvdavp angqtpiqrt |
| 1861 | ikpdvrsyti tglqpgtdyk iylytlndna rsspvvidas taidapsnlr flattpnsll |
| 1921 | vswqpprari tgyiikyekp gspprevvpr prpgvteati tglepgteyt iyvialknnq |
| 1981 | ksepligrkk tvqktpfvth pgydtgngiq lpgtsgqqps vgqqmifeeh gfrrttpptt |
| 2041 | atpirhrprp yppnvgqeal sqttiswapf qdtseyiisc hpvgtdeepl qfrvpgtsts |
| 2101 | atltgltrga tyniivealk dqqrhkvree vvtvgnsvne glnqptddsc fdpytvshya |
| 2161 | vgdewermse sgfkllcqcl gfgsghfrcd ssrwchdngv nykigekwdr qgengqmmsc |
| 2221 | tclgngkgef kcdpheatcy ddgktyhvge qwqkeylgai csctcfggqr gwrcdncrrp |
| 2281 | ggepspegtt gqsynqysqr yhqrtntnvn cpiecfmpld vqadredsre |
| Fibronectin 1, isoform 5 precursor, NP_997641.1 | |
| 1 | mlrgpgpgll llavqclgta vpstgasksk rqaqqmvqpq spvavsqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipavppp tdlrftnigp dtmrvtwapp psidltnflv ryspvkneed vaelsispsd |
| 1321 | navvltnllp gteyvvsvss vyeqhestpl rgrqktglds ptgidfsdit ansftvhwia |
| 1381 | pratitgyri rhhpehfsgr predrvphsr nsitltnltp gteyvvsiva lngreespll |
| 1441 | igqqstvsdv prdlevvaat ptslliswda pavtvryyri tygetggnsp vqeftvpgsk |
| 1501 | statisglkp gvdytitvya vtgrgdspas skpisinyrt eidkpsqmqv tdvqdnsisv |
| 1561 | kwlpssspvt gyrvtttpkn gpgptktkta gpdqtemtie glqptveyvv svyaqnpsge |
| 1621 | sqplvqtavt tipaptdlkf tqvtptslsa qwtppnvqlt gyrvrvtpke ktgpmkeinl |
| 1681 | apdsssvvvs glmvatkyev svyalkdtlt srpaqgvvtt lenvspprra rvtdatetti |
| 1741 | tiswrtktet itgfqvdavp angqtpiqrt ikpdvrsyti tglqpgtdyk iylytlndna |
| 1801 | rsspvvidas taidapsnlr flattpnsll vswqpprari tgyiikyekp gspprevvpr |
| 1861 | prpgvteati tglepgteyt iyvialknnq ksepligrkk tdelpqlvtl phpnlhgpei |
| 1921 | ldvpstvqkt pfvthpgydt gngiqlpgts gqqpsvgqqm ifeehgfrrt tppttatpir |
| 1981 | hrprpyppnv geeiqighip redvdyhlyp hgpglnpnas tgqealsqtt iswapfqdts |
| 2041 | eyiischpvg tdeeplqfrv pgtstsatlt gltrgatyni ivealkdqqr hkvreevvtv |
| 2101 | gnsvneglnq ptddscfdpy tvshyavgde wermsesgfk llcqclgfgs ghfrcdssrw |
| 2161 | chdngvnyki gekwdrqgen gqmmsctclg ngkgefkcdp heatcyddgk tyhvgeqwqk |
| 2221 | eylgaicsct cfggqrgwrc dncrrpggep spegttgqsy nqysgryhqr tntnvncpie |
| 2281 | cfmpldvqad redsre |
| Fibronectin 1, isoform 6 precursor, NP_997639.1 | |
| 1 | mlrgpgpgll llavqclgta vpstgasksk rqaqqmvqpq spvavsgskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | ragitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipavppp tdlrftnigp dtmrvtwapp psidltnflv ryspvkneed vaelsispsd |
| 1321 | navvltnllp gteyvvsyss vyeqhestpl rgrqktglds ptgidfsdit ansftvhwia |
| 1381 | pratitgyri rhhpehfsgr predrvphsr nsitltnltp gteyvvsiva lngreespll |
| 1441 | igqqstvsdv prdlevvaat ptslliswda pavtvryyri tygetggnsp vqeftvpgsk |
| 1501 | statisglkp gvdytitvya vtgrgdspas skpisinyrt eidkpsqmqv tdvqdnsisv |
| 1561 | kwlpssspvt gyrvtttpkn gpgptktkta gpdqtemtie glqptveyvv svyaqnpsge |
| 1621 | sqplvqtavt tipaptdlkf tqvtptslsa qwtppnvqlt gyrvrvtpke ktgpmkeinl |
| 1681 | apdsssvvvs glmvatkyev svyalkdtlt srpaqgvvtt lenvspprra rvtdatetti |
| 1741 | tiswrtktet itgfqvdavp angqtpiqrt ikpdvrsyti tglqpgtdyk iylytlndna |
| 1801 | rsspvvidas taidapsnlr flattpnsll vswqpprari tgyiikyekp gspprevvpr |
| 1861 | prpgvteati tglepgteyt iyvialknnq ksepligrkk tgqealsqtt iswapfqdts |
| 1921 | eyiischpvg tdeeplqfrv pgtstsatlt gltrgatyni ivealkdqqr hkvreevvtv |
| 1981 | gnsvneglnq ptddscfdpy tvshyavgde wermsesgfk llcqclgfgs ghfrcdssrw |
| 2041 | chdngvnyki gekwdrqgen gqmmsctclg ngkgefkcdp heatcyddgk tyhvgeqwqk |
| 2101 | eylgaicsct cfggqrgwrc dncrrpggep spegttgqsy nqysqryhqr tntnvncpie |
| 2161 | cfmpldvqad redsre |
| Fibronectin 1, isoform 7 precursor, NP_473375.2 | |
| 1 | mlrgpgpgll llavqclgta vpstgasksk rqaqqmvqpq spvavsqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpvsi pprnlgy |
| Fibronectin 1, isoform 8 precursor, NP_001293058.1 | |
| 1 | mlrgpgpgll llavqclgta vpstgasksk rqaqqmvqpq spvavsqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipevpql tdlsfvditd ssiglrwtpl nsstiigyri tvvaagegip ifedfvdssv |
| 1321 | gyytvtglep gidydisvit linggesapt tltqqtavpp ptdlrftnig pdtmrvtwap |
| 1381 | ppsidltnfl vryspvknee dvaelsisps dnavvltnll pgteyvvsvs svyeqhestp |
| 1441 | lrgrqktgld sptgidfsdi tansftvhwi apratitgyr irhhpehfsg rpredrvphs |
| 1501 | rnsitltnlt pgteyvvsiv alngreespl ligqqstvsd vprdlevvaa tptslliswd |
| 1561 | apavtvryyr itygetggns pvqeftvpgs kstatisglk pgvdytitvy avtgrgdspa |
| 1621 | sskpisinyr teidkpsqmq vtdvqdnsis vkwlpssspv tgyrvtttpk ngpgptktkt |
| 1681 | agpdqtemti eglqptveyv vsvyaqnpsg esqplvqtav tnidrpkgla ftdvdvdsik |
| 1741 | iawespqgqv sryrvtyssp edgihelfpa pdgeedtael qglrpgseyt vsvvalhddm |
| 1801 | esqpligtqs taipaptdlk ftqvtptsls aqwtppnvql tgyrvrvtpk ektgpmkein |
| 1861 | lapdsssvvv sglmvatkye vsvyalkdtl tsrpaqgvvt tlenvspprr arvtdatett |
| 1921 | itiswrtkte titgfqvdav pangqtpiqr tikpdvrsyt itglqpgtdy kiylytlndn |
| 1981 | arsspvvida staidapsnl rflattpnsl lvswqpprar itgyiikyek pgspprevvp |
| 2041 | rprpgvteat itglepgtey tiyvialknn qksepligrk ktdelpqlvt lphpnlhgpe |
| 2101 | ildvpstvqk tpfvthpgyd tgngiqlpgt sgqqpsvgqq mifeehgfrr ttppttatpi |
| 2161 | rhrprpyppn vgqealsqtt iswapfqdts eyiischpvg tdeeplqfrv pgtstsatlt |
| 2221 | gltrgatyni ivealkdqqr hkvreevvtv gnsvneglnq ptddscfdpy tvshyavgde |
| 2281 | wermsesgfk llcqclgfgs ghfrcdssrw chdngvnyki gekwdrqgen gqmmsctclg |
| 2341 | ngkgefkcdp heatcyddgk tyhvgeqwqk eylgaicsct cfggqrgwrc dncrrpggep |
| 2401 | spegttgqsy nqysqryhqr tntnvncpie cfmpldvqad redsre |
| Fibronectin 1, isoform 9 precursor, NP_001293059.1 | |
| 1 | mlrgpgpgll llavqclgta vpstgasksk rqaqqmvqpq spvavsqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipevpql tdlsfvditd ssiglrwtpl nsstiigyri tvvaagegip ifedfvdssv |
| 1321 | gyytvtglep gidydisvit linggesapt tltqqtavpp ptdlrftnig pdtmrvtwap |
| 1381 | ppsidltnfl vryspvknee dvaelsisps dnavvltnll pgteyvvsvs svyeqhestp |
| 1441 | lrgrqktgld sptgidfsdi tansftvhwi apratitgyr irhhpehfsg rpredrvphs |
| 1501 | rnsitltnlt pgteyvvsiv alngreespl ligqqstvsd vprdlevvaa tptslliswd |
| 1561 | apavtvryyr itygetggns pvqeftvpgs kstatisglk pgvdytitvy avtgrgdspa |
| 1621 | sskpisinyr teidkpsqmq vtdvqdnsis vkwlpssspv tgyrvtttpk ngpgptktkt |
| 1681 | agpdqtemti eglqptveyv vsvyaqnpsg esqplvqtav ttipaptdlk ftqvtptsls |
| 1741 | aqwtppnvql tgyrvrvtpk ektgpmkein lapdsssvvv sglmvatkye vsvyalkdtl |
| 1801 | tsrpaqgvvt tlenvspprr arvtdatett itiswrtkte titgfqvdav pangqtpiqr |
| 1861 | tikpdvrsyt itglqpgtdy kiylytlndn arsspvvida staidapsnl rflattpnsl |
| 1921 | lvswqpprar itgyiikyek pgspprevvp rprpgvteat itglepgtey tiyvialknn |
| 1981 | qksepligrk ktgqealsqt tiswapfqdt seyiischpv gtdeeplqfr vpgtstsatl |
| 2041 | tgltrgatyn iivealkdqq rhkvreevvt vgnsvnegln qptddscfdp ytvshyavgd |
| 2101 | ewermsesgf kllcqclgfg sghfrcdssr wchdngvnyk igekwdrqge ngqmmsctcl |
| 2161 | gngkgefkcd pheatcyddg ktyhvgeqwq keylgaicsc tcfggqrgwr cdncrrpgge |
| 2221 | pspegttgqs yngysgryhq rtntnvncpi ecfmpldvqa dredsre |
| Fibronectin 1, isoform 10 precursor, NP_001293060.1 | |
| 1 | mlrgpgpgll llavqclgta vpstgasksk rqaqqmvqpq spvavsqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsvsk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipavppp tdlrftnigp dtmrvtwapp psidltnflv ryspvkneed vaelsispsd |
| 1321 | navvltnllp gteyvvsvss vyeqhestpl rgrqktglds ptgidfsdit ansftvhwia |
| 1381 | pratitgyri rhhpehfsgr predrvphsr nsitltnltp gteyvvsiva lngreespll |
| 1441 | igqqstvsdv prdlevvaat ptslliswda pavtvryyri tygetggnsp vqeftvpgsk |
| 1501 | statisglkp gvdytitvya vtgrgdspas skpisinyrt eidkpsqmqv tdvqdnsisv |
| 1561 | kwlpssspvt gyrvtttpkn gpgptktkta gpdqtemtie glqptveyvv svyagnpsge |
| 1621 | sqplvqtavt tipaptdlkf tqvtptslsa qwtppnvqlt gyrvrvtpke ktgpmkeinl |
| 1681 | apdsssvvvs glmvatkyev svyalkdtlt srpaqgvvtt lenvspprra rvtdatetti |
| 1741 | tiswrtktet itgfqvdavp angqtpiqrt ikpdvrsyti tglqpgtdyk iylytlndna |
| 1801 | rsspvvidas taidapsnlr flattpnsll vswqpprari tgyiikyekp gspprevvpr |
| 1861 | prpgvteati tglepgteyt iyvialknnq ksepligrkk tdelpqlvtl phpnlhgpei |
| 1921 | ldvpstvqkt pfvthpgydt gngiqlpgts gqqpsvgqqm ifeehgfrrt tppttatpir |
| 1981 | hrprpyppnv gqealsqtti swapfqdtse yiischpvgt deeplqfrvp gtstsatltg |
| 2041 | ltrgatynii vealkdqqrh kvreevvtvg nsvneglnqp tddscfdpyt vshyavgdew |
| 2101 | ermsesgfkl lcqclgfgsg hfrcdssrwc hdngvnvkig ekwdrqgeng qmmsctclgn |
| 2161 | gkgefkcdph eatcyddgkt yhvgeqwqke ylgaicsctc fggqrgwrcd ncrrpggeps |
| 2221 | pegttgqsyn qysqryhqrt ntnvncpiec fmpldvqadr edsre |
| Fibronectin 1, isoform 11 precursor, NP_001293061.1 | |
| 1 | mlrgpgpgll llavqclgta vpstgasksk rqaqqmvqpq spvavsqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsvsk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipavppp tdlrftnigp dtmrvtwapp psidltnflv ryspvkneed vaelsispsd |
| 1321 | navvltnllp gteyvvsyss vyeqhestpl rgrqktglds ptgidfsdit ansftvhwia |
| 1381 | pratitgyri rhhpehfsgr predrvphsr nsitltnltp gteyvvsiva lngreespll |
| 1441 | igqqstvsdv prdlevvaat ptslliswda pavtvryyri tygetggnsp vqeftvpgsk |
| 1501 | statisglkp gvdytitvya vtgrgdspas skpisinyrt eidkpsqmqv tdvqdnsisv |
| 1561 | kwlpssspvt gyrvtttpkn gpgptktkta gpdqtemtie glqptveyvv svyaqnpsge |
| 1621 | sqplvqtavt tipaptdlkf tqvtptslsa qwtppnvqlt gyrvrvtpke ktgpmkeinl |
| 1681 | apdsssvvvs glmvatkyev svyalkdtlt srpaqgvvtt lenvspprra rvtdatetti |
| 1741 | tiswrtktet itgfqvdavp angqtpiqrt ikpdvrsyti tglqpgtdyk iylytlndna |
| 1801 | rsspvvidas taidapsnlr flattpnsll vswqpprari tgyiikyekp gspprevvpr |
| 1861 | prpgvteati tglepgteyt iyvialknnq ksepligrkk tvqktpfvth pgydtgngiq |
| 1921 | lpgtsgqqps vgqqmifeeh gfrrttpptt atpirhrprp yppnvgqeal sqttiswapf |
| 1981 | qdtseyiisc hpvgtdeepl qfrvpgtsts atltgltrga tyniivealk dqqrhkvree |
| 2041 | vvtvgnsvne glnqptddsc fdpytvshya vgdewermse sgfkllcqcl gfgsghfrcd |
| 2101 | ssrwchdngv nykigekwdr qgengqmmsc tclgngkgef kcdpheatcy ddgktyhvge |
| 2161 | qwqkeylgai csctcfggqr gwrcdncrrp ggepspegtt gqsynqysqr yhqrtntnvn |
| 2221 | cpiecfmpld vqadredsre |
| Major histocompatibility complex, class II, DR beta 1, precursor, | |
| NP_001230894.1 | |
| 1 | mvclrlpggs cmavltvtlm vlssplalag dtrprfleys tsechffngt ervryldryf |
| 61 | hnqeenvrfd sdvgefravt elgrpdaeyw nsqkdlleqk rgrvdnycrh nygvvesftv |
| 121 | qrrvhpkvtv ypsktqplqh hnllvcsvsg fypgsievrw frngqeektg vvstglihng |
| 181 | dwtfqtlvml etvprsgevy tcqvehpsvt spltvewrar sesaqskmls gvggfvlgll |
| 241 | flgaglfiyf rnqkghsglq prgfls |
| Major histocompatibility complex, class II, DR beta 1, precursor, | |
| NP_001346122.1 | |
| 1 | mvclklpggs cmaaltvtlm vlssplalag dtqprflwqg kykchffngt ervqflerlf |
| 61 | ynqeefvrfd sdvgeyravt elgrpvaesw nsqkdiledr rgqvdtvcrh nygvgesftv |
| 121 | qrrvhpevtv ypaktqplqh hnllvcsvsg fypgsievrw frngqeekag vvstgliqng |
| 181 | dwtfqtlvml etvprsgevy tcqvehpsvm spltvewrar sesaqskmls gvggfvlgll |
| 241 | flgaglfiyf rnqkghsglq ptgfls |
| Major histocompatibility complex, class II, DR beta 1, precursor, | |
| NP_001346123.1 | |
| 1 | mvclkfpggs cmaaltvtlm vlssplalag dtrprfleqv khechffngt ervrfldryf |
| 61 | yhqeeyvrfd sdvgeyravt elgrpdaeyw nsqkdlleqr raevdtycrh nygvvesftv |
| 121 | qrrvypevtv ypaktqplqh hnllvcsvng fypgsievrw frngqeektg vvstgliqng |
| 181 | dwtfqtlvml etvprsgevy tcqvehpslt spltvewrar sesaqskmls gvggfvlgll |
| 241 | flgaglfiyf rnqkghsglq ptgfls |
| Major histocompatibility complex, class II, DR beta 1, precursor, | |
| NP_002115.2 | |
| 1 | mvclklpggs cmtaltvtlm vlssplalsg dtrprflwqp krechffngt ervrfldryf |
| 61 | ynqeesvrfd sdvgefravt elgrpdaeyw nsqkdileqa raavdtycrh nygvvesftv |
| 121 | qrrvqpkvtv ypsktqplqh hnllvcsvsg fypgsievrw flngqeekag mvstgliqng |
| 181 | dwtfqtlvml etvprsgevy tcqvehpsvt spltvewrar sesaqskmls gvggfvlgll |
| 241 | flgaglfiyf rnqkghsglq ptgfls |
| Major histocompatibility complex, class II, DR beta 5, precursor, | |
| NP_002116.2 | |
| 1 | mvclklpggs ymakltvtlm vlssplalag dtrprflqqd kyechffngt ervrflhrdi |
| 61 | ynqeedlrfd sdvgeyravt elgrpdaeyw nsqkdfledr raavdtycrh nygvgesftv |
| 121 | qrrvepkvtv ypartqtlqh hnllvcsvng fypgsievrw frnsqeekag vvstgliqng |
| 181 | dwtfqtlvml etvprsgevy tcqvehpsvt spltvewraq sesaqskmls gvggfvlgll |
| 241 | flgaglfiyf knqkghsglh ptglvs |
| Hydroxysteroid 17-beta dehydrogenase 3, NP_000188.1 | |
| 1 | mgdvleqffi ltgllvclac lakcvrfsrc vllnywkvlp ksflrsmgqw avitgagdgi |
| 61 | gkaysfelak rglnvvlisr tlekleaiat eierttgrsv kiiqadftkd diyehikekl |
| 121 | agleigilvn nvgmlpnllp shflnapdei qslihcnits vvkmtqlilk hmesrqkgli |
| 181 | lnissgialf pwplysmysa skafvcafsk alqeeykake viiqvltpya vstamtkyln |
| 241 | tnvitktade fvkeslnyvt iggetcgcla heilagflsl ipawafysga fqrlllthyv |
| 301 | aylklntkvr |
| Insulin degrading enzyme, isoform 1, NP_004960.2 | |
| 1 | mryrlawllh palpstfrsv lgarlppper lcgfqkktys kmnnpaikri gnhitksped |
| 61 | kreyrglela ngikvllisd pttdkssaal dvhigslsdp pniaglshfc ehmlflgtkk |
| 121 | ypkeneysqf lsehagssna ftsgehtnyy fdvshehleg aldrfaqffl cplfdesckd |
| 181 | revnavdseh eknvmndawr lfqlekatgn pkhpfskfgt gnkytletrp nqegidvrqe |
| 241 | llkfhsayys snlmavcvlg reslddltnl vvklfseven knvplpefpe hpfqeehlkq |
| 301 | lykivpikdi rnlyvtfpip dlqkyyksnp ghylghligh egpgsllsel kskgwvntlv |
| 361 | ggqkegargf mffiinvdlt eegllhvedi ilhmfqyiqk lraegpqewv fqeckdlnav |
| 421 | afrfkdkerp rgytskiagi lhyypleevl taeylleefr pdliemvldk lrpenvrvai |
| 481 | vsksfegktd rteewygtqy kqeaipdevi kkwqnadlng kfklptknef iptnfeilpl |
| 541 | ekeatpypal ikdtamsklw fkqddkfflp kaclnfeffs pfayvdplhc nmaylylell |
| 601 | kdslneyaya aelaglsydl qntiygmyls vkgyndkqpi llkkiiekma tfeidekrfe |
| 661 | iikeaymrsl nnfraeqphq hamyylrllm tevawtkdel kealddvtlp rlkafipqll |
| 721 | srlhieallh gnitkqaalg imqmvedtli ehahtkpllp sqlvryrevq lpdrgwfvyq |
| 781 | qrnevhnncg ieiyyqtdmq stsenmflel fcqiisepcf ntlrtkeqlg yivfsgprra |
| 841 | ngiqglrfii qsekpphyle srveaflitm eksiedmtee afqkhiqala irrldkpkkl |
| 901 | saecakywge iisqqynfdr dntevaylkt ltkediikfy kemlavdapr rhkvsvhvla |
| 961 | remdscpvvg efpcqndinl sqapalpqpe viqnmtefkr glplfplvkp hinfmaakl |
| Insulin degrading enzyme, isoform 2, NP_001159418.1 | |
| 1 | msklwfkqdd kfflpkacln feffspfayv dplhcnmayl ylellkdsln eyayaaelag |
| 61 | lsydlqntiy gmylsvkgyn dkqpillkki iekmatfeid ekrfeiikea ymrslnnfra |
| 121 | eqphqhamyy lrllmtevaw tkdelkeald dvtlprlkaf ipqllsrlhi eallhgnitk |
| 181 | qaalgimqmv edtliehaht kpllpsqlvr yrevqlpdrg wfvyqqrnev hnncgieiyy |
| 241 | qtdmqstsen mflelfcqii sepcfntlrt keqlgyivfs gprrangiqg lrfiiqsekp |
| 301 | phylesrvea flitmeksie dmteeafqkh iqalairrld kpkklsaeca kywgeiisqq |
| 361 | ynfdrdntev aylktltked iikfykemla vdaprrhkvs vhvlaremds cpvvgefpcq |
| 421 | ndinlsqapa lpqpeviqnm tefkrglplf plvkphinfm aakl |
| Insulin degrading enzyme, isoform 3, NP_001309722.1 | |
| 1 | mryrlawllh palpstfrsv lgarlppper lcgfqkktys kmnnpaikri gnhitksped |
| 61 | kreyrglela ngikvllisd pttdkssaal dvhigslsdp pniaglshfc ehmlflgtkk |
| 121 | ypkeneysqf lsehagssna ftsgehtnyy fdvshehleg aldrfaqffl cplfdesckd |
| 181 | revnavdseh eknvmndawr lfqlekatgn pkhpfskfgt gnkytletrp nqegidvrqe |
| 241 | llkfhsayys snlmavcvlg reslddltnl vvklfseven knvplpefpe hpfqeehlkq |
| 301 | lykivpikdi rnlyvtfpip dlqkyyksnp ghylghligh egpgsllsel kskgwvntlv |
| 361 | ggqkegargf mffiinvdlt eegllhvedi ilhmfqyiqk lraegpqewv fqeckdlnav |
| 421 | afrfkdkerp rgytskiagi lhyypleevl taeylleefr pdliemvldk lrpenvrvai |
| 481 | vsksfegktd rteewygtqy kqeaipdevi kkwqnadlng kfklptknef iptnfeilpl |
| 541 | ekeatpypal ikdtamsklw fkqddkfflp kaclnfeffs ryiyadplhc nmtylfirll |
| 601 | kddlkeytya arlsglsygi asgmnaills vkgyndkqpi llkkiiekma tfeidekrfe |
| 661 | iikeaymrsl nnfraeqphq hamyylrllm tevawtkdel kealddvtlp rlkafipqll |
| 721 | srlhieallh gnitkqaalg imqmvedtli ehahtkpllp sqlvryrevq lpdrgwfvyq |
| 781 | qrnevhnncg ieiyyqtdmq stsenmflel fcqiisepcf ntlrtkeqlg yivfsgprra |
| 841 | ngiqglrfii qsekpphyle srveaflitm eksiedmtee afqkhiqala irrldkpkkl |
| 901 | saecakywge iisqqynfdr dntevaylkt ltkediikfy kemlavdapr rhkvsvhvla |
| 961 | remdscpvvg efpcqndinl sqapalpqpe viqnmtefkr glplfplvkp hinfmaakl |
| Insulin degrading enzyme, isoform 4, NP_001309723.1 | |
| 1 | mryrlawllh palpstfrsv lgarlppper lcgfqkktys kmnnpaikri gnhitksped |
| 61 | kreyrglela ngikvllisd pttdkssaal dvhigslsdp pniaglshfc ehmlflgtkk |
| 121 | ypkeneysqf lsehagssna ftsgehtnyy fdvshehleg aldrfaqffl cplfdesckd |
| 181 | revnavdseh eknvmndawr lfqlekatgn pkhpfskfgt greslddltn lvvklfseve |
| 241 | nknvplpefp ehpfqeehlk qlykivpikd irnlyvtfpi pdlqkyyksn pghylghlig |
| 301 | hegpgsllse lkskgwvntl vggqkegarg fmffiinvdl teegllhved iilhmfqyiq |
| 361 | klraegpqew vfqeckdlna vafrfkdker prgytskiag ilhyypleev ltaeylleef |
| 421 | rpdliemvld klrpenvrva ivsksfegkt drteewygtq ykqeaipdev ikkwqnadln |
| 481 | gkfklptkne fiptnfeilp lekeatpypa likdtamskl wfkqddkffl pkaclnfeff |
| 541 | spfayvdplh cnmaylylel lkdslneyay aaelaglsyd lqntiygmyl svkgyndkqp |
| 601 | illkkiiekm atfeidekrf eiikeaymrs lnnfraeqph ghamyylrll mtevawtkde |
| 661 | lkealddvtl prlkafipql lsrlhieall hgnitkqaal gimqmvedtl iehahtkpll |
| 721 | psqlvryrev qlpdrgwfvy qqrnevhnnc gieiyyqtdm qstsenmfle lfcqiisepc |
| 781 | fntlrtkeql gyivfsgprr angiqglrfi igsekpphyl esrveaflit meksiedmte |
| 841 | eafqkhiqal airrldkpkk lsaecakywg eiisqqynfd rdntevaylk tltkediikf |
| 901 | ykemlavdap rrhkvsvhvl aremdscpvv gefpcqndin lsqapalpqp eviqnmtefk |
| 961 | rglplfplvk phinfmaakl |
| Insulin degrading enzyme, isoform 5, NP_001309724.1, NP_001309725.1 | |
| 1 | mnnpaikrig nhitkspedk reyrglelan gikvllisdp ttdkssaald vhigslsdpp |
| 61 | niaglshfce hmlflgtkky pkeneysqfl sehagssnaf tsgehtnyyf dvshehlega |
| 121 | ldrfaqfflc plfdesckdr evnavdsehe knvmndawrl fqlekatgnp khpfskfgtg |
| 181 | nkytletrpn qegidvrqel lkfhsayyss nlmavcvlgr eslddltnlv vklfsevenk |
| 241 | nvplpefpeh pfqeehlkql ykivpikdir nlyvtfpipd lqkyyksnpg hylghlighe |
| 301 | gpgsllselk skgwvntlvg gqkegargfm ffiinvdlte egllhvedii lhmfqyiqkl |
| 361 | raegpqewvf qeckdlnava frfkdkerpr gytskiagil hyypleevlt aeylleefrp |
| 421 | dliemvldkl rpenvrvaiv sksfegktdr teewygtqyk qeaipdevik kwqnadlngk |
| 481 | fklptknefi ptnfeilple keatpypali kdtamsklwf kqddkfflpk aclnfeffsp |
| 541 | fayvdplhcn maylylellk dslneyayaa elaglsydlq ntiygmylsv kgyndkqpil |
| 601 | lkkiiekmat feidekrfei ikeaymrsln nfraeqphqh amyylrllmt evawtkdelk |
| 661 | ealddvtlpr lkafipqlls rlhieallhg nitkqaalgi mqmvedtlie hahtkpllps |
| 721 | qlvryrevql pdrgwfvyqq rnevhnncgi eiyyqtdmqs tsenmflelf cqiisepcfn |
| 781 | tlrtkeqlgy ivfsgprran giqglrfiiq sekpphyles rveaflitme ksiedmteea |
| 841 | fqkhiqalai rrldkpkkls aecakywgei isqqynfdrd ntevaylktl tkediikfyk |
| 901 | emlavdaprr hkvsvhvlar emdscpvvge fpcqndinls qapalpqpev iqnmtefkrg |
| 961 | lplfplvkph infmaakl |
| Insulin degrading enzyme, isoform 6, NP_001309726.1 | |
| 1 | msklwfkqdd kfflpkacln feffsryiya dplhcnmtyl firllkddlk eytyaarlsg |
| 61 | lsygiasgmn aillsvkgyn dkqpillkki iekmatfeid ekrfeiikea ymrslnnfra |
| 121 | eqphqhamyy lrllmtevaw tkdelkeald dvtlprlkaf ipqllsrlhi eallhgnitk |
| 181 | qaalgimqmv edtliehaht kpllpsqlvr yrevqlpdrg wfvyqqrnev hnncgieiyy |
| 241 | qtdmqstsen mflelfcqii sepcfntlrt keqlgyivfs gprrangiqg lrfiiqsekp |
| 301 | phylesrvea flitmeksie dmteeafqkh iqalairrld kpkklsaeca kywgeiisqq |
| 361 | ynfdrdntev aylktltked iikfykemla vdaprrhkvs vhvlaremds cpvvgefpcq |
| 421 | ndinlsqapa lpqpeviqnm tefkrglplf plvkphinfm aakl |
| Indoleamine 2,3-dioxygenase 1, NP_002155.1 | |
| 1 | mahamenswt iskeyhidee vgfalpnpqe nlpdfyndwm fiakhlpdli esgqlrerve |
| 61 | klnmlsidhl tdhksqrlar lvlgcitmay vwgkghgdvr kvlprniavp ycqlskklel |
| 121 | ppilvyadcv lanwkkkdpn kpltyenmdv lfsfrdgdcs kgfflvsllv eiaaasaikv |
| 181 | iptvfkamqm gerdtllkal leiascleka lqvfhqihdh vnpkaffsvl riylsgwkgn |
| 241 | pqlsdglvye gfwedpkefa ggsagqssvf qcfdvllgiq qtaggghaaq flqdmrrymp |
| 301 | pahrnflcsl esnpsvrefv lskgdaglre aydacvkalv slrsyhlqiv tkyilipasq |
| 361 | qpkenktsed pskleakgtg gtdlmnflkt vrstteksll keg |
| Insulin like growth factor binding protein 5, precursor, NP_000590.1 | |
| 1 | mvlltavlll laayagpaqs lgsfvhcepc dekalsmcpp splgcelvke pgcgccmtca |
| 61 | laegqscgvy tercaqglrc lprqdeekpl hallhgrgvc lneksyreqv kierdsrehe |
| 121 | epttsemaee tyspkifrpk htriselkae avkkdrrkkl tqskfvggae ntahpriisa |
| 181 | pemrqeseqg pcrrhmeasl qelkasprmv pravylpncd rkgfykrkqc kpsrgrkrgi |
| 241 | cwcvdkygmk lpgmeyvdgd fqchtfdssn ve |
| Insulin like growth factor binding protein 7, isoform 1 precursor, | |
| NP_001544.1 | |
| 1 | merpslrall lgaaglllll lplssssssd tcgpcepasc pplpplgcll getrdacgcc |
| 61 | pmcargegep cggggagrgy capgmecvks rkrrkgkaga aaggpgvsgv cvcksrypvc |
| 121 | gsdgttypsg cqlraasqra esrgekaitq vskgtceqgp sivtppkdiw nvtgaqvyls |
| 181 | cevigiptpv liwnkvkrgh ygvqrtellp gdrdnlaiqt rggpekhevt gwvlvsplsk |
| 241 | edageyecha snsqgqasas akitvvdalh eipvkkgega el |
| Insulin like growth factor binding protein 7, isoform 2 precursor, | |
| NP_001240764.1 | |
| 1 | merpslrall lgaaglllll lplssssssd tcgpcepasc pplpplgcll getrdacgcc |
| 61 | pmcargegep cggggagrgy capgmecvks rkrrkgkaga aaggpgvsgv cvcksrypvc |
| 121 | gsdgttypsg cqlraasqra esrgekaitq vskgtceqgp sivtppkdiw nvtgaqvyls |
| 181 | cevigiptpv liwnkvkrgh ygvqrtellp gdrdnlaiqt rggpekhevt gwvlvsplsk |
| 241 | edageyecha snsqgqasas akitvvdalh eipvkkgtq |
| Potassium two pore domain channel subfamily K member 1, NP_002236.1 | |
| 1 | mlqslagssc vrlverhrsa wcfgflvlgy llylvfgavv fssvelpyed llrqelrklk |
| 61 | rrfleehecl seqqleqflg rvleasnygv svlsnasgnw nwdftsalff astvlsttgy |
| 121 | ghtvplsdgg kafciiysvi gipftllflt avvqritvhv trrpvlyfhi rwgfskqvva |
| 181 | ivhavllgfv tvscfffipa avfsvleddw nflesfyfcf islstiglgd yvpgegynqk |
| 241 | frelykigit cylllgliam lvvletfcel helkkfrkmf yvkkdkdedq vhiiehdqls |
| 301 | fssitdqaag mkedqkqnep fvatqssacv dgpanh |
| Lysosomal associated membrane protein 3, precursor, NP_055213.2 | |
| 1 | mprqlsaaaa lfaslavilh dgsqmrakaf petrdysqpt aaatvqdikk pvqqpakqap |
| 61 | hqtlaarfmd ghitfqtaat vkiptttpat tkntattspi tytlvttqat pnnshtappv |
| 121 | tevtvgpsla pyslpptitp pahttgtsss tvshttgntt qpsnqttlpa tlsialhkst |
| 181 | tgqkpvqpth apgttaaahn ttrtaapast vpgptlapqp ssvktgiyqv lngsrlcika |
| 241 | emgiqlivqd kesvfsprry fnidpnatqa sgncgtrksn lllnfqggfv nitftkdees |
| 301 | yyisevgayl tvsdpetiyq gikhavvmfq tavghsfkcv seqslqlsah lqvkttdvql |
| 361 | qafdfeddhf gnvdecssdy tivlpvigai vvglclmgmg vykirlrcqs sgyqri |
| MAGE family member B2, NP_002355.2 | |
| 1 | mprgqksklr arekrrkard etrglnvpqv teaeeeeapc csssvsggaa ssspaagipq |
| 61 | epqrapttaa aaaagvsstk skkgakshqg eknasssqas tstkspsedp ltrksgslvq |
| 121 | fllykykikk svtkgemlki vgkrfrehfp eilkkasegl svvfglelnk vnpnghtytf |
| 181 | idkvdltdee sllsswdfpr rkllmpllgv iflngnsate eeiweflnml gvydgeehsv |
| 241 | fgepwklitk dlvqekyley kqvpssdppr fqflwgpray aetskmkvle flakvngttp |
| 301 | cafpthyeea lkdeekagv |
| Mitogen-activated protein kinase 13, NP_002745.1 | |
| 1 | mslirkkgfy kqdvnktawe lpktyvspth vgsgaygsvc saidkrsgek vaikklsrpf |
| 61 | qseifakray rellllkhmq henviglldv ftpasslrnf ydfylvmpfm qtdlqkimgm |
| 121 | efseekiqyl vyqmlkglky ihsagvvhrd lkpgnlavne dcelkildfg larhadaemt |
| 181 | gyvvtrwyra pevilswmhy nqtvdiwsvg cimaemltgk tlfkgkdyld qltqilkvtg |
| 241 | vpgtefvqkl ndkaaksyiq slpqtprkdf tqlfpraspq aadllekmle ldvdkrltaa |
| 301 | qalthpffep frdpeeetea qqpfddsleh ekltvdewkq hiykeivnfs piarkdsrrr |
| 361 | sgmkl |
| Macrophage receptor with collagenous structure, NP_006761.1 | |
| 1 | mrnkkilked ellsetqqaa fhqiamepfe invpkpkrrn gvnfslavvv iylilltaga |
| 61 | gllvvqvlnl qarlrvlemy flndtlaaed spsfsllqsa hpgehlaqga srlqvlqaql |
| 121 | twvrvshehl lqrvdnftqn pgmfrikgeq gapglqghkg amgmpgapgp pgppaekgak |
| 181 | gamgrdgatg psgpqgppgv kgeaglqgpq gapgkqgatg tpgpqgekgs kgdggligpk |
| 241 | getgtkgekg dlglpgskgd rgmkgdagvm gppgaqgskg dfgrpgppgl agfpgakgdq |
| 301 | gqpglqgvpg ppgavghpga kgepgsagsp graglpgspg spgatglkgs kgdtglqgqq |
| 361 | grkgesgvpg pagvkgeqgs pglagpkgap gqagqkgdqg vkgssgeqgv kgekgergen |
| 421 | svsvrivgss nrgraevyys gtwgticdde wqnsdaivfc rmlgyskgra lykvgagtgq |
| 481 | iwldnvqcrg testlwsctk nswghhdcsh eedagvecsv |
| Malic enzyme 1, NADP-dependent malic enzyme, NP_002386.1 | |
| 1 | mepeaprrrh thqrgylltr nphlnkdlaf tleerqqlni hgllppsfns qeiqvlrvvk |
| 61 | nfehlnsdfd rylllmdlqd rneklfyrvl tsdiekfmpi vytptvglac qqyslvfrkp |
| 121 | rglfitihdr ghiasvlnaw pedvikaivv tdgerilglg dlgcngmgip vgklalytac |
| 181 | ggmnpqeclp vildvgtene ellkdplyig lrqrrvrgse yddfldefme avsskygmnc |
| 241 | liqfedfanv nafrllnkyr nqyctfnddi qgtasvavag llaalritkn klsdqtilfq |
| 301 | gageaalgia hlivmaleke glpkekaikk iwlvdskgli vkgrasltqe kekfahehee |
| 361 | mknleaivqe ikptaligva aiggafseqi lkdmaafner piifalsnpt skaecsaeqc |
| 421 | ykitkgraif asgspfdpvt lpngqtlypg qgnnsyvfpg valgvvacgl rqitdniflt |
| 481 | taeviaqqvs dkhleegrly pplntirdvs lkiaekivkd ayqektatvy pepqnkeafv |
| 541 | rsqmystdyd qilpdcyswp eevqkiqtkv dq |
| Migration and invasion inhibitory protein, NP_068752.2 | |
| 1 | mveaeelaql rllnlellrq lwvgqdavrr svaraasess lessssynse tpstpetsst |
| 61 | slstscprgr ssvwgppdac rgdlrdvars gvaslppakc qhqeslgrpr phsapslgts |
| 121 | slrdpepsgr lgdpgpqeaq tprsilaqqs klskprvtfs eesavpkrsw rlrpylgydw |
| 181 | iagsldtsss itsqpeaffs klqefretnk eecicshpep qlpglressg sgveedhecv |
| 241 | ycyrvnrrlf pvpvdpgtpc rlcrtprdqq gpgtlaqpah vrvsiplsil epphryhihr |
| 301 | rksfdasdtl alprhcllgw difppkseks saprnldlws svsaeaqhqk lsgtsspfhp |
| 361 | aspmqmlppt ptwsvpqvpr phvprqkp |
| Matrix metallopeptidase 12, macrophage metalloelastase preproprotein, | |
| NP_002417.2 | |
| 1 | mkfllilllq atasgalpln sstsleknnv lfgerylekf ygleinklpv tkmkysgnlm |
| 61 | kekiqemqhf lglkvtgqld tstlemmhap rcgvpdvhhf rempggpvwr khyityrinn |
| 121 | ytpdmnredv dyairkafqv wsnvtplkfs kintgmadil vvfargahgd fhafdgkggi |
| 181 | lahafgpgsg iggdahfded efwtthsggt nlfltavhei ghslglghss dpkavmfpty |
| 241 | kyvdintfrl saddirgiqs lygdpkenqr lpnpdnsepa lcdpnlsfda vttvgnkiff |
| 301 | fkdrffwlkv serpktsvnl isslwptlps gieaayeiea rnqvflfkdd kywlisnlrp |
| 361 | epnypksihs fgfpnfvkki daavfnprfy rtyffvdnqy wryderrqmm dpgypklitk |
| 421 | nfqgigpkid avfysknkyy yffqgsnqfe ydfllqritk tlksnswfgc |
| Matrix metallopeptidase 7, matrilysin preproprotein, NP_002414.1 | |
| 1 | mrltvlcavc llpgslalpl pqeaggmsel qweqaqdylk rfylydsetk nansleaklk |
| 61 | emqkffglpi tgmlnsrvie imqkprcgvp dvaeyslfpn spkwtskvvt yrivsytrdl |
| 121 | phitvdrlvs kalnmwgkei plhfrkvvwg tadimigfar gahgdsypfd gpgntlahaf |
| 181 | apgtglggda hfdederwtd gsslginfly aathelghsl gmghssdpna vmyptygngd |
| 241 | pqnfklsqdd ikgiqklygk rsnsrkk |
| Myelin protein zero like 1, myelin protein zero-like protein 1 isoform | |
| a precursor, NP_003944.1 | |
| 1 | maasagagav iaapdsrrwl wsvlaaalgl ltagvsalev ytpkeifvan gtqgkltckf |
| 61 | kststtgglt svswsfqpeg adttvsffhy sqgqvylgny ppfkdriswa gdldkkdasi |
| 121 | nienmqfihn gtyicdvknp pdivvqpghi rlyvvekenl pvfpvwvvvg ivtavvlglt |
| 181 | llismilavl yrrknskrdy tgcstsesls pvkqaprksp sdteglvksl psgshqgpvi |
| 241 | yaqldhsggh hsdkinkses vvyadirkn |
| Myelin protein zero like 1, myelin protein zero-like protein 1 isoform | |
| b precursor, NP_078845.3 | |
| 1 | maasagagav iaapdsrrwl wsvlaaalgl ltagvsalev ytpkeifvan gtqgkltckf |
| 61 | kststtgglt svswsfqpeg adttvsffhy sqgqvylgny ppfkdriswa gdldkkdasi |
| 121 | nienmqfihn gtyicdvknp pdivvqpghi rlyvvekenl pvfpvwvvvg ivtavvlglt |
| 181 | llismilavl yrrknskrdy tgaqsymhs |
| Myelin protein zero like 1, myelin protein zero-like protein 1 isoform | |
| c precursor, NP_001139663.1 | |
| 1 | maasagagav iaapdsrrwl wsvlaaalgl ltagvsalev ytpkeifvan gtqgkltckf |
| 61 | kststtgglt svswsfqpeg adttvsgpvi yaqldhsggh hsdkinkses vvyadirkn |
| Macrophage scavenger receptor 1, macrophage scavenger receptor types I | |
| and II isoform type 1, NP_619729.1 | |
| 1 | meqwdhfhnq qedtdscses vkfdarsmta llppnpknsp slqeklksfk aalialyllv |
| 61 | favlipligi vaaqllkwet kncsvsstna nditqsltgk gndseeemrf qevfmehmsn |
| 121 | mekriqhild meanlmdteh fqnfsmttdq rfndillqls tlfssvqghg naideisksl |
| 181 | islnttlldl qlnienlngk iqentfkqqe eiskleervy nvsaeimamk eeqvhleqei |
| 241 | kgevkvlnni tndlrlkdwe hsqtlrnitl iqgppgppge kgdrgptges gprgfpgpig |
| 301 | ppglkgdrga igfpgsrglp gyagrpgnsg pkgqkgekgs gntltpftkv rlvggsgphe |
| 361 | grveilhsgq wgticddrwe vrvgqvvcrs lgypgvqavh kaahfgqgtg piwlnevfcf |
| 421 | gressieeck irqwgtracs hsedagvtct l |
| Macrophage scavenger receptor 1, macrophage scavenger receptor types I | |
| and II isoform type 2, NP_002436.1 | |
| 1 | meqwdhfhnq qedtdscses vkfdarsmta llppnpknsp slqeklksfk aalialyllv |
| 61 | favlipligi vaaqllkwet kncsvsstna nditqsltgk gndseeemrf qevfmehmsn |
| 121 | mekriqhild meanlmdteh fqnfsmttdq rfndillqls tlfssvqghg naideisksl |
| 181 | islnttlldl qlnienlngk igentfkqqe eiskleervy nvsaeimamk eeqvhleqei |
| 241 | kgevkvlnni tndlrlkdwe hsqtlrnitl iqgppgppge kgdrgptges gprgfpgpig |
| 301 | ppglkgdrga igfpgsrglp gyagrpgnsg pkgqkgekgs gntlrpvqlt dhiragps |
| Macrophage scavenger receptor 1, macrophage scavenger receptor types I | |
| and II isoform type 3, NP_619730.1 | |
| 1 | meqwdhfhnq qedtdscses vkfdarsmta llppnpknsp slqeklksfk aalialyllv |
| 61 | favlipligi vaaqllkwet kncsvsstna nditqsltgk gndseeemrf qevfmehmsn |
| 121 | mekriqhild meanlmdteh fqnfsmttdq rfndillqls tlfssvqghg naideisksl |
| 181 | islnttlldl qlnienlngk iqentfkqqe eiskleervy nvsaeimamk eeqvhleqei |
| 241 | kgevkvlnni tndlrlkdwe hsqtlrnitl iqgppgppge kgdrgptges gprgfpgpig |
| 301 | ppglkgdrga igfpgsrglp gyagrpgnsg pkgqkgekgs gntlstgpiw lnevfcfgre |
| 361 | ssieeckirq wgtracshse dagvtctl |
| Myoneurin, isoform A, NP_001172047.1, NP_061127.1 | |
| 1 | mqyshhcehl lerlnkqrea gflcdctivi gefqfkahrn vlasfseyfg aiyrstsenn |
| 61 | vfldqsqvka dgfqkllefi ytgtlnldsw nvkeihqaad ylkveevvtk ckikmedfaf |
| 121 | ianpssteis sitgnielnq qtclltlrdy nnreksevst dliqanpkqg alakkssqtk |
| 181 | kkkkafnspk tgqnktvqyp sdilenasve lfldanklpt pvveqvaqin dnseleltsv |
| 241 | ventfpaqdi vhtvtvkrkr gksqpncalk ehsmsniasv kspyeaensg eeldqryska |
| 301 | kpmcntcgkv fseasslrrh mrihkgvkpy vchlcgkaft qcnqlkthvr thtgekpykc |
| 361 | elcdkgfaqk cqlvfhsrmh hgeekpykcd vcnlqfatss nlkiharkhs gekpyvcdrc |
| 421 | gqrfagastl tyhvrrhtge kpyvcdtcgk afavssslit hsrkhtgekp yicgicgksf |
| 481 | issgelnkhf rshtgerpfi celcgnsytd iknlkkhktk vhsgadktld ssaedhtlse |
| 541 | qdsiqkspls etmdvkpsdm tlplalplgt edhhmllpvt dtqsptsdtl lrstvngyse |
| 601 | pqliflqqly |
| Myoneurin, isoform B, NP_001172048.1 | |
| 1 | mqyshhcehl lerlnkqrea gflcdctivi gefqfkahrn vlasfseyfg aiyrstsenn |
| 61 | vfldqsqvka dgfqkllefi ytgtlnldsw nvkeihqaad ylkveevvtk ckikmedfaf |
| 121 | ianpssteis sitgnielnq qtclltlrdy nnreksevst dliqanpkqg alakkssqtk |
| 181 | kkkkafnspk tgqnktvqyp sdilenasve lfldanklpt pvveqvaqin dnseleltsv |
| 241 | ventfpaqdi vhtvtvkrkr gksqpncalk ehsmsniasv kspyeaensg eeldqryska |
| 301 | kpmcntcgkv fseasslrrh mrihkgvkpy vchlcgkaft qcnqlkthvr thtgekpykc |
| 361 | elcdkgfaqk cqlvfhsrmh hgeekpykcd vcnlqfatss nlkiharkhs gekpyvcdrc |
| 421 | gqrfagastl tyhvrrhtge kpyvcdtcgk afavssslit hsrkhtgekp yicgicgksf |
| 481 | issgelnkhf rshtgadktl dssaedhtls eqdsiqkspl setmdvkpsd mtlplalplg |
| 541 | tedhhmllpv tdtqsptsdt llrstvngys epqliflqql y |
| N-acetylglucosamine kinase, isoform 1, NP_060037.3 | |
| 1 | mrtrtgsqla arevtgsgav prqlegrrcq agrdanggts sdgsssmaai yggvegggtr |
| 61 | sevllvsedg kilaeadgls tnhwligtdk cverinemvn rakrkagvdp lvplrslgls |
| 121 | lsggdqedag rilieelrdr fpylsesyli ttdaagsiat atpdggvvli sgtgsncrli |
| 181 | npdgsesgcg gwghmmgdeg saywiahqav kivfdsidnl eaaphdigyv kqamfhyfqv |
| 241 | pdrlgilthl yrdfdkcrfa gfcrkiaega qqgdplsryi frkagemlgr hivavlpeid |
| 301 | pvlfqgkigl pilcvgsvwk swellkegfl laltqgreiq aqnffssftl mklrhssalg |
| 361 | gaslgarhig hllpmdysan aiafysytfs |
| N-acetylglucosamine kinase, isoform 2, NP_001317354.1, NP_001317355.1 | |
| 1 | mvnrakrkag vdplvplrsl glslsggdqe dagrilieel rdrfpylses ylittdaags |
| 61 | iatatpdggv vlisgtgsnc rlinpdgses gcggwghmmg degsaywiah qavkivfdsi |
| 121 | dnleaaphdi gyvkqamfhy fqvpdrlgil thlyrdfdkc rfagfcrkia egaqqgdpls |
| 181 | ryifrkagem lgrhivavlp eidpvlfqgk iglpilcvgs vwkswellke gfllaltqgr |
| 241 | eiqaqnffss ftlmklrhss alggaslgar highllpmdy sanaiafysy tfs |
| Napsin A aspartic peptidase, preproprotein, NP_004842.1 | |
| 1 | mspppllqpl llllpllnve psgatlirip lhrvqpgrri lnllrgwrep aelpklgaps |
| 61 | pgdkpifvpl snyrdvqyfg eiglgtppqn ftvafdtgss nlwvpsrrch ffsvpcwlhh |
| 121 | rfdpkasssf qangtkfaiq ygtgrvdgil sedkltiggi kgasvifgea lwepslvfaf |
| 181 | ahfdgilglg fpilsvegvr ppmdvlveqg lldkpvfsfy lnrdpeepdg gelvlggsdp |
| 241 | ahyippltfv pvtvpaywqi hmervkvgpg ltlcakgcaa ildtgtslit gpteeiralh |
| 301 | aaiggiplla geyiilcsei pklpavsfll ggvwfnltah dyviqttrng vrlclsgfqa |
| 361 | ldvpppagpf wilgdvflgt yvavfdrgdm kssarvglar artrgadlgw getaqaqfpg |
| Nuclear transcription factor Y subunit gamma, isoform 1, | |
| NP_001136060.1 | |
| 1 | msteggfggt sssdaqqslq sfwprvmeei rnltvkdfrv qelplarikk imkldedvkm |
| 61 | isaeapvlfa kaaqifitel tlrawihted nkrrtlqrnd iamaitkfdq fdflidivpr |
| 121 | delkppkrqe evrqsvtpae pvqyyftlaq qptavqvqgq qqgqqttsst ttiqpgqiii |
| 181 | aqpqqgqttp vtmqvgegqq vqivqaqpqg qaqqaqsgtg qtmqvmqqii tntgeiqqip |
| 241 | vqlnagqlqy irlaqpvsgt qvvqgqiqtl atnaqqgqrn asqgkprrcl ketlqitqte |
| 301 | vqqgqqqfsq ftdgqqlyqi qqvtmpagqd laqpmfiqsa nqpsdgqapq vtgd |
| Nuclear transcription factor Y subunit gamma, isoform 2, NP_055038.2 | |
| 1 | msteggfggt sssdaqqslq sfwprvmeei rnltvkdfrv qelplarikk imkldedvkm |
| 61 | isaeapvlfa kaaqifitel tlrawihted nkrrtlqrnd iamaitkfdq fdflidivpr |
| 121 | delkppkrqe evrqsvtpae pvqyyftlaq qptavqvqgq qqgqqttsst ttiqpgqiii |
| 181 | aqpqqgqttp vtmqvgegqq vqivqaqpqg qaqqaqsgtg qtmqvmqqii tntgeiqqip |
| 241 | vqlnagqlqy irlaqpvsgt qvvqgqiqtl atnaqqitqt evqqgqqqfs qftdgqqlyq |
| 301 | iqqvtmpagq dlaqpmfiqs anqpsdgqap qvtgd |
| Nuclear transcription factor Y subunit gamma, isoform 3, | |
| NP_001136059.1 | |
| 1 | msteggfggt sssdaqqslq sfwprvmeei rnltvkdfrv qelplarikk imkldedvkm |
| 61 | isaeapvlfa kaaqifitel tlrawihted nkrrtlqrnd iamaitkfdq fdflidivpr |
| 121 | delkppkrqe evrqsvtpae pvqyyftlaq qptavqvqgq qqgqqttsst ttiqpgqiii |
| 181 | aqpqqgqttp vtmqvgegqq vqivqaqpqg qaqqaqsgtg qtmqvmqqii tntgeiqqip |
| 241 | vqlnagqlqy irlaqpvsgt qvvqgqiqtl atnaqqitqt evqqgqqqfs qftdgqlyqi |
| 301 | qqvtmpagqd laqpmfiqsa nqpsdgqapq vtgd |
| Nuclear transcription factor Y subunit gamma, isoform 4, | |
| NP_001136061.1 | |
| 1 | msteggfggt sssdaqqslq sfwprvmeei rnltvkdfrv qelplarikk imkldedvkr |
| 61 | ndiamaitkf dqfdflidiv prdelkppkr qeevrqsvtp aepvqyyftl aqqptavqvq |
| 121 | gqqqgqqtts stttiqpgqi iiaqpqqgqt tpvtmqvgeg qqvqivqaqp qgqaqqaqsg |
| 181 | tgqtmqvmqq iitntgeiqq ipvqlnagql gyirlaqpvs gtqvvqgqiq tlatnaqqit |
| 241 | qtevqqgqqq fsqftdgqql yqiqqvtmpa gqdlaqpmfi qsanqpsdgq apqvtgd |
| Nuclear transcription factor Y subunit gamma, isoform 5, | |
| NP_001136062.1 | |
| 1 | msteggfggt sssdaqqslq sfwprvmeei rnltvkdfrv qelplarikk imkldedvkm |
| 61 | isaeapvlfa kaaqifitel tlrawihted nkrrtlqrnd iamaitkfdq fdflidivpr |
| 121 | delkppkrqe evrqsvtpae pvqyyftlaq qptavqvqgq qqgqqttsst ttiqpgqiii |
| 181 | aqpqqgqtmq vmqqiitntg eiqqipvqln agqlqyirla qpvsgtqvvq gqiqtlatna |
| 241 | qqitqtevqq gqqqfsqftd gqqlyqiqqv tmpagqdlaq pmfiqsanqp sdgqapqvtg |
| 301 | d |
| Nuclear transcription factor Y subunit gamma, isoform 6, | |
| NP_001295043.1 | |
| 1 | msteggfggt sssdaqqslq sfwprvmeei rnltvkdfrv qelplarikk imkldedvkm |
| 61 | isaeapvlfa kaaqifitel tlrawihted nkrrtlqrnd iamaitkfdq fdflidivpr |
| 121 | delkppkrqe evrqsvtpae pvqyyftlaq qptavqvqgq qqgqqttsst ttiqpgqiii |
| 181 | aqpqqgqttp vtmqvgegqq vqivqaqpqg qaqqaqsgtg qtmqvmqqii tntgeiqqip |
| 241 | vqlnagqlqy irlaqpvsgt qvvqgqiqtl atnaqqgqrn asqgkprrcl ketlqitqte |
| 301 | vqqgqqqfsq ftdgqrnsvq qarvseltge aeprevkatg nstpctsslp tthppshrag |
| 361 | ascvccsqpq qsstspppsd alqwvvvevs gtpnqlethr elhaplpgmt slsplhpsqq |
| 421 | lyqiqqvtmp agqdlaqpmf iqsanqpsdg qapqvtgd |
| Nuclear transcription factor Y subunit gamma, isoform 7, | |
| NP_001295044.1 | |
| 1 | msteggfggt sssdaqqslq sfwprvmeei rnltvkdfrv qelplarikk imkldedvkm |
| 61 | isaeapvlfa kaaqifitel tlrawihted nkrrtlqrnd iamaitkfdq fdflidivpr |
| 121 | delkppkrqe evrqsvtpae pvqyyftlaq qptavqvqgq qqgqqttsst ttiqpgqiii |
| 181 | aqpqqgqttp vtmqvgegqq vqivqaqpqg qaqqaqsgtg qtmqvmqqii tntgeiqqip |
| 241 | vqlnagqlqy irlaqpvsgt qvvqgqiqtl atnaqqitqt evqqgqqqfs qftdgqrnsv |
| 301 | qqarvseltg eaeprevkat gnstpctssl ptthppshra gascvccsqp qqsstsppps |
| 361 | dalqwvvvev sgtpnqleth relhaplpgm tslsplhpsq qlyqiqqvtm pagqdlaqpm |
| 421 | fiqsanqpsd gqapqvtgd |
| NFKB repressing factor, isoform 1, NP_001166958.1 | |
| 1 | mgfmlplifr ysprlmekil qmaegidige mpsydlvlsk pskgqkrhls tcdgqnppkk |
| 61 | qagskfharp rfepvhfvas sskderqedp ygpqtkevne qthfasmprd iygdytqdsf |
| 121 | siqdgnsqyc dssgfiltkd qpvtanmyfd sgnpapstts qqansqstpe pspsqtfpes |
| 181 | vvaekqyfie kltatiwknl snpemtsgsd kinytymltr ciqacktnpe yiyaplkeip |
| 241 | padipknkkl ltdgyacevr cqniylttgy agskngsrdr atelavkllq krievrvvrr |
| 301 | kfkhtfgedl vvcqigmssy efppalkppe dlvvlgkdas gqpifnasak hwtnfviten |
| 361 | andaigilnn sasfnkmsie ykyemmpnrt wrcrvflqdh claegygtkk tskhaaadea |
| 421 | lkilqktqpt ypsvkssqch tgssprgsgk kkdikdlvvy enssnpvctl ndtaqfnrmt |
| 481 | veyvyermtg lrwkckvile seviaeavgv kktvkyeaag eavktlkktq ptvinnlkkg |
| 541 | avedvisrne iqgrsaeeay kqqikednig nqllrkmgwt ggglgksgeg irepisvkeq |
| 601 | hkreglgldv ervnkiakrd ieqiirnyar seshtdltfs reltnderkq ihqiaqkygl |
| 661 | kskshgvghd rylvvgrkrr kedlldqlkq egqvghyelv mpqan |
| NFKB repressing factor, isoform 2, NP_001166959.1, NP_060014.2 | |
| 1 | mekilqmaeg idigempsyd lvlskpskgq krhlstcdgq nppkkqagsk fharprfepv |
| 61 | hfvassskde rqedpygpqt kevneqthfa smprdiyqdy tqdsfsiqdg nsqycdssgf |
| 121 | iltkdqpvta nmyfdsgnpa psttsqqans qstpepspsq tfpesvvaek qyfiekltat |
| 181 | iwknlsnpem tsgsdkinyt ymltrciqac ktnpeyiyap lkeippadip knkklltdgy |
| 241 | acevrcqniy lttgyagskn gsrdratela vkllqkriev rvvrrkfkht fgedlvvcqi |
| 301 | gmssyefppa lkppedlvvl gkdasgqpif nasakhwtnf vitenandai gilnnsasfn |
| 361 | kmsieykyem mpnrtwrcrv flqdhclaeg ygtkktskha aadealkilq ktqptypsvk |
| 421 | ssqchtgssp rgsgkkkdik dlvvyenssn pvctlndtaq fnrmtveyvy ermtglrwkc |
| 481 | kvilesevia eavgvkktvk yeaageavkt lkktqptvin nlkkgavedv isrneiqgrs |
| 541 | aeeaykqqik ednignqllr kmgwtggglg ksgegirepi svkeqhkreg lgldvervnk |
| 601 | iakrdieqii rnyarsesht dltfsreltn derkqihqia qkyglksksh gvghdrylvv |
| 661 | grkrrkedll dqlkqegqvg hyelvmpqan |
| Plasminogen activator, urokinase, urokinase-type plasminogen activator | |
| isoform 1 preproprotein, NP_002649.1 | |
| 1 | mrallarlll cvlvvsdskg snelhqvpsn cdclnggtcv snkyfsnihw cncpkkfggq |
| 61 | hceidksktc yegnghfyrg kastdtmgrp clpwnsatvl qqtyhahrsd alqlglgkhn |
| 121 | ycrnpdnrrr pwcyvqvglk plvqecmvhd cadgkkpssp peelkfqcgq ktlrprfkii |
| 181 | ggefttienq pwfaaiyrrh rggsvtyvcg gslispcwvi sathcfidyp kkedyivylg |
| 241 | rsrlnsntqg emkfevenli lhkdysadtl ahhndiallk irskegrcaq psrtiqticl |
| 301 | psmyndpqfg tsceitgfgk enstdylype qlkmtvvkli shrecqqphy ygsevttkml |
| 361 | caadpqwktd scqgdsggpl vcslqgrmtl tgivswgrgc alkdkpgvyt rvshflpwir |
| 421 | shtkeengla l |
| Plasminogen activator, urokinase, urokinase-type plasminogen activator | |
| isoform 2, NP_001138503.1 | |
| 1 | mvfhlrtrye qancdclngg tcvsnkyfsn ihwcncpkkf ggqhceidks ktcyegnghf |
| 61 | yrgkastdtm grpclpwnsa tvlqqtyhah rsdalqlglg khnycrnpdn rrrpwcyvqv |
| 121 | glkplvqecm vhdcadgkkp ssppeelkfq cgqktlrprf kiiggeftti enqpwfaaiy |
| 181 | rrhrggsvty vcggslispc wvisathcfi dypkkedyiv ylgrsrlnsn tqgemkfeve |
| 241 | nlilhkdysa dtlahhndia llkirskegr caqpsrtiqt iclpsmyndp qfgtsceitg |
| 301 | fgkenstdyl ypeqlkmtvv klishrecqq phyygsevtt kmlcaadpqw ktdscqgdsg |
| 361 | gplvcslqgr mtltgivswg rgcalkdkpg vytrvshflp wirshtkeen glal |
| Plasminogen activator, urokinase, urokinase-type plasminogen activator | |
| isoform 3, NP_001306120.1 | |
| 1 | mgrpclpwns atvlqqtyha hrsdalqlgl gkhnycrnpd nrrrpwcyvq vglkplvqec |
| 61 | mvhdcadgkk pssppeelkf qcgqktlrpr fkiiggeftt ienqpwfaai yrrhrggsvt |
| 121 | yvcggslisp cwvisathcf idypkkedyi vylgrsrlns ntqgemkfev enlilhkdys |
| 181 | adtlahhndi allkirskeg rcaqpsrtiq ticlpsmynd pqfgtsceit gfgkenstdy |
| 241 | lypeqlkmtv vklishrecq qphyygsevt tkmlcaadpq wktdscqgds ggplvcslqg |
| 301 | rmtltgivsw grgcalkdkp gvytrvshfl pwirshtkee nglal |
| Receptor tyrosine kinase like orphan receptor 1, inactive tyrosine- | |
| protein kinase transmembrane receptor ROR1 isoform 1 precursor, | |
| NP_005003.2 | |
| 1 | mhrprrrgtr ppllallaal llaargaaaq etelsvsael vptsswniss elnkdsyltl |
| 61 | depmnnitts lgqtaelhck vsgnppptir wfkndapvvq eprrlsfrst iygsrlrirn |
| 121 | ldttdtgyfq cvatngkevv sstgvlfvkf gppptaspgy sdeyeedgfc qpyrgiacar |
| 181 | fignrtvyme slhmqgeien qitaaftmig tsshlsdkcs qfaipslchy afpycdetss |
| 241 | vpkprdlcrd eceilenvlc qteyifarsn pmilmrlklp ncedlpqpes peaancirig |
| 301 | ipmadpinkn hkcynstgvd yrgtvsvtks grqcqpwnsq yphthtftal rfpelngghs |
| 361 | ycrnpgnqke apwcftlden fksdlcdipa cdskdskekn kmeilyilvp svaiplaial |
| 421 | lffficvcrn nqksssapvq rqpkhvrgqn vemsmlnayk pkskakelpl savrfmeelg |
| 481 | ecafgkiykg hlylpgmdha qlvaiktlkd ynnpqqwtef qqeaslmael hhpnivcllg |
| 541 | avtqeqpvcm lfeyinqgdl heflimrsph sdvgcssded gtvkssldhg dflhiaiqia |
| 601 | agmeylsshf fvhkdlaarn iligeqlhvk isdlglsrei ysadyyrvqs ksllpirwmp |
| 661 | peaimygkfs sdsdiwsfgv vlweifsfgl qpyygfsnqe viemvrkrql lpcsedcppr |
| 721 | myslmtecwn eipsrrprfk dihvrlrswe glsshtsstt psggnattqt tslsaspvsn |
| 781 | lsnprypnym fpsqgitpqg qiagfigppi pqnqrfipin gypippgyaa fpaahyqptg |
| 841 | pprviqhcpp pksrspssas gststghvts lpssgsnqea nipllphmsi pnhpggmgit |
| 901 | vfgnksqkpy kidskqasll gdanihghte smisael |
| Receptor tyrosine kinase like orphan receptor 1, inactive tyrosine- | |
| protein kinase transmembrane receptor ROR1 isoform 2 precursor, | |
| NP_001077061.1 | |
| 1 | mhrprrrgtr ppllallaal llaargaaaq etelsvsael vptsswniss elnkdsyltl |
| 61 | depmnnitts lgqtaelhck vsgnppptir wfkndapvvq eprrlsfrst iygsrlrirn |
| 121 | ldttdtgyfq cvatngkevv sstgvlfvkf gppptaspgy sdeyeedgfc qpyrgiacar |
| 181 | fignrtvyme slhmqgeien qitaaftmig tsshlsdkcs qfaipslchy afpycdetss |
| 241 | vpkprdlcrd eceilenvlc qteyifarsn pmilmrlklp ncedlpqpes peaancirig |
| 301 | ipmadpinkn hkcynstgvd yrgtvsvtks grqcqpwnsq yphthtftal rfpelngghs |
| 361 | ycrnpgnqke apwcftlden fksdlcdipa cgk |
| Runt related transcription factor 1, runt-related transcription factor | |
| 1 isoform AML1a, NP_001116079.1 | |
| 1 | mripvdasts rrftppstal spgkmsealp lgapdagaal agklrsgdrs mvevladhpg |
| 61 | elvrtdspnf lcsvlpthwr cnktlpiafk vvalgdvpdg tlvtvmagnd enysaelrna |
| 121 | taamknqvar fndlrfvgrs grgksftlti tvftnppqva tyhraikitv dgpreprrhr |
| 181 | qklddqtkpg slsfserlse leqlrrtamr vsphhpaptp npraslnhst afnpqpqsqm |
| 241 | qeedtapwrc |
| Runt related transcription factor 1, runt-related transcription factor | |
| 1 isoform AML1b, NP_001001890.1 | |
| 1 | mripvdasts rrftppstal spgkmsealp lgapdagaal agklrsgdrs mvevladhpg |
| 61 | elvrtdspnf lcsvlpthwr cnktlpiafk vvalgdvpdg tlvtvmagnd enysaelrna |
| 121 | taamknqvar fndlrfvgrs grgksftlti tvftnppqva tyhraikitv dgpreprrhr |
| 181 | qklddqtkpg slsfserlse leqlrrtamr vsphhpaptp npraslnhst afnpqpqsqm |
| 241 | qdtrqiqpsp pwsydqsyqy lgsiaspsvh patpispgra sgmttlsael ssrlstapdl |
| 301 | tafsdprqfp alpsisdprm hypgaftysp tpvtsgigig msamgsatry htylpppypg |
| 361 | ssqaqggpfq asspsyhlyy gasagsyqfs mvggersppr ilppctnast gsallnpslp |
| 421 | nqsdvveaeg shsnsptnma psarleeavw rpy |
| Runt related transcription factor 1, runt-related transcription factor | |
| 1 isoform AML1c, NP_001745.2 | |
| 1 | masdsifesf psypqcfmre cilgmnpsrd vhdastsrrf tppstalspg kmsealplga |
| 61 | pdagaalagk lrsgdrsmve vladhpgelv rtdspnflcs vlpthwrcnk tlpiafkvva |
| 121 | lgdvpdgtlv tvmagndeny saelrnataa mknqvarfnd lrfvgrsgrg ksftltitvf |
| 181 | tnppqvatyh raikitvdgp reprrhrqkl ddqtkpgsls fserlseleq lrrtamrvsp |
| 241 | hhpaptpnpr aslnhstafn pqpqsqmqdt rqiqpsppws ydqsyqylgs iaspsvhpat |
| 301 | pispgrasgm ttlsaelssr lstapdltaf sdprqfpalp sisdprmhyp gaftysptpv |
| 361 | tsgigigmsa mgsatryhty lpppypgssq aqggpfqass psyhlyygas agsyqfsmvg |
| 421 | gerspprilp pctnastgsa llnpslpnqs dvveaegshs nsptnmapsa rleeavwrpy |
| Surfactant protein A1, pulmonary surfactant-associated protein A1 | |
| isoform 1 precursor, NP_001158116.1, NP_001158119.1, NP_005402.3 | |
| 1 | mwlcplalnl ilmaasgavc evkdvcvgsp gipgtpgshg lpgrdgrdgl kgdpgppgpm |
| 61 | gppgempcpp gndglpgapg ipgecgekge pgergppglp ahldeelqat lhdfrhqilq |
| 121 | trgalslqgs imtvgekvfs sngqsitfda iqeacaragg riavprnpee neaiasfvkk |
| 181 | yntyayvglt egpspgdfry sdgtpvnytn wyrgepagrg keqcvemytd gqwndrncly |
| 241 | srlticef |
| Surfactant protein A1, pulmonary surfactant-associated protein A1 | |
| isoform 2 precursor, NP_001087239.2 | |
| 1 | mrpcqvpgaa tgpramwlcp lalnlilmaa sgavcevkdv cvgspgipgt pgshglpgrd |
| 61 | grdglkgdpg ppgpmgppge mpcppgndgl pgapgipgec gekgepgerg ppglpahlde |
| 121 | elqatlhdfr hqilqtrgal slqgsimtvg ekvfssngqs itfdaiqeac araggriavp |
| 181 | rnpeeneaia sfvkkyntya yvgltegpsp gdfrysdgtp vnytnwyrge pagrgkeqcv |
| 241 | emytdgqwnd rnclysrlti cef |
| Surfactant protein A1, pulmonary surfactant-associated protein A1 | |
| isoform 3 precursor, NP_001158117.1 | |
| 1 | mrpcqvpgaa tgpramwlcp lalnlilmaa sgavcevkdv cvgtpgipge cgekgepger |
| 61 | gppglpahld eelqatlhdf rhqilqtrga lslqgsimtv gekvfssngq sitfdaiqea |
| 121 | caraggriav prnpeeneai asfvkkynty ayvgltegps pgdfrysdgt pvnytnwyrg |
| 181 | epagrgkeqc vemytdgqwn drnclysrlt icef |
| Surfactant protein A1, pulmonary surfactant-associated protein A1 | |
| isoform 4 precursor, NP_001158118.1 | |
| 1 | mwlcplalnl ilmaasgavc evkdvcvgtp gipgecgekg epgergppgl pahldeelqa |
| 61 | tlhdfrhqil qtrgalslqg simtvgekvf ssngqsitfd aiqeacarag griavprnpe |
| 121 | eneaiasfvk kyntyayvgl tegpspgdfr ysdgtpvnyt nwyrgepagr gkeqcvemyt |
| 181 | dgqwndrncl ysrlticef |
| Surfactant protein A2, pulmonary surfactant-associated protein A2 | |
| isoform 1 precursor, NP_001092138.1, NP_001307742.1 | |
| 1 | mwlcplaltl ilmaasgaac evkdvcvgsp gipgtpgshg lpgrdgrdgv kgdpgppgpm |
| 61 | gppgetpcpp gnnglpgapg vpgergekge agergppglp ahldeelqat lhdfrhqilq |
| 121 | trgalslqgs imtvgekvfs sngqsitfda iqeacaragg riavprnpee neaiasfvkk |
| 181 | yntyayvglt egpspgdfry sdgtpvnytn wyrgepagrg keqcvemytd gqwndrncly |
| 241 | srlticef |
| Surfactant protein A2, pulmonary surfactant-associated protein A2 | |
| isoform 2 precursor, NP_001307743.1 | |
| 1 | mpgaatgpra mwlcplaltl ilmaasgaac evkdvcvgsp gipgtpgshg lpgrdgrdgv |
| 61 | kgdpgppgpm gppgetpcpp gnnglpgapg vpgergekge agergppglp ahldeelqat |
| 121 | lhdfrhqilq trgalslqgs imtvgekvfs sngqsitfda iqeacaragg riavprnpee |
| 181 | neaiasfvkk yntyayvglt egpspgdfry sdgtpvnytn wyrgepagrg keqcvemytd |
| 241 | gqwndrncly srlticef |
| Surfactant protein B, pulmonary surfactant-associated protein B | |
| precursor, NP_000533.3, NP_942140.2 | |
| 1 | mhqagypgcr gamaeshllq wlllllptlc gpgtaawtts slacaqgpef wcqsleqalq |
| 61 | cralghclqe vwghvgaddl cqecedivhi lnkmakeaif qdtmrkfleq ecnvlplkll |
| 121 | mpqcnqvldd yfplvidyfq nqtdsngicm hlglcksrqp epeqepgmsd plpkplrdpl |
| 181 | pdplldklvl pvlpgalqar pgphtqdlse qqfpiplpyc wlcralikri qamipkgala |
| 241 | vavaqvcrvv plvaggicqc laerysvill dtllgrmlpq lvcrlvlrcs mddsagprsp |
| 301 | tgewlprdse chlcmsvttq agnsseqaip qamlqacvgs wldrekckqf veghtpqllt |
| 361 | lvprgwdaht tcqalgvcgt mssplqcihs pdl |
| Surfactant protein C, pulmonary surfactant-associated protein C | |
| isoform 1 precursor, NP_001165881.1, NP_003009.2 | |
| 1 | mdvgskevlm esppdysaap rgrfgipccp vhlkrllivv vvvvlivvvi vgallmglhm |
| 61 | sqkhtemvle msigapeaqq rlalsehlvt tatfsigstg lvvydyqqll iaykpapgtc |
| 121 | cyimkiapes ipslealtrk vhnfqmecsl qakpavptsk lgqaegrdag sapsggdpaf |
| 181 | lgmavstlcg evplyyi |
| Surfactant protein C, pulmonary surfactant-associated protein C | |
| isoform 2 precursor, NP_001165828.1, NP_001304707.1, NP_001304709.1 | |
| 1 | mdvgskevlm esppdysaap rgrfgipccp vhlkrllivv vvvvlivvvi vgallmglhm |
| 61 | sqkhtemvle msigapeaqq rlalsehlvt tatfsigstg lvvydyqqll iaykpapgtc |
| 121 | cyimkiapes ipslealtrk vhnfqakpav ptsklgqaeg rdagsapsgg dpaflgmavs |
| 181 | tlcgevplyy i |
| Surfactant protein C, pulmonary surfactant-associated protein C | |
| isoform 3 precursor, NP_001304708.1 | |
| 1 | mdvgskevlm esppvlemsi gapeaqqrla lsehlvttat fsigstglvv ydyqqlliay |
| 61 | kpapgtccyi mkiapesips lealtrkvhn fqmecslqak pavptsklgq aegrdagsap |
| 121 | sggdpaflgm aystlcgevp lyyi |
| Surfactant protein D, pulmonary surfactant-associated protein D | |
| precursor, NP_003010.4 | |
| 1 | mllfllsalv lltqplgyle aemktyshrt mpsactlvmc ssvesglpgr dgrdgregpr |
| 61 | gekgdpglpg aagqagmpgq agpvgpkgdn gsvgepgpkg dtgpsgppgp pgvpgpagre |
| 121 | gplgkqgnig pqgkpgpkge agpkgevgap gmqgsagarg lagpkgergv pgergvpgnt |
| 181 | gaagsagamg pqgspgargp pglkgdkgip gdkgakgesg lpdvaslrqq vealqgqvqh |
| 241 | lqaafsqykk velfpngqsv gekifktagf vkpfteaqll ctqaggqlas prsaaenaal |
| 301 | qqlvvaknea aflsmtdskt egkftyptge slvysnwapg epnddggsed cveiftngkw |
| 361 | ndracgekrl vvcef |
| Solute carrier family 2 member 5, solute carrier family 2, facilitated | |
| glucose transporter member 5 isoform 1, NP_001315548.1, NP_003030.1 | |
| 1 | meqqdqsmke grltlvlala tliaafgssf qygynvaavn spallmqqfy netyygrtge |
| 61 | fmedfpltll wsvtvsmfpf ggfigsllvg plvnkfgrkg allfnnifsi vpailmgcsr |
| 121 | vatsfeliii srllvgicag vssnvvpmyl gelapknlrg algvvpqlfi tvgilvaqif |
| 181 | glrnllanvd gwpillgltg vpaalqllll pffpespryl liqkkdeaaa kkalqtlrgw |
| 241 | dsvdrevaei rqedeaekaa gfisvlklfr mrslrwqlls iivlmggqql sgvnaiyyya |
| 301 | dqiylsagvp eehvqyvtag tgavnvvmtf cavfvvellg rrlllllgfs icliaccvlt |
| 361 | aalalqdtvs wmpyisivcv isyvighalg pspipallit eiflqssrps afmvggsvhw |
| 421 | lsnftvglif pfiqeglgpy sfivfavicl lttiyifliv petkaktfie inqiftkmnk |
| 481 | vsevypekee lkelppvtse q |
| Solute carrier family 2 member 5, solute carrier family 2, facilitated | |
| glucose transporter member 5 isoform 2, NP_001129057.1 | |
| 1 | meqqdqsmke grltlvlala tliaafgssf qygynvaavn spallmqqfy netyygrtge |
| 61 | fmedfpltll wsvtvsmfpf ggfigsllvg plvnkfgrkg allfnnifsi vpailmgcsr |
| 121 | vatsfeliii srllvgicag vssnvvpmyl gelapknlrg algvvpqlfi tvgilvaqif |
| 181 | glrnllanvd gefrtsrehp hpftttlgpl lvfqshhhrt glsadwsllt gwmslggpsc |
| 241 | pept |
| Solute carrier family 2 member 5, solute carrier family 2, facilitated | |
| glucose transporter member 5 isoform 3, NP_001315549.1 | |
| 1 | mgttwllstp qhwtgefmed fpltllwsvt vsmfpfggfi gsllvgplvn kfgrkgallf |
| 61 | nnifsivpai lmgcsrvats feliiisrll vgicagvssn vvpmylgela pknlrgalgv |
| 121 | vpqlfitvgi lvaqifglrn llanvdgwpi llgltgvpaa lqllllpffp esprylliqk |
| 181 | kdeaaakkal qtlrgwdsvd revaeirqed eaekaagfis vlklfrmrsl rwqllsiivl |
| 241 | mggqqlsgvn aiyyyadqiy lsagvpeehv qyvtagtgav nvvmtfcavf vvellgrrll |
| 301 | lllgfsicli accvltaala lqdtvswmpy isivcvisyv ighalgpspi palliteifl |
| 361 | qssrpsafmv ggsvhwlsnf tvglifpfiq eglgpysfiv faviclltti yiflivpetk |
| 421 | aktfieinqi ftkmnkvsev ypekeelkel ppvtseq |
| Solute carrier family 2 member 5, solute carrier family 2, facilitated | |
| glucose transporter member 5 isoform 4, NP_001315550.1 | |
| 1 | mylgelapkn lrgalgvvpq lfitvgilva qifglrnlla nvdgwpillg ltgvpaalql |
| 61 | lllpffpesp rylliqkkde aaakkalqtl rgwdsvdrev aeirqedeae kaagfisvlk |
| 121 | lfrmrslrwq llsiivlmgg qqlsgvnaiy yyadqiylsa gvpeehvqyv tagtgavnvv |
| 181 | mtfcavfvve llgrrlllll gfsicliacc vltaalalqd tvswmpyisi vcvisyvigh |
| 241 | algpspipal liteiflqss rpsafmvggs vhwlsnftvg lifpfiqegl gpysfivfav |
| 301 | icllttiyif livpetkakt fieinqiftk mnkvsevype keelkelppv tseq |
| Sperm associated antigen 9, C-Jun-amino-terminal kinase-interacting | |
| protein 4 isoform 1, NP_001124000.1 | |
| 1 | meledgvvyq eepggsgavm servsglags iyreferlig rydeevvkel mplvvavlen |
| 61 | ldsvfaqdqe hqvelellrd dneqlitgye rekalrkhae ekfiefedsq eqekkdlqtr |
| 121 | veslesqtrq lelkaknyad qisrleerea elkkeynalh qrhtemihny mehlertklh |
| 181 | qlsgsdqles tahsrirker pislgifplp agdglltpda qkggetpgse qwkfqelsqp |
| 241 | rshtslkvsn spepqkaveq edelsdvsqg gskattpast ansdvatipt dtplkeeneg |
| 301 | fvkvtdapnk seiskhievq vaqetrnvst gsaeneekse vqaiiestpe ldmdkdlsgy |
| 361 | kgsstptkgi enkafdrnte slfeelssag sgligdvdeg adllgmgrev enlilentql |
| 421 | letknalniv kndliakvde ltcekdvlqg eleavkqakl kleeknrele eelrkaraea |
| 481 | edarqkakdd ddsdiptaqr krftrvemar vlmernqyke rlmelqeavr wtemirasre |
| 541 | npamqekkrs siwqffsrlf ssssnttkkp eppvnlkyna ptshvtpsvk krsstlsqlp |
| 601 | gdkskafdfl seeteaslas rreqkreqyr qvkahvqked grvqafgwsl pqkykqvtng |
| 661 | qgenkmknlp vpvylrplde kdtsmklwca vgvnlsggkt rdggsvvgas vfykdvagld |
| 721 | tegskqrsas qssldkldqe lkeqqkelkn qeelsslvwi ctsthsatkv liidavqpgn |
| 781 | ildsftvcns hvlciasvpg aretdypage dlsesgqvdk aslcgsmtsn ssaetdsllg |
| 841 | gitvvgcsae gvtgaatsps tngaspvmdk ppemeaense vdenvptaee ateategnag |
| 901 | saedtvdisq tgvytehvft dplgvqiped lspvyqssnd sdaykdqisv lpneqdlvre |
| 961 | eaqkmssllp tmwlgaqngc lyvhssvaqw rkclhsiklk dsilsivhvk givlvaladg |
| 1021 | tlaifhrgvd gqwdlsnyhl ldlgrphhsi rcmtvvhdkv wcgyrnkiyv vqpkamkiek |
| 1081 | sfdahprkes qvrqlawvgd gvwvsirlds tlrlyhahty qhlqdvdiep yvskmlgtgk |
| 1141 | lgfsfvrita lmvscnrlwv gtgngviisi pltetnktsg vpgnrpgsvi rvygdensdk |
| 1201 | vtpgtfipyc smahaqlcfh ghrdavkffv avpgqvispq ssssgtdltg dkagpsaqep |
| 1261 | gsqtplksml visggegyid frmgdegges ellgedlple psvtkaersh livwqvmygn |
| 1321 | e |
| Sperm associated antigen 9, C-Jun-amino-terminal kinase-interacting | |
| protein 4 isoform 2, NP_001123999.1 | |
| 1 | meledgvvyq eepggsgavm servsglags iyreferlig rydeevvkel mplvvavlen |
| 61 | ldsvfaqdqe hqvelellrd dneqlitqye rekalrkhae ekfiefedsq eqekkdlqtr |
| 121 | veslesqtrq lelkaknyad qisrleerea elkkeynalh qrhtemihny mehlertklh |
| 181 | qlsgsdqles tahsrirker pislgifplp agdglltpda qkggetpgse qwkfqelsqp |
| 241 | rshtslkdel sdvsqggska ttpastansd vatiptdtpl keenegfvkv tdapnkseis |
| 301 | khievqvaqe trnvstgsae neeksevqai iestpeldmd kdlsgykgss tptkgienka |
| 361 | fdrnteslfe elssagsgli gdvdegadll gmgrevenli lentqlletk nalnivkndl |
| 421 | iakvdeltce kdvlqgelea vkqaklklee knreleeelr karaeaedar qkakddddsd |
| 481 | iptaqrkrft rvemarvlme rnqykerlme lqeavrwtem irasrenpam qekkrssiwq |
| 541 | fvptrfsrlf ssssnttkkp eppvnlkyna ptshvtpsvk krsstlsqlp gdkskafdfl |
| 601 | seeteaslas rreqkreqyr qvkahvqked grvqafgwsl pqkykqvtng qgenkmknlp |
| 661 | vpvylrplde kdtsmklwca vgvnlsggkt rdggsvvgas vfykdvagld tegskqrsas |
| 721 | qssldkldqe lkeqqkelkn geelsslvwi ctsthsatkv liidavqpgn ildsftvcns |
| 781 | hvlciasvpg aretdypage dlsesgqvdk aslcgsmtsn ssaetdsllg gitvvgcsae |
| 841 | gvtgaatsps tngaspvmdk ppemeaense vdenvptaee ateategnag saedtvdisq |
| 901 | tgvytehvft dplgvqiped lspvyqssnd sdaykdqisv lpneqdlvre eaqkmssllp |
| 961 | tmwlgaqngc lyvhssvaqw rkclhsiklk dsilsivhvk givlvaladg tlaifhrgvd |
| 1021 | gqwdlsnyhl ldlgrphhsi rcmtvvhdkv wcgyrnkiyv vqpkamkiek sfdahprkes |
| 1081 | qvrqlawvgd gvwvsirlds tlrlyhahty qhlqdvdiep yvskmlgtgk lgfsfvrita |
| 1141 | lmvscnrlwv gtgngviisi pltetnktsg vpgnrpgsvi rvygdensdk vtpgtfipyc |
| 1201 | smahaqlcfh ghrdavkffv avpgqvispq ssssgtdltg dkagpsaqep gsqtplksml |
| 1261 | visggegyid frmgdegges ellgedlple psvtkaersh livwqvmygn e |
| Sperm associated antigen 9, C-Jun-amino-terminal kinase-interacting | |
| protein 4 isoform 3, NP_003962.3 | |
| 1 | meledgvvyq eepggsgavm servsglags iyreferlig rydeevvkel mplvvavlen |
| 61 | ldsvfaqdqe hqvelellrd dneqlitqye rekalrkhae ekfiefedsq eqekkdlqtr |
| 121 | veslesqtrq lelkaknyad qisrleerea elkkeynalh qrhtemihny mehlertklh |
| 181 | qlsgsdqles tahsrirker pislgifplp agdglltpda qkggetpgse qwkfqelsqp |
| 241 | rshtslkdel sdvsqggska ttpastansd vatiptdtpl keenegfvkv tdapnkseis |
| 301 | khievqvaqe trnvstgsae neeksevqai iestpeldmd kdlsgykgss tptkgienka |
| 361 | fdrnteslfe elssagsgli gdvdegadll gmgrevenli lentqlletk nalnivkndl |
| 421 | iakvdeltce kdvlqgelea vkqaklklee knreleeelr karaeaedar qkakddddsd |
| 481 | iptaqrkrft rvemarvlme rnqykerlme lqeavrwtem irasrenpam qekkrssiwq |
| 541 | ffsrlfssss nttkkpeppv nlkynaptsh vtpsvkkrss tlsqlpgdks kafdflseet |
| 601 | easlasrreq kreqyrqvka hvqkedgrvq afgwslpqky kqvtngqgen kmknlpvpvy |
| 661 | lrpldekdts mklwcavgvn lsggktrdgg svvgasvfyk dvagldtegs kqrsasqssl |
| 721 | dkldqelkeq qkelknqeel sslvwictst hsatkvliid avqpgnilds ftvcnshvlc |
| 781 | iasvpgaret dypagedlse sgqvdkaslc gsmtsnssae tdsllggitv vgcsaegvtg |
| 841 | aatspstnga spvmdkppem eaensevden vptaeeatea tegnagsaed tvdisqtgvy |
| 901 | tehvftdplg vqipedlspv yqssndsday kdqisvlpne qdlvreeaqk mssllptmwl |
| 961 | gaqngclyvh ssvaqwrkcl hsiklkdsil sivhvkgivl valadgtlai fhrgvdgqwd |
| 1021 | lsnyhlldlg rphhsircmt vvhdkvwcgy rnkiyvvqpk amkieksfda hprkesqvrq |
| 1081 | lawvgdgvwv sirldstlrl yhahtyqhlq dvdiepyvsk mlgtgklgfs fvritalmvs |
| 1141 | cnrlwvgtgn gviisiplte tnktsgvpgn rpgsvirvyg densdkvtpg tfipycsmah |
| 1201 | aqlcfhghrd avkffvavpg qvispqssss gtdltgdkag psaqepgsqt plksmlvisg |
| 1261 | gegyidfrmg deggesellg edlplepsvt kaershlivw qvmygne |
| Sperm associated antigen 9, C-Jun-amino-terminal kinase-interacting | |
| protein 4 isoform 4, NP_001238900.1 | |
| 1 | mspgcmllfv fgfvggavvi nsailvslsv lllvhfsist gvpaltqnlp rilrkerpis |
| 61 | lgifplpagd glltpdaqkg getpgseqwk fqelsqprsh tslkdelsdv sqggskattp |
| 121 | astansdvat iptdtplkee negfvkvtda pnkseiskhi evqvaqetrn vstgsaenee |
| 181 | ksevqaiies tpeldmdkdl sgykgsstpt kgienkafdr nteslfeels sagsgligdv |
| 241 | degadllgmg revenlilen tqlletknal nivkndliak vdeltcekdv lqgeleavkq |
| 301 | aklkleeknr eleeelrkar aeaedarqka kddddsdipt aqrkrftrve marvlmernq |
| 361 | ykerlmelqe avrwtemira srenpamqek krssiwqffs rlfssssntt kkpeppvnlk |
| 421 | ynaptshvtp svkkrsstls qlpgdkskaf dflseeteas lasrreqkre qyrqvkahvq |
| 481 | kedgrvqafg wslpqkykqv tngqgenkmk nlpvpvylrp ldekdtsmkl wcavgvnlsg |
| 541 | gktrdggsvv gasvfykdva gldtegskqr sasqssldkl dqelkeqqke lknqeelssl |
| 601 | vwictsthsa tkvliidavq pgnildsftv cnshvlcias vpgaretdyp agedlsesgq |
| 661 | vdkaslcgsm tsnssaetds llggitvvgc saegvtgaat spstngaspv mdkppemeae |
| 721 | nsevdenvpt aeeateateg nagsaedtvd isqtgvyteh vftdplgvqi pedlspvyqs |
| 781 | sndsdaykdq isvlpneqdl vreeaqkmss llptmwlgaq ngclyvhssv aqwrkclhsi |
| 841 | klkdsilsiv hvkgivlval adgtlaifhr gvdgqwdlsn yhlldlgrph hsircmtvvh |
| 901 | dkvwcgyrnk iyvvqpkamk ieksfdahpr kesqvrqlaw vgdgvwvsir ldstlrlyha |
| 961 | htyqhlqdvd iepyvskmlg tgklgfsfvr italmvscnr lwvgtgngvi isipltetvi |
| 1021 | lhqgrllglr anktsgvpgn rpgsvirvyg densdkvtpg tfipycsmah aqlcfhghrd |
| 1081 | avkffvavpg qvispqssss gtdltgdkag psaqepgsqt plksmlvisg gegyidfrmg |
| 1141 | deggesellg edlplepsvt kaershlivw qvmygne |
| SGT1 homolog, MIS12 kinetochore complex assembly cochaperone, protein | |
| SGT1 homolog isoform A, NP_006695.1 | |
| 1 | maaaaagtat sqrffqsfsd alidedpqaa leeltkaleq kpddaqyycq raychillgn |
| 61 | ycvavadakk slelnpnnst amlrkgicey heknyaaale tftegqklds adanfsvwik |
| 121 | rcqeaqngse sevwthqski kydwyqtesq vvitlmiknv qkndvnvefs ekelsalvkl |
| 181 | psgedynlkl ellhpiipeq stfkvlstki eiklkkpeav rweklegqgd vptpkqfvad |
| 241 | vknlypsssp ytrnwdklvg eikeeeknek legdaalnrl fqqiysdgsd evkramnksf |
| 301 | mesggtvlst nwsdvgkrkv einppddmew kky |
| SGT1 homolog, MIS12 kinetochore complex assembly cochaperone, protein | |
| SGT1 homolog isoform B, NP_001124384.1 | |
| 1 | maaaaagtat sqrffqsfsd alidedpqaa leeltkaleq kpddaqyycq raychillgn |
| 61 | ycvavadakk slelnpnnst amlrkgicey heknyaaale tftegqkldi etgfhrvgqa |
| 121 | glqlltssdp paldsqsagi tgadanfsvw ikrcqeaqng sesevwthqs kikydwyqte |
| 181 | sqvvitlmik nvqkndvnve fsekelsalv klpsgedynl klellhpiip eqstfkvlst |
| 241 | kieiklkkpe avrweklegq gdvptpkqfv advknlypss spytrnwdkl vgeikeeekn |
| 301 | eklegdaaln rlfqqiysdg sdevkramnk sfmesggtvl stnwsdvgkr kveinppddm |
| 361 | ewkky |
| SGT1 homolog, MIS12 kinetochore complex assembly cochaperone, protein | |
| SGT1 homolog isoform C, NP_001307760.1 | |
| 1 | mlsqkevava dakkslelnp nnstamlrkg iceyheknya aaletftegq kldsadanfs |
| 61 | vwikrcqeaq ngsesevwth gskikydwyq tesqvvitlm iknvqkndvn vefsekelsa |
| 121 | lvklpsgedy nlklellhpi ipeqstfkvl stkieiklkk peavrwekle gqgdvptpkq |
| 181 | fvadvknlyp ssspytrnwd klvgeikeee kneklegdaa lnrlfqqiys dgsdevkram |
| 241 | nksfmesggt vlstnwsdvg krkveinppd dmewkky |
| Sulfotransferase family 1C member 2, sulfotransferase 1C2 isoform a, | |
| NP_001047.1 | |
| 1 | maltsdlgkq iklkevegtl lqpatvdnws qiqsfeakpd dllictypka gttwiqeivd |
| 61 | mieqngdvek cqraiiqhrh pfiewarppq psgvekakam psprilkthl stqllppsfw |
| 121 | ennckflyva rnakdcmvsy yhfqrmnhml pdpgtweeyf etfingkvvw gswfdhvkgw |
| 181 | wemkdrhqil flfyedikrd pkheirkvmq fmgkkvdetv ldkivqetsf ekmkenpmtn |
| 241 | rstvsksild qsissfmrkg tvgdwknhft vaqnerfdei yrrkmegtsi nfcmel |
| Sulfotransferase family 1C member 2, sulfotransferase 1C2 isoform b, | |
| NP_789795.1 | |
| 1 | maltsdlgkq iklkevegtl lqpatvdnws qiqsfeakpd dllictypka gttwiqeivd |
| 61 | mieqngdvek cqraiiqhrh pfiewarppq psetgfhhva qaglkllsss nppastsqsa |
| 121 | kitdllppsf wennckflyv arnakdcmvs yyhfqrmnhm lpdpgtweey fetfingkvv |
| 181 | wgswfdhvkg wwemkdrhqi lflfyedikr dpkheirkvm qfmgkkvdet vldkivqets |
| 241 | fekmkenpmt nrstvsksil dqsissfmrk gtvgdwknhf tvaqnerfde iyrrkmegts |
| 301 | infcmel |
| Transmembrane protein 52B, isoform 1, NP_694567.1 | |
| 1 | mswrpqpcci sscclttdwv hlwyiwllvv igallllcgl tslcfrcccl srqqngedgg |
| 61 | pppcevtvia fdhdstlqst itslqsvfgp aarrilavah shsslgqlps sldtlpgyee |
| 121 | alhmsrftva mcgqkapdlp pvpeekqlpp tekestrivd swn |
| Transmembrane protein 52B, isoform 2 precursor, NP_001073283.1 | |
| 1 | mgvrvhvvaa sallyfills gtrceencgn pehclttdwv hlwyiwllvv igallllcgl |
| 61 | tslcfrcccl srqqngedgg pppcevtvia fdhdstlqst itslqsvfgp aarrilavah |
| 121 | shsslgqlps sldtlpgyee alhmsrftva mcgqkapdlp pvpeekqlpp tekestrivd |
| 181 | swn |
| Exportin 7, NP_055839.3 | |
| 1 | madhvqslaq lenlckqlye ttdtttrlqa ekalveftns pdclskcqll lergsssysq |
| 61 | llaatcltkl vsrtnnplpl eqridirnyv lnylatrpkl atfvtqaliq lyaritklgw |
| 121 | fdcqkddyvf rnaitdvtrf lqdsveycii gvtilsqltn einqadtthp ltkhrkiass |
| 181 | frdsslfdif tlscnllkqa sgknlnlnde sqhgllmqll klthnclnfd figtstdess |
| 241 | ddlctvqipt swrsafldss tlqlffdlyh sippsfsplv lsclvqiasv rrslfnnaer |
| 301 | akflshlvdg vkrilenpqs lsdpnnyhef crllarlksn yqlgelvkve nypevirlia |
| 361 | nftvtslqhw efapnsvhyl lslwqrlaas vpyvkateph mletytpevt kayitsrles |
| 421 | vhiilrdgle dpledtglvq qqldqlstig rceyektcal lvqlfdqsaq syqellqsas |
| 481 | aspmdiavqe grltwlvyii gaviggrvsf astdeqdamd gelvcrvlql mnitdsrlaq |
| 541 | agneklelam lsffeqfrki yigdqvqkss klyrrlsevl glndetmvls vfigkiitnl |
| 601 | kywgrcepit sktlqllndl sigyssvrkl vklsavqfml nnhtsehfsf lginnqsnlt |
| 661 | dmrcrttfyt algrllmvdl gededqyeqf mlpltaafea vaqmfstnsf neqeakrtlv |
| 721 | glvrdlrgia fafnaktsfm mlfewiypsy mpilqraiel wyhdpacttp vlklmaelvh |
| 781 | nrsqrlqfdv sspngillfr etskmitmyg nriltlgevp kdqvyalklk gisicfsmlk |
| 841 | aalsgsyvnf gvfrlygdda ldnalqtfik lllsiphsdl ldypklsqsy ysllevltqd |
| 901 | hmnfiaslep hvimyilssi segltaldtm vctgccscld hivtylfkql srstkkrttp |
| 961 | lnqesdrflh imqqhpemiq qmlstvlnii ifedcrnqws msrpllglil lnekyfsdlr |
| 1021 | nsivnsqppe kqqamhlcfe nlmegiernl ltknrdrftq nlsafrrevn dsmknstygv |
| 1081 | nsndmms |
| YES proto-oncogene 1, Src family tyrosine kinase, tyrosine-protein | |
| kinase Yes, NP_005424.1 | |
| 1 | mgcikskenk spaikyrpen tpepvstsvs hygaepttvs pcpsssakgt avnfsslsmt |
| 61 | pfggssgvtp fggasssfsv vpssypaglt ggvtifvaly dyearttedl sfkkgerfqi |
| 121 | inntegdwwe arsiatgkng yipsnyvapa dsiqaeewyf gkmgrkdaer lllnpgnqrg |
| 181 | iflvresett kgayslsird wdeirgdnvk hykirkldng gyyittraqf dtlqklvkhy |
| 241 | tehadglchk lttvcptvkp qtqglakdaw eipreslrle vklgqgcfge vwmgtwngtt |
| 301 | kvaiktlkpg tmmpeaflqe aqimkklrhd klvplyavvs eepiyivtef mskgslldfl |
| 361 | kegdgkylkl pqlvdmaaqi adgmayierm nyihrdlraa nilvgenlvc kiadfglarl |
| 421 | iedneytarq gakfpikwta peaalygrft iksdvwsfgi lqtelvtkgr vpypgmvnre |
| 481 | vleqvergyr mpcpqgcpes lhelmnlcwk kdpderptfe yiqsfledyf tatepqyqpg |
| 541 | enl |
| Coiled-coil domain containing 80, coiled-coil domain-containing 80 | |
| precursor, NP_955805.1, NP_955806.1 | |
| 1 | mtwrmgprft mllamwlvcg sephphatir gshggrkvpl vspdssrpar flrhtgrsrg |
| 61 | ierstleepn lqplqrrrsv pvlrlarpte pparsdinga avrpeqrpaa rgspremird |
| 121 | egssarsrml rfpsgssspn ilasfagknr vwvisaphas egyyrlmmsl lkddvycela |
| 181 | erhiqqivlf hqageeggkv rritsegqil eqpldpslip klmsflklek gkfgmvllkk |
| 241 | tlqveerypy pvrleamyev idqgpirrie kirqkgfvqk ckasgvegqv vaegndgggg |
| 301 | agrpslgsek kkedprraqy pptresrvkv lrklaatapa lpqppstpra ttlppapatt |
| 361 | vtrstsravt vaarpmttta fpttqrpwtp spshrppttt evitarrpsv senlyppsrk |
| 421 | dqhrerpqtt rrpskatsle sftnapptti sepstraagp grfrdnrmdr rehghrdpnv |
| 481 | vpgppkpake kppkkkaqdk ilsneyeeky dlsrptasql edelqvgnvp lkkakeskkh |
| 541 | eklekpekek kkkmknenad kllksekqmk ksekkskqek ekskkkkggk teqdgyqkpt |
| 601 | nkhftqspkk svadllgsfe gkrrlllita pkaennmyvq qrdeylesfc kmatrkisvi |
| 661 | tifgpvnnst mkidhfqldn ekpmrvvdde dlvdqrlise lrkeygmtyn dffmvltdvd |
| 721 | lrvkqyyevp itmksvfdli dtfqsrikdm ekqkkegivc kedkkqslen flsrfrwrrr |
| 781 | llvisapnde dwaysqqlsa lsgqacnfgl rhitilkllg vgeevggvle lfpingssvv |
| 841 | eredvpahlv kdirnyfqvs peyfsmllvg kdgnvkswyp spmwsmvivy dlidsmqlrr |
| 901 | qemaiqqslg mrcpedeyag ygyhsyhqgy qdgyqddyrh hesyhhgypy |
| Acrosin-binding protein precursor NP_115878.2 | |
| 1 | mrkpaagflp sllkvlllpl apaaaqdstq astpgsplsp teyerffall tptwkaettc |
| 61 | rlrathgcrn ptlvqldqye nhglvpdgav csnlpyaswf esfcqfthyr csnhvyyakr |
| 121 | vlcsqpvsil spntlkeiea saevspttmt spisphftvt erqtfqpwpe rlsnnveell |
| 181 | qsslslggqe qapehkqeqg vehrqeptqe hkqeegqkqe eqeeeqeeeg kqeegqgtke |
| 241 | greaysqlqt dsepkfhses lssnpssfap rvrevestpm imeniqelir saqeidemne |
| 301 | iydensywrn qnpgsllqlp hteallvlcy siventciit ptakawkyme eeilgfgksv |
| 361 | cdslgrrhms tcalcdfcsl kleqchseas lqrqqcdtsh ktpfvsplla sqslsignqv |
| 421 | gspesgrfyg ldlygglhmd fwcarlatkg cedvrvsgwl qteflsfqdg dfptkicdtd |
| 481 | yiqypnycsf ksqqclmrnr nrkvsrmrcl qnetysalsp gksedvvlrw sqefstltlg |
| 541 | qfg |
| Alpha-fetoprotein, isoform 1 NP_001125.1 | |
| 1 | mkwvesifli fllnftesrt lhrneygias ildsyqctae isladlatif faqfvqeaty |
| 61 | kevskmvkda ltaiekptgd eqssgclenq lpafleelch ekeilekygh sdccsqseeg |
| 121 | rhncflahkk ptpasiplfq vpepvtscea yeedretfmn kfiyeiarrh pflyaptill |
| 181 | waarydkiip scckaenave cfqtkaatvt kelresslln qhacavmknf gtrtfqaitv |
| 241 | tklsqkftkv nfteiqklvl dvahvhehcc rgdvldclqd gekimsyics qqdtlsnkit |
| 301 | eccklttler gqciihaend ekpeglspnl nrflgdrdfn qfssgeknif lasfvheysr |
| 361 | rhpqlavsvi lrvakgyqel lekcfqtenp lecqdkgeee lqkyiqesqa lakrscglfq |
| 421 | klgeyylqna flvaytkkap qltsselmai trkmaataat ccqlsedkll acgegaadii |
| 481 | ighlcirhem tpvnpgvgqc ctssyanrrp cfsslvvdet yvppafsddk fifhkdlcqa |
| 541 | qgvalqtmkq eflinlvkqk pqiteeqlea viadfsglle kccqgqeqev cfaeegqkli |
| 601 | sktraalgv |
| Alpha-fetoprotein, isoform 2 NP_001341646.1 | |
| 1 | mnkfiyeiar rhpflyapti llwaarydki ipscckaena vecfqtkaat vtkelressl |
| 61 | lnqhacavmk nfgtrtfqai tvtklsqkft kvnfteiqkl vldvahvheh ccrgdvldcl |
| 121 | qdgerimsyi csqqdtlsnk iteccklttl ergqciihae ndekpeglsp nlnrflgdrd |
| 181 | fnqfssgekn iflasfvhey srrhpqlavs vilrvakgyq ellekcfqte nplecqdkge |
| 241 | eelqkyiqes qalakrscgl fqklgeyylq naflvaytkk apqltsselm aitrkmaata |
| 301 | atccqlsedk llacgegaad iiighlcirh emtpvnpgvg qcctssyanr rpcfsslvvd |
| 361 | etyvppafsd dkfifhkdlc qaqgvalqtm kqeflinlvk qkpqiteeql eaviadfsgl |
| 421 | lekccqgqeq evcfaeegqk lisktraalg v |
| Absent in melanoma 1 protein NP_001615.2 | |
| 1 | mplsppaqgd pgepsperpp kkhttfhlwr skkkqqpapp dcgvfvphpl papagearal |
| 61 | dvvdgkyvvr dsqefplhcg esqffhttse algslllesg ifkksraqpp ednrrkpvlg |
| 121 | klgtlftagr rrnsrngles ptrsnakpls pkdvvaspkl peresersrs qssqlkqtdt |
| 181 | seegsprenp reaegelpes ggpaappdae lsprwsssaa avavqqchen dspqleplea |
| 241 | egepfpdatt takqlhsspg nssrqenaet parspgedas pgagheqeaf lgvrgapgsp |
| 301 | tqerpagglg eapngapsvc aeegslgprn arsqppkgas dlpgeppaeg aahtassaqa |
| 361 | dctarpkgha hpakvltldi ylsktegaqv depvvitpra edcgdwddme krssgrrsgr |
| 421 | rrgsqkstds pgadaelpes aarddavfdd evapnaasdn asaekkvksp raaldggvas |
| 481 | aaspeskpsp gtkgqlrges drskqpppas sptkrkgrsr aleavpappa sgprapakes |
| 541 | ppkrvpdpsp vtkgtaaesg eeaaraipre lpvksssllp eikpehkrgp lpnhfngrae |
| 601 | ggrsrelgra agapgasdad glkprnhfgv grstvttkvt lpakpkhvel nlktpknlds |
| 661 | lgnehnpfsq pvhkgntatk islfenkrtn ssprhtdirg qrntpasskt fvgraklnla |
| 721 | kkakemeqpe kkvmpnspqn gvlvketaie tkvtvseeei lpatrgmngd ssenqalgpq |
| 781 | pnqddkadvq tdagclsepv asalipvkdh kllekedsea adskslvlen vtdtaqdipt |
| 841 | tvdtkdlppt ampkpqhtfs dsqspaessp gpslslsapa pgdvpkdtcv qspissfpct |
| 901 | dlkvsenhkg cvlpvsrqnn ekmpllelgg ettpplster speavgsecp srvlvqvrsf |
| 961 | vlpvestqdv ssqvipesse vrevqlptch snepevvsva scappqeevl gnehshctae |
| 1021 | laaksgpqvi ppasektlpi qaqsqgsrtp lmaessptns pssgnhlatp qrpdqtvtng |
| 1081 | qdspasllni sagsddsvfd sssdmekfte iikqmdsavc mpmkrkkarm pnspaphfam |
| 1141 | ppihedhlek vfdpkvftfg lgkkkesqpe mspalhlmqn ldtksklrpk rasaeqsvlf |
| 1201 | kslhtntngn seplvmpein dkenrdvtng gikrsrleks alfssllssl pqdkifspsv |
| 1261 | tsvntmttaf stsqngslsq ssvsqptteg appcglnkeq snllpdnslk vfnfnsssts |
| 1321 | hsslkspshm ekypqkektk edldsrsnlh lpetkfsels klknddmeka nhiesviksn |
| 1381 | lpncansdtd fmglfkssry dpsisfsgms lsdtmtlrgs vqnklnprpg kvviysepdv |
| 1441 | sekcievfsd iqdcsswsls pvilikvvrg cwilyeqpnf eghsipleeg elelsglwgi |
| 1501 | edilerheea esdkpvvigs irhvvqdyrv shidlftepe glgilssyfd dteemqgfgv |
| 1561 | mqktcsmkvh wgtwliyeep gfqgvpfile pgeypdlsfw dteeayigsm rplkmggrkv |
| 1621 | efptdpkvvv yekpffegkc veletgmcsf vmeggeteea tgddhlpfts vgsmkvlrgi |
| 1681 | wvayekpgft ghqylleege yrdwkawggy ngelqslrpi lgdfsnahmi myseknfgsk |
| 1741 | gssidvlgiv anlketgygv ktqsinvlsg vwvayenpdf tgeqyildkg fytsfedwgg |
| 1801 | knckissvqp icldsftgpr rrnqihlfse pqfqghsqsf eettsqidds fstkscrvsg |
| 1861 | gswvvydgen ftgnqyvlee ghypclsamg cppgatfksl rfidvefsep tiilferedf |
| 1921 | kgkkielnae tvnlrslgfn tqirsvqvig giwvtyeygs yrgrqfllsp aevpnwyefs |
| 1981 | gcrqigslrp fvqkriyfrl rnkatglfms tngnledlkl lriqvmedvg addqiwiyqe |
| 2041 | gcikcriaed ccltivgslv tsgsklglal dqnadsqfws lksdgriysk lkpnlvldik |
| 2101 | ggtqydqnhi ilntvskekf tqvweamvly t |
| A-kinase anchoring protein 4, isoform 1 NP_003877.2 | |
| 1 | mmaysdttmm sddidwlrsh rgvckvdlyn pegqqdqdrk vicfvdvstl nvedkdykda |
| 61 | assssegnln lgsleekeii vikdtekkdq sktegsvclf kqapsdpvsv lnwllsdlqk |
| 121 | yalgfqhals pststckhkv gdtegeyhra ssencysvya dqvnidylmn rpqnlrlemt |
| 181 | aakntnnnqs psappakpps tqravispdg ecsiddlsfy vnrlsslviq mahkeikekl |
| 241 | egkskclhhs icpspgnker isprtpaski asemayeave ltaaemrgtg eesreggqks |
| 301 | flyselsnks ksgdkqmsqr eskefadsis kglmvyanqv asdmmvslmk tlkvhssgkp |
| 361 | ipasvvlkrv llrhtkeivs dlidscmknl hnitgvlmtd sdfvsavkrn lfnqwkqnat |
| 421 | dimeamlkrl vsaligeeke tksqslsyas lkagshdpkc rnqslefstm kaemkerdkg |
| 481 | kmksdpcksl tsaekvgehi lkegltiwnq kqgnsckvat kacsnkdekg ekinastdsl |
| 541 | akdlivsalk liqyhltqqt kgkdtceedc pgstmgymaq stqyekcggg qsakalsvkq |
| 601 | leshrapgps tcqkenqhld sqkmdmsniv lmliqkllne npfkcedpce genkcsepra |
| 661 | skaasmsnrs dkaeeqcqeh qeldctsgmk qangqfidkl vesvmklcli makysndgaa |
| 721 | laeleeqaas ankpnfrgtr cihsgampqn yqdslghevi vnnqcstnsl qkqlqavlqw |
| 781 | iaasqfnvpm lyfmgdkdgq leklpqvsak aaekgysvgg llgevmkfak erqpdeavgk |
| 841 | varkqlldwl lanl |
| A-kinase anchoring protein 4, isoform 2 NP_647450.1 | |
| 1 | msddidwlrs hrgvckvdly npegqqdqdr kvicfvdvst lnvedkdykd aassssegnl |
| 61 | nlgsleekei ivikdtekkd qsktegsvcl fkqapsdpvs vinwllsdlq kyalgfqhal |
| 121 | spststckhk vgdtegeyhr assencysvy adqvnidylm nrpqnlrlem taakntnnnq |
| 181 | spsappakpp stqravispd gecsiddlsf yvnrlsslvi qmahkeikek legkskclhh |
| 241 | sicpspgnke risprtpask iasemayeav eltaaemrgt geesreggqk sflyselsnk |
| 301 | sksgdkqmsq reskefadsi skglmvyanq vasdmmvslm ktlkvhssgk pipasvvlkr |
| 361 | vllrhtkeiv sdlidscmkn lhnitgvlmt dsdfvsavkr nlfnqwkqna tdimeamlkr |
| 421 | lvsaligeek etksgslsya slkagshdpk crnqslefst mkaemkerdk gkmksdpcks |
| 481 | ltsaekvgeh ilkegltiwn qkqgnsckva tkacsnkdek gekinastds lakdlivsal |
| 541 | kliqyhltqq tkgkdtceed cpgstmgyma qstqyekcgg gqsakalsvk qleshrapgp |
| 601 | stcqkenqhl dsqkmdmsni vlmliqklln enpfkcedpc egenkcsepr askaasmsnr |
| 661 | sdkaeeqcqe hqeldctsgm kqangqfidk lvesvmklcl imakysndga alaeleeqaa |
| 721 | sankpnfrgt rcihsgampq nyqdslghev ivnnqcstns lqkqlqavlq wiaasqfnvp |
| 781 | mlyfmgdkdg qleklpqvsa kaaekgysvg gllqevmkfa kerqpdeavg kvarkqlldw |
| 841 | llanl |
| ALK tryrosine kinase receptor, isoform 1 NP_004295.2 | |
| 1 | mgaigllwll plllstaavg sgmgtgqrag spaagpplqp replsysrlq rkslavdfvv |
| 61 | pslfrvyard lllppsssel kagrpeargs laldcapllr llgpapgvsw tagspapaea |
| 121 | rtlsrvlkgg svrklrrakq lvlelgeeai legcvgppge aavgllqfnl selfswwirq |
| 181 | gegrlrirlm pekkasevgr egrlsaaira sqprllfqif gtghsslesp tnmpspspdy |
| 241 | ftwnltwimk dsfpflshrs ryglecsfdf pceleysppl hdlrnqswsw rripseeasq |
| 301 | mdlldgpgae rskemprgsf lllntsadsk htilspwmrs ssehctlavs vhrhlqpsgr |
| 361 | yiaqllphne aareillmpt pgkhgwtvlq grigrpdnpf rvaleyissg nrslsavdff |
| 421 | alkncsegts pgskmalqss ftcwngtvlq lgqacdfhqd caqgedesqm crklpvgfyc |
| 481 | nfedgfcgwt qgtlsphtpq wqvrtlkdar fqdhqdhall lsttdvpase satvtsatfp |
| 541 | apiksspcel rmswlirgvl rgnvslvlve nktgkeqgrm vwhvaayegl slwqwmvlpl |
| 601 | ldvsdrfwlq mvawwgqgsr aivafdnisi sldcyltisg edkilqntap ksrnlfernp |
| 661 | nkelkpgens prqtpifdpt vhwlfttcga sgphgptqaq cnnayqnsnl svevgsegpl |
| 721 | kgiqiwkvpa tdtysisgyg aaggkggknt mmrshgvsvl gifnlekddm lyilvgqqge |
| 781 | dacpstnqli qkvcigennv ieeeirvnrs vhewaggggg gggatyvfkm kdgvpvplii |
| 841 | aaggggrayg aktdtfhper lennssvlgl ngnsgaaggg ggwndntsll wagkslqega |
| 901 | tgghscpqam kkwgwetrgg fggggggcss ggggggyigg naasnndpem dgedgvsfis |
| 961 | plgilytpal kvmeghgevn ikhylncshc evdechmdpe shkvicfcdh gtvlaedgvs |
| 1021 | civsptpeph lplslilsvv tsalvaalvl afsgimivyr rkhqelqamq melqspeykl |
| 1081 | sklrtstimt dynpnycfag ktssisdlke vprknitlir glghgafgev yegqvsgmpn |
| 1141 | dpsplqvavk tlpevcseqd eldflmeali iskfnhqniv rcigvslqsl prfillelma |
| 1201 | ggdlksflre trprpsqpss lamldllhva rdiacgcqyl eenhfihrdi aarnclltcp |
| 1261 | gpgrvakigd fgmardiyra syyrkggcam lpvkwmppea fmegiftskt dtwsfgvllw |
| 1321 | eifslgympy psksnqevle fvtsggrmdp pkncpgpvyr imtqcwqhqp edrpnfaiil |
| 1381 | erieyctqdp dvintalpie ygplveeeek vpvrpkdpeg vppllvsqqa kreeerspaa |
| 1441 | ppplpttssg kaakkptaae isvrvprgpa vegghvnmaf sqsnppselh kvhgsrnkpt |
| 1501 | slwnptygsw ftekptkknn piakkephdr gnlglegsct vppnvatgrl pgasllleps |
| 1561 | sltanmkevp lfrlrhfpcg nvnygyqqqg lpleaatapg aghyedtilk sknsmnqpgp |
| ALK tyrosin kinese receptor, isoform 2 NP_001340694.1 | |
| 1 | mqmelqspey klsklrtsti mtdynpnycf agktssisdl kevprknitl irglghgafg |
| 61 | evyegqvsgm pndpsplqva vktlpevcse qdeldflmea liiskfnhqn ivrcigvslq |
| 121 | slprfillel maggdlksfl retrprpsqp sslamldllh vardiacgcq yleenhfihr |
| 181 | diaarncllt cpgpgrvaki gdfgmardiy rasyyrkggc amlpvkwmpp eafmegifts |
| 241 | ktdtwsfgvl lweifslgym pypsksnqev lefvtsggrm dppkncpgpv yrimtqcwqh |
| 301 | qpedrpnfai ilerieyctq dpdvintalp ieygplveee ekvpvrpkdp egvppllvsq |
| 361 | qakreeersp aappplptts sgkaakkpta aeisvrvprg pavegghvnm afsqsnppse |
| 421 | lhkvhgsrnk ptslwnptyg swftekptkk nnpiakkeph drgnlglegs ctvppnvatg |
| 481 | rlpgasllle pssltanmke vplfrlrhfp cgnvnygyqq qglpleaata pgaghyedti |
| 541 | lksknsmnqp gp |
| Angiopoietin-2, isoform a NP_001138.1 | |
| 1 | mwqivfftls cdlvlaaayn nfrksmdsig kkqyqvqhgs csytfllpem dncrsssspy |
| 61 | vsnavqrdap leyddsvqrl qvlenimenn tqwlmkleny iqdnmkkemv eiqqnavqnq |
| 121 | tavmieigtn llnqtaeqtr kltdveaqvl nqttrlelql lehslstnkl ekqildqtse |
| 181 | inklqdknsf lekkvlamed khiiqlqsik eekdqlqvlv skqnsiieel ekkivtatvn |
| 241 | nsvlqkqqhd lmetvnnllt mmstsnsakd ptvakeeqis frdcaevfks ghttngiytl |
| 301 | tfpnsteeik aycdmeaggg gwtiiqrred gsvdfqrtwk eykvgfgnps geywlgnefv |
| 361 | sqltnqqryv lkihlkdweg neayslyehf ylsseelnyr ihlkgltgta gkissisqpg |
| 421 | ndfstkdgdn dkcickcsqm ltggwwfdac gpsnlngmyy pqrqntnkfn gikwyywkgs |
| 481 | gyslkattmm irpadf |
| Angiopoietin-2, isoform b NP_001112359.1 | |
| 1 | mwqivfftls cdlvlaaayn nfrksmdsig kkqyqvqhgs csytfllpem dncrsssspy |
| 61 | vsnavqrdap leyddsvqrl qvlenimenn tqwlmkleny iqdnmkkemv eiqqnavqnq |
| 121 | tavmieigtn llnqtaeqtr kltdveaqvl nqttrlelql lehslstnkl ekqildqtse |
| 181 | inklqdknsf lekkvlamed khiiqlqsik eekdqlqvlv skqnsiieel ekkivtatvn |
| 241 | nsvlqkqqhd lmetvnnllt mmstsnskdp tvakeeqisf rdcaevfksg httngiytlt |
| 301 | fpnsteeika ycdmeagggg wtiiqrredg svdfqrtwke ykvgfgnpsg eywlgnefvs |
| 361 | qltnqqryvl kihlkdwegn eayslyehfy lsseelnyri hlkgltgtag kissisqpgn |
| 421 | dfstkdgdnd kcickcsqml tggwwfdacg psnlngmyyp qrqntnkfng ikwyywkgsg |
| 481 | yslkattmmi rpadf |
| Angiopoietin-2, isoform c NP_001112360.1 | |
| 1 | mwqivfftls cdlvlaaayn nfrksmdsig kkqyqvqhgs csytfllpem dncrsssspy |
| 61 | vsnavqrdap leyddsvqrl qvlenimenn tqwlmkvlnq ttrlelqlle hslstnklek |
| 121 | qildqtsein klqdknsfle kkvlamedkh iiqlqsikee kdqlqvlvsk qnsiieelek |
| 181 | kivtatvnns vlqkqqhdlm etvnnlltmm stsnsakdpt vakeeqisfr dcaevfksgh |
| 241 | ttngiytltf pnsteeikay cdmeaggggw tiiqrredgs vdfqrtwkey kvgfgnpsge |
| 301 | ywlgnefvsq ltnqqryvlk ihlkdwegne ayslyehfyl sseelnyrih lkgltgtagk |
| 361 | issisqpgnd fstkdgdndk cickcsqmlt ggwwfdacgp snlngmyypq rqntnkfngi |
| 421 | kwyywkgsgy slkattmmir padf |
| Angiopoietin-1, isoform 1 precursor NP_001137.2 | |
| 1 | mtvflsfafl aailthigcs nqrrspensg rrynriqhgq caytfilpeh dgncresttd |
| 61 | qyntnalqrd aphvepdfss qklqhlehvm enytqwlqkl enyivenmks emaqiqqnav |
| 121 | qnhtatmlei gtsllsqtae qtrkltdvet qvlnqtsrle iqllenslst yklekqllqq |
| 181 | tneilkihek nsllehkile megkhkeeld tlkeekenlq glvtrqtyii qelekqlnra |
| 241 | ttnnsvlqkq qlelmdtvhn lvnlctkegv llkggkreee kpfrdcadvy qagfnksgiy |
| 301 | tiyinnmpep kkvfcnmdvn gggwtviqhr edgsldfqrg wkeykmgfgn psgeywlgne |
| 361 | fifaitsqrq ymlrielmdw egnraysqyd rfhignekqn yrlylkghtg tagkqsslil |
| 421 | hgadfstkda dndncmckca lmltggwwfd acgpsnlngm fytagqnhgk lngikwhyfk |
| 481 | gpsyslrstt mmirpldf |
| Angiopoietin-1, isoform 2 precursor NP_001186788.1 | |
| 1 | mtvflsfafl aailthigcs nqrrspensg rrynriqhgq caytfilpeh dgncresttd |
| 61 | qyntnalqrd aphvepdfss qklqhlehvm enytqwlqkl enyivenmks emaqiqqnav |
| 121 | qnhtatmlei gtsllsqtae qtrkltdvet qvlnqtsrle iqllenslst yklekqllqq |
| 181 | tneilkihek nsllehkile megkhkeeld tlkeekenlq glvtrqtyii qelekqlnra |
| 241 | ttnnsvlqkq qlelmdtvhn lvnlctkevl lkggkreeek pfrdcadvyq agfnksgiyt |
| 301 | iyinnmpepk kvfcnmdvng ggwtviqhre dgsldfqrgw keykmgfgnp sgeywlgnef |
| 361 | ifaitsqrqy mlrielmdwe gnraysqydr fhignekqny rlylkghtgt agkqsslilh |
| 421 | gadfstkdad ndncmckcal mltggwwfda cgpsnlngmf ytagqnhgkl ngikwhyfkg |
| 481 | psyslrsttm mirpldf |
| Angiopoietin-1, isoform 3 precursor NP_001300980.1 | |
| 1 | megkhkeeld tlkeekenlq glvtrqtyii qelekqlnra ttnnsvlqkq qlelmdtvhn |
| 61 | lvnlctkegv llkggkreee kpfrdcadvy qagfnksgiy tiyinnmpep kkvfcnmdvn |
| 121 | gggwtviqhr edgsldfqrg wkeykmgfgn psgeywlgne fifaitsqrq ymlrielmdw |
| 181 | egnraysqyd rfhignekqn yrlylkghtg tagkqsslil hgadfstkda dndncmckca |
| 241 | lmltggwwfd acgpsnlngm fytagqnhgk lngikwhyfk gpsyslrstt mmirpldf |
| Ankyrin repeat domain-containing protein 30A NP_443723.2 | |
| 1 | mtkrkktinl niqdaqkrta lhwacvnghe evvtflvdrk cqldvldgeh rtplmkalqc |
| 61 | hqeacanili dsgadinlvd vygntalhya vyseilsvva kllshgavie vhnkasltpl |
| 121 | llsitkrseq ivefllikna nanavnkykc talmlavchg sseivgmllq qnvdvfaadi |
| 181 | cgvtaehyav tcgfhhiheq imeyirklsk nhqntnpegt sagtpdeaap laertpdtae |
| 241 | slvektpdea aplvertpdt aeslvektpd eaaslvegts dkiqclekat sgkfeqsaee |
| 301 | tpreitspak etsekftwpa kgrprkiawe kkedtpreim spaketsekf twaakgrprk |
| 361 | iawekketpv ktgcvarvts nktkvlekgr skmiacptke sstkasandq rfpseskqee |
| 421 | deeyscdsrs lfessakiqv cipesiyqkv meinreveep pkkpsafkpa iemqnsvpnk |
| 481 | afelknegtl radpmfppes kqkdyeensw dseslcetvs qkdvclpkat hqkeidking |
| 541 | kleespnkdg llkatcgmkv siptkalelk dmqtfkaepp gkpsafepat emqksvpnka |
| 601 | lelkneqtlr adeilpsesk qkdyeenswd teslcetvsq kdvclpkaah qkeidkingk |
| 661 | legspvkdgl lkancgmkvs iptkalelmd mqtfkaeppe kpsafepaie mqksvpnkal |
| 721 | elkneqtlra deilpseskq kdyeesswds eslcetvsqk dvclpkathq keidkingkl |
| 781 | eespdndgfl kapcrmkvsi ptkalelmdm qtfkaeppek psafepaiem qksvpnkale |
| 841 | lkneqtlrad qmfpseskqk kveenswdse slretvsqkd vcvpkathqk emdkisgkle |
| 901 | dstslskild tvhscerare lqkdhceqrt gkmeqmkkkf cvlkkklsea keiksqlenq |
| 961 | kvkweqelcs vrltlnqeee krrnadilne kireelgrie eqhrkelevk qqleqalriq |
| 1021 | dielksvesn lnqvshthen enyllhencm lkkeiamlkl eiatlkhqyq ekenkyfedi |
| 1081 | kilkeknael qmtlklkees ltkrasqysg qlkvliaent mltsklkekq dkeileaeie |
| 1141 | shhprlasav qdhdqivtsr ksqepafhia gdaclqrkmn vdvsstiynn evlhqplsea |
| 1201 | qrkskslkin lnyagdalre ntlvsehaqr dqretqcqmk eaehmyqneq dnvnkhteqq |
| 1261 | esldqklfql qsknmwlqqq lvhahkkadn kskitidihf lerkmqhhll kekneeifny |
| 1321 | nnhlknriyq yekekaeten s |
| Androgen receptor, isoform 1 NP_000035.2 | |
| 1 | mevqlglgrv yprppsktyr gafqnlfqsv reviqnpgpr hpeaasaapp gasllllqqq |
| 61 | qqqqqqqqqq qqqqqqqqqq etsprqqqqq qgedgspqah rrgptgylvl deeqqpsqpq |
| 121 | salechperg cvpepgaava askglpqqlp appdeddsaa pstlsllgpt fpglsscsad |
| 181 | lkdilseast mqllqqqqqe avsegsssgr areasgapts skdnylggts tisdnakelc |
| 241 | kavsvsmglg vealehlspg eqlrgdcmya pllgvppavr ptpcaplaec kgsllddsag |
| 301 | kstedtaeys pfkggytkgl egeslgcsgs aaagssgtle lpstlslyks galdeaaayq |
| 361 | srdyynfpla lagppppppp phpharikle npldygsawa aaaaqcrygd laslhgagaa |
| 421 | gpgsgspsaa assswhtlft aeegqlygpc gggggggggg gggggggggg gggeagavap |
| 481 | ygytrppqgl agqesdftap dvwypggmvs rvpypsptcv ksemgpwmds ysgpygdmrl |
| 541 | etardhvlpi dyyfppqktc licgdeasgc hygaltcgsc kvffkraaeg kqkylcasrn |
| 601 | dctidkfrrk ncpscrlrkc yeagmtlgar klkklgnlkl qeegeasstt spteettqkl |
| 661 | tvshiegyec qpiflnvlea iepgvvcagh dnnqpdsfaa llsslnelge rqlvhvvkwa |
| 721 | kalpgfrnlh vddqmaviqy swmglmvfam gwrsftnvns rmlyfapdlv fneyrmhksr |
| 781 | mysqcvrmrh lsqefgwlqi tpqeflcmka lllfsiipvd glknqkffde lrmnyikeld |
| 841 | riiackrknp tscsrrfyql tklldsvqpi arelhqftfd llikshmvsv dfpemmaeii |
| 901 | svqvpkilsg kvkpiyfhtq |
| Androgen receptor, isoform 2 NP_001011645.1 | |
| 1 | milwlhslet ardhvlpidy yfppqktcli cgdeasgchy galtcgsckv ffkraaegkq |
| 61 | kylcasrndc tidkfrrknc pscrlrkcye agmtlgarkl kklgnlklqe egeassttsp |
| 121 | teettqkltv shiegyecqp iflnvleaie pgvvcaghdn nqpdsfaall sslnelgerq |
| 181 | lvhvvkwaka lpgfrnlhvd dqmaviqysw mglmvfamgw rsftnvnsrm lyfapdlvfn |
| 241 | eyrmhksrmy sqcvrmrhls qefgwlqitp qeflcmkall lfsiipvdgl knqkffdelr |
| 301 | mnyikeldri iackrknpts csrrfyqltk lldsvqpiar elhqftfdll ikshmvsvdf |
| 361 | pemmaeiisv qvpkilsgkv kpiyfhtq |
| Androgen receptor, isoform 3 NP_001334990.1 | |
| 1 | mevqlglgry yprppsktyr gafqnlfqsv reviqnpgpr hpeaasaapp gasllllqqq |
| 61 | qqqqqqqqqq qqqqqqqqqq etsprqqqqq qgedgspqah rrgptgylvl deeqqpsqpq |
| 121 | salechperg cvpepgaava askglpqqlp appdeddsaa pstlsllgpt fpglsscsad |
| 181 | lkdilseast mqllqqqqqe avsegsssgr areasgapts skdnylggts tisdnakelc |
| 241 | kavsvsmglg vealehlspg eqlrgdcmya pllgvppavr ptpcaplaec kgsllddsag |
| 301 | kstedtaeys pfkggytkgl egeslgcsgs aaagssgtle lpstlslyks galdeaaayq |
| 361 | srdyynfpla lagppppppp phpharikle npldygsawa aaaaqcrygd laslhgagaa |
| 421 | gpgsgspsaa assswhtlft aeegqlygpc gggggggggg gggggggggg gggeagavap |
| 481 | ygytrppqgl agqesdftap dvwypggmvs rvpypsptcv ksemgpwmds ysgpygdmrl |
| 541 | etardhvlpi dyyfppqktc licgdeasgc hygaltcgsc kvffkraaeg kqkylcasrn |
| 601 | dctidkfrrk ncpscrlrkc yeagmtlgek frvgnckhlk mtrp |
| Androgen receptor, isoform 4 NP_001334992.1 | |
| 1 | mevqlglgrv yprppsktyr gafqnlfqsv reviqnpgpr hpeaasaapp gasllllqqq |
| 61 | qqqqqqqqqq qqqqqqqqqq etsprqqqqq qgedgspqah rrgptgylvl deeqqpsqpq |
| 121 | salechperg cvpepgaava askglpqqlp appdeddsaa pstlsllgpt fpglsscsad |
| 181 | lkdilseast mqllqqqqqe avsegsssgr areasgapts skdnylggts tisdnakelc |
| 241 | kavsvsmglg vealehlspg eqlrgdcmya pllgvppavr ptpcaplaec kgsllddsag |
| 301 | kstedtaeys pfkggytkgl egeslgcsgs aaagssgtle lpstlslyks galdeaaayq |
| 361 | srdyynfpla lagppppppp phpharikle npldygsawa aaaaqcrygd laslhgagaa |
| 421 | gpgsgspsaa assswhtlft aeegqlygpc gggggggggg gggggggggg gggeagavap |
| 481 | ygytrppqgl agqesdftap dvwypggmvs rvpypsptcv ksemgpwmds ysgpygdmrl |
| 541 | etardhvlpi dyyfppqktc licgdeasgc hygaltcgsc kvffkraaeg kqkylcasrn |
| 601 | dctidkfrrk ncpscrlrkc yeagmtlgaa vvvserilrv fgvsewlp |
| Androgen receptor, isoform 5 NP_001334993.1 | |
| 1 | mevqlglgrv yprppsktyr gafqnlfqsv reviqnpgpr hpeaasaapp gasllllqqq |
| 61 | qqqqqqqqqq qqqqqqqqqq etsprqqqqq qgedgspqah rrgptgylvl deeqqpsqpq |
| 121 | salechperg cvpepgaava askglpqqlp appdeddsaa pstlsllgpt fpglsscsad |
| 181 | lkdilseast mqllqqqqqe avsegsssgr areasgapts skdnylggts tisdnakelc |
| 241 | kavsvsmglg vealehlspg eqlrgdcmya pllgvppavr ptpcaplaec kgsllddsag |
| 301 | kstedtaeys pfkggytkgl egeslgcsgs aaagssgtle lpstlslyks galdeaaayq |
| 361 | srdyynfpla lagppppppp phpharikle npldygsawa aaaaqcrygd laslhgagaa |
| 421 | gpgsgspsaa assswhtlft aeegqlygpc gggggggggg gggggggggg gggeagavap |
| 481 | ygytrppqgl agqesdftap dvwypggmvs rvpypsptcv ksemgpwmds ysgpygdmrn |
| 541 | trrkrlwkli irsinscics pretevpvrq qk |
| ATPase H+ transporting accessory protein 1 NP_001174.2 | |
| 1 | mmaamatarv rmgprcaqal wrmpwlpvfl slaaaaaaaa aeqqvplvlw ssdrdlwapa |
| 61 | adtheghits dlqlstyldp alelgprnvl lflqdklsie dftayggvfg nkqdsafsnl |
| 121 | enaldlapss lvlpavdwya vstlttylqe klgasplhvd latlrelkln aslpalllir |
| 181 | lpytassglm aprevltgnd evigqvlstl ksedvpytaa ltavrpsrva rdvavvaggl |
| 241 | grqllqkqpv spvihppvsy ndtaprilfw aqnfsvaykd qwedltpltf gvqelnltgs |
| 301 | fwndsfarls ltyerlfgtt vtfkfilanr lypvsarhwf tmerlevhsn gsvayfnasq |
| 361 | vtgpsiysfh ceyvsslskk gsllvartqp spwqmmlqdf qiqafnvmge qfsyasdcas |
| 421 | ffspgiwmgl ltslfmlfif tyglhmilsl ktmdrfddhk gptisltqiv |
| B melanoma antigen 1 precursor NP_001178.1 | |
| 1 | maaravflal saqllqarlm keespvvswr lepedgtalc fif |
| BCR/ABL fusion protein e14ab NG_050673.1 | |
| 1 | gcacctgcag ggagggcagg cagctagcct gaaggctgat ccccccttcc tgttagcact |
| 61 | tttgatggga ctagtggact ttggttcaga aggaagagct atgcttgtta gggcctcttg |
| 121 | tctcctccca ggagtggaca aggtgggtta ggagcagttt ctccctgagt ggctgctgct |
| 181 | gggtggttga ggagatgcac ggcttctgtt cctagtcaca aggctgcagc agacgctcct |
| 241 | cagatgctct gtgccttgga tctggcccca ctcccgtcct cccagccctc ctctcctcca |
| 301 | gctacctgcc agccggcact tttggtcaag ctgttttgca ttcactgttg cacatatgct |
| 361 | cagtcacaca cacagcatac gctatgcaca tgtgtccaca cacaccccac ccacatccca |
| 421 | catcaccccg accccctctg ctgtccttgg aaccttatta cacttcgagt cactggtttg |
| 481 | cctgtattgt gaaaccagct ggatcctgag atccccaaga cagaaatcat gatgagtatg |
| 541 | tttttggccc atgacactgg cttaccttgt gccaggcaga tggcagccac acagtgtcca |
| 601 | ccggatggtt gattttgaag cagagttagc ttgtcacctg cctccctttc ccgggacaac |
| 661 | agaagctgac ctctttgatc tcttgcgcag atgatgagtc tccggggctc tatgggtttc |
| 721 | tgaatgtcat cgtccactca gccactggat ttaagcagag ttcaagtaag tactggtttg |
| 781 | gggaggaggg ttgcagcggc cgagccaggg tctccaccca ggaaggactc atcgggcagg |
| 841 | gtgtggggaa acagggaggt tgttcagatg accacgggac acctttgacc ctggccgctg |
| 901 | tggagtgttt gtgctggttg atgccttctg ggtgtggaat tgtttttccc ggagtggcct |
| 961 | ctgccctctc ccctagcctg tctcagatcc tgggagctgg tgagctgccc cctgcaggtg |
| 1021 | gatcgagtaa ttgcaggggt ttggcaagga ctttgacaga catccccagg ggtgcccggg |
| 1081 | agtgtggggt ccaagccagg agggctgtca gcagtgcacc ttcaccccac agcagagcag |
| 1141 | atttggctgc tctgtcgagc tggatggata ctactttttt tttcctttcc ctctaagtgg |
| 1201 | gggtctcccc cagctactgg agctgtcaga acagtgaagg ctggtaacac atgagttgca |
| 1261 | ctgtgtaagt ttctcgaggc cgggcgcagt ggctcatgcc tgtaatccca gcactttggg |
| 1321 | aggctgaggc aggtggatcg cttgagctca ggagttggag accagcctga ccaacatggt |
| 1381 | gaaaccctgt gtctactaaa aatacaaaga ttagccgggc taggcagtgg gcacctgtaa |
| 1441 | tcacaactgc ttgggaggct gagggaagag aatcgcttga acccaggagg cggaggttgc |
| 1501 | agtgagccga gcttgtgcca ctgcattcca gcctgggcga cagagcaaga ctccgcctca |
| 1561 | aaaaaaaaaa aaaaaagttc ctagaaacag caaaatgtgg agacagaaag cttaccaggg |
| 1621 | attgttgggg aatggggttg ggagagagga ctaactgcag atgaacccaa gggggacttt |
| 1681 | ttaggtgaga gcagtgtcgt gaaaagactg tggtgctgtt tgcgctcaca tttacatttc |
| 1741 | ctaaaattct ttaaacccta cacttggaat ggatgaatta catgacatgc agattgcacc |
| 1801 | ttcataacat aatctttctc ctgggcccct gtctctggct gcctcataaa cgctggtgtt |
| 1861 | tccctcgtgg gcctccctgc atccctgcat ctcctcccgg gtcctgtctg tgagcaatac |
| 1921 | agcgtgacac cctacgctgc cccgtggtcc cgggcttgtc tctccttgcc tccctgttac |
| 1981 | ctttctttct atctcttcct tgccccgtgc actcaacctt gcatccccaa accaaaccta |
| 2041 | ttattcatgg accccaaact tgttcctctt atgtcctgtc cctttgaggg gcaccaccat |
| 2101 | ccacccgcat ggccaagcca gaaaccgtgg tctgctctcc ctccgttaaa tgccattctc |
| 2161 | catcagtgag gcttcttagt catctctggc tgcctggcca ggccctggct gtggcctcct |
| 2221 | ccctggtctt tgtagctctg gatatccctg cagaaagggt ccccactacc aggcctctcc |
| 2281 | atccccagtc tcaggtagtt tttctaaaat gcaaacccca ccctgcaact taccgcccac |
| 2341 | agcccagccc actcttctcc aggcctcgcc tccctccctt ccccctgcac cccacgactt |
| 2401 | ctccagcact gagctgcttc ctgtgcccca cagtggcctg gagtcccctt tgccttaact |
| 2461 | ctttgcccca tagtacagcg gggtctgctc tgattgtagg ggcttcccac atcccccagg |
| 2521 | atggctgccc tctgctgtgg catcactgtg taacaatggc gtgtacacct ctctgtcccc |
| 2581 | accagtgcag ggcccttctc atcgtagggg ctttagctgg ggtttgtgga tcgactgagt |
| 2641 | gaacgaatgt tgtgggaagt cccgtttccc agccgcaccc agggaaattc cacagagcgg |
| 2701 | gcaggggcat cgcatgaggt gctggtgttc acgccagacc acaattaggt gtttaatttt |
| 2761 | taaaaagaaa gttacaacct ttttttttta tttttatttt ttctgattct gcaaataaca |
| 2821 | cctgctctta cagaccatgt gggtgatgtg gaaaagacct gtgaccttct ccatgtccac |
| 2881 | ttctccccac agatctgtac tgcaccctgg aggtggattc ctttgggtat tttgtgaata |
| 2941 | aagcaaagac gcgcgtctac agggacacag ctgagcca |
| Serine/threonine-protein kinase B-raf, isoform 1 NP_004324.2 | |
| 1 | maalsggggg gaepgqalfn gdmepeagag agaaassaad paipeevwni kqmikltqeh |
| 61 | iealldkfgg ehnppsiyle ayeeytskld alqqreqqll eslgngtdfs vsssasmdtv |
| 121 | tsssssslsv lpsslsvfqn ptdvarsnpk spqkpivrvf lpnkgrtvvp arcgvtvrds |
| 181 | lkkalmmrgl ipeccavyri qdgekkpigw dtdiswltge elhvevlenv pltthnfvrk |
| 241 | tfftlafcdf crkllfqgfr cqtcgykfhq rcstevplmc vnydqldllf vskffehhpi |
| 301 | pqeeaslaet altsgsspsa pasdsigpqi ltspspsksi pipqpfrpad edhrnqfgqr |
| 361 | drsssapnvh intiepvnid dlirdqgfrg dggsttglsa tppaslpgsl tnvkalqksp |
| 421 | gpqrerksss ssedrnrmkt lgrrdssddw eipdgqitvg qrigsgsfgt vykgkwhgdv |
| 481 | avkmlnvtap tpqqlqafkn evgvlrktrh vnillfmgys tkpqlaivtq wcegsslyhh |
| 541 | lhiietkfem iklidiarqt aqgmdylhak siihrdlksn niflhedltv kigdfglatv |
| 601 | ksrwsgshqf eqlsgsilwm apevirmqdk npysfqsdvy afgivlyelm tgqlpysnin |
| 661 | nrdqiifmvg rgylspdlsk vrsncpkamk rlmaeclkkk rderplfpqi lasiellars |
| 721 | lpkihrsase pslnragfqt edfslyacas pktpiqaggy gafpvh |
| Serine/threonine-protein kinase B-raf, isoform 2 NP_001341538.1 | |
| 1 | maalsggggg gaepgqalfn gdmepeagag agaaassaad paipeevwni kqmikltqeh |
| 61 | iealldkfgg ehnppsiyle ayeeytskld alqqreqqll eslgngtdfs vsssasmdtv |
| 121 | tsssssslsv lpsslsvfqn ptdvarsnpk spqkpivrvf lpnkqrtvvp arcgvtvrds |
| 181 | lkkalmmrgl ipeccavyri qdgekkpigw dtdiswltge elhvevlenv pltthnfvrk |
| 241 | tfftlafcdf crkllfqgfr cqtcgykfhq rcstevplmc vnydqldllf vskffehhpi |
| 301 | pqeeaslaet altsgsspsa pasdsigpqi ltspspsksi pipqpfrpad edhrnqfgqr |
| 361 | drsssapnvh intiepvnid dlirdqgfrg dggsttglsa tppaslpgsl tnvkalqksp |
| 421 | gpqrerksss ssedrnrmkt lgrrdssddw eipdgqitvg qrigsgsfgt vykgkwhgdv |
| 481 | avkmlnvtap tpqqlqafkn evgvlrktrh vnillfmgys tkpqlaivtq wcegsslyhh |
| 541 | lhiietkfem iklidiarqt aqgmdylhak siihrdlksn niflhedltv kigdfglatv |
| 601 | ksrwsgshqf eqlsgsilwm apevirmqdk npysfqsdvy afgivlyelm tgqlpysnin |
| 661 | nrdqiifmvg rgylspdlsk vrsncpkamk rlmaeclkkk rderplfpqi lasiellars |
| 721 | lpkihrsase pslnragfqt edfslyacas pktpiqaggy gefaafk |
| Carbonic anhydrase 9 precursor NP_001207.2 | |
| 1 | maplcpspwl pllipapapg ltvqlllsll llvpvhpqrl prmqedsplg ggssgeddpl |
| 61 | geedlpseed spreedppge edlpgeedlp geedlpevkp kseeegslkl edlptveapg |
| 121 | dpqepqnnah rdkegddqsh wryggdppwp rvspacagrf qspvdirpql aafcpalrpl |
| 181 | ellgfqlppl pelrlrnngh svqltlppgl emalgpgrey ralqlhlhwg aagrpgseht |
| 241 | veghrfpaei hvvhlstafa rvdealgrpg glavlaafle egpeensaye qllsrleeia |
| 301 | eegsetqvpg ldisallpsd fsryfqyegs lttppcaqgv iwtvfnqtvm lsakqlhtls |
| 361 | dtlwgpgdsr lqlnfratqp lngrvieasf pagvdsspra aepvqlnscl aagdilalvf |
| 421 | gllfavtsva flvqmrrqhr rgtkggvsyr paevaetga |
| G/mitotic-specific cyclin-B1, isoform 1 NP_114172.1 | |
| 1 | malrvtrnsk inaenkakin magakrvpta paatskpglr prtalgdign kvseqlqakm |
| 61 | pmkkeakpsa tgkvidkklp kplekvpmlv pvpvsepvpe pepepepepv keeklspepi |
| 121 | lvdtaspspm etsgcapaee dlcqafsdvi lavndvdaed gadpnlcsey vkdiyaylrq |
| 181 | leeeqavrpk yllgrevtgn mrailidwlv qvqmkfrllq etmymtvsii drfmqnncvp |
| 241 | kkmlqlvgvt amfiaskyee myppeigdfa fvtdntytkh qirqmemkil ralnfglgrp |
| 301 | lplhflrras kigevdveqh tlakylmelt mldydmvhfp psqiaagafc lalkildnge |
| 361 | wtptlqhyls yteesllpvm qhlaknvvmv nqgltkhmtv knkyatskha kistlpqlns |
| 421 | alvqdlakav akv |
| G/mitotic-specific cyclin-B1, isoform 2 NP_001341773.1 | |
| 1 | malrvtrnsk inaenkakin magakrvpta paatskpglr prtalgdign kvseqlqakm |
| 61 | pmkkeakpsa tgkvidkklp kplekvpmlv pvpvsepvpe pepepepepv keeklspepi |
| 121 | lvdtaspspm etsgcapaee dlcqafsdvi lavndvdaed gadpnlcsey vkdiyaylrq |
| 181 | leeeqavrpk yllgrevtgn mrailidwlv qvqmkfrllq etmymtvsii drfmqnncvp |
| 241 | kkmlqlvgvt amfiaskyee myppeigdfa fvtdntytkh qirqmemkil ralnfglgrp |
| 301 | lplhflrras kigevdveqh tlakylmelt mldydmvhfp psqiaagafc lalkildnge |
| 361 | wtvknkyats khakistlpq lnsalvqdla kavakv |
| G/mitotic-specific cyclin-B1, isoform 3 NP_001341774.1 | |
| 1 | malrvtrnsk inaenkakin magakrvpta paatskpglr prtalgdign kvseqlqakm |
| 61 | pmkkeakpsa tgkvidkklp kplekvpmlv pvpvsepvpe pepepepepv keeklspepi |
| 121 | lvdtaspspm etsgcapaee dlcqafsdvi lavndvdaed gadpnlcsey vkdiyaylrq |
| 181 | lenncvpkkm lqlvgvtamf iaskyeemyp peigdfafvt dntytkhqir qmemkilral |
| 241 | nfglgrplpl hflrraskig evdveqhtla kylmeltmld ydmvhfppsq iaagafclal |
| 301 | kildngewtp tlqhylsyte esllpvmqhl aknvvmvnqg ltkhmtvknk yatskhakis |
| 361 | tlpqlnsalv qdlakavakv |
| CD276, isoform a precursor NP_001019907.1 | |
| 1 | mlrrrgspgm gvhvgaalga lwfcltgale vqvpedpvva lvgtdatlcc sfspepgfsl |
| 61 | aqlnliwqlt dtkqlvhsfa egqdqgsaya nrtalfpdll aqgnaslrlq rvrvadegsf |
| 121 | tcfvsirdfg saavslqvaa pyskpsmtle pnkdlrpgdt vtitcssyqg ypeaevfwqd |
| 181 | gqgvpltgnv ttsqmaneqg lfdvhsilrv vlgangtysc lvrnpvlqqd ahssvtitpq |
| 241 | rsptgavevq vpedpvvalv gtdatlrcsf spepgfslaq lnliwqltdt kqlvhsfteg |
| 301 | rdqgsayanr talfpdllaq gnaslrlqrv rvadegsftc fvsirdfgsa avslqvaapy |
| 361 | skpsmtlepn kdlrpgdtvt itcssyrgyp eaevfwqdgq gvpltgnvtt sqmaneqglf |
| 421 | dvhsvlrvvl gangtysclv rnpvlqqdah gsvtitgqpm tfppealwvt vglsvclial |
| 481 | lvalafvcwr kikqsceeen agaedqdgeg egsktalqpl khsdskeddg qeia |
| CD276, isoform b precursor NP_001316557.1, NP_079516.1 | |
| 1 | mlrrrgspgm gvhvgaalga lwfcltgale vqvpedpvva lvgtdatlcc sfspepgfsl |
| 61 | aqlnliwqlt dtkqlvhsfa egqdqgsaya nrtalfpdll aqgnaslrlq rvrvadegsf |
| 121 | tcfvsirdfg saavslqvaa pyskpsmtle pnkdlrpgdt vtitcssyrg ypeaevfwqd |
| 181 | gqgvpltgnv ttsqmaneqg lfdvhsvlrv vlgangtysc lvrnpvlqqd ahgsvtitgq |
| 241 | pmtfppealw vtvglsvcli allvalafvc wrkikqscee enagaedqdg egegsktalq |
| 301 | plkhsdsked dgqeia |
| CD276, isoform c NP_001316558.1 | |
| 1 | mtlepnkdlr pgdtvtitcs syqgypeaev fwqdgqgvpl tgnvttsqma neqglfdvhs |
| 61 | ilrvvlgang tysclvrnpv lqqdahssvt itpqrsptga vevqvpedpv valvgtdatl |
| 121 | rcsfspepgf slaqlnliwq ltdtkqlvhs ftegrdqgsa yanrtalfpd llaqgnaslr |
| 181 | lqrvrvadeg sftcfvsird fgsaavslqv aapyskpsmt lepnkdlrpg dtvtitcssy |
| 241 | rgypeaevfw qdgqgvpltg nvttsqmane qglfdvhsvl rvvlgangty sclvrnpvlq |
| 301 | qdahgsvtit gqpmtfppea lwvtvglsvc liallvalaf vcwrkikgsc eeenagaedq |
| 361 | dgegegskta lqplkhsdsk eddgqeia |
| Carcinoembryonic antigen-related cell adhesion molecule 3, isoform 1 | |
| precursor NP_001806.2 | |
| 1 | mgppsasphr ecipwqglll tasllnfwnp pttaklties mplsvaegke vlllvhnlpq |
| 61 | hlfgyswykg ervdgnsliv gyvigtqqat pgaaysgret iytnaslliq nvtqndigfy |
| 121 | tlqviksdlv neeatgqfhv yqenapglpv gavagivtgv lvgvalvaal vcflllaktg |
| 181 | rtsiqrdlke qqpqalapgr gpshssafsm splstaqapl pnprtaasiy eellkhdtni |
| 241 | ycrmdhkaev as |
| Carcinoembryonic antigen-related cell adhesion molecule 3, isoform 2 | |
| precursor NP_001264092.1 | |
| 1 | mgppsasphr ecipwqglll tasllnfwnp pttaklties mplsvaegke vlllvhnlpq |
| 61 | hlfgyswykg ervdgnsliv gyvigtqqat pgaaysgret iytnaslliq nvtqndigfy |
| 121 | tlqviksdlv neeatgqfhv yqenapglpv gavagivtgv lvgvalvaal vcflllaktg |
| 181 | rpwslpqlcl ldvpslhcpg pptqpqdssf hl |
| Carcinoembryonic antigen-related cell adhesion molecule 5, isoform 1 | |
| preprotein NP_001278413.1, NP_004354.3 | |
| 1 | mespsapphr wcipwqrlll taslltfwnp pttaklties tpfnvaegke vlllvhnlpq |
| 61 | hlfgyswykg ervdgnrqii gyvigtqqat pgpaysgrei iypnaslliq niiqndtgfy |
| 121 | tlhviksdlv neeatgqfrv ypelpkpsis snnskpvedk davaftcepe tqdatylwwv |
| 181 | nnqslpvspr lqlsngnrtl tlfnvtrndt asykcetqnp vsarrsdsvi lnvlygpdap |
| 241 | tisplntsyr sgenlnlsch aasnppaqys wfvngtfqqs tqelfipnit vnnsgsytcq |
| 301 | ahnsdtglnr ttvttitvya eppkpfitsn nsnpvededa valtcepeiq nttylwwvnn |
| 361 | qslpvsprlq lsndnrtltl lsvtrndvgp yecgiqnels vdhsdpviln vlygpddpti |
| 421 | spsytyyrpg vnlslschaa snppaqyswl idgniqqhtq elfisnitek nsglytcqan |
| 481 | nsasghsrtt vktitvsael pkpsissnns kpvedkdava ftcepeaqnt tylwwvngqs |
| 541 | lpvsprlqls ngnrtltlfn vtrndarayv cgiqnsvsan rsdpvtldvl ygpdtpiisp |
| 601 | pdssylsgan lnlschsasn pspqyswrin gipqqhtqvl fiakitpnnn gtyacfvsnl |
| 661 | atgrnnsivk sitvsasgts pglsagatvg imigvlvgva li |
| Carcinoembryonic antigen-related cell adhesion molecule 5, isoform 2 | |
| preprotein NP_001295327.1 | |
| 1 | mespsapphr wcipwqr111 taslltfwnp pttaklties tpfnvaegke vlllvhnlpq |
| 61 | hlfgyswykg ervdgnrqii gyvigtqqat pgpaysgrei iypnaslliq niiqndtgfy |
| 121 | tlhviksdlv neeatgqfrv ypelpkpsis snnskpvedk davaftcepe tqdatylwwv |
| 181 | nnqslpvspr lqlsngnrtl tlfnvtrndt asykcetqnp vsarrsdsvi lnvlygpdap |
| 241 | tisplntsyr sgenlnlsch aasnppaqys wfvngtfqqs tqelfipnit vnnsgsytcq |
| 301 | ahnsdtglnr ttvttitvye ppkpfitsnn snpvededav altcepeiqn ttylwwvnnq |
| 361 | slpvsprlql sndnrtltll svtrndvgpy ecgiqnelsv dhsdpvilnv lygpddptis |
| 421 | psytyyrpgv nlslschaas nppaqyswli dgniqqhtqe lfisnitekn sglytcqann |
| 481 | sasghsrttv ktitvsaelp kpsissnnsk pvedkdavaf tcepeaqntt ylwwvngqsl |
| 541 | pvsprlqlsn gnrtltlfnv trndarayvc giqnsysanr sdpvtldvly gpdtpiispp |
| 601 | dssylsganl nlschsasnp spqyswring ipqqhtqvlf iakitpnnng tyacfvsnla |
| 661 | tgrnnsivks itvsasgtsp glsagatvgi migvlvgval i |
| Baculoviral IAP repeat containing 2, isoform 1 NP_001157.1, | |
| NP_001243092.1 | |
| 1 | mhktasqrlf pgpsyqniks imedstilsd wtnsnkqkmk ydfscelyrm stystfpagv |
| 61 | pvserslara gfyytgvndk vkcfccglml dnwklgdspi qkhkqlypsc sfiqnlvsas |
| 121 | lgstskntsp mrnsfahsls ptlehsslfs gsysslspnp lnsravedis ssrtnpysya |
| 181 | msteearflt yhmwpltfls pselaragfy yigpgdrvac facggklsnw epkddamseh |
| 241 | rrhfpncpfl ensletlrfs isnlsmqtha armrtfmywp ssvpvqpeql asagfyyvgr |
| 301 | nddvkcfccd gglrcwesgd dpwvehakwf prceflirmk gqefvdeiqg ryphlleqll |
| 361 | stsdttgeen adppiihfgp gesssedavm mntpvvksal emgfnrdlvk qtvqskiltt |
| 421 | genyktvndi vsallnaede kreeekekqa eemasddlsl irknrmalfq qltcvlpild |
| 481 | nllkanvink qehdiikqkt qiplqareli dtilvkgnaa anifknclke idstlyknlf |
| 541 | vdknmkyipt edvsglslee qlrrlqeert ckvcmdkevs vvfipcghlv vcqecapslr |
| 601 | kcpicrgiik gtvrtfls |
| Baculoviral IAP repeat containing 2, isoform 2 NP_001243095.1 | |
| 1 | mstystfpag vpvserslar agfyytgvnd kvkcfccglm ldnwklgdsp iqkhkqlyps |
| 61 | csfiqnlvsa slgstsknts pmrnsfahsl sptlehsslf sgsysslspn plnsravedi |
| 121 | sssrtnpysy amsteearfl tyhmwpltfl spselaragf yyigpgdrva cfacggklsn |
| 181 | wepkddamse hrrhfpncpf lensletlrf sisnlsmqth aarmrtfmyw pssvpvqpeq |
| 241 | lasagfyyvg rnddvkcfcc dgglrcwesg ddpwvehakw fprceflirm kgqefvdeiq |
| 301 | gryphlleql lstsdttgee nadppiihfg pgesssedav mmntpvvksa lemgfnrdlv |
| 361 | kqtvqskilt tgenyktvnd ivsallnaed ekreeekekq aeemasddls lirknrmalf |
| 421 | qqltcvlpil dnllkanvin kqehdiikqk tqiplqarel idtilvkgna aanifknclk |
| 481 | eidstlyknl fvdknmkyip tedvsglsle eqlrrlqeer tckvcmdkev svvfipcghl |
| 541 | vvcqecapsl rkcpicrgii kgtvrtfls |
| Chondrosarcoma-associated gene 2/3 protein, isoform X1 XP_006724920.1 | |
| 1 | mwmgliqlve gvkrkdqgfl ekefyhktni kmrceflacw paftvlgeaw rdqvdwsrll |
| 61 | rdtglvkmsr kprassplsn nhpptpkrrg sgrhplnpgp ealskfprqp grekgpikev |
| 121 | pgtkgsp |
| Chondrosarcoma-associated gene 2/3 protein, isoform X2 XP_016885512.1 | |
| 1 | mwmgliqlve gvkrkdqgfl ekefyhktni kmrceflacw paftvlgeaw rdqvdwsrll |
| 61 | rdtglvkmsr kprassplsn nhpptpkrfp rqpgrekgpi kevpgtkgsp |
| Chondroitin sulfate proteoglycan 4 precursor NP_001888.2 | |
| 1 | mqsgprpplp apglalaltl tmlarlasaa sffgenhlev pvataltdid lqlqfstsqp |
| 61 | eallllaagp adhlllqlys grlqvrlvlg qeelrlqtpa etllsdsiph tvvltvvegw |
| 121 | atlsvdgfln assavpgapl evpyglfvgg tgtlglpylr gtsrplrgcl haatlngrsl |
| 181 | lrpltpdvhe gcaeefsasd dvalgfsgph slaafpawgt qdegtleftl ttqsrqapla |
| 241 | fqaggrrgdf iyvdifeghl ravvekgqgt vllhnsvpva dgqphevsvh inahrleisv |
| 301 | dqypthtsnr gvlsyleprg slllggldae asrhlqehrl gltpeatnas llgcmedlsv |
| 361 | ngqrrglrea lltrnmaagc rleeeeyedd ayghyeafst lapeawpame lpepcvpepg |
| 421 | lppvfanftq lltisplvva eggtawlewr hvqptldlme aelrksqvlf svtrgarhge |
| 481 | leldipgaqa rkmftlldvv nrkarfihdg sedtsdqlvl evsvtarvpm psclrrgqty |
| 541 | llpiqvnpvn dpphiifphg slmvilehtq kplgpevfqa ydpdsacegl tfqvlgtssg |
| 601 | lpverrdqpg epatefscre leagslvyvh rggpaqdltf rvsdglqasp patlkvvair |
| 661 | paiqihrstg lrlaqgsamp ilpanlsvet navgqdvsvl frvtgalqfg elqkqgaggv |
| 721 | egaewwatqa fhqrdveqgr vrylstdpqh haydtvenla levqvgqeil snlsfpvtiq |
| 781 | ratvwmlrle plhtqntqqe tlttahleat leeagpsppt fhyevvqapr kgnlqlqgtr |
| 841 | lsdgqgftqd diqagrvtyg ataraseave dtfrfrvtap pyfsplytfp ihiggdpdap |
| 901 | vltnvllvvp eggegvlsad hlfvkslnsa sylyevmerp rhgrlawrgt qdkttmvtsf |
| 961 | tnedllrgrl vyqhddsett eddipfvatr qgessgdmaw eevrgvfrva iqpvndhapv |
| 1021 | qtisrifhva rggrrllttd dvafsdadsg fadaqlvltr kdllfgsiva vdeptrpiyr |
| 1081 | ftqedlrkrr vlfvhsgadr gwiqlqvsdg qhqatallev qasepylrva ngsslvvpqg |
| 1141 | gqgtidtavl hldtnldirs gdevhyhvta gprwgqlvra gqpatafsqq dlldgavlys |
| 1201 | hngslsprdt mafsveagpv htdatlqvti alegplaplk lvrhkkiyvf qgeaaeirrd |
| 1261 | qleaaqeavp padivfsvks ppsagylvmv srgaladepp sldpvqsfsq eavdtgrvly |
| 1321 | lhsrpeawsd afsldvasgl gaplegvlve levlpaaipl eaqnfsvpeg gsltlappll |
| 1381 | rvsgpyfptl lglslqvlep pqhgalqked gpqartlsaf swrmveeqli ryvhdgsetl |
| 1441 | tdsfvlmana semdrqshpv aftvtvlpvn dqppilttnt glqmwegata pipaealrst |
| 1501 | dgdsgsedlv ytieqpsngr vvlrgapgte vrsftqaqld gglvlfshrg tldggfrfrl |
| 1561 | sdgehtspgh ffrvtaqkqv llslkgsqtl tvcpgsvqpl ssqtlrasss agtdpqllly |
| 1621 | rvvrgpqlgr lfhaqqdstg ealvnftqae vyagnilyeh emppepfwea hdtlelqlss |
| 1681 | ppardvaatl avavsfeaac pqrpshlwkn kglwvpegqr aritvaalda snllasvpsp |
| 1741 | qrsehdvlfq vtqfpsrgql lvseeplhag qphflqsqla agqlvyahgg ggtqqdgfhf |
| 1801 | rahlqgpaga svagpqtsea faitvrdvne rppqpqasvp lrltrgsrap israqlsvvd |
| 1861 | pdsapgeiey evqraphngf lslvggglgp vtrftqadvd sgrlafvang ssvagifqls |
| 1921 | msdgaspplp mslavdilps aievqlrapl evpqalgrss lsqqqlrvvs dreepeaayr |
| 1981 | liqgpqyghl lvggrptsaf sqfqidqgev vfaftnfsss hdhfrvlala rgvnasavvn |
| 2041 | vtvrallhvw aggpwpqgat lrldptvlda gelanrtgsv prfrllegpr hgrvvrvpra |
| 2101 | rtepggsqlv eqftqqdled grlglevgrp egrapgpagd sltlelwaqg vppavasldf |
| 2161 | atepynaarp ysvallsvpe aarteagkpe sstptgepgp masspepava kggflsflea |
| 2221 | nmfsviipmc lvllllalil pllfylrkrn ktgkhdvqvl takprnglag dtetfrkvep |
| 2281 | gqaipltavp gqgpppggqp dpellqfcrt pnpalkngqy wv |
| Cancer/testis antigen 2 isoform LAGE-1a NP_758965.2 | |
| 1 | mqaegrgtgg stgdadgpgg pgipdgpggn aggpgeagat ggrgprgaga arasgprgga |
| 61 | prgphggaas aqdgrcpcga rrpdsrllel hitmpfsspm eaelvrrils rdaaplprpg |
| 121 | avlkdftvsg nllfirltaa dhrqlqlsis sclqqlsllm witqcflpvf laqapsgqrr |
| Cancer/testis antigen 2 isoform LAGE-1b NP_066274.2 | |
| 1 | mqaegrgtgg stgdadgpgg pgipdgpggn aggpgeagat ggrgprgaga arasgprgga |
| 61 | prgphggaas aqdgrcpcga rrpdsrllel hitmpfsspm eaelvrrils rdaaplprpg |
| 121 | avlkdftvsg nllfmsvrdq dregagrmrv vgwglgsasp egqkardlrt pkhkvseqrp |
| 181 | gtpgppppeg aqgdgcrgva fnvmfsaphi |
| Transcriptional repressor CTCFL, isoform 1 NP_001255969.1, | |
| NP_001255970.1, NP_542185.2 | |
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveasklkrh |
| 361 | vrshtgerpf qccqcsyasr dtyklkrhmr thsgekpyec hichtrftqs gtmkihilqk |
| 421 | hgenvpkyqc phcatiiark sdlrvhmrnl haysaaelkc rycsavfher yaliqhqkth |
| 481 | knekrfkckh csyackqerh mtahirthtg ekpftclscn kcfrqkqlln ahfrkyhdan |
| 541 | fiptvykcsk cgkgfsrwin lhrhsekcgs geaksaasgk grrtrkrkqt ilkeatkgqk |
| 601 | eaakgwkeaa ngdeaaaeea sttkgeqfpg emfpvacret tarvkeevde gvtcemllnt |
| 661 | mdk |
| Transcriptional repressor CTCFL, isoform 2 NP_001255971.1 | |
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveasklkrh |
| 361 | vrshtgerpf qccqcsyasr dtyklkrhmr thsgekpyec hichtrftqs gtmkihilqk |
| 421 | hgenvpkyqc phcatiiark sdlrvhmrnl haysaaelkc rycsavfher yaliqhqkth |
| 481 | knekrfkckh csyackqerh mtahirthtg ekpftclscn kcfrqkqlln ahfrkyhdan |
| 541 | fiptvykcsk cgkgfsrwin lhrhsekcgs geaksaasgk grrtrkrkqt ilkeatkgqk |
| 601 | eaakgwkeaa ngdaaaeeas ttkgeqfpge mfpvacrett arvkeevdeg vtcemllntm |
| 661 | dk |
| Transcriptional repressor CTCFL, isoform 3 NP_001255972.1 | |
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveasklkrh |
| 361 | vrshtgerpf qccqcsyasr dtyklkrhmr thsgekpyec hichtrftqs gtmkihilqk |
| 421 | hgenvpkyqc phcatiiark sdlrvhmrnl haysaaelkc rycsavfher yaliqhqkth |
| 481 | knekrfkckh csyackqerh mtahirthtg ekpftclscn kcfrqkqlln ahfrkyhdan |
| 541 | fiptvykcsk cgkgfsrwin lhrhsekcgs geaksaasgk grrtrkrkqt ilkeatkgqk |
| 601 | eaakgwkeaa ngdeaaaeea sttkgeqfpg emfpvacret tarvkeevde gvtcemllnt |
| 661 | mdnsagctgr mmlvsawllg rpqetynqgr rrrgsrrvtw |
| Transcriptional repressor CTCFL, isoform 4 NP_001255973.1 | |
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveasklkrh |
| 361 | vrshtgerpf qccqcsyasr dtyklkrhmr thsgekpyec hichtrftqs gtmkihilqk |
| 421 | hgenvpkyqc phcatiiark sdlrvhmrnl haysaaelkc rycsavfher yaliqhqkth |
| 481 | knekrfkckh csyackqerh mtahirthtg ekpftclscn kcfrqkqlln ahfrkyhdan |
| 541 | fiptvykcsk cgkgfsrwin lhrhsekcgs geaksaasgk grrtrkrkqt ilkeatkgqk |
| 601 | eaakgwkeaa ngdgvisahr nlcllgssds hasvsgagit darhhawliv llflvemgfy |
| 661 | hvshs |
| Transcriptional repressor CTCFL, isoform 5 NP_001255974.1 | |
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveasklkrh |
| 361 | vrshtgerpf qccqcsyasr dtyklkrhmr thsgekpyec hichtrftqs gtmkihilqk |
| 421 | hgenvpkyqc phcatiiark sdlrvhmrnl haysaaelkc rycsavfher yaliqhqkth |
| 481 | knekrfkckh csyackqerh mtahirthtg ekpftclscn kcfrqkqlln ahfrkyhdan |
| 541 | fiptvykcsk cgkgfsrwil wvgnsevael ggpgsgpllr lqsgcppglh hpkaglgped |
| 601 | plpgqlrhtt agtglssllq gplcraa |
| Transcriptional repressor CTCFL, isoform 6 NP_001255975.1 | |
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveasklkrh |
| 361 | vrshtgerpf qccqcsyasr dtyklkrhmr thsgvhmrnl haysaaelkc rycsavfher |
| 421 | yaliqhqkth knekrfkckh csyackqerh mtahirthtg ekpftclscn kcfrqkqlln |
| 481 | ahfrkyhdan fiptvykcsk cgkgfsrwin lhrhsekcgs geaksaasgk grrtrkrkqt |
| 541 | ilkeatkgqk eaakgwkeaa ngdeaaaeea sttkgeqfpg emfpvacret tarvkeevde |
| 601 | gvtcemllnt mdk |
| Transcriptional repressor CTCFL, isoform 7 NP_001255976.1 | |
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveasklkrh |
| 361 | vrshtgerpf qccqcsyasr dtyklkrhmr thsgekpyec hichtrftqs gtmkihilqk |
| 421 | hgenvpkyqc phcatiiark sdlrvhmrnl haysaaelkc rycsavfher yaliqhqkth |
| 481 | knekrfkckh csyackqerh mtahirthtg ekpftclscn kcfrqkqlln ahfrkyhdan |
| 541 | fiptvykcsk cgkgfsrwit skwsglkpqt fit |
| Transcriptional repressor CTCFL, isoform 8 NP_001255977.1 | |
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveerhmtah |
| 361 | irthtgekpf tclscnkcfr qkqllnahfr kyhdanfipt vykcskcgkg fsrwilwvgn |
| 421 | sevaelggpg sgpllrlqsg cppglhhpka glgpedplpg qlrhttagtg lssllqgplc |
| 481 | raa |
| Transcriptional repressor CTCFL, isoform 9 NP_001255978.1 | |
| 1 | msgdersdei vltvsnsnve eqedqptagq adaekakstk nqrktkgakg tfhcdvcmft |
| 61 | ssrmssfnrh mkthtsekph lchlclktfr tvtllrnhvn thtgtrpykc ndcnmafvts |
| 121 | gelvrhrryk hthekpfkcs mckyasveas klkrhvrsht gerpfqccqc syasrdtykl |
| 181 | krhmrthsge kpyechicht rftqsgtmki hilqkhgenv pkyqcphcat iiarksdlry |
| 241 | hmrnlhaysa aelkcrycsa vfheryaliq hqkthknekr fkckhcsyac kqerhmtahi |
| 301 | rthtgekpft clscnkcfrq kqllnahfrk yhdanfiptv ykcskcgkgf srwinlhrhs |
| 361 | ekcgsgeaks aasgkgrrtr krkqtilkea tkgqkeaakg wkeaangdgv isahrnlcll |
| 421 | gssdshasvs gagitdarhh awlivllflv emgfyhvshs |
| Transcriptional repressor CTCFL, isoform 10 NP_001255979.1 | |
| 1 | msgdersdei vltvsnsnve eqedqptagq adaekakstk nqrktkgakg tfhcdvcmft |
| 61 | ssrmssfnrh mkthtsekph lchlclktfr tvtllrnhvn thtgtrpykc ndcnmafvts |
| 121 | gelvrhrryk hthekpfkcs mckyasveas klkrhvrsht gerpfqccqc syasrdtykl |
| 181 | krhmrthsge kpyechicht rftqsgtmki hilqkhgenv pkyqcphcat iiarksdlrv |
| 241 | hmrnlhaysa aelkcrycsa vfheryaliq hqkthknekr fkckhcsyac kqerhmtahi |
| 301 | rthtgekpft clscnkcfrq kqllnahfrk yhdanfiptv ykcskcgkgf srwilwvgns |
| 361 | evaelggpgs gpllrlqsgc ppglhhpkag lgpedplpgq lrhttagtgl ssllqgplcr |
| 421 | aa |
| Transcriptional repressor CTCFL, isoform 11 NP_001255980.1, | |
| NP_001255981.1 | |
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya svevkpfldl |
| 361 | klhgilveaa vqvtpsvtns ricykqafyy sykiyagnnm hsll |
| Transcriptional repressor CTCFL, isoform 12 NP_001255983.1 | |
| 1 | mftssrmssf nrhmkthtse kphlchlclk tfrtvtllrn hvnthtgtrp ykcndcnmaf |
| 61 | vtsgelvrhr rykhthekpf kcsmckyasv easklkrhvr shtgerpfqc cqcsyasrdt |
| 121 | yklkrhmrth sgekpyechi chtrftqsgt mkihilqkhg envpkyqcph catiiarksd |
| 181 | lrvhmrnlha ysaaelkcry csavfherya liqhqkthkn ekrfkckhcs yackqerhmt |
| 241 | ahirthtgek pftclscnkc frqkqllnah frkyhdanfi ptvykcskcg kgfsrwinlh |
| 301 | rhsekcgsge aksaasgkgr rtrkrkqtil keatkgqkea akgwkeaang dgvisahrnl |
| 361 | cllgssdsha sysgagitda rhhawlivll flvemgfyhv shs |
| Transcriptional repressor CTCFL, isoform 13 NP_001255984.1 | |
| 1 | mftssrmssf nrhmkthtse kphlchlclk tfrtvtllrn hvnthtgtrp ykcndcnmaf |
| 61 | vtsgelvrhr rykhthekpf kcsmckyasv easklkrhvr shtgerpfqc cqcsyasrdt |
| 121 | yklkrhmrth sgekpyechi chtrftqsgt mkihilqkhg envpkyqcph catiiarksd |
| 181 | lrvhmrnlha ysaaelkcry csavfherya liqhqkthkn ekrfkckhcs yackqerhmt |
| 241 | ahirthtgek pftclscnkc frqkqllnah frkyhdanfi ptvykcskcg kgfsrwvly |
| Cytochrome P450 1B1 NP_000095.2 | |
| 1 | mgtslspndp wplnplsiqq ttlllllsvl atvhvgqrll rqrrrqlrsa ppgpfawpli |
| 61 | gnaaavgqaa hlsfarlarr ygdvfqirlg scpivvlnge raihqalvqq gsafadrpaf |
| 121 | asfrvvsggr smafghyseh wkvqrraahs mmrnfftrqp rsrqvleghv lsearelval |
| 181 | lvrgsadgaf ldprpltvva vanvmsavcf gcryshddpe frellshnee fgrtvgagsl |
| 241 | vdvmpwlqyf pnpvrtvfre feqlnrnfsn fildkflrhc eslrpgaapr dmmdafilsa |
| 301 | ekkaagdshg ggarldlenv patitdifga sqdtlstalq wllllftryp dvqtrvqael |
| 361 | dqvvgrdrlp cmgdqpnlpy vlaflyeamr fssfvpvtip hattantsvl gyhipkdtvv |
| 421 | fvnqwsvnhd plkwpnpenf dparfldkdg linkdltsrv mifsvgkrrc igeelskmql |
| 481 | flfisilahq cdfranpnep akmnfsyglt ikpksfkvnv tlresmelld savqnlqake |
| 541 | tcq |
| Epidermal growth factor receptor, isoform a precursor NP_005219.2 | |
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qeilhgavrf snnpalcnve siqwrdivss dflsnmsmdf |
| 181 | qnhlgscqkc dpscpngscw gageencqkl tkiicaqqcs grcrgkspsd cchnqcaagc |
| 241 | tgpresdclv crkfrdeatc kdtcpplmly npttyqmdvn pegkysfgat cvkkcprnyv |
| 301 | vtdhgscvra cgadsyemee dgvrkckkce gpcrkvcngi gigefkdsls inatnikhfk |
| 361 | nctsisgdlh ilpvafrgds fthtppldpq eldilktvke itgflliqaw penrtdlhaf |
| 421 | enleiirgrt kqhgqfslav vslnitslgl rslkeisdgd viisgnknlc yantinwkkl |
| 481 | fgtsgqktki isnrgensck atgqvchalc spegcwgpep rdcvscrnvs rgrecvdkcn |
| 541 | llegeprefv enseciqchp eclpqamnit ctgrgpdnci qcahyidgph cvktcpagvm |
| 601 | genntlvwky adaghvchlc hpnctygctg pglegcptng pkipsiatgm vgalllllvv |
| 661 | algiglfmrr rhivrkrtlr rllqerelve pltpsgeapn qallrilket efkkikvlgs |
| 721 | gafgtvykgl wipegekvki pvaikelrea tspkankeil deayvmasvd nphvcrllgi |
| 781 | cltstvqlit qlmpfgclld yvrehkdnig sqyllnwcvq iakgmnyled rrlvhrdlaa |
| 841 | rnvlvktpqh vkitdfglak llgaeekeyh aeggkvpikw malesilhri ythqsdvwsy |
| 901 | gvtvwelmtf gskpydgipa seissilekg erlpqppict idvymimvkc wmidadsrpk |
| 961 | freliiefsk mardpqrylv iqgdermhlp sptdsnfyra lmdeedmddv vdadeylipq |
| 1021 | qgffsspsts rtpllsslsa tsnnstvaci drnglqscpi kedsflqrys sdptgalted |
| 1081 | siddtflpvp eyinqsvpkr pagsvqnpvy hnqplnpaps rdphyqdphs tavgnpeyln |
| 1141 | tvqptcvnst fdspahwaqk gshqisldnp dyqqdffpke akpngifkgs taenaeylrv |
| 1201 | apqssefiga |
| Epidermal growth factor receptor, isoform b precursor NP_958439.1 | |
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qeilhgavrf snnpalcnve siqwrdivss dflsnmsmdf |
| 181 | qnhlgscqkc dpscpngscw gageencqkl tkiicaqqcs grcrgkspsd cchnqcaagc |
| 241 | tgpresdclv crkfrdeatc kdtcpplmly npttyqmdvn pegkysfgat cvkkcprnyv |
| 301 | vtdhgscvra cgadsyemee dgvrkckkce gpcrkvcngi gigefkdsls inatnikhfk |
| 361 | nctsisgdlh ilpvafrgds fthtppldpq eldilktvke itgflliqaw penrtdlhaf |
| 421 | enleiirgrt kqhgqfslav vslnitslgl rslkeisdgd viisgnknlc yantinwkkl |
| 481 | fgtsgqktki isnrgensck atgqvchalc spegcwgpep rdcvscrnvs rgrecvdkcn |
| 541 | llegeprefv enseciqchp eclpqamnit ctgrgpdnci qcahyidgph cvktcpagvm |
| 601 | genntlvwky adaghvchlc hpnctygs |
| Epidermal growth factor receptor, isoform c precursor NP_958440.1 | |
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qeilhgavrf snnpalcnve siqwrdivss dflsnmsmdf |
| 181 | qnhlgscqkc dpscpngscw gageencqkl tkiicaqqcs grcrgkspsd cchnqcaagc |
| 241 | tgpresdclv crkfrdeatc kdtcpplmly npttyqmdvn pegkysfgat cvkkcprnyv |
| 301 | vtdhgscvra cgadsyemee dgvrkckkce gpcrkvcngi gigefkdsls inatnikhfk |
| 361 | nctsisgdlh ilpvafrgds fthtppldpq eldilktvke itgls |
| Epidermal growth factor receptor, isoform d precursor NP_958441.1 | |
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qeilhgavrf snnpalcnve siqwrdivss dflsnmsmdf |
| 181 | qnhlgscqkc dpscpngscw gageencqkl tkiicaqqcs grcrgkspsd cchnqcaagc |
| 241 | tgpresdclv crkfrdeatc kdtcpplmly npttyqmdvn pegkysfgat cvkkcprnyv |
| 301 | vtdhgscvra cgadsyemee dgvrkckkce gpcrkvcngi gigefkdsls inatnikhfk |
| 361 | nctsisgdlh ilpvafrgds fthtppldpq eldilktvke itgflliqaw penrtdlhaf |
| 421 | enleiirgrt kqhgqfslav vslnitslgl rslkeisdgd viisgnknlc yantinwkkl |
| 481 | fgtsgqktki isnrgensck atgqvchalc spegcwgpep rdcvscrnvs rgrecvdkcn |
| 541 | llegeprefv enseciqchp eclpqamnit ctgrgpdnci qcahyidgph cvktcpagvm |
| 601 | genntlvwky adaghvchlc hpnctygpgn eslkamlfcl fklsscnqsn dgsvshqsgs |
| 661 | paaqesclgw ipsllpsefq lgwggcshlh awpsasviit assch |
| Epidermal growth factor receptor, isoform e precursor NP_001333826.1 | |
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qgqkcdpscp ngscwgagee ncqkltkiic aqqcsgrcrg |
| 181 | kspsdcchnq caagctgpre sdclvcrkfr deatckdtcp plmlynptty qmdvnpegky |
| 241 | sfgatcvkkc prnyvvtdhg scvracgads yemeedgvrk ckkcegperk vcngigigef |
| 301 | kdslsinatn ikhfknctsi sgdlhilpva frgdsfthtp pldpqeldil ktvkeitgfl |
| 361 | liqawpenrt dlhafenlei irgrtkqhgq fslavvslni tslglrslke isdgdviisg |
| 421 | nknlcyanti nwkklfgtsg qktkiisnrg ensckatgqv chalcspegc wgpeprdcvs |
| 481 | crnvsrgrec vdkcnllege prefvensec iqchpeclpq amnitctgrg pdnciqcahy |
| 541 | idgphcvktc pagvmgennt lvwkyadagh vchlchpnct ygctgpgleg cptngpkips |
| 601 | iatgmvgall lllvvalgig lfmrrrhivr krtlrrllqe relvepltps geapnqallr |
| 661 | ilketefkki kvlgsgafgt vykglwipeg ekvkipvaik elreatspka nkeildeayv |
| 721 | masvdnphvc rllgicltst vqlitqlmpf gclldyvreh kdnigsqyll nwcvqiakgm |
| 781 | nyledrrlvh rdlaarnvlv ktpqhvkitd fglakllgae ekeyhaeggk vpikwmales |
| 841 | ilhriythqs dvwsygvtvw elmtfgskpy dgipaseiss ilekgerlpq ppictidvym |
| 901 | imvkcwmida dsrpkfreli iefskmardp qrylviqgde rmhlpsptds nfyralmdee |
| 961 | dmddvvdade ylipqqgffs spstsrtpll sslsatsnns tvacidrngl qscpikedsf |
| 1021 | lqryssdptg altedsiddt flpvpgewlv wkqscsstss thsaaaslqc psqvlppasp |
| 1081 | egetvadlqt q |
| Epidermal growth factor receptor, isoform f precursor NP_001333827.1 | |
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qeilhgavrf snnpalcnve siqwrdivss dflsnmsmdf |
| 181 | qnhlgscqkc dpscpngscw gageencqkl tkiicaqqcs grcrgkspsd cchnqcaagc |
| 241 | tgpresdclv crkfrdeatc kdtcpplmly npttyqmdvn pegkysfgat cvkkcprnyv |
| 301 | vtdhgscvra cgadsyemee dgvrkckkce gpcrkvcngi gigefkdsls inatnikhfk |
| 361 | nctsisgdlh ilpvafrgds fthtppldpq eldilktvke itgflliqaw penrtdlhaf |
| 421 | enleiirgrt kqhgqfslav vslnitslgl rslkeisdgd viisgnknlc yantinwkkl |
| 481 | fgtsgqktki isnrgensck atgqvchalc spegcwgpep rdcvscrnvs rgrecvdkcn |
| 541 | llegeprefv enseciqchp eclpqamnit ctgrgpdnci qcahyidgph cvktcpagvm |
| 601 | genntlvwky adaghvchlc hpnctygctg pglegcptng pkipsiatgm vgalllllvv |
| 661 | algiglfmrr rhivrkrtlr rllqerelve pltpsgeapn qallrilket efkkikvlgs |
| 721 | gafgtvykgl wipegekvki pvaikelrea tspkankeil deayvmasvd nphvcrllgi |
| 781 | cltstvqlit qlmpfgclld yvrehkdnig sqyllnwcvq iakgmnyled rrlvhrdlaa |
| 841 | rnvlvktpqh vkitdfglak llgaeekeyh aeggkvpikw malesilhri ythqsdvwsy |
| 901 | gvtvwelmtf gskpydgipa seissilekg erlpqppict idvymimvkc wmidadsrpk |
| 961 | freliiefsk mardpqrylv iqgdermhlp sptdsnfyra lmdeedmddv vdadeylipq |
| 1021 | qgffsspsts rtpllsslsa tsnnstvaci drnglqscpi kedsflqrys sdptgalted |
| 1081 | siddtflpvp gewlvwkqsc sstssthsaa aslqcpsqvl ppaspegetv adlqtq |
| Epidermal growth factor receptor, isoform g precursor NP_001333828.1 | |
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qgqkcdpscp ngscwgagee ncqkltkiic aqqcsgrcrg |
| 181 | kspsdcchnq caagctgpre sdclvcrkfr deatckdtcp plmlynptty qmdvnpegky |
| 241 | sfgatcvkkc prnyvvtdhg scvracgads yemeedgvrk ckkcegpcrk vcngigigef |
| 301 | kdslsinatn ikhfknctsi sgdlhilpva frgdsfthtp pldpqeldil ktvkeitgfl |
| 361 | liqawpenrt dlhafenlei irgrtkqhgq fslavvslni tslglrslke isdgdviisg |
| 421 | nknlcyanti nwkklfgtsg qktkiisnrg ensckatgqv chalcspegc wgpeprdcvs |
| 481 | crnvsrgrec vdkcnllege prefvensec iqchpeclpq amnitctgrg pdnciqcahy |
| 541 | idgphcvktc pagvmgennt lvwkyadagh vchlchpnct ygctgpgleg cptngpkips |
| 601 | iatgmvgall lllvvalgig lfmrrrhivr krtlrrllqe relvepltps geapnqallr |
| 661 | ilketefkki kvlgsgafgt vykglwipeg ekvkipvaik elreatspka nkeildeayv |
| 721 | masvdnphvc rllgicltst vqlitqlmpf gclldyvreh kdnigsqyll nwcvqiakgm |
| 781 | nyledrrlvh rdlaarnvlv ktpqhvkitd fglakllgae ekeyhaeggk vpikwmales |
| 841 | ilhriythqs dvwsygvtvw elmtfgskpy dgipaseiss ilekgerlpq ppictidvym |
| 901 | imvkcwmida dsrpkfreli iefskmardp qrylviqgde rmhlpsptds nfyralmdee |
| 961 | dmddvvdade ylipqqgffs spstsrtpll sslsatsnns tvacidrngl qscpikedsf |
| 1021 | lqryssdptg altedsiddt flpvpeyinq svpkrpagsv qnpvyhnqpl npapsrdphy |
| 1081 | qdphstavgn peylntvqpt cvnstfdspa hwaqkgshqi sldnpdyqqd ffpkeakpng |
| 1141 | ifkgstaena eylrvapqss efiga |
| Epidermal growth factor receptor, isoform h NP_001333829.1 | |
| 1 | mfnncevvlg nleityvqrn ydlsflktiq evagyvlial ntveriplen lqiirgnmyy |
| 61 | ensyalavls nydanktglk elpmrnlqei lhgavrfsnn palcnvesiq wrdivssdfl |
| 121 | snmsmdfqnh lgscqkcdps cpngscwgag eencqkltki icaqqcsgrc rgkspsdcch |
| 181 | nqcaagctgp resdclvcrk frdeatckdt cpplmlynpt tyqmdvnpeg kysfgatcvk |
| 241 | kcprnyvvtd hgscvracga dsyemeedgv rkckkcegpc rkvcngigig efkdslsina |
| 301 | tnikhfknct sisgdlhilp vafrgdsfth tppldpqeld ilktvkeitg flliqawpen |
| 361 | rtdlhafenl eiirgrtkqh gqfslavvsl nitslglrsl keisdgdvii sgnknlcyan |
| 421 | tinwkklfgt sgqktkiisn rgensckatg qvchalcspe gcwgpeprdc vscrnvsrgr |
| 481 | ecvdkcnlle geprefvens eciqchpecl pqamnitctg rgpdnciqca hyidgphcvk |
| 541 | tcpagvmgen ntlvwkyada ghvchlchpn ctygctgpgl egcptngpki psiatgmvga |
| 601 | lllllvvalg iglfmrrrhi vrkrtlrrll qerelveplt psgeapnqal lrilketefk |
| 661 | kikvlgsgaf gtvykglwip egekvkipva ikelreatsp kankeildea yvmasvdnph |
| 721 | vcrllgiclt stvqlitqlm pfgclldyvr ehkdnigsqy llnwcvqiak gmnyledrrl |
| 781 | vhrdlaarnv lvktpqhvki tdfglakllg aeekeyhaeg gkvpikwmal esilhriyth |
| 841 | qsdvwsygvt vwelmtfgsk pydgipasei ssilekgerl pqppictidv ymimvkcwmi |
| 901 | dadsrpkfre liiefskmar dpqrylviqg dermhlpspt dsnfyralmd eedmddvvda |
| 961 | deylipqqgf fsspstsrtp llsslsatsn nstvacidrn glqscpiked sflqryssdp |
| 1021 | tgaltedsid dtflpvpeyi nqsvpkrpag svqnpvyhnq pinpapsrdp hyqdphstav |
| 1081 | gnpeylntvq ptcvnstfds pahwaqkgsh qisldnpdyq qdffpkeakp ngifkgstae |
| 1141 | naeylrvapq ssefiga |
| Epidermal growth factor receptor, isoform i precursor NP_001333870.1 | |
| 1 | mrpsgtagaa llallaalcp asraleekkg nyvvtdhgsc vracgadsye meedgvrkck |
| 61 | kcegpcrkvc ngigigefkd slsinatnik hfknctsisg dlhilpvafr gdsfthtppl |
| 121 | dpqeldilkt vkeitgflli qawpenrtdl hafenleiir grtkqhgqfs lavvslnits |
| 181 | lglrslkeis dgdviisgnk nlcyantinw kklfgtsgqk tkiisnrgen sckatgqvch |
| 241 | alcspegcwg peprdcvscr nvsrgrecvd kcnllegepr efvenseciq chpeclpqam |
| 301 | nitctgrgpd nciqcahyid gphcvktcpa gvmgenntlv wkyadaghvc hlchpnctyg |
| 361 | ctgpglegcp tngpkipsia tgmvgallll lvvalgiglf mrrrhivrkr tlrrllqere |
| 421 | lvepltpsge apnqallril ketefkkikv lgsgafgtvy kglwipegek vkipvaikel |
| 481 | reatspkank eildeayvma svdnphvcrl lgicltstvq litqlmpfgc lldyvrehkd |
| 541 | nigsqyllnw cvqiakgmny ledrrlvhrd laarnvlvkt pqhvkitdfg lakllgaeek |
| 601 | eyhaeggkvp ikwmalesil hriythqsdv wsygvtvwel mtfgskpydg ipaseissil |
| 661 | ekgerlpqpp ictidvymim vkcwmidads rpkfreliie fskmardpqr ylviqgderm |
| 721 | hlpsptdsnf yralmdeedm ddvvdadeyl ipqqgffssp stsrtpllss lsatsnnstv |
| 781 | acidrnglqs cpikedsflq ryssdptgal tedsiddtfl pvpeyinqsv pkrpagsvqn |
| 841 | pvyhnqplnp apsrdphyqd phstavgnpe ylntvqptcv nstfdspahw aqkgshqisl |
| 901 | dnpdyqqdff pkeakpngif kgstaenaey lrvapqssef iga |
| Epithelial cell adhesion molecule NP_002345.2 | |
| 1 | mappqvlafg lllaaatatf aaaqeecvce nyklavncfv nnnrqcqcts vgaqntvics |
| 61 | klaakclvmk aemngsklgr rakpegalqn ndglydpdcd esglfkakqc ngtsmcwcvn |
| 121 | tagvrrtdkd teitcservr tywiiielkh karekpydsk slrtalqkei ttryqldpkf |
| 181 | itsilyennv itidlvqnss qktqndvdia dvayyfekdv kgeslfhskk mdltvngeql |
| 241 | dldpgqtliy yvdekapefs mqglkagvia vivvvviavv agivvlvisr kkrmakyeka |
| 301 | eikemgemhr elna |
| Ephrin type-A receptor 2, isoform 1 precursor NP_004422.2 | |
| 1 | melqaaracf allwgcalaa aaaaqgkevv lldfaaagge lgwlthpygk gwdlmqnimn |
| 61 | dmpiymysvc nvmsgdqdnw lrtnwvyrge aerifielkf tvrdcnsfpg gasscketfn |
| 121 | lyyaesdldy gtnfqkrlft kidtiapdei tvssdfearh vklnveersv gpltrkgfyl |
| 181 | afqdigacva llsvrvyykk cpellqglah fpetiagsda pslatvagtc vdhavvppgg |
| 241 | eeprmhcavd gewlvpigqc lcqagyekve dacqacspgf fkfeasespc lecpehtlps |
| 301 | pegatscece egffrapqdp asmpctrpps aphyltavgm gakvelrwtp pqdsggredi |
| 361 | vysvtceqcw pesgecgpce asvrysepph gltrtsvtvs dlephmnytf tvearngvsg |
| 421 | lvtsrsfrta svsinqtepp kvrlegrstt slsvswsipp pqqsrvwkye vtyrkkgdsn |
| 481 | synvrrtegf svtlddlapd ttylvqvqal tqegqgagsk vhefqtlspe gsgnlavigg |
| 541 | vavgvvlllv lagvgffihr rrknqrarqs pedvyfskse qlkplktyvd phtyedpnqa |
| 601 | vlkftteihp scvtrqkvig agefgevykg mlktssgkke vpvaiktlka gytekqrvdf |
| 661 | lgeagimgqf shhniirleg viskykpmmi iteymengal dkflrekdge fsvlqlvgml |
| 721 | rgiaagmkyl anmnyvhrdl aarnilvnsn lvckvsdfgl srvleddpea tyttsggkip |
| 781 | irwtapeais yrkftsasdv wsfgivmwev mtygerpywe lsnhevmkai ndgfrlptpm |
| 841 | dcpsaiyqlm mqcwqqerar rpkfadivsi ldklirapds lktladfdpr vsirlpstsg |
| 901 | segvpfrtvs ewlesikmqq ytehfmaagy taiekvvqmt nddikrigvr lpghqkriay |
| 961 | sllglkdqvn tvgipi |
| Ephrin type-A receptor 2, isoform 2 NP_001316019.1 | |
| 1 | mqnimndmpi ymysvcnvms gdqdnwlrtn wvyrgeaeri fielkftvrd cnsfpggass |
| 61 | cketfnlyya esdldygtnf qkrlftkidt iapdeitvss dfearhvkln veersvgplt |
| 121 | rkgfylafqd igacvallsv rvyykkcpel lqglahfpet iagsdapsla tvagtcvdha |
| 181 | vvppggeepr mhcavdgewl vpigqclcqa gyekvedacq acspgffkfe asespclecp |
| 241 | ehtlpspega tsceceegff rapqdpasmp ctrppsaphy ltavgmgakv elrwtppqds |
| 301 | ggredivysv tceqcwpesg ecgpceasvr ysepphgltr tsvtvsdlep hmnytftvea |
| 361 | rngvsglvts rsfrtasvsi nqteppkvrl egrsttslsv swsipppqqs rvwkyevtyr |
| 421 | kkgdsnsynv rrtegfsvtl ddlapdttyl vqvqaltqeg qgagskvhef qtlspegsgn |
| 481 | laviggvavg vvlllvlagv gffihrrrkn qrarqspedv yfskseqlkp lktyvdphty |
| 541 | edpnqavlkf tteihpscvt rqkvigagef gevykgmlkt ssgkkevpva iktlkagyte |
| 601 | kqrvdflgea gimgqfshhn iirlegvisk ykpmmiitey mengaldkfl rekdgefsvl |
| 661 | qlvgmlrgia agmkylanmn yvhrdlaarn ilvnsnlvck vsdfglsrvl eddpeatytt |
| 721 | sggkipirwt apeaisyrkf tsasdvwsfg ivmwevmtyg erpywelsnh evmkaindgf |
| 781 | rlptpmdcps aiyqlmmqcw qqerarrpkf adivsildkl irapdslktl adfdprvsir |
| 841 | lpstsgsegv pfrtvsewle sikmqqyteh fmaagytaie kvvqmtnddi krigvrlpgh |
| 901 | qkriaysllg lkdqvntvgi pi |
| Receptor-tyrosine-protein kinase erbB-2, isoform a precursor | |
| NP_004439.2 | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytmrrl lqetelvepl tpsgampnqa qmrilketel |
| 721 | rkvkvlgsga fgtvykgiwi pdgenvkipv aikvlrents pkankeilde ayvmagvgsp |
| 781 | yvsrllgicl tstvqlvtql mpygclldhv renrgrlgsq dllnwcmqia kgmsyledvr |
| 841 | lvhrdlaarn vlvkspnhvk itdfglarll dideteyhad ggkvpikwma lesilrrrft |
| 901 | hqsdvwsygv tvwelmtfga kpydgipare ipdllekger lpqppictid vymimvkcwm |
| 961 | idsecrprfr elvsefsrma rdpqrfvviq nedlgpaspl dstfyrslle dddmgdlvda |
| 1021 | eeylvpqqgf fcpdpapgag gmvhhrhrss strsgggdlt lglepseeea prsplapseg |
| 1081 | agsdvfdgdl gmgaakglqs lpthdpsplq rysedptvpl psetdgyvap ltcspqpeyv |
| 1141 | nqpdvrpqpp spregplpaa rpagatlerp ktlspgkngv vkdvfafgga venpeyltpq |
| 1201 | ggaapqphpp pafspafdnl yywdqdpper gappstfkgt ptaenpeylg ldvpv |
| Receptor-tyrosine-protein kinase erbB-2, isoform b NP_001005862.1 | |
| 1 | mklrlpaspe thldmlrhly qgcqvvqgnl eltylptnas lsflqdiqev qgyvliahnq |
| 61 | vrqvplqrlr ivrgtqlfed nyalavldng dplnnttpvt gaspgglrel qlrslteilk |
| 121 | ggvliqrnpq lcyqdtilwk difhknnqla ltlidtnrsr achpcspmck gsrcwgesse |
| 181 | dcqsltrtvc aggcarckgp lptdccheqc aagctgpkhs dclaclhfnh sgicelhcpa |
| 241 | lvtyntdtfe smpnpegryt fgascvtacp ynylstdvgs ctlvcplhnq evtaedgtqr |
| 301 | cekcskpcar vcyglgmehl revravtsan iqefagckki fgslaflpes fdgdpasnta |
| 361 | plqpeqlqvf etleeitgyl yisawpdslp dlsvfqnlqv irgrilhnga ysltlqglgi |
| 421 | swlglrslre lgsglalihh nthlcfvhtv pwdqlfrnph qallhtanrp edecvgegla |
| 481 | chqlcarghc wgpgptqcvn csqflrgqec veecrvlqgl preyvnarhc lpchpecqpq |
| 541 | ngsvtcfgpe adqcvacahy kdppfcvarc psgvkpdlsy mpiwkfpdee gacqpcpinc |
| 601 | thscvdlddk gcpaeqrasp ltsiisavvg illvvvlgvv fgilikrrqq kirkytmrrl |
| 661 | lqetelvepl tpsgampnqa qmrilketel rkvkvlgsga fgtvykgiwi pdgenvkipv |
| 721 | aikvlrents pkankeilde ayvmagvgsp yvsrllgicl tstvqlvtql mpygclldhv |
| 781 | renrgrlgsq dllnwcmqia kgmsyledvr lvhrdlaarn vlvkspnhvk itdfglarll |
| 841 | dideteyhad ggkvpikwma lesilrrrft hqsdvwsygv tvwelmtfga kpydgipare |
| 901 | ipdllekger lpqppictid vymimvkcwm idsecrprfr elvsefsrma rdpqrfvviq |
| 961 | nedlgpaspl dstfyrslle dddmgdlvda eeylvpqqgf fcpdpapgag gmvhhrhrss |
| 1021 | strsgggdlt lglepseeea prsplapseg agsdvfdgdl gmgaakglqs lpthdpsplq |
| 1081 | rysedptvpl psetdgyvap ltcspqpeyv nqpdvrpqpp spregplpaa rpagatlerp |
| 1141 | ktlspgkngv vkdvfafgga venpeyltpq ggaapqphpp pafspafdnl yywdqdpper |
| 1201 | gappstfkgt ptaenpeylg ldvpv |
| Receptor-tyrosine-protein kinase erbB-2, isoform c NP_001276865.1 | |
| 1 | mprgswkpqv ctgtdmklrl paspethldm lrhlyqgcqv vqgnleltyl ptnaslsflq |
| 61 | diqevqgyvl iahnqvrqvp lqrlrivrgt qlfednyala vldngdplnn ttpvtgaspg |
| 121 | glrelqlrsl teilkggvli qrnpqlcyqd tilwkdifhk nnqlaltlid tnrsrachpc |
| 181 | spmckgsrcw gessedcqsl trtvcaggca rckgplptdc cheqcaagct gpkhsdclac |
| 241 | lhfnhsgice lhcpalvtyn tdtfesmpnp egrytfgasc vtacpynyls tdvgsctlvc |
| 301 | plhnqevtae dgtqrcekcs kpcarvcygl gmehlrevra vtsaniqefa gckkifgsla |
| 361 | flpesfdgdp asntaplqpe qlqvfetlee itgylyisaw pdslpdlsvf qnlqvirgri |
| 421 | lhngaysltl qglgiswlgl rslrelgsgl alihhnthlc fvhtvpwdql frnphqallh |
| 481 | tanrpedecv geglachqlc arghcwgpgp tqcvncsqfl rgqecveecr vlqglpreyv |
| 541 | narhclpchp ecqpqngsvt cfgpeadqcv acahykdppf cvarcpsgvk pdlsympiwk |
| 601 | fpdeegacqp cpincthscv dlddkgcpae qraspltsii savvgillvv vlgvvfgili |
| 661 | krrqqkirky tmrrllqete lvepltpsga mpnqaqmril ketelrkvkv lgsgafgtvy |
| 721 | kgiwipdgen vkipvaikvl rentspkank eildeayvma gvgspyvsrl lgicltstvq |
| 781 | lvtqlmpygc lldhvrenrg rlgsqdllnw cmqiakgmsy ledvrlvhrd laarnvlvks |
| 841 | pnhvkitdfg larlldidet eyhadggkvp ikwmalesil rrrfthqsdv wsygvtvwel |
| 901 | mtfgakpydg ipareipdll ekgerlpqpp ictidvymim vkcwmidsec rprfrelvse |
| 961 | fsrmardpqr fvviqnedlg paspldstfy rslledddmg dlvdaeeylv pqqgffcpdp |
| 1021 | apgaggmvhh rhrssstrsg ggdltlglep seeeaprspl apsegagsdv fdgdlgmgaa |
| 1081 | kglqslpthd psplqrysed ptvplpsetd gyvapltcsp qpeyvnqpdv rpqppspreg |
| 1141 | plpaarpaga tlerpktlsp gkngvvkdvf afggavenpe yltpqggaap qphpppafsp |
| 1201 | afdnlyywdq dppergapps tfkgtptaen peylgldvpv |
| Receptor-tyrosine-protein kinase erbB-2, isoform d NP_001276866.1 | |
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytmrrl lqetelvepl tpsgampnqa qmrilketel |
| 721 | rkvkvlgsga fgtvykgiwi pdgenvkipv aikvlrents pkankeilde ayvmagvgsp |
| 781 | yvsrllgicl tstvqlvtql mpygclldhv renrgrlgsq dllnwcmqia kgmsyledvr |
| 841 | lvhrdlaarn vlvkspnhvk itdfglarll dideteyhad ggkvpikwma lesilrrrft |
| 901 | hqsdvwsygv tvwelmtfga kpydgipare ipdllekger lpqppictid vymimvkcwm |
| 961 | idsecrprfr elvsefsrma rdpqrfvviq nedlgpaspl dstfyrslle dddmgdlvda |
| 1021 | eeylvpqqgf fcpdpapgag gmvhhrhrss strnm |
| Receptor-tyrosine-protein kinase erbB-2, isoform e NP_001276867.1 | |
| 1 | mklrlpaspe thldmlrhly qgcqvvqgnl eltylptnas lsflqdiqev qgyvliahnq |
| 61 | vrqvplqrlr ivrgtqlfed nyalavldng dplnnttpvt gaspgglrel qlrslteilk |
| 121 | ggvliqrnpq lcyqdtilwk difhknnqla ltlidtnrsr achpcspmck gsrcwgesse |
| 181 | dcqsltrtvc aggcarckgp lptdccheqc aagctgpkhs dclaclhfnh sgicelhcpa |
| 241 | lvtyntdtfe smpnpegryt fgascvtacp ynylstdvgs ctlvcplhnq evtaedgtqr |
| 301 | cekcskpcar vcyglgmehl revravtsan iqefagckki fgslaflpes fdgdpasnta |
| 361 | plqpeqlqvf etleeitgyl yisawpdslp dlsvfqnlqv irgrilhnga ysltlqglgi |
| 421 | swlglrslre lgsglalihh nthlcfvhtv pwdqlfrnph qallhtanrp edecvgegla |
| 481 | chqlcarghc wgpgptqcvn csqflrgqec veecrvlqgl preyvnarhc lpchpecqpq |
| 541 | ngsvtcfgpe adqcvacahy kdppfcvarc psgvkpdlsy mpiwkfpdee gacqpcpinc |
| 601 | ths |
| Receptor tyrosine-protein kinase erbB-4, isoform JM-a/CVT-1 precursor | |
| NP_005226.1 | |
| 1 | mkpatglwvw vsllvaagtv qpsdsqsvca gtenklssls dleqqyralr kyyencevvm |
| 61 | gnleitsieh nrdlsflrsv revtgyvlva lnqfrylple nlriirgtkl yedryalaif |
| 121 | lnyrkdgnfg lqelglknlt eilnggvyvd qnkflcyadt ihwqdivrnp wpsnltlvst |
| 181 | ngssgcgrch ksctgrcwgp tenhcqtltr tvcaeqcdgr cygpyvsdcc hrecaggcsg |
| 241 | pkdtdcfacm nfndsgacvt qcpqtfvynp ttfqlehnfn akytygafcv kkcphnfvvd |
| 301 | ssscvracps skmeveengi kmckpctdic pkacdgigtg slmsaqtvds snidkfinct |
| 361 | kingnliflv tgihgdpyna ieaidpekln vfrtvreitg flniqswppn mtdfsvfsnl |
| 421 | vtiggrvlys glsllilkqq gitslqfqsl keisagniyi tdnsnlcyyh tinwttlfst |
| 481 | inqrivirdn rkaenctaeg mvcnhlcssd gcwgpgpdqc lscrrfsrgr iciescnlyd |
| 541 | gefrefengs icvecdpqce kmedglltch gpgpdnctkc shfkdgpncv ekcpdglqga |
| 601 | nsfifkyadp drechpchpn ctqgcngpts hdciyypwtg hstlpqhart pliaagvigg |
| 661 | lfilvivglt favyvrrksi kkkralrrfl etelvepltp sgtapnqaql rilketelkr |
| 721 | vkvlgsgafg tvykgiwvpe getvkipvai kilnettgpk anvefmdeal imasmdhphl |
| 781 | vrllgvclsp tiqlvtqlmp hgclleyvhe hkdnigsqll lnwcvqiakg mmyleerrlv |
| 841 | hrdlaarnvl vkspnhvkit dfglarlleg dekeynadgg kmpikwmale cihyrkfthq |
| 901 | sdvwsygvti welmtfggkp ydgiptreip dllekgerlp qppictidvy mvmvkcwmid |
| 961 | adsrpkfkel aaefsrmard pqrylviqgd drmklpspnd skffqnllde edledmmdae |
| 1021 | eylvpqafni pppiytsrar idsnrseigh spppaytpms gnqfvyrdgg faaeqgvsvp |
| 1081 | yraptstipe apvaqgatae ifddsccngt lrkpvaphvq edsstqrysa dptvfapers |
| 1141 | prgeldeegy mtpmrdkpkq eylnpveenp fvsrrkngdl qaldnpeyhn asngppkaed |
| 1201 | eyvneplyln tfantlgkae ylknnilsmp ekakkafdnp dywnhslppr stlqhpdylq |
| 1261 | eystkyfykq ngrirpivae npeylsefsl kpgtvlpppp yrhrntvv |
| Receptor tyrosine-protein kinase erbB-4, isoform JM-a/CVT-2 precursor | |
| NP_001036064.1 | |
| 1 | mkpatglwvw vsllvaagtv qpsdsqsvca gtenklssls dleqqyralr kyyencevvm |
| 61 | gnleitsieh nrdlsflrsv revtgyvlva lnqfrylple nlriirgtkl yedryalaif |
| 121 | lnyrkdgnfg lqelglknlt eilnggvyvd qnkflcyadt ihwqdivrnp wpsnltlvst |
| 181 | ngssgcgrch ksctgrcwgp tenhcqtltr tvcaeqcdgr cygpyvsdcc hrecaggcsg |
| 241 | pkdtdcfacm nfndsgacvt qcpqtfvynp ttfqlehnfn akytygafcv kkcphnfvvd |
| 301 | ssscvracps skmeveengi kmckpctdic pkacdgigtg slmsaqtvds snidkfinct |
| 361 | kingnliflv tgihgdpyna ieaidpekln vfrtvreitg flniqswppn mtdfsvfsnl |
| 421 | vtiggrvlys glsllilkqq gitslqfqsl keisagniyi tdnsnlcyyh tinwttlfst |
| 481 | inqrivirdn rkaenctaeg mvcnhlcssd gcwgpgpdqc lscrrfsrgr iciescnlyd |
| 541 | gefrefengs icvecdpqce kmedglltch gpgpdnctkc shfkdgpncv ekcpdglqga |
| 601 | nsfifkyadp drechpchpn ctqgcngpts hdciyypwtg hstlpqhart pliaagvigg |
| 661 | lfilvivglt favyvrrksi kkkralrrfl etelvepltp sgtapnqaql rilketelkr |
| 721 | vkvlgsgafg tvykgiwvpe getvkipvai kilnettgpk anvefmdeal imasmdhphl |
| 781 | vrllgvclsp tiqlvtqlmp hgclleyvhe hkdnigsqll lnwcvqiakg mmyleerrlv |
| 841 | hrdlaarnvl vkspnhvkit dfglarlleg dekeynadgg kmpikwmale cihyrkfthq |
| 901 | sdvwsygvti welmtfggkp ydgiptreip dllekgerlp qppictidvy mvmvkcwmid |
| 961 | adsrpkfkel aaefsrmard pqrylviqgd drmklpspnd skffqnllde edledmmdae |
| 1021 | eylvpqafni pppiytsrar idsnrnqfvy rdggfaaeqg vsvpyrapts tipeapvaqg |
| 1081 | ataeifddsc cngtlrkpva phvqedsstq rysadptvfa persprgeld eegymtpmrd |
| 1141 | kpkqeylnpv eenpfvsrrk ngdlqaldnp eyhnasngpp kaedeyvnep lylntfantl |
| 1201 | gkaeylknni lsmpekakka fdnpdywnhs lpprstlqhp dylqeystky fykqngrirp |
| 1261 | ivaenpeyls efslkpgtvl ppppyrhrnt vv |
| Prolyl endopeptidase FAP, isoform 1 NP_004451.2 | |
| 1 | mktwvkivfg vatsavlall vmcivlrpsr vhnseentmr altlkdilng tfsyktffpn |
| 61 | wisgqeylhq sadnnivlyn ietgqsytil snrtmksvna snyglspdrq fvylesdysk |
| 121 | lwrysytaty yiydlsngef vrgnelprpi qylcwspvgs klayvyqnni ylkqrpgdpp |
| 181 | fqitfngren kifngipdwv yeeemlatky alwwspngkf layaefndtd ipviaysyyg |
| 241 | deqyprtini pypkagaknp vvrifiidtt ypayvgpqev pvpamiassd yyfswltwvt |
| 301 | dervclqwlk rvqnvsvlsi cdfredwqtw dcpktqehie esrtgwaggf fvstpvfsyd |
| 361 | aisyykifsd kdgykhihyi kdtvenaiqi tsgkweaini frvtqdslfy ssnefeeypg |
| 421 | rrniyrisig syppskkcvt chlrkercqy ytasfsdyak yyalvcygpg ipistlhdgr |
| 481 | tdqeikilee nkelenalkn iqlpkeeikk levdeitlwy kmilppqfdr skkyplliqv |
| 541 | yggpcsqsvr svfavnwisy laskegmvia lvdgrgtafq gdkllyavyr klgvyevedq |
| 601 | itavrkfiem gfidekriai wgwsyggyvs slalasgtgl fkcgiavapv ssweyyasvy |
| 661 | terfmglptk ddnlehykns tvmaraeyfr nvdyllihgt addnvhfqns aqiakalvna |
| 721 | qvdfqamwys dqnhglsgls tnhlythmth flkqcfslsd |
| Prolyl endopeptidase FAP, isoform 2 NP_001278736.1 | |
| 1 | mktwvkivfg vatsavlall vmcivlrpsr vhnseentmr altlkdilng tfsyktffpn |
| 61 | wisgqeylhq sadnnivlyn ietgqsytil snrtmlwrys ytatyyiydl sngefvrgne |
| 121 | lprpiqylcw spvgsklayv yqnniylkqr pgdppfqitf ngrenkifng ipdwvyeeem |
| 181 | latkyalwws pngkflayae fndtdipvia ysyygdeqyp rtinipypka gaknpvvrif |
| 241 | iidttypayv gpqevpvpam iassdyyfsw ltwvtdervc lqwlkrvqnv svlsicdfre |
| 301 | dwqtwdcpkt qehieesrtg waggffvstp vfsydaisyy kifsdkdgyk hihyikdtve |
| 361 | naiqitsgkw eainifrvtq dslfyssnef eeypgrrniy risigsypps kkcvtchlrk |
| 421 | ercqyytasf sdyakyyalv cygpgipist lhdgrtdqei kileenkele nalkniqlpk |
| 481 | eeikklevde itlwykmilp pqfdrskkyp lliqvyggpc sqsvrsvfav nwisylaske |
| 541 | gmvialvdgr gtafqgdkll yavyrklgvy evedqitavr kfiemgfide kriaiwgwsy |
| 601 | ggyvsslala sgtglfkcgi avapvsswey yasvyterfm glptkddnle hyknstvmar |
| 661 | aeyfrnvdyl lihgtaddnv hfqnsaqiak alvnaqvdfq amwysdqnhg lsglstnhly |
| 721 | thmthflkqc fslsd |
| Glutamate carboxypeptidase 2, isoform 1 NP_004467.1 | |
| 1 | mwnllhetds avatarrprw lcagalvlag gffllgflfg wfikssneat nitpkhnmka |
| 61 | fldelkaeni kkflynftqi phlagteqnf qlakqiqsqw kefgldsvel ahydvllsyp |
| 121 | nkthpnyisi inedgneifn tslfeppppg yenvsdivpp fsafspqgmp egdlvyvnya |
| 181 | rtedffkler dmkincsgki viarygkvfr gnkvknaqla gakgvilysd padyfapgvk |
| 241 | sypdgwnlpg ggvqrgniln lngagdpltp gypaneyayr rgiaeavglp sipvhpigyy |
| 301 | daqkllekmg gsappdsswr gslkvpynvg pgftgnfstq kvkmhihstn evtriynvig |
| 361 | tlrgavepdr yvilgghrds wvfggidpqs gaavvheivr sfgtlkkegw rprrtilfas |
| 421 | wdaeefgllg stewaeensr llqergvayi nadssiegny tlrvdctplm yslvhnltke |
| 481 | lkspdegfeg kslyeswtkk spspefsgmp risklgsgnd fevffqrlgi asgrarytkn |
| 541 | wetnkfsgyp lyhsvyetye lvekfydpmf kyhltvaqvr ggmvfelans ivlpfdcrdy |
| 601 | avvlrkyadk iysismkhpq emktysysfd slfsavknft eiaskfserl qdfdksnpiv |
| 661 | lrmmndqlmf lerafidplg lpdrpfyrhv iyapsshnky agesfpgiyd alfdieskvd |
| 721 | pskawgevkr qiyvaaftvq aaaetlseva |
| Glutamate carboxypeptidase 2, isoform 2 NP_001014986.1 | |
| 1 | mwnllhetds avatarrprw lcagalvlag gffllgflfg wfikssneat nitpkhnmka |
| 61 | fldelkaeni kkflynftqi phlagteqnf qlakqiqsqw kefgldsvel ahydvllsyp |
| 121 | nkthpnyisi inedgneifn tslfeppppg yenvsdivpp fsafspqgmp egdlvyvnya |
| 181 | rtedffkler dmkincsgki viarygkvfr gnkvknaqla gakgvilysd padyfapgvk |
| 241 | sypdgwnlpg ggvqrgniln lngagdpltp gypaneyayr rgiaeavglp sipvhpigyy |
| 301 | daqkllekmg gsappdsswr gslkvpynvg pgftgnfstq kvkmhihstn evtriynvig |
| 361 | tlrgavepdr yvilgghrds wvfggidpqs gaavvheivr sfgtlkkegw rprrtilfas |
| 421 | wdaeefgllg stewaeensr llqergvayi nadssiegny tlrvdctplm yslvhnltke |
| 481 | lkspdegfeg kslyeswtkk spspefsgmp risklgsgnd fevffqrlgi asgrarytkn |
| 541 | wetnkfsgyp lyhsvyetye lvekfydpmf kyhltvaqvr ggmvfelans ivlpfdcrdy |
| 601 | avvlrkyadk iysismkhpq emktysvsfd slfsavknft eiaskfserl qdfdkskhvi |
| 661 | yapsshnkya gesfpgiyda lfdieskvdp skawgevkrq iyvaaftvqa aaetlseva |
| Glutamate carboxypeptidase 2, isoform 3 NP_001180400.1 | |
| 1 | mtagssyplf laayactgcl aerlgwfiks sneatnitpk hnmkafldel kaenikkfly |
| 61 | nftqiphlag teqnfqlakq iqsqwkefgl dsvelahydv llsypnkthp nyisiinedg |
| 121 | neifntslfe ppppgyenvs divppfsafs pqgmpegdlv yvnyartedf fklerdmkin |
| 181 | csgkiviary gkvfrgnkvk naqlagakgv ilysdpadyf apgvksypdg wnlpgggvqr |
| 241 | gnilnlngag dpltpgypan eyayrrgiae avglpsipvh pigyydaqkl lekmggsapp |
| 301 | dsswrgslkv pynvgpgftg nfstqkvkmh ihstnevtri ynvigtlrga vepdryvilg |
| 361 | ghrdswvfgg idpqsgaavv heivrsfgtl kkegwrprrt ilfaswdaee fgllgstewa |
| 421 | eensrllqer gvayinadss iegnytlrvd ctplmyslvh nltkelkspd egfegkslye |
| 481 | swtkkspspe fsgmpriskl gsgndfevff qrlgiasgra rytknwetnk fsgyplyhsv |
| 541 | yetyelvekf ydpmfkyhlt vaqvrggmvf elansivlpf dcrdyavvlr kyadkiysis |
| 601 | mkhpqemkty svsfdslfsa vknfteiask fserlqdfdk snpivlrmmn dqlmfleraf |
| 661 | idplglpdrp fyrhviyaps shnkyagesf pgiydalfdi eskvdpskaw gevkrqiyva |
| 721 | aftvqaaaet lseva |
| Glutamate carboxypeptidase 2, isoform 4 NP_001180401.1 | |
| 1 | mtagssyplf laayactgcl aerlgwfiks sneatnitpk hnmkafldel kaenikkfly |
| 61 | nftqiphlag teqnfqlakq iqsqwkefgl dsvelahydv llsypnkthp nyisiinedg |
| 121 | neifntslfe ppppgyenvs divppfsafs pqgmpegdlv yvnyartedf fklerdmkin |
| 181 | csgkiviary gkvfrgnkvk naqlagakgv ilysdpadyf apgvksypdg wnlpgggvqr |
| 241 | gnilnlngag dpltpgypan eyayrrgiae avglpsipvh pigyydaqkl lekmggsapp |
| 301 | dsswrgslkv pynvgpgftg nfstqkvkmh ihstnevtri ynvigtlrga vepdryvilg |
| 361 | ghrdswvfgg idpqsgaavv heivrsfgtl kkegwrprrt ilfaswdaee fgllgstewa |
| 421 | eensrllqer gvayinadss iegnytlrvd ctplmyslvh nltkelkspd egfegkslye |
| 481 | swtkkspspe fsgmpriskl gsgndfevff qrlgiasgra rytknwetnk fsgyplyhsv |
| 541 | yetyelvekf ydpmfkyhlt vaqvrggmvf elansivlpf dcrdyavvlr kyadkiysis |
| 601 | mkhpqemkty svsfdslfsa vknfteiask fserlqdfdk skhviyapss hnkyagesfp |
| 661 | giydalfdie skvdpskawg evkrqiyvaa ftvqaaaetl seva |
| Glutamate carboxypeptidase 2, isoform 5 NP_001180402.1 | |
| 1 | mggsappdss wrgslkvpyn vgpgftgnfs tqkvkmhihs tnevtriynv igtlrgavep |
| 61 | dryvilgghr dswvfggidp qsgaavvhei vrsfgtlkke gwrprrtilf aswdaeefgl |
| 121 | lgstewaeen srllqergva yinadssieg nytlrvdctp lmyslvhnlt kelkspdegf |
| 181 | egkslyeswt kkspspefsg mprisklgsg ndfevffqrl giasgraryt knwetnkfsg |
| 241 | yplyhsvyet yelvekfydp mfkyhltvaq vrggmvfela nsivlpfdcr dyavvlrkya |
| 301 | dkiysismkh pqemktysvs fdslfsavkn fteiaskfse rlqdfdksnp ivlrmmndql |
| 361 | mflerafidp lglpdrpfyr hviyapsshn kyagesfpgi ydalfdiesk vdpskawgev |
| 421 | krqiyvaaft vqaaaetlse va |
| Glutamate carboxypeptidase 2, isoform 6 NP_001338165.1 | |
| 1 | mkafldelka enikkflynf tqiphlagte qnfqlakqiq sqwkefglds velahydvll |
| 61 | sypnkthpny isiinedgne ifntslfepp ppgyenvsdi vppfsafspq gmpegdlvyv |
| 121 | nyartedffk lerdmkincs gkiviarygk vfrgnkvkna qlagakgvil ysdpadyfap |
| 181 | gvksypdgwn lpgggvqrgn ilnlngagdp ltpgypaney ayrrgiaeav glpsipvhpi |
| 241 | gyydaqklle kmggsappds swrgslkvpy nvgpgftgnf stqkvkmhih stnevtriyn |
| 301 | vigtlrgave pdryvilggh rdswvfggid pgsgaavvhe ivrsfgtlkk egwrprrtil |
| 361 | faswdaeefg llgstewaee nsrllqergv ayinadssie gnytlrvdct plmyslvhnl |
| 421 | tkelkspdeg fegkslyesw tkkspspefs gmprisklgs gndfevffqr lgiasgrary |
| 481 | tknwetnkfs gyplyhsvye tyelvekfyd pmfkyhltva qvrggmvfel ansivlpfdc |
| 541 | rdyavvlrky adkiysismk hpqemktysv sfdslfsavk nfteiaskfs erlqdfdksk |
| 601 | hviyapsshn kyagesfpgi ydalfdiesk vdpskawgev krqiyvaaft vqaaaetlse |
| 661 | va |
| Fos-related antigen 1, isoform 1 NP_005429.1 | |
| 1 | mfrdfgepgp ssgngggygg paqppaaaqa aqqkfhlvps intmsgsqel qwmvqphflg |
| 61 | pssyprplty pqysppqprp gviralgppp gvrrrpceqi speeeerrrv rrernklaaa |
| 121 | kcrnrrkelt dflqaetdkl edeksglqre ieelqkqker lelvleahrp ickipegake |
| 181 | gdtgstsgts sppapcrpvp cislspgpvl epealhtptl mttpsltpft pslvftypst |
| 241 | pepcasahrk sssssgdpss dplgsptlla l |
| Fos-related antigen 1, isoform 2 NP_001287773.1 | |
| 1 | mfrdfgepgp ssgngggygg paqppaaaqa aqqkfhlvps intmsgsqel qwmvqphflg |
| 61 | pssyprplty pqysppqprp gviralgppp gvrrrpceqe tdkledeksg lqreieelqk |
| 121 | qkerlelvle ahrpickipe gakegdtgst sgtssppapc rpvpcislsp gpvlepealh |
| 181 | tptlmttpsl tpftpslvft ypstpepcas ahrksssssg dpssdplgsp tllal |
| Fos-related antigen 1, isoform 3 NP_001287784.1 | |
| 1 | mfrdfgepgp ssgngggygg paqppaaaqa aqqkfhlvps intmsgsqel qwmvqphflg |
| 61 | pssyprplty pqysppqprp gviralgppp gvrrrpceqp ggrgappska raeqagcgqv |
| 121 | qepeegtdrl paggd |
| Fos-related antigen 1, isoform 4 NP_001287785.1 | |
| 1 | mfrdfgepgp ssgngggygg paqppaaaqa aqqispeeee rrrvrrernk laaakcrnrr |
| 61 | keltdflqae tdkledeksg lqreieelqk qkerlelvle ahrpickipe gakegdtgst |
| 121 | sgtssppapc rpvpcislsp gpvlepealh tptlmttpsl tpftpslvft ypstpepcas |
| 181 | ahrksssssg dpssdplgsp tllal |
| Fos-related antigen 1, isoform 5 NP_001287786.1 | |
| 1 | mfrdfgepgp ssgngggygg paqppaaaqa aqqetdkled eksglqreie elqkqkerle |
| 61 | lvleahrpic kipegakegd tgstsgtssp papcrpvpci slspgpvlep ealhtptlmt |
| 121 | tpsltpftps lvftypstpe pcasahrkss sssgdpssdp lgsptllal |
| G antigen 1 NP_001035753.1 | |
| 1 | mswrgrstyy wprprryvqp pemigpmrpe qfsdevepat peegepatqr qdpaaaqege |
| 61 | degasagqgp kpeadsqeqg hpqtgceced gpdgqemdpp npeevktpee gegqsqc |
| G antigen 12I NP_001465.1 | |
| 1 | mswrgrstyy wprprryvqp pemigpmrpe qfsdevepat peegepatqr qdpaaaqege |
| 61 | degasagqgp kpeadsqeqg hpqtgceced gpdgqemdpp npeevktpee gekqsqc |
| Galectin-1 NP_002296.1 | |
| 1 | macglvasnl nlkpgeclrv rgevapdaks fvlnlgkdsn nlclhfnprf nahgdantiv |
| 61 | cnskdggawg teqreavfpf qpgsvaevci tfdqanltvk lpdgyefkfp nrlnleainy |
| 121 | maadgdfkik cvafd |
| Galectin-3 isoform 1 NP_002297.2 | |
| 1 | madnfslhda lsgsgnpnpq gwpgawgnqp agaggypgas ypgaypgqap pgaypgqapp |
| 61 | gaypgapgay pgapapgvyp gppsgpgayp ssgqpsatga ypatgpygap agplivpynl |
| 121 | plpggvvprm litilgtvkp nanrialdfq rgndvafhfn prfnennrrv ivcntkldnn |
| 181 | wgreerqsvf pfesgkpfki qvlvepdhfk vavndahllq ynhrvkklne isklgisgdi |
| 241 | dltsasytmi |
| Galectin-3, isoform 3 NP_001344607.1 | |
| 1 | mhsktpcgcf kpwkmadnfs lhdalsgsgn pnpqgwpgaw gnqpagaggy pgasypgayp |
| 61 | gqappgaypg qappgaypga pgaypgapap gvypgppsgp gaypssgqps atgaypatgp |
| 121 | ygapagpliv pynlplpggv vprmlitilg tvkpnanria ldfqrgndva fhfnprfnen |
| 181 | nrrvivcntk ldnnwgreer qsvfpfesgk pfkiqvlvep dhfkvavnda hllqynhrvk |
| 241 | klneisklgi sgdidltsas ytmi |
| Galectin-9 short NP_002299.2 | |
| 1 | mafsgsqapy lspavpfsgt iqgglqdglq itvngtvlss sgtrfavnfq tgfsgndiaf |
| 61 | hfnprfedgg yvvcntrqng swgpeerkth mpfqkgmpfd lcflvqssdf kvmvngilfv |
| 121 | qyfhrvpfhr vdtisvngsv qlsyisfqpp gvwpanpapi tqtvihtvqs apgqmfstpa |
| 181 | ippmmyphpa ypmpfittil gglypsksil lsgtvlpsaq rfhinlcsgn hiafhlnprf |
| 241 | denavvrntq idnswgseer slprkmpfvr gqsfsvwilc eahclkvavd gqhlfeyyhr |
| 301 | lrnlptinrl evggdiqlth vqt |
| Galectin-9 long NP_033665.1 | |
| 1 | mafsgsqapy lspavpfsgt iqgglqdglq itvngtvlss sgtrfavnfq tgfsgndiaf |
| 61 | hfnprfedgg yvvcntrqng swgpeerkth mpfqkgmpfd lcflvqssdf kvmvngilfv |
| 121 | qyfhrvpfhr vdtisvngsv qlsyisfqnp rtvpvqpafs tvpfsqpvcf pprprgrrqk |
| 181 | ppgvwpanpa pitqtvihtv qsapgqmfst paippmmyph paypmpfitt ilgglypsks |
| 241 | illsgtvlps aqrfhinlcs gnhiafhlnp rfdenavvrn tqidnswgse erslprkmpf |
| 301 | vrgqsfsvwi lceahclkva vdgqhlfeyy hrlrnlptin rlevggdiql thvqt |
| Galectin-9 isoform 3 NP_001317092.1 | |
| 1 | mafsgsqapy lspavpfsgt iqgglqdglq itvngtvlss sgtrfavnfq tgfsgndiaf |
| 61 | hfnprfedgg yvvcntrqng swgpeerkth mpfqkgmpfd lcflvqssdf kvmvngilfv |
| 121 | qyfhrvpfhr vdtisvngsv qlsyisfqpp gvwpanpapi tqtvihtvqs apgqmfstpa |
| 181 | ippmmyphpa ypmpfittil gglypsksil lsgtvlpsaq rcgscvklta srwpwmvstc |
| 241 | lnttia |
| Premelanosome protein, isoform 1 preprotein NP_001186983.1 | |
| 1 | mdlvlkrcll hlavigalla vgatkvprnq dwlgvsrqlr tkawnrqlyp ewteaqrldc |
| 61 | wrggqvslkv sndgptliga nasfsialnf pgsqkvlpdg qviwvnntii ngsqvwggqp |
| 121 | vypqetddac ifpdggpcps gswsqkrsfv yvwktwgqyw qvlggpvsgl sigtgramlg |
| 181 | thtmevtvyh rrgsrsyvpl ahsssaftit dqvpfsvsvs qlraldggnk hflrnqpltf |
| 241 | alqlhdpsgy laeadlsytw dfgdssgtli sralvvthty lepgpvtaqv vlqaaiplts |
| 301 | cgsspvpgtt dghrptaeap nttagqvptt evvgttpgqa ptaepsgtts vqvpttevis |
| 361 | tapvqmptae stgmtpekvp vsevmgttla emstpeatgm tpaevsivvl sgttaaqvtt |
| 421 | tewvettare lpipepegpd assimstesi tgslgplldg tatlrlvkrq vpldcvlyry |
| 481 | gsfsvtldiv qgiesaeilq avpsgegdaf eltvscqggl pkeacmeiss pgcqppaqrl |
| 541 | cqpvlpspac qlvlhqilkg gsgtyclnvs ladtnslavv stqlimpvpg illtgqeagl |
| 601 | gqvplivgil lvlmavvlas liyrrrlmkq dfsvpqlphs sshwlrlpri fcscpigens |
| 661 | pllsgqqv |
| Premelanosome protein, isoform 2 precursor NP_001186982.1 | |
| 1 | mdlvlkrcll hlavigalla vgatkgsqvw ggqpvypqet ddacifpdgg pcpsgswsqk |
| 61 | rsfvyvwktw gqywqvlggp vsglsigtgr amlgthtmev tvyhrrgsrs yvplahsssa |
| 121 | ftitdqvpfs vsvsqlrald ggnkhflrnq pltfalqlhd psgylaeadl sytwdfgdss |
| 181 | gtlisralvv thtylepgpv taqvvlqaai pltscgsspv pgttdghrpt aeapnttagq |
| 241 | vpttevvgtt pgqaptaeps gttsvqvptt evistapvqm ptaestgmtp ekvpvsevmg |
| 301 | ttlaemstpe atgmtpaevs ivvlsgttaa qvtttewvet tarelpipep egpdassims |
| 361 | tesitgslgp lldgtatlrl vkrqvpldcv lyrygsfsvt ldivqgiesa eilqavpsge |
| 421 | gdafeltvsc qgglpkeacm eisspgcqpp aqrlcqpvlp spacqlvlhq ilkggsgtyc |
| 481 | lnvsladtns lavvstqlim pgqeaglgqv plivgillvl mavvlasliy rrrlmkqdfs |
| 541 | vpqlphsssh wlrlprifcs cpigenspll sgqqv |
| Premelanosome protein, isoform 3 preprotein NP_008859.1 | |
| 1 | mdlvlkrcll hlavigalla vgatkvprnq dwlgvsrqlr tkawnrqlyp ewteaqrldc |
| 61 | wrggqvslkv sndgptliga nasfsialnf pgsqkvlpdg qviwvnntii ngsqvwggqp |
| 121 | vypqetddac ifpdggpcps gswsqkrsfv yvwktwgqyw qvlggpvsgl sigtgramlg |
| 181 | thtmevtvyh rrgsrsyvpl ahsssaftit dqvpfsvsvs qlraldggnk hflrnqpltf |
| 241 | alqlhdpsgy laeadlsytw dfgdssgtli sralvvthty lepgpvtaqv vlqaaiplts |
| 301 | cgsspvpgtt dghrptaeap nttagqvptt evvgttpgqa ptaepsgtts vqvpttevis |
| 361 | tapvqmptae stgmtpekvp vsevmgttla emstpeatgm tpaevsivvl sgttaaqvtt |
| 421 | tewvettare lpipepegpd assimstesi tgslgplldg tatlrlvkrq vpldcvlyry |
| 481 | gsfsvtldiv qgiesaeilq avpsgegdaf eltvscqggl pkeacmeiss pgcqppaqrl |
| 541 | cqpvlpspac qlvlhqilkg gsgtyclnvs ladtnslavv stqlimpgqe aglgqvpliv |
| 601 | gillvlmavv lasliyrrrl mkqdfsvpql phssshwlrl prifcscpig enspllsgqq |
| 661 | v |
| Premelanosome protein, isoform 4 preprotein NP_001307050.1 | |
| 1 | mdlvlkrcll hlavigalla vgatkvprnq dwlgvsrqlr tkawnrqlyp ewteaqrldc |
| 61 | wrggqvslkv sndgptliga nasfsialnf pgsqkvlpdg qviwvnntii ngsqvwggqp |
| 121 | vypqetddac ifpdggpcps gswsqkrsfv yvwktwgqyw qvlggpvsgl sigtgramlg |
| 181 | thtmevtvyh rrgsrsyvpl ahsssaftit dqvpfsvsvs qlraldggnk hflrnqpltf |
| 241 | alqlhdpsgy laeadlsytw dfgdssgtli sralvvthty lepgpvtaqv vlqaaiplts |
| 301 | cgsspvpgtt dghrptaeap nttagqvptt evvgttpgqa ptaepsgtts vqvpttevis |
| 361 | tapvqmptae staaqvttte wvettarelp ipepegpdas simstesitg slgplldgta |
| 421 | tlrlvkrqvp ldcvlyrygs fsvtldivqg iesaeilqav psgegdafel tvscqgglpk |
| 481 | eacmeisspg cqppaqrlcq pvlpspacql vlhqilkggs gtyclnvsla dtnslavvst |
| 541 | qlimpvpgil ltgqeaglgq vplivgillv lmavvlasli yrrrlmkqdf svpqlphsss |
| 601 | hwlrlprifc scpigenspl lsgqqv |
| Premelanosome protein, isoform 5 preprotein NP_001307051.1 | |
| 1 | mdlvlkrcll hlavigalla vgatkvprnq dwlgvsrqlr tkawnrqlyp ewteaqrldc |
| 61 | wrggqvslkv sndgptliga nasfsialnf pgsqkvlpdg qviwvnntii ngsqvwggqp |
| 121 | vypqetddac ifpdggpcps gswsqkrsfv yvwktwgqyw qvlggpvsgl sigtgramlg |
| 181 | thtmevtvyh rrgsrsyvpl ahsssaftit dqvpfsvsvs qlraldggnk hflrnqpltf |
| 241 | alqlhdpsgy laeadlsytw dfgdssgtli sralvvthty lepgpvtaqv vlqaaiplts |
| 301 | cgsspvpgtt dghrptaeap nttagqvptt evvgttpgqa ptaepsgtts vqvpttevis |
| 361 | tapvqmptae staaqvttte wvettarelp ipepegpdas simstesitg slgplldgta |
| 421 | tlrlvkrqvp ldcvlyrygs fsvtldivqg iesaeilqav psgegdafel tvscqgglpk |
| 481 | eacmeisspg cqppaqrlcq pvlpspacql vlhqilkggs gtyclnvsla dtnslavvst |
| 541 | qlimpgqeag lgqvplivgi llvlmavvla sliyrrrlmk qdfsvpqlph ssshwlrlpr |
| 601 | ifcscpigen spllsgqqv |
| Glutamate receptor ionotropic,NMDA 2A, isoform 1 precursor NP_000824.1, | |
| NP_001127879.1 | |
| 1 | mgrvgywtll vlpallvwrg papsaaaekg ppalniavml ghshdvtere lrtlwgpeqa |
| 61 | aglpldvnvv allmnrtdpk slithvcdlm sgarihglvf gddtdqeava qmldfissht |
| 121 | fvpilgihgg asmimadkdp tstffqfgas iqqqatvmlk imqdydwhvf slvttifpgy |
| 181 | refisfvktt vdnsfvgwdm qnvitldtsf edaktqvqlk kihssvilly cskdeavlil |
| 241 | searslgltg ydffwivpsl vsgntelipk efpsglisvs yddwdyslea rvrdgigilt |
| 301 | taassmlekf syipeakasc ygqmerpevp mhtlhpfmvn vtwdgkdlsf teegyqvhpr |
| 361 | lvvivlnkdr ewekvgkwen htlslrhavw pryksfsdce pddnhlsivt leeapfvive |
| 421 | didpltetcv rntvpcrkfv kinnstnegm nvkkcckgfc idilkklsrt vkftydlylv |
| 481 | tngkhgkkvn nvwngmigev vyqravmavg sltineerse vvdfsvpfve tgisvmvsrs |
| 541 | ngtvspsafl epfsasvwvm mfvmllivsa iavfvfeyfs pvgynrnlak gkaphgpsft |
| 601 | igkaiwllwg lvfnnsvpvq npkgttskim vsvwaffavi flasytanla afmiqeefvd |
| 661 | qvtglsdkkf qrphdysppf rfgtvpngst ernirnnypy mhqymtkfnq kgvedalvsl |
| 721 | ktgkldafiy daavlnykag rdegcklvti gsgyifattg ygialqkgsp wkrqidlall |
| 781 | qfvgdgemee letlwltgic hneknevmss qldidnmagv fymlaaamal slitfiwehl |
| 841 | fywklrfcft gvcsdrpgll fsisrgiysc ihgvhieekk kspdfnltgs qsnmlkllrs |
| 901 | aknissmsnm nssrmdspkr aadfiqrgsl imdmvsdkgn lmysdnrsfq gkesifgdnm |
| 961 | nelqtfvanr qkdnlnnyvf qgqhpltlne snpntvevav steskansrp rqlwkksvds |
| 1021 | irqdslsqnp vsqrdeatae nrthslkspr ylpeemahsd isetsnratc hrepdnsknh |
| 1081 | ktkdnfkrsv askypkdcse vertylktks ssprdkiyti dgekepgfhl dppqfvenvt |
| 1141 | lpenvdfpdp yqdpsenfrk gdstlpmnrn plhneeglsn ndqyklyskh ftlkdkgsph |
| 1201 | setseryrqn sthcrsclsn mptysghftm rspfkcdacl rmgnlydide dqmlqetgnp |
| 1261 | atgeqvyqqd waqnnalqlq knklrisrqh sydnivdkpr eldlsrpsrs islkdrerll |
| 1321 | egnfygslfs vpssklsgkk sslfpqgled skrsksllpd htsdnpflhs hrddqrlvig |
| 1381 | rcpsdpykhs lpsqavndsy lrsslrstas ycsrdsrghn dvyisehvmp yaanknnmys |
| 1441 | tprvlnscsn rrvykkmpsi esdv |
| Glutamate receptor ionotropic,NMDA 2A, isoform 2 precursor | |
| NP_001127880.1 | |
| 1 | mgrvgywtll vlpallvwrg papsaaaekg ppalniavml ghshdvtere lrtlwgpeqa |
| 61 | aglpldvnvv allmnrtdpk slithvcdlm sgarihglvf gddtdqeava qmldfissht |
| 121 | fvpilgihgg asmimadkdp tstffqfgas iqqqatvmlk imqdydwhvf slvttifpgy |
| 181 | refisfvktt vdnsfvgwdm qnvitldtsf edaktqvqlk kihssvilly cskdeavlil |
| 241 | searslgltg ydffwivpsl vsgntelipk efpsglisvs yddwdyslea rvrdgigilt |
| 301 | taassmlekf syipeakasc ygqmerpevp mhtlhpfmvn vtwdgkdlsf teegyqvhpr |
| 361 | lvvivlnkdr ewekvgkwen htlslrhavw pryksfsdce pddnhlsivt leeapfvive |
| 421 | didpltetcv rntvpcrkfv kinnstnegm nvkkcckgfc idilkklsrt vkftydlylv |
| 481 | tngkhgkkvn nvwngmigev vyqravmavg sltineerse vvdfsvpfve tgisvmvsrs |
| 541 | ngtvspsafl epfsasvwvm mfvmllivsa iavfvfeyfs pvgynrnlak gkaphgpsft |
| 601 | igkaiwllwg lvfnnsvpvq npkgttskim vsvwaffavi flasytanla afmiqeefvd |
| 661 | qvtglsdkkf qrphdysppf rfgtvpngst ernirnnypy mhqymtkfnq kgvedalvsl |
| 721 | ktgkldafiy daavlnykag rdegcklvti gsgyifattg ygialqkgsp wkrqidlall |
| 781 | qfvgdgemee letlwltgic hneknevmss qldidnmagv fymlaaamal slitfiwehl |
| 841 | fywklrfcft gvcsdrpgll fsisrgiysc ihgvhieekk kspdfnltgs qsnmlkllrs |
| 901 | aknissmsnm nssrmdspkr aadfiqrgsl imdmvsdkgn lmysdnrsfq gkesifgdnm |
| 961 | nelqtfvanr qkdnlnnyvf qgqhpltlne snpntvevav steskansrp rqlwkksvds |
| 1021 | irqdslsqnp vsqrdeatae nrthslkspr ylpeemahsd isetsnratc hrepdnsknh |
| 1081 | ktkdnfkrsv askypkdcse vertylktks ssprdkiyti dgekepgfhl dppqfvenvt |
| 1141 | lpenvdfpdp yqdpsenfrk gdstlpmnrn plhneeglsn ndqyklyskh ftlkdkgsph |
| 1201 | setseryrqn sthcrsclsn mptysghftm rspfkcdacl rmgnlydide dqmlqetgmt |
| 1261 | nawllgdapr tltntrchpr r |
| Metabotropic glutamate receptor 3 precursor NP_000831.2 | |
| 1 | mkmltrlqvl tlalfskgfl lslgdhnflr reikiegdlv lgglfpinek gtgteecgri |
| 61 | nedrgiqrle amlfaidein kddyllpgvk lgvhildtcs rdtyaleqsl efvrasltkv |
| 121 | deaeymcpdg syaiqenipl liagviggsy ssvsiqvanl lrlfqipqis yastsaklsd |
| 181 | ksrydyfart vppdfyqaka maeilrffnw tyvstvaseg dygetgieaf eqearlrnic |
| 241 | iataekvgrs nirksydsvi rellqkpnar vvvlfmrsdd sreliaaasr anasftwvas |
| 301 | dgwgaqesii kgsehvayga itlelasqpv rqfdryfqsl npynnhrnpw frdfweqkfq |
| 361 | cslqnkrnhr rvcdkhlaid ssnyeqeski mfvvnavyam ahalhkmqrt lcpnttklcd |
| 421 | amkildgkkl ykdyllkinf tapfnpnkda dsivkfdtfg dgmgrynvfn fqnvggkysy |
| 481 | lkvghwaetl sldvnsihws rnsvptsqcs dpcapnemkn mqpgdvccwi cipcepyeyl |
| 541 | adeftcmdcg sgqwptadlt gcydlpedyi rwedawaigp vtiaclgfmc tcmvvtvfik |
| 601 | hnntplvkas grelcyillf gvglsycmtf ffiakpspvi calrrlglgs sfaicysall |
| 661 | tktnciarif dgvkngaqrp kfispssqvf iclglilvqi vmvsvwlile apgtrrytla |
| 721 | ekretvilkc nvkdssmlis ltydvilvil ctvyafktrk cpenfneakf igftmyttci |
| 781 | iwlaflpify vtssdyrvqt ttmcisvsls gfvvlgclfa pkvhiilfqp qknvvthrlh |
| 841 | lnrfsvsgtg ttysqssast yvptvcngre vldsttssl |
| HPV E6 concoprotein, NP_041325.1 | |
| 1 | mhqkrtamfq dpqerprklp qlctelqtti hdiilecvyc kqqllrrevy dfafrdlciv |
| 61 | yrdgnpyavc dkclkfyski seyrhycysl ygttleqqyn kplcdllirc incqkplcpe |
| 121 | ekqrhldkkq rfhnirgrwt grcmsccrss rtrretql |
| HPV E7 Oncoprotein, NP_041326.1 | |
| 1 | mhgdtptlhe ymldlqpett dlycyeqlnd sseeedeidg pagqaepdra hynivtfcck |
| 61 | cdstlrlcvq sthvdirtle dllmgtlgiv cpicsqkp |
| GTPase HRas, isoform 1 NP_001123914.1, NP_005334.1 | |
| 1 | mteyklvvvg aggvgksalt iqliqnhfvd eydptiedsy rkqvvidget clldildtag |
| 61 | qeeysamrdq ymrtgegflc vfainntksf edihqyreqi krvkdsddvp mvlvgnkcdl |
| 121 | aartvesrqa qdlarsygip yietsaktrq gvedafytlv reirqhklrk lnppdesgpg |
| 181 | cmsckcvls |
| GTPase HRas, isoform 3 NP_001304983.1 | |
| 1 | mtcpwcwwgt svtwlhalwn lgrlrtspea tasptsrprp rpgraaalal apapgpsgtp |
| 61 | rdpcdpaapr agvedafytl vreirqhklr klnppdesgp gcmsckcvls |
| GTPase HRas, isoform 2 NP_789765.1 | |
| 1 | mteyklvvvg aggvgksalt iqliqnhfvd eydptiedsy rkqvvidget clldildtag |
| 61 | qeeysamrdq ymrtgegflc vfainntksf edihqyreqi krvkdsddvp mvlvgnkcdl |
| 121 | aartvesrqa qdlarsygip yietsaktrq gsrsgsssss gtlwdppgpm |
| Vascular endothelial growth factor receptor 2 precursor NP_002244.1 | |
| 1 | mqskvllava lwlcvetraa svglpsvsld lprlsiqkdi ltikanttlq itcrgqrdld |
| 61 | wlwpnnqsgs eqrvevtecs dglfcktlti pkvigndtga ykcfyretdl asviyvyvqd |
| 121 | yrspfiasvs dqhgvvyite nknktvvipc lgsisnlnvs lcarypekrf vpdgnriswd |
| 181 | skkgftipsy misyagmvfc eakindesyq simyivvvvg yriydvvlsp shgielsvge |
| 241 | klvlnctart elnvgidfnw eypsskhqhk klvnrdlktq sgsemkkfls tltidgvtrs |
| 301 | dqglytcaas sglmtkknst fvrvhekpfv afgsgmeslv eatvgervri pakylgyppp |
| 361 | eikwykngip lesnhtikag hvltimevse rdtgnytvil tnpiskekqs hvvslvvyvp |
| 421 | pqigekslis pvdsyqygtt qtltctvyai ppphhihwyw qleeecanep sqavsvtnpy |
| 481 | pceewrsved fqggnkievn knqfaliegk nktvstiviq aanvsalykc eavnkvgrge |
| 541 | rvisfhvtrg peitlqpdmq pteqesvslw ctadrstfen ltwyklgpqp lpihvgelpt |
| 601 | pvcknldtlw klnatmfsns tndilimelk naslqdqgdy vclaqdrktk krhcvvrqlt |
| 661 | vlervaptit gnlenqttsi gesievscta sgnpppqimw fkdnetlved sgivlkdgnr |
| 721 | nltirrvrke deglytcqac svlgcakvea ffiiegaqek tnleiiilvg taviamffwl |
| 781 | llviilrtvk ranggelktg ylsivmdpde lpldehcerl pydaskwefp rdrlklgkpl |
| 841 | grgafgqvie adafgidkta tcrtvavkml kegathsehr almselkili highhlnvvn |
| 901 | llgactkpgg plmvivefck fgnlstylrs krnefvpykt kgarfrqgkd yvgaipvdlk |
| 961 | rrldsitssq ssassgfvee kslsdveeee apedlykdfl tlehlicysf qvakgmefla |
| 1021 | srkcihrdla arnillsekn vvkicdfgla rdiykdpdyv rkgdarlplk wmapetifdr |
| 1081 | vytiqsdvws fgvllweifs lgaspypgvk ideefcrrlk egtrmrapdy ttpemyqtml |
| 1141 | dcwhgepsqr ptfselvehl gnllqanaqq dgkdyivlpi setlsmeeds glslptspvs |
| 1201 | cmeeeevcdp kfhydntagi sqylqnskrk srpvsvktfe dipleepevk vipddnqtds |
| 1261 | gmvlaseelk tledrtklsp sfggmvpsks resvasegsn qtsgyqsgyh sddtdttvys |
| 1321 | seeaellkli eigvqtgsta qilqpdsgtt lssppv |
| Mast/stem cell growth acor receptor KIT, isoform 1 precursor | |
| NP_000213.1 | |
| 1 | mrgargawdf lcvlllllrv qtgssqpsvs pgepsppsih pgksdlivrv gdeirllctd |
| 61 | pgfvkwtfei ldetnenkqn ewitekaeat ntgkytctnk hglsnsiyvf vrdpaklflv |
| 121 | drslygkedn dtlvrcpltd pevtnyslkg cqgkplpkdl rfipdpkagi miksvkrayh |
| 181 | rlclhcsvdq egksvlsekf ilkvrpafka vpvvsvskas yllregeeft vtctikdvss |
| 241 | svystwkren sqtklqekyn swhhgdfnye rqatltissa rvndsgvfmc yanntfgsan |
| 301 | vtttlevvdk gfinifpmin ttvfvndgen vdliveyeaf pkpehqqwiy mnrtftdkwe |
| 361 | dypksenesn iryvselhlt rlkgteggty tflvsnsdvn aaiafnvyvn tkpeiltydr |
| 421 | lvngmlqcva agfpeptidw yfcpgteqrc sasvlpvdvq tlnssgppfg klvvqssids |
| 481 | safkhngtve ckayndvgkt sayfnfafkg nnkeqihpht lftplligfv ivagmmciiv |
| 541 | miltykylqk pmyevqwkvv eeingnnyvy idptqlpydh kwefprnrls fgktlgagaf |
| 601 | gkvveatayg liksdaamtv avkmlkpsah lterealmse lkvlsylgnh mnivnllgac |
| 661 | tiggptlvit eyccygdlln flrrkrdsfi cskqedhaea alyknllhsk esscsdstne |
| 721 | ymdmkpgvsy vvptkadkrr svrigsyier dvtpaimedd elaldledll sfsyqvakgm |
| 781 | aflaskncih rdlaarnill thgritkicd fglardiknd snyvvkgnar lpvkwmapes |
| 841 | ifncvytfes dvwsygiflw elfslgsspy pgmpvdskfy kmikegfrml spehapaemy |
| 901 | dimktcwdad plkrptfkqi vqliekqise stnhiysnla ncspnrqkpv vdhsvrinsv |
| 961 | gstasssqpl lvhddv |
| Mast/stem cell growth acor receptor KIT, isoform 2 precursor | |
| NP_001087241.1 | |
| 1 | mrgargawdf lcvlllllrv qtgssqpsvs pgepsppsih pgksdlivrv gdeirllctd |
| 61 | pgfvkwtfei ldetnenkqn ewitekaeat ntgkytctnk hglsnsiyvf vrdpaklflv |
| 121 | drslygkedn dtlvrcpltd pevtnyslkg cqgkplpkdl rfipdpkagi miksvkrayh |
| 181 | rlclhcsvdq egksvlsekf ilkvrpafka vpvvsvskas yllregeeft vtctikdvss |
| 241 | svystwkren sqtklqekyn swhhgdfnye rqatltissa rvndsgvfmc yanntfgsan |
| 301 | vtttlevvdk gfinifpmin ttvfvndgen vdliveyeaf pkpehqqwiy mnrtftdkwe |
| 361 | dypksenesn iryvselhlt rlkgteggty tflvsnsdvn aaiafnvyvn tkpeiltydr |
| 421 | lvngmlqcva agfpeptidw yfcpgteqrc sasvlpvdvq tlnssgppfg klvvqssids |
| 481 | safkhngtve ckayndvgkt sayfnfafke qihphtlftp lligfvivag mmciivmilt |
| 541 | ykylqkpmye vqwkvveein gnnyvyidpt qlpydhkwef prnrlsfgkt lgagafgkvv |
| 601 | eataygliks daamtvavkm lkpsahlter ealmselkvl sylgnhmniv nllgactigg |
| 661 | ptlviteycc ygdllnflrr krdsficskq edhaeaalyk nllhskessc sdstneymdm |
| 721 | kpgvsyvvpt kadkrrsvri gsyierdvtp aimeddelal dledllsfsy qvakgmafla |
| 781 | skncihrdla arnillthgr itkicdfgla rdikndsnyv vkgnarlpvk wmapesifnc |
| 841 | vytfesdvws ygiflwelfs lgsspypgmp vdskfykmik egfrmlspeh apaemydimk |
| 901 | tcwdadplkr ptfkqivqli ekqisestnh iysnlancsp nrqkpvvdhs vrinsvgsta |
| 961 | sssqpllvhd dv |
| Plasma kallikrein isoform 1 preprotein NP_001639.1 | |
| 1 | mwvpvvfltl svtwigaapl ilsrivggwe cekhsqpwqv lvasrgravc ggvlvhpqwv |
| 61 | ltaahcirnk svillgrhsl fhpedtgqvf qvshsfphpl ydmsllknrf lrpgddsshd |
| 121 | lmllrlsepa eltdavkvmd lptqepalgt tcyasgwgsi epeefltpkk lqcvdlhvis |
| 181 | ndvcaqvhpq kvtkfmlcag rwtggkstcs gdsggplvcn gvlqgitswg sepcalperp |
| 241 | slytkvvhyr kwikdtivan p |
| Plasma kallikrein isoform 3 preprotein NP_001025218.1 | |
| 1 | mwvpvvfltl svtwigaapl ilsrivggwe cekhsqpwqv lvasrgravc ggvlvhpqwv |
| 61 | ltaahcirnk svillgrhsl fhpedtgqvf qvshsfphpl ydmsllknrf lrpgddsshd |
| 121 | lmllrlsepa eltdavkvmd lptqepalgt tcyasgwgsi epeefltpkk lqcvdlhvis |
| 181 | ndvcaqvhpq kvtkfmlcag rwtggkstcs wviliteltm palpmvlhgs lvpwrggv |
| Plasma kallikrein isoform 4 preprotein NP_001025219.1 | |
| 1 | mwvpvvfltl svtwigaapl ilsrivggwe cekhsqpwqv lvasrgravc ggvlvhpqwv |
| 61 | ltaahcirkp gddsshdlml lrlsepaelt davkvmdlpt qepalgttcy asgwgsiepe |
| 121 | efltpkklqc vdlhvisndv caqvhpqkvt kfmlcagrwt ggkstcsgds ggplvcngvl |
| 181 | qgitswgsep calperpsly tkvvhyrkwi kdtivanp |
| Tyrosine-protein kinase LCK, isoform a NP_001036236.1, NP_005347.3 | |
| 1 | mgcgcsshpe ddwmenidvc enchypivpl dgkgtllirn gsevrdplvt yegsnppasp |
| 61 | lqdnlvialh syepshdgdl gfekgeqlri leqsgewwka qslttgqegf ipfnfvakan |
| 121 | slepepwffk nlsrkdaerq llapgnthgs fliresesta gsfslsvrdf dqnqgevvkh |
| 181 | ykirnldngg fyispritfp glhelvrhyt nasdglctrl srpcqtqkpq kpwwedewev |
| 241 | pretlklver lgagqfgevw mgyynghtkv avkslkqgsm spdaflaean lmkqlqhqrl |
| 301 | vrlyavvtqe piyiiteyme ngslvdflkt psgikltink lldmaaqiae gmafieerny |
| 361 | ihrdlraani lvsdtlscki adfglarlie dneytarega kfpikwtape ainygtftik |
| 421 | sdvwsfgill teivthgrip ypgmtnpevi qnlergyrmv rpdncpeely qlmrlcwker |
| 481 | pedrptfdyl rsvledffta tegqyqpqp |
| Tyrosine-protein kinase LCK, isoform b NP_001317397.1 | |
| 1 | mgcgcsshpe ddwmenidvc enchypivpl dgkgtllirn gsevrdplvt yegsnppasp |
| 61 | lqdnlvialh syepshdgdl gfekgeqlri leqsgewwka qslttgqegf ipfnfvakan |
| 121 | slepepwffk nlsrkdaerq llapgnthgs fliresesta gsfslsvrdf dqnqgevvkh |
| 181 | ykirnldngg fyispritfp glhelvrhyt ryynghtkva vkslkqgsms pdaflaeanl |
| 241 | mkqlqhqrlv rlyavvtqep iyiiteymen gslvdflktp sgikltinkl ldmaaqiaeg |
| 301 | mafieernyi hrdlraanil vsdtlsckia dfglarlied neytaregak fpikwtapea |
| 361 | inygtftiks dvwsfgillt eivthgripy pgmtnpeviq nlergyrmvr pdncpeelyq |
| 421 | lmrlcwkerp edrptfdylr svledfftat egqyqpqp |
| Legumain preprotein NP_001008530.1, NP_005597.3 | |
| 1 | mvwkvavfls valgigavpi ddpedggkhw vvivagsngw ynyrhqadac hayqiihrng |
| 61 | ipdeqivvmm yddiaysedn ptpgivinrp ngtdvyqgvp kdytgedvtp qnflavlrgd |
| 121 | aeavkgigsg kvlksgpqdh vfiyftdhgs tgilvfpned lhvkdlneti hymykhkmyr |
| 181 | kmvfyieace sgsmmnhlpd ninvyattaa npressyacy ydekrstylg dwysvnwmed |
| 241 | sdvedltket lhkqyhlvks htntshvmqy gnktistmkv mqfqgmkrka sspvplppvt |
| 301 | hldltpspdv pltimkrklm ntndleesrq lteeiqrhld arhlieksvr kivsllaase |
| 361 | aeveqllser apltghscyp eallhfrthc fnwhsptyey alrhlyvlvn lcekpyplhr |
| 421 | iklsmdhvcl ghy |
| Macrophage migration inhibitory factor NP_002406.1 | |
| 1 | mpmfivntnv prasvpdgfl seltqqlaqa tgkppgyiav hvvpdqlmaf ggssepcalc |
| 61 | slhsigkigg agnrsyskll cgllaerlri spdrvyinyy dmnaanvgwn nstfa |
| MAGE family member A1 NP_004979.3 | |
| 1 | msleqrslhc kpeealeaqq ealglvcvqa atssssplvl gtleevptag stdppqspqg |
| 61 | asafpttinf trqrqpsegs ssreeegpst scileslfra vitkkvadlv gflllkyrar |
| 121 | epvtkaemle sviknykhcf peifgkases lqlvfgidvk eadptghsyv lvtclglsyd |
| 181 | gllgdnqimp ktgfliivlv miamegghap eeeiweelsv mevydgrehs aygeprkllt |
| 241 | qdlvqekyle yrqvpdsdpa ryeflwgpra laetsyvkvl eyvikvsarv rfffpslrea |
| 301 | alreeeegv |
| Melanoma-associated antigen 10 NP_001011543.2, NP_001238757.1, | |
| NP_066386.2 | |
| 1 | mprapkrqrc mpeedlqsqs etqglegaqa plaveedass ststsssfps sfpsssssss |
| 61 | sscyplipst peevsaddet pnppqsaqia csspsvvasl pldqsdegss sqkeespstl |
| 121 | qvlpdseslp rseidekvtd lvqfllfkyq mkepitkaei lesvirnyed hfpllfseas |
| 181 | ecmllvfgid vkevdptghs fvlvtslglt ydgmlsdvqs mpktgilili lsiifiegyc |
| 241 | tpeeviweal nmmglydgme hliygeprkl ltqdwvqeny leyrqvpgsd paryeflwgp |
| 301 | rahaeirkms llkflakvng sdprsfplwy eealkdeeer aqdriattdd ttamasasss |
| 361 | atgsfsype |
| Melanoma-associated antigen 12 NP_001159858.1, NP_001159859.1, | |
| NP_005358.2 | |
| 1 | mpleqrsqhc kpeegleaqg ealglvgaqa pateeqetas ssstlvevtl revpaaesps |
| 61 | pphspqgast lpttinytlw sqsdegssne eqegpstfpd letsfqvals rkmaelvhfl |
| 121 | llkyrarepf tkaemlgsvi rnfqdffpvi fskaseylql vfgievvevv righlyilvt |
| 181 | clglsydgll gdnqivpktg lliivlaiia kegdcapeek iweelsvlea sdgredsvfa |
| 241 | hprklltqdl vqenyleyrq vpgsdpacye flwgpralve tsyvkvlhhl lkisggphis |
| 301 | ypplhewafr egee |
| Melanoma-associated antigen 2 NP_001269430.1, NP_001269431.1, | |
| NP_001269433.1, NP_001269434.1, NP_005352.1, NP_786884.1, NP_786885.1 | |
| 1 | mpleqrsqhc kpeeglearg ealglvgaqa pateeqqtas ssstivevtl gevpaadsps |
| 61 | pphspqgass fsttinytlw rqsdegssnq eeegprmfpd lesefqaais rkmvelvhfl |
| 121 | llkyrarepv tkaemlesvl rncqdffpvi fskaseylql vfgievvevv pishlyilvt |
| 181 | clglsydgll gdnqvmpktg lliivlaiia iegdcapeek iweelsmlev fegredsvfa |
| 241 | hprkllmqdl vqenyleyrq vpgsdpacye flwgpralie tsyvkvlhht lkiggephis |
| 301 | ypplheralr egee |
| MAGE family member A3 NP_005353.1 | |
| 1 | mpleqrsqhc kpeeglearg ealglvgaqa pateeqeaas ssstlvevtl gevpaaespd |
| 61 | ppqspqgass lpttmnyplw sqsyedssnq eeegpstfpd lesefqaals rkvaelvhfl |
| 121 | llkyrarepv tkaemlgsvv gnwqyffpvi fskassslql vfgielmevd pighlyifat |
| 181 | clglsydgll gdnqimpkag lliivlaiia regdcapeek iweelsvlev fegredsilg |
| 241 | dpkklltqhf vqenyleyrq vpgsdpacye flwgpralve tsyvkvlhhm vkisggphis |
| 301 | ypplhewvlr egee |
| Melanoma-associated antigen 4 NP_001011548.1, NP_001011549.1, | |
| NP_001011550.1, NP_002353.3 | |
| 1 | msseqksqhc kpeegveaqe ealglvgaqa ptteeqeaav ssssplvpgt leevpaaesa |
| 61 | gppqspqgas alpttisftc wrqpnegsss qeeegpstsp daeslfreal snkvdelahf |
| 121 | llrkyrakel vtkaemlerv iknykrcfpv ifgkaseslk mifgidvkev dpasntytlv |
| 181 | tclglsydgl lgnnqifpkt glliivlgti amegdsasee eiweelgvmg vydgrehtvy |
| 241 | geprklltqd wvqenyleyr qvpgsnpary eflwgprala etsyvkvleh vvrvnarvri |
| 301 | aypslreaal leeeegv |
| Melanoma-associated antigen 6 NP_005354.1, NP_787064.1 | |
| 1 | mpleqrsqhc kpeeglearg ealglvgaqa pateeqeaas ssstlvevtl gevpaaespd |
| 61 | ppqspqgass lpttmnyplw sqsyedssnq eeegpstfpd lesefqaals rkvaklvhfl |
| 121 | llkyrarepv tkaemlgsvv gnwqyffpvi fskasdslql vfgielmevd pighvyifat |
| 181 | clglsydgll gdnqimpktg fliiilaiia kegdcapeek iweelsvlev fegredsifg |
| 241 | dpkklltqyf vqenyleyrq vpgsdpacye flwgpralie tsyvkvlhhm vkisggpris |
| 301 | ypllhewalr egee |
| Melanoma-associated antigen 9 NP_005356.1 | |
| 1 | msleqrsphc kpdedleaqg edlglmgaqe ptgeeeetts ssdskeeevs aagsssppqs |
| 61 | pqggasssis vyytlwsqfd egsssqeeee psssvdpaql efmfqealkl kvaelvhfll |
| 121 | hkyrvkepvt kaemlesvik nykryfpvif gkasefmqvi fgtdvkevdp aghsyilvta |
| 181 | lglscdsmlg dghsmpkaal liivlgvilt kdncapeevi wealsvmgvy vgkehmfyge |
| 241 | prklltqdwv qenyleyrqv pgsdpahyef lwgskahaet syekvinylv mlnarepicy |
| 301 | pslyeevlge eqegv |
| Melanoma-associated antigen C2 NP_057333.1 | |
| 1 | mppvpgvpfr nvdndsptsv eledwvdaqh ptdeeeeeas sasstlylvf spssfstsss |
| 61 | lilggpeeee vpsgvipnlt esipssppqg ppqgpsqspl ssccssfsws sfseesssqk |
| 121 | gedtgtcqgl pdsessftyt ldekvaelve flllkyeaee pvteaemlmi vikykdyfpv |
| 181 | ilkrarefme llfglaliev gpdhfcvfan tvgltdegsd degmpensll iiilsvifik |
| 241 | gncaseeviw evlnavgvya grehfvygep relltkvwvq ghyleyrevp hssppyyefl |
| 301 | wgprahsesi kkkvleflak lnntvpssfp swykdalkdv eervqatidt addatvmase |
| 361 | slsvmssnvs fse |
| Melanoma-associated antigen D1, isoform a NP_001005333.1 | |
| 1 | maqkmdcgag llgfqnpdac ravchplpqp pastlplsaf ptlcdppysq lrdppavlsc |
| 61 | yctplgaspa paeasvedsa llmqtlmeai qiseapptnq ataaaspqss qpptanemad |
| 121 | iqvsaaaarp ksafkvqnat tkgpngvydf sqahnakdvp ntqpkaafks qnatpkgpna |
| 181 | aydfsqaatt gelaanksem afkaqnattk vgpnatynfs qslnandlan srpktpfkaw |
| 241 | ndttkaptad tqtqnvnqak matsqadiet dpgisepdga taqtsadgsq aqnlesrtii |
| 301 | rgkrtrkinn lnveenssgd qrraplaagt wrsapvpvtt qnppgappnv lwqtplawqn |
| 361 | psgwqnqtar qtpparqspp arqtppawqn pvawqnpviw pnpviwqnpv iwpnpivwpg |
| 421 | pvvwpnplaw qnppgwqtpp gwqtppgwqg ppdwqgppdw plppdwplpp dwplptdwpl |
| 481 | ppdwipadwp ippdwqnlrp spnlrpspns rasqnpgaaq prdvallqer anklvkylml |
| 541 | kdytkvpikr semlrdiire ytdvypeiie racfvlekkf giqlkeidke ehlyilistp |
| 601 | eslagilgtt kdtpklglll vilgvifmng nraseavlwe alrkmglrpg vrhpllgdlr |
| 661 | klltyefvkq kyldyrrvpn snppeyeflw glrsyhetsk mkvlrfiaev qkrdprdwta |
| 721 | qfmeaadeal daldaaaaea earaeartrm gigdeavsgp wswddiefel ltwdeegdfg |
| 781 | dpwsripftf waryhqnars rfpqtfagpi igpggtasan faanfgaigf fwve |
| Melanoma-associated antigen D1, isoform b NP_001005332.1, NP_008917.3 | |
| 1 | maqkmdcgag llgfqaeasv edsallmqtl meaiqiseap ptnqataaas pqssqpptan |
| 61 | emadiqvsaa aarpksafkv qnattkgpng vydfsqahna kdvpntqpka afksqnatpk |
| 121 | gpnaaydfsq aattgelaan ksemafkaqn attkvgpnat ynfsqslnan dlansrpktp |
| 181 | fkawndttka ptadtqtqnv nqakmatsqa dietdpgise pdgataqtsa dgsqaqnles |
| 241 | rtiirgkrtr kinnlnveen ssgdqrrapl aagtwrsapv pvttqnppga ppnvlwqtpl |
| 301 | awqnpsgwqn qtarqtppar qspparqtpp awqnpvawqn pviwpnpviw qnpviwpnpi |
| 361 | vwpgpvvwpn plawqnppgw qtppgwqtpp gwqgppdwqg ppdwplppdw plppdwplpt |
| 421 | dwplppdwip adwpippdwq nlrpspnlrp spnsrasqnp gaaqprdval lqeranklvk |
| 481 | ylmlkdytkv pikrsemlrd iireytdvyp eiieracfvl ekkfgiqlke idkeehlyil |
| 541 | istpeslagi lgttkdtpkl glllvilgvi fmngnrasea vlwealrkmg lrpgvrhpll |
| 601 | gdlrklltye fvkqkyldyr rvpnsnppey eflwglrsyh etskmkvlrf iaevqkrdpr |
| 661 | dwtaqfmeaa dealdaldaa aaeaearaea rtrmgigdea vsgpwswddi efelltwdee |
| 721 | gdfgdpwsri pftfwaryhq narsrfpqtf agpiigpggt asanfaanfg aigffwve |
| Mitogen-activated protein kinase kinase kinase 5 NP_005914.1 | |
| 1 | msteadegit fsvppfapsg fctipeggic rrggaaavge geehqlpppp pgsfwnvesa |
| 61 | aapgigcpaa tssssatrgr gssvgggsrr ttvayvinea sqgqlvvaes ealqslreac |
| 121 | etvgatletl hfgkldfget tvldrfynad iavvemsdaf rqpslfyhlg vresfsmann |
| 181 | iilycdtnsd slqslkeiic qkntmctgny tfvpymitph nkvyccdssf mkgltelmqp |
| 241 | nfelllgpic lplvdrfiql lkvaqasssq yfresilndi rkarnlytgk elaaelarir |
| 301 | qrvdnievlt adivinllls yrdiqdydsi vklvetlekl ptfdlashhh vkfhyafaln |
| 361 | rrnlpgdrak aldimipmvq segqvasdmy clvgriykdm fldsnftdte srdhgaswfk |
| 421 | kafeseptlq sginyavlll aaghqfessf elrkvgvkls sllgkkgnle klqsywevgf |
| 481 | flgasvland hmrviqasek lfklktpawy lksivetili ykhfvkltte qpvakqelvd |
| 541 | fwmdflveat ktdvtvvrfp vlileptkiy qpsylsinne veektisiwh vlpddkkgih |
| 601 | ewnfsassvr gvsiskfeer ccflyvlhns ddfqiyfcte lhckkffemv ntiteekgrs |
| 661 | teegdcesdl leydyeyden gdrvvlgkgt ygivyagrdl snqvriaike iperdsrysq |
| 721 | plheeialhk hlkhknivqy lgsfsengfi kifmeqvpgg slsallrskw gplkdneqti |
| 781 | gfytkqileg lkylhdnqiv hrdikgdnvl intysgvlki sdfgtskrla ginpctetft |
| 841 | gtlqymapei idkgprgygk aadiwslgct iiematgkpp fyelgepqaa mfkvgmfkvh |
| 901 | peipesmsae akafilkcfe pdpdkracan dllvdeflkv sskkkktqpk lsalsagsne |
| 961 | ylrsislpvp vlvedtssss eygsvspdte lkvdpfsfkt rakscgerdv kgirtlflgi |
| 1021 | pdenfedhsa ppspeekdsg ffmlrkdser ratlhrilte dqdkivrnlm eslaqgaeep |
| 1081 | klkwehittl iaslrefvrs tdrkiiattl sklkleldfd shgisqvqvv lfgfqdavnk |
| 1141 | vlrnhnikph wmfaldsiir kavqtaitil vpelrphfsl asesdtadqe dldveddhee |
| 1201 | qpsnqtvrrp qaviedavat sgvstlsstv shdsqsahrs lnvqlgrmki etnrlleelv |
| 1261 | rkekelqall hraieekdqe ikhlklksqp ieipelpvfh lnssgtnted seltdwlrvn |
| 1321 | gadedtisrf laedytlldv lyyvtrddlk clrlrggmlc tlwkaiidfr nkqt |
| Mitogen-activated protein kinase kinase kinase 9, isoform 1 | |
| NP_149132.2 | |
| 1 | mepsrallgc lasaaaaapp gedgagagae eeeeeeeeaa aavgpgelgc daplpywtav |
| 61 | feyeaagede ltlrlgdvve vlskdsqvsg degwwtgqln qrvgifpsny vtprsafssr |
| 121 | cqpggedpsc yppiqlleid faeltleeii giggfgkvyr afwigdevav kaarhdpded |
| 181 | isqtienvrq eaklfamlkh pniialrgvc lkepnlclvm efarggplnr vlsgkrippd |
| 241 | ilvnwavqia rgmnylhdea ivpiihrdlk ssnililqkv engdlsnkil kitdfglare |
| 301 | whrttkmsaa gtyawmapev irasmfskgs dvwsygvllw elltgevpfr gidglavayg |
| 361 | vamnklalpi pstcpepfak lmedcwnpdp hsrpsftnil dqlttieesg ffempkdsfh |
| 421 | clqdnwkhei qemfdqlrak ekelrtweee ltraalqqkn qeellrrreq elaereidil |
| 481 | erelniiihq lcqekprvkk rkgkfrksrl klkdgnrisl psdfqhkftv qasptmdkrk |
| 541 | slinsrsspp asptiiprlr aiqltpgess ktwgrssvvp keegeeeekr apkkkgrtwg |
| 601 | pgtlgqkela sgdegspqrr ekanglstps esphfhlglk slvdgykqws ssapnlvkgp |
| 661 | rsspalpgft slmemallaa swvvpidiee dedsegpgsg esrlqhspsq sylcipfprg |
| 721 | edgdgpssdg iheeptpvns atstpqltpt nslkrggahh rrcevallgc gavlaatglg |
| 781 | fdlleagkcq llpleepepp areekkrreg lfqrssrprr stsppsrklf kkeepmlllg |
| 841 | dpsasltlls lssisecnst rsllrsdsde ivvyempvsp veapplspct hnplvnvrve |
| 901 | rfkrdpnqsl tpthvtlttp sqpsshrrtp sdgalkpetl lasrspssng lspspgagml |
| 961 | ktpspsrdpg efprlpdpnv vfpptprrwn tqqdstlerp ktleflprpr psanrqrldp |
| 1021 | wwfvspshar stspanssst etpsnldscf asssstveer pglpallpfq agplpptert |
| 1081 | lldldaegqs qdstvplcra elnthrpapy eiqqefws |
| Mitogen-activated protein kinase kinase kinase 9, isoform 2 | |
| NP_001271159.1 | |
| 1 | mepsrallgc lasaaaaapp gedgagagae eeeeeeeeaa aavgpgelgc daplpywtav |
| 61 | feyeaagede ltlrlgdvve vlskdsqvsg degwwtgqln qrvgifpsny vtprsafssr |
| 121 | cqpggedpsc yppiqlleid faeltleeii giggfgkvyr afwigdevav kaarhdpded |
| 181 | isqtienvrq eaklfamlkh pniialrgvc lkepnlclvm efarggplnr vlsgkrippd |
| 241 | ilvnwavqia rgmnylhdea ivpiihrdlk ssnililqkv engdlsnkil kitdfglare |
| 301 | whrttkmsaa gtyawmapev irasmfskgs dvwsygvllw elltgevpfr gidglavayg |
| 361 | vamnklalpi pstcpepfak lmedcwnpdp hsrpsftnil dqlttieesg ffempkdsfh |
| 421 | clqdnwkhei qemfdqlrak ekelrtweee ltraalqqkn qeellrrreq elaereidil |
| 481 | erelniiihq lcgekprvkk rkgkfrksrl klkdgnrisl psdfqhkftv qasptmdkrk |
| 541 | slinsrsspp asptiiprlr aiqltpgess ktwgrssvvp keegeeeekr apkkkgrtwg |
| 601 | pgtlgqkela sgdegspqrr ekanglstps esphfhlglk slvdgykqws ssapnlvkgp |
| 661 | rsspalpgft slmemededs egpgsgesrl qhspsqsylc ipfprgedgd gpssdgihee |
| 721 | ptpvnsatst pqltptnslk rggahhrrce vallgcgavl aatglgfdll eagkcqllpl |
| 781 | eepepparee kkrreglfqr ssrprrstsp psrklfkkee pmlllgdpsa sltllslssi |
| 841 | secnstrsll rsdsdeivvy empvspveap plspcthnpl vnvrverfkr dpnqsltpth |
| 901 | vtlttpsqps shrrtpsdga lkpetllasr spssnglsps pgagmlktps psrdpgefpr |
| 961 | lpdpnvvfpp tprrwntqqd stlerpktle flprprpsan rqrldpwwfv spsharstsp |
| 1021 | anssstetps nldscfasss stveerpglp allpfqagpl pptertlldl daegqsqdst |
| 1081 | vplcraelnt hrpapyeiqq efws |
| Mitogen-activated protein kinase kinase kinase 9, isoform 3 | |
| NP_001271160.1 | |
| 1 | meltgleval vlilqkveng dlsnkilkit dfglarewhr ttkmsaagty awmapevira |
| 61 | smfskgsdvw sygvllwell tgevpfrgid glavaygvam nklalpipst cpepfaklme |
| 121 | dcwnpdphsr psftnildql ttieesgffe mpkdsfhclq dnwkheiqem fdqlrakeke |
| 181 | lrtweeeltr aalqqknqee llrrreqela ereidilere lniiihqlcq ekprvkkrkg |
| 241 | kfrksrlklk dgnrislpsd fqhkftvqas ptmdkrksli nsrssppasp tiiprlraiq |
| 301 | cetvsqiswg qntqghlspa lsshrlvqac sihnfchlss tmciymhilt pgessktwgr |
| 361 | ssvvpkeege eeekrapkkk grtwgpgtlg qkelasgdeg lkslvdgykq wsssapnlvk |
| 421 | gprsspalpg ftslmemall aaswvvpidi eededsegpg sgesrlqhsp sqsylcipfp |
| 481 | rgedgdgpss dgiheeptpv nsatstpqlt ptnslkrgga hhrrcevall gcgavlaatg |
| 541 | lgfdlleagk cqllpleepe ppareekkrr eglfqrssrp rrstsppsrk lfkkeepmll |
| 601 | lgdpsasltl lslssisecn strsllrsds deivvyempv spveapplsp cthnplvnvr |
| 661 | verfkrdpnq sltpthvtlt tpsqpsshrr tpsdgalkpe tllasrspss nglspspgag |
| 721 | mlktpspsrd pgefprlpdp nvvfpptprr wntqqdstle rpktleflpr prpsanrqrl |
| 781 | dpwwfvspsh arstspanss stetpsnlds cfasssstve erpglpallp fqagplppte |
| 841 | rtlldldaeg qsqdstvplc raelnthrpa pyeiqqefws |
| Mitogen-activated protein kinase kinase kinase 9, isoform 4 | |
| NP_001271161.1 | |
| 1 | msaagtyawm apevirasmf skgsdvwsyg vllwelltge vpfrgidgla vaygvamnkl |
| 61 | alpipstcpe pfaklmedcw npdphsrpsf tnildqltti eesgffempk dsfhclqdnw |
| 121 | kheiqemfdq lrakekelrt weeeltraal qqknqeellr rreqelaere idilerelni |
| 181 | iihqlcqekp rvkkrkgkfr ksrlklkdgn rislpsdfqh kftvqasptm dkrkslinsr |
| 241 | ssppasptii prlraiqcet vsqiswgqnt qghlspalss hrlvqacsih nfchlsstmc |
| 301 | iymhiltpge ssktwgrssv vpkeegeeee krapkkkgrt wgpgtlgqke lasgdeglks |
| 361 | lvdgykqwss sapnlvkgpr sspalpgfts lmemallaas wvvpidieed edsegpgsge |
| 421 | srlqhspsqs ylcipfprge dgdgpssdgi heeptpvnsa tstpqltptn slkrggahhr |
| 481 | rcevallgcg avlaatglgf dlleagkcql lpleepeppa reekkrregl fqrssrprrs |
| 541 | tsppsrklfk keepmlllgd psasltllsl ssisecnstr sllrsdsdei vvyempvspv |
| 601 | eapplspcth nplvnvrver fkrdpnqslt pthvtlttps qpsshrrtps dgalkpetll |
| 661 | asrspssngl spspgagmlk tpspsrdpge fprlpdpnvv fpptprrwnt qqdstlerpk |
| 721 | tleflprprp sanrqrldpw wfvspshars tspanssste tpsnldscfa sssstveerp |
| 781 | glpallpfqa gplpptertl ldldaegqsq dstvplcrae lnthrpapye iqqefws |
| Mitogen-activated protin kinase 1 NP_002736.3, NP_620407.1 | |
| 1 | maaaaaagag pemvrgqvfd vgprytnlsy igegaygmvc saydnvnkvr vaikkispfe |
| 61 | hqtycqrtlr eikillrfrh eniigindii raptieqmkd vyivqdlmet dlykllktqh |
| 121 | lsndhicyfl yqilrglkyi hsanvlhrdl kpsnlllntt cdlkicdfgl arvadpdhdh |
| 181 | tgflteyvat rwyrapeiml nskgytksid iwsvgcilae mlsnrpifpg khyldqlnhi |
| 241 | lgilgspsqe dlnciinlka rnyllslphk nkvpwnrlfp nadskaldll dkmltfnphk |
| 301 | rieveqalah pyleqyydps depiaeapfk fdmelddlpk eklkelifee tarfqpgyrs |
| Melan-A NP_005502.1 | |
| 1 | mpredahfiy gypkkghghs yttaeeaagi giltvilgvl lligcwycrr rngyralmdk |
| 61 | slhvgtqcal trrcpqegfd hrdskvslqe kncepvvpna ppayeklsae qspppysp |
| Melanotransferrin, isoform 1 preprotein NP_005920.2 | |
| 1 | mrgpsgalwl llalrtvlgg mevrwcatsd peqhkcgnms eafreagiqp sllcvrgtsa |
| 61 | dhcvqliaaq eadaitldgg aiyeagkehg lkpvvgevyd qevgtsyyav avvrrsshvt |
| 121 | idtlkgvksc htginrtvgw nvpvgylves grlsvmgcdv lkavsdyfgg scvpgagets |
| 181 | yseslcrlcr gdssgegvcd kspleryydy sgafrclaeg agdvafvkhs tvlentdgkt |
| 241 | lpswgqalls qdfellcrdg sradvtewrq chlarvpaha vvvradtdgg lifrllnegq |
| 301 | rlfshegssf qmfsseaygq kdllfkdsts elvpiatqty eawlgheylh amkgllcdpn |
| 361 | rlppylrwcv lstpeiqkcg dmavafrrqr lkpeiqcvsa kspqhcmeri qaeqvdavtl |
| 421 | sgediytagk tyglvpaage hyapedssns yyvvavvrrd sshaftldel rgkrschagf |
| 481 | gspagwdvpv galiqrgfir pkdcdvltav seffnascvp vnnpknypss lcalcvgdeq |
| 541 | grnkcvgnsq eryygyrgaf rclvenagdv afvrhttvfd ntnghnsepw aaelrsedye |
| 601 | llcpngarae vsqfaacnla qipphavmvr pdtniftvyg lldkaqdlfg ddhnkngfkm |
| 661 | fdssnyhgqd llfkdatvra vpvgekttyr gwlgldyvaa legmssqqcs gaaapapgap |
| 721 | llplllpala arllppal |
| Melanotransferrin, isoform 2 precursor NP_201573.1 | |
| 1 | mrgpsgalwl llalrtvlgg mevrwcatsd peqhkcgnms eafreagiqp sllcvrgtsa |
| 61 | dhcvqliaaq eadaitldgg aiyeagkehg lkpvvgevyd qevgtsyyav avvrrsshvt |
| 121 | idtlkgvksc htginrtvgw nvpvgylves grlsvmgcdv lkavsdyfgg scvpgagets |
| 181 | yseslcrlcr gdssgegvcd kspleryydy sgafrclaeg agdvafvkhs tvlentdesp |
| 241 | srrqtwtrse eeegecpahe earrtmrssa gqawkwapvh rpqdesdkge fgkraksrdm |
| 301 | lg |
| Baculoviral IAP repeat containing 7, isoform alpha NP_647478.1 | |
| 1 | mgpkdsakcl hrgpqpshwa agdgptqerc gprslgspvl gldtcrawdh vdgqilgqlr |
| 61 | plteeeeeeg agatlsrgpa fpgmgseelr lasfydwplt aevppellaa agffhtghqd |
| 121 | kvrcffcygg lqswkrgddp wtehakwfps cqfllrskgr dfvhsvqeth sqllgswdpw |
| 181 | eepedaapva psvpasgype lptprrevqs esaqepggvs paeaqrawwv leppgardve |
| 241 | aqlrrlqeer tckvcldrav sivfvpcghl vcaecapglq lcpicrapvr srvrtfls |
| Baculoviral IAP repeat containing 7, isoform beta NP_071444.1 | |
| 1 | mgpkdsakcl hrgpqpshwa agdgptqerc gprslgspvl gldtcrawdh vdgqilgqlr |
| 61 | plteeeeeeg agatlsrgpa fpgmgseelr lasfydwplt aevppellaa agffhtghqd |
| 121 | kvrcffcygg lqswkrgddp wtehakwfps cqfllrskgr dfvhsvqeth sqllgswdpw |
| 181 | eepedaapva psvpasgype lptprrevqs esaqepgard veaqlrrlqe ertckvcldr |
| 241 | avsivfvpcg hlvcaecapg lqlcpicrap vrsrvrtfls |
| Neutrophil collagenase, isoform 1 preprotein NP_002415.1 | |
| 1 | mfslktlpfl lllhvqiska fpvsskeknt ktvqdylekf yqlpsnqyqs trkngtnviv |
| 61 | eklkemqrff glnvtgkpne etldmmkkpr cgvpdsggfm ltpgnpkwer tnltyrirny |
| 121 | tpqlseaeve raikdafelw svaspliftr isqgeadini afyqrdhgdn spfdgpngil |
| 181 | ahafqpgqgi ggdahfdaee twtntsanyn lflvaahefg hslglahssd pgalmypnya |
| 241 | fretsnyslp qddidgiqai yglssnpiqp tgpstpkpcd psltfdaitt lrgeilffkd |
| 301 | ryfwrrhpql qrvemnfisl fwpslptgiq aayedfdrdl iflfkgnqyw alsgydilqg |
| 361 | ypkdisnygf pssvqaidaa vfyrsktyff vndqfwrydn qrqfmepgyp ksisgafpgi |
| 421 | eskvdavfqq ehffhvfsgp ryyafdliaq rvtrvargnk wlncryg |
| Neutrophil collagenase, isoform 2 NP_001291370.1, NP_001291371.1 | |
| 1 | mqqipqeksi ndylekfyql psnqyqstrk ngtnvivekl kemqrffgln vtgkpneetl |
| 61 | dmmkkprcgv pdsggfmltp gnpkwertnl tyrirnytpq lseaeverai kdafelwsva |
| 121 | spliftrisq geadiniafy qrdhgdnspf dgpngilaha fqpgqgiggd ahfdaeetwt |
| 181 | ntsanynlfl vaahefghsl glahssdpga lmypnyafre tsnyslpqdd idgiqaiygl |
| 241 | ssnpiqptgp stpkpcdpsl tfdaittlrg eilffkdryf wrrhpqlqrv emnfislfwp |
| 301 | slptgiqaay edfdrdlifl fkgnqywals gydilqgypk disnygfpss vqaidaavfy |
| 361 | rsktyffvnd qfwrydnqrq fmepgypksi sgafpgiesk vdavfqqehf fhvfsgpryy |
| 421 | afdliaqrvt rvargnkwln cryg |
| Mesothelin, isoform 1 preprotein NP_001170826.1, NP_005814.2 | |
| 1 | malptarpll gscgtpalgs llfllfslgw vqpsrtlage tgqeaapldg vlanppniss |
| 61 | lsprqllgfp caevsglste rvrelavala qknvklsteq lrclahrlse ppedldalpl |
| 121 | dlllflnpda fsgpqactrf fsritkanvd llprgaperq rllpaalacw gvrgsllsea |
| 181 | dvralgglac dlpgrfvaes aevllprlvs cpgpldqdqq eaaraalqgg gppygppstw |
| 241 | svstmdalrg llpvlgqpii rsipqgivaa wrqrssrdps wrqpertilr prfrrevekt |
| 301 | acpsgkkare ideslifykk weleacvdaa llatqmdrvn aipftyeqld vlkhkldely |
| 361 | pqgypesviq hlgylflkms pedirkwnvt sletlkalle vnkghemspq vatlidrfvk |
| 421 | grgqldkdtl dtltafypgy lcslspeels svppssiwav rpqdldtcdp rqldvlypka |
| 481 | rlafqnmngs eyfvkiqsfl ggaptedlka lsqqnvsmdl atfmklrtda vlpltvaevq |
| 541 | kllgphvegl kaeerhrpvr dwilrqrqdd ldtlglglqg gipngylvld lsmqealsgt |
| 601 | pcllgpgpvl tvlalllast la |
| Mesothelin, isoform 2 preprotein NP_037536.2 | |
| 1 | malptarpll gscgtpalgs llfllfslgw vqpsrtlage tgqeaapldg vlanppniss |
| 61 | lsprqllgfp caevsglste rvrelavala qknvklsteq lrclahrlse ppedldalpl |
| 121 | dlllflnpda fsgpqactrf fsritkanvd llprgaperq rllpaalacw gvrgsllsea |
| 181 | dvralgglac dlpgrfvaes aevllprlvs cpgpldqdqq eaaraalqgg gppygppstw |
| 241 | systmdalrg llpvlgqpii rsipqgivaa wrqrssrdps wrqpertilr prfrrevekt |
| 301 | acpsgkkare ideslifykk weleacvdaa llatqmdrvn aipftyeqld vlkhkldely |
| 361 | pqgypesviq hlgylflkms pedirkwnvt sletlkalle vnkghemspq aprrplpqva |
| 421 | tlidrfvkgr gqldkdtldt ltafypgylc slspeelssv ppssiwavrp qdldtcdprq |
| 481 | ldvlypkarl afqnmngsey fvkiqsflgg aptedlkals qqnvsmdlat fmklrtdavl |
| 541 | pltvaevqkl lgphveglka eerhrpvrdw ilrqrqddld tlglglqggi pngylvldls |
| 601 | mqealsgtpc llgpgpvltv lalllastla |
| Mucin-1, isoform 1 precursor NP_002447.4 | |
| 1 | mtpgtqspff llllltvltv vtgsghasst pggeketsat qrssvpsste knalstgvsf |
| 61 | fflsfhisnl qfnssledps tdyyqelqrd isemflqiyk qggflglsni kfrpgsvvvq |
| 121 | ltlafregti nvhdvetqfn qykteaasry nltisdvsvs dvpfpfsaqs gagvpgwgia |
| 181 | llvlvcvlva laivyliala vcqcrrknyg qldifpardt yhpmseypty hthgryvpps |
| 241 | stdrspyekv sagnggssls ytnpavaats anl |
| Mucin-1, isoform 2 precursor NP_001018016.1 | |
| 1 | mtpgtqspff llllltvlta ttapkpatvv tgsghasstp ggeketsatq rssvpsstek |
| 61 | nafnssledp stdyyqelqr disemflqiy kqggflglsn ikfrpgsvvv qltlafregt |
| 121 | invhdvetqf nqykteaasr ynltisdvsv sdvpfpfsaq sgagvpgwgi allvlvcvlv |
| 181 | alaivylial avcqcrrkny gqldifpard tyhpmseypt yhthgryvpp sstdrspyek |
| 241 | vsagnggssl sytnpavaat sanl |
| Mucin-1, isoform 3 precursor NP_001018017.1 | |
| 1 | mtpgtqspff llllltvltv vtgsghasst pggeketsat qrssvpsste knafnssled |
| 61 | pstdyyqelq rdisemflqi ykqggflgls nikfrpgsvv vqltlafreg tinvhdvetq |
| 121 | fnqykteaas rynltisdvs vsdvpfpfsa qsgagvpgwg iallvlvcvl valaivylia |
| 181 | lavcqcrrkn ygqldifpar dtyhpmseyp tyhthgryvp psstdrspye kvsagnggss |
| 241 | lsytnpavaa tsanl |
| Mucin-1, isoform 5 precursor NP_001037855.1 | |
| 1 | mtpgtqspff llllltvltv vtgsghasst pggeketsat qrssvpsste knaipapttt |
| 61 | kscretflkc fcrfinkgvf waspilssvs dvpfpfsaqs gagvpgwgia llvlvcvlva |
| 121 | laivyliala vcqcrrknyg qldifpardt yhpmseypty hthgryvpps stdrspyekv |
| 181 | sagnggssls ytnpavaats anl |
| Mucin-1, isoform 6 precursor NP_001037856.1 | |
| 1 | mtpgtqspff llllltvltv vtgsghasst pggeketsat qrssvpsste knafnssled |
| 61 | pstdyyqelq rdisemavcq crrknygqld ifpardtyhp mseyptyhth gryvppsstd |
| 121 | rspyekvsag nggsslsytn pavaatsanl |
| Mucin-1, isoform 7 precursor NP_001037857.1 | |
| 1 | mtpgtqspff llllltvlta ttapkpatvv tgsghasstp ggeketsatq rssvpsstek |
| 61 | nafnssledp stdyyqelqr disemavcqc rrknygqldi fpardtyhpm seyptyhthg |
| 121 | ryvppsstdr spyekvsagn ggsslsytnp avaatsanl |
| Mucin-1, isoform 8 precursor NP_001037858.1 | |
| 1 | mtpgtqspff llllltvltv vtgsghasst pggeketsat qrssvpsste knaipapttt |
| 61 | kscretflkc fcrfinkgvf waspilssvw gwgarlghra agaglcsgca ghclshclgc |
| 121 | lsvppkelra aghlsspgyl psyervphlp hpwalcap |
| Mucin-1, isoform 9 precursor NP_001191214.1 | |
| 1 | mtpgtqspff llllltvltv vtgsghasst pggeketsat qrssvpsste knavsmtssv |
| 61 | lsshspgsgs sttqgqdvtl apatepasgs aatwgqdvts vpvtrpalgs ttppandvts |
| 121 | apdnkpapgs tappahgvts apdtrpapgs tappahgvts apdnrpalgs tappvhnvts |
| 181 | asgsasgsas tlvhngtsar atttpaskst pfsipshhsd tpttlashst ktdassthhs |
| 241 | tvppltssnh stspqlstgv sffflsfhis nlqfnssled pstdyyqelq rdisemflqi |
| 301 | ykqggflgls nikfrpgsvv vqltlafreg tinvhdvetq fnqykteaas rynltisdvs |
| 361 | vsdvpfpfsa qsgagvpgwg iallvlvcvl valaivylia lavcqcrrkn ygqldifpar |
| 421 | dtyhpmseyp tyhthgryvp psstdrspye kvsagnggss lsytnpavaa tsanl |
| Mucin-1, isoform 10 precursor NP_001191215.1 | |
| 1 | mtpgtqspff llllltvlta ttapkpatvv tgsghasstp ggeketsatq rssvpsstek |
| 61 | navsmtssvl sshspgsgss ttqgqdvtla patepasgsa atwgqdvtsv pvtrpalgst |
| 121 | tppandvtsa pdnkpapgst appahgvtsa pdtrpapgst appahgvtsa pdnrpalgst |
| 181 | appvhnvtsa sgsasgsast lvhngtsara tttpaskstp fsipshhsdt pttlashstk |
| 241 | tdassthhst vppltssnhs tspqlstgvs ffflsfhisn lqfnssledp stdyyqelqr |
| 301 | disemflqiy kqggflglsn ikfrpgsvvv qltlafregt invhdvetqf nqykteaasr |
| 361 | ynltisdvsv sdvpfpfsaq sgagvpgwgi allvlvcvlv alaivylial avcqcrrkny |
| 421 | gqldifpard tyhpmseypt yhthgryvpp sstdrspyek vsagnggssl sytnpavaat |
| 481 | sanl |
| Mucin-1, isoform 11 precursor NP_001191216.1 | |
| 1 | mtpgtqspff llllltvlta ttapkpatvv tgsghasstp ggeketsatq rssvpsstek |
| 61 | nalstgvsff flsfhisnlq fnssledpst dyyqelqrdi semflqiykq ggflglsnik |
| 121 | frpgsvvvql tlafregtin vhdvetqfnq ykteaasryn ltisdvsvsd vpfpfsaqsg |
| 181 | agvpgwgial lvlvcvlval aivylialav cqcrrknygq ldifpardty hpmseyptyh |
| 241 | thgryvppss tdrspyekvs agnggsslsy tnpavaatsa nl |
| Mucin-1, isoform 12 precursor NP_001191217.1 | |
| 1 | mtpgtqspff llllltvlta ttapkpatvv tgsghasstp ggeketsatq rssvpsstek |
| 61 | nafnssledp stdyyqelqr disemflqiy kqggflglsn ikfrpgsvvv qltlafregt |
| 121 | invhdvetqf nqykteaasr ynltisdvsv wgwgarlghr aagaglcsgc aghclshclg |
| 181 | clsvppkelr aaghlsspgy lpsyervphl phpwalcap |
| Mucin-1, isoform 13 precursor NP_001191218.1 | |
| 1 | mtpgtqspff llllltvlta ttapkpatvv tgsghasstp ggeketsatq rssvpsstek |
| 61 | naiykqggfl glsnikfrpg svvvqltlaf regtinvhdv etqfnqykte aasrynltis |
| 121 | dvsvsdvpfp fsaqsgagvp gwgiallvlv cvlvalaivy lialavcqcr rknygqldif |
| 181 | pardtyhpms eyptyhthgr yvppsstdrs pyekvsagng gsslsytnpa vaatsanl |
| Mucin-1, isoform 14 precursor NP_001191219.1 | |
| 1 | mtpgtqspff llllltvltg geketsatqr ssvpsstekn aiykqggflg lsnikfrpgs |
| 61 | vvvqltlafr egtinvhdve tqfnqyktea asrynltisd vsvsdvpfpf saqsgagvpg |
| 121 | wgiallvlvc vlvalaivyl ialavcqcrr knygqldifp ardtyhpmse yptyhthgry |
| 181 | vppsstdrsp yekvsagngg sslsytnpav aatsanl |
| Mucin-1, isoform 15 precursor NP_001191220.1 | |
| 1 | mtpgtqspff llllltvlta ttapkpatvv tgsghasstp ggeketsatq rssvpsstek |
| 61 | naflqiykqg gflglsnikf rpgsvvvqlt lafregtinv hdvetqfnqy kteaasrynl |
| 121 | tisdvsvsdv pfpfsaqsga gvpgwgiall vlvcvlvala ivylialavc qcrrknygql |
| 181 | difpardtyh pmseyptyht hgryvppsst drspyekvsa gnggsslsyt npavaatsan |
| 241 | l |
| Mucin-1, isoform 16 precursor NP_001191221.1 | |
| 1 | mtpgtqspff llllltvlta ttapkpatvv tgsghasstp ggeketsatq rssvpsstek |
| 61 | naipaptttk scretflkwp gsvvvqltla fregtinvhd vetqfnqykt eaasrynlti |
| 121 | sdvsvsdvpf pfsaqsgagv pgwgiallvl vcvlvalaiv ylialavcqc rrknygqldi |
| 181 | fpardtyhpm seyptyhthg ryvppsstdr spyekvsagn ggsslsytnp avaatsanl |
| Mucin-1, isoform 17 precursor NP_001191222.1 | |
| 1 | mtpgtqspff llllltvltv vtgsghasst pggeketsat qrssvpsste knalstgvsf |
| 61 | fflsfhisnl qfnssledps tdyyqelqrd isemflqiyk qggflglsni kfrpgsvvvq |
| 121 | ltlafregti nvhdvetqfn qykteaasry nitisdvsgc lsvppkelra aghlsspgyl |
| 181 | psyervphlp hpwalcap |
| Mucin-1, isoform 18 precursor NP_001191223.1 | |
| 1 | mtpgtqspff llllltvltv vtgsghasst pggeketsat qrssvpsste knaipapttt |
| 61 | kscretflkw pgsvvvqltl afregtinvh dvetqfnqyk teaasrynlt isdvsvsdvp |
| 121 | fpfsaqsgag vpgwgiallv lvcvlvalai vylialavcq crrknygqld ifpardtyhp |
| 181 | mseyptyhth gryvppsstd rspyekvsag nggsslsytn pavaatsanl |
| Mucin-1, isoform 19 precursor NP_001191224.1 | |
| 1 | mtpgtqspff llllltvlta ttapkpatvv tgsghasstp ggeketsatq rssvpsstek |
| 61 | nafnssledp stdyyqelqr disemsgagv pgwgiallvl vcvlvalaiv ylialavcqc |
| 121 | rrknygqldi fpardtyhpm seyptyhthg ryvppsstdr spyekvsagn ggsslsytnp |
| 181 | avaatsanl |
| Mucin-1, isoform 20 precursor NP_001191225.1 | |
| 1 | mtpgtqspff llllltvlta ttapkpatvv tgsghasstp ggeketsatq rssvpsstek |
| 61 | naipaptttk scretflkcf crfinkgvfw aspilssvsd vpfpfsaqsg agvpgwgial |
| 121 | lvlvcvlval aivylialav cqcrrknygq ldifpardty hpmseyptyh thgryvppss |
| 181 | tdrspyekvs agnggsslsy tnpavaatsa nl |
| Mucin-1, isoform 21 precursor NP_001191226.1 | |
| 1 | mtpgtqspff llllltvlta ttapkpatvv tgsghasstp ggeketsatq rssvpsstek |
| 61 | nalstgvsff flsfhisnlq fnssledpst dyyqelqrdi semavcqcrr knygqldifp |
| 121 | ardtyhpmse yptyhthgry vppsstdrsp yekvsagngg sslsytnpav aatsanl |
| N-myc proto-oncogene protein, isoform 1 NP_001280157.1, NP_005369.2 | |
| 1 | mpscststmp gmicknpdle fdslqpcfyp deddfyfggp dstppgediw kkfellptpp |
| 61 | lspsrgfaeh sseppswvte mllenelwgs paeedafglg glggltpnpv ilqdcmwsgf |
| 121 | sareklerav seklqhgrgp ptagstaqsp gagaaspagr ghggaagagr agaalpaela |
| 181 | hpaaecvdpa vvfpfpvnkr epapvpaapa sapaagpava sgagiaapag apgvapprpg |
| 241 | grqtsggdhk alstsgedtl sdsddeddee edeeeeidvv tvekrrsssn tkavttftit |
| 301 | vrpknaalgp graqsselil krclpihqqh nyaapspyve sedappqkki kseasprplk |
| 361 | svippkaksl sprnsdseds errrnhnile rqrrndlrss fltlrdhvpe lvknekaakv |
| 421 | vilkkateyv hslqaeehql llekeklqar qqqllkkieh artc |
| N-myc proto-oncogene protein, isoform 2 NP_001280160.1 | |
| 1 | mrgapgncvg aeqalarrkr aqtvairghp rppgppgdtr aesppdplqs agddeddeee |
| 61 | deeeeidvvt vekrrsssnt kavttftitv rpknaalgpg raqsselilk rclpihqqhn |
| 121 | yaapspyves edappqkkik seasprplks vippkaksls prnsdsedse rrrnhniler |
| 181 | qrrndlrssf ltlrdhvpel vknekaakvv ilkkateyvh slqaeehqll lekeklqarq |
| 241 | qqllkkieha rtc |
| N-myc proto-oncogene protein, isoform 3 NP_001280162.1 | |
| 1 | mrgapgncvg aeqalarrkr aqtvairghp rppgppgdtr aesppdplqs agvlevgagp |
| 61 | rlprppregs tpgiktngae rspqspagrr adaellhvhh aghdlqeprp rv |
| Cancer/testis antigen 1B NP_001318.1 | |
| 1 | mqaegrgtgg stgdadgpgg pgipdgpggn aggpgeagat ggrgprgaga arasgpggga |
| 61 | prgphggaas glngccrcga rgpesrllef ylampfatpm eaelarrsla qdapplpvpg |
| 121 | vllkeftvsg niltirltaa dhrqlqlsis sclqqlsllm witqcflpvf laqppsgqrr |
| Opioid growth factor receptor NP_031372.2 | |
| 1 | mddpdcdstw eedeedaeda ededcedgea agardadagd edeeseepra arpssfqsrm |
| 61 | tgsrnwratr dmcryrhnyp dlverdcngd tpnlsfyrne irflpngcfi edilqnwtdn |
| 121 | ydllednhsy iqwlfplrep gvnwhakplt lrevevfkss qeiqerlvra yelmlgfygi |
| 181 | rledrgtgtv graqnyqkrf qnlnwrshnn lritrilksl gelglehfqa plvrffleet |
| 241 | lvrrelpgvr qsaldyfmfa vrcrhqrrql vhfawehfrp rckfvwgpqd klrrfkpssl |
| 301 | phplegsrkv eeegspgdpd heastqgrtc gpehskgggr vdegpqprsv epqdagpler |
| 361 | sqgdeagghg edrpeplspk eskkrklels rreqpptepg pqsaseveki alnlegcals |
| 421 | qgslrtgtqe vggqdpgeav qpcrqplgar vadkvrkrrk vdegagdsaa vasggaqtla |
| 481 | lagspapsgh pkaghsengv eedtegrtgp kegtpgspse tpgpspagpa gdepaespse |
| 541 | tpgprpagpa gdepaespse tpgprpagpa gdepaespse tpgpspagpt rdepaespse |
| 601 | tpgprpagpa gdepaespse tpgprpagpa gdepaespse tpgpspagpt rdepakagea |
| 661 | aelqdaeves saksgkp |
| P antigen family member 4 NP_001305806.1, NP_008934.1 | |
| 1 | msarvrsrsr grgdgqeapd vvafvapges qqeepptdnq diepgqereg tppieerkve |
| 61 | gdcqemdlek trsergdgsd vkektppnpk haktkeagdg qp |
| Paired box protein Pax-3, isoform PAX3a NP_000429.2 | |
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkqvt tpdvekkiee |
| 121 | ykrenpgmfs weirdkllkd avcdrntvps vssisrilrs kfgkgeeeea dlerkeaees |
| 181 | ekkakhsidg ilsergkrwr lgrrtcwvtw rasas |
| Paired box protein Pax-3, isoform PAX3i NP_001120838.1 | |
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkvtt pdvekkieey |
| 121 | krenpgmfsw eirdkllkda vcdrntvpsv ssisrilrsk fgkgeeeead lerkeaeese |
| 181 | kkakhsidgi lserasapqs degsdidsep dlplkrkqrr srttftaeql eelerafert |
| 241 | hypdiytree laqrakltea rvqvwfsnrr arwrkqagan qlmafnhlip ggfpptampt |
| 301 | lptyqlsets yqptsipqav sdpsstvhrp qplppstvhq stipsnpdss sayclpstrh |
| 361 | gfssytdsfv ppsgpsnpmn ptignglspq vmglltnhgg vphqpqtdya lspltgglep |
| 421 | tttvsascsq rldhmkslds lptsqsycpp tysttgysmd pvtgyqygqy gqsafhylkp |
| 481 | dia |
| Paired box protein Pax-3, isoform PAX3b NP_039230.1 | |
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkqvt tpdvekkiee |
| 121 | ykrenpgmfs weirdkllkd avcdrntvps vssisrilrs kfgkgeeeea dlerkeaees |
| 181 | ekkakhsidg ilsergkalv sgvssh |
| Paired box protein Pax-3, isoform PAX3 NP_852122.1 | |
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkqvt tpdvekkiee |
| 121 | ykrenpgmfs weirdkllkd avcdrntvps vssisrilrs kfgkgeeeea dlerkeaees |
| 181 | ekkakhsidg ilserasapq sdegsdidse pdlplkrkqr rsrttftaeq leelerafer |
| 241 | thypdiytre elaqraklte arvqvwfsnr rarwrkqaga nqlmafnhli pggfpptamp |
| 301 | tlptyqlset syqptsipqa vsdpsstvhr pqplppstvh qstipsnpds ssayclpstr |
| 361 | hgfssytdsf vppsgpsnpm nptignglsp qvmglltnhg gvphqpqtdy alspltggle |
| 421 | ptttvsascs qrldhmksld slptsqsycp ptysttgysm dpvtgyqygq ygqskpwtf |
| Paired box protein Pax-3, isoform PAX3d NP_852123.1 | |
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkqvt tpdvekkiee |
| 121 | ykrenpgmfs weirdkllkd avcdrntvps vssisrilrs kfgkgeeeea dlerkeaees |
| 181 | ekkakhsidg ilserasapq sdegsdidse pdlplkrkqr rsrttftaeq leelerafer |
| 241 | thypdiytre elaqraklte arvqvwfsnr rarwrkqaga nqlmafnhli pggfpptamp |
| 301 | tlptyqlset syqptsipqa vsdpsstvhr pqplppstvh qstipsnpds ssayclpstr |
| 361 | hgfssytdsf vppsgpsnpm nptignglsp qvmglltnhg gvphqpqtdy alspltggle |
| 421 | ptttvsascs qrldhmksld slptsqsycp ptysttgysm dpvtgyqygq ygqsafhylk |
| 481 | pdia |
| Paired box protein Pax-3, isoform PAX3e NP_852124.1 | |
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkqvt tpdvekkiee |
| 121 | ykrenpgmfs weirdkllkd avcdrntvps vssisrilrs kfgkgeeeea dlerkeaees |
| 181 | ekkakhsidg ilserasapq sdegsdidse pdlplkrkqr rsrttftaeq leelerafer |
| 241 | thypdiytre elaqraklte arvqvwfsnr rarwrkqaga nqlmafnhli pggfpptamp |
| 301 | tlptyqlset syqptsipqa vsdpsstvhr pqplppstvh qstipsnpds ssayclpstr |
| 361 | hgfssytdsf vppsgpsnpm nptignglsp qvmglltnhg gvphqpqtdy alspltggle |
| 421 | ptttvsascs qrldhmksld slptsqsycp ptysttgysm dpvtgyqygq ygqsafhylk |
| 481 | pdiawfqill ntfdkssgee edleq |
| Paired box protein Pax-3, isoform PAX3h NP_852125.1 | |
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkqvt tpdvekkiee |
| 121 | ykrenpgmfs weirdkllkd avcdrntvps vssisrilrs kfgkgeeeea dlerkeaees |
| 181 | ekkakhsidg ilserasapq sdegsdidse pdlplkrkqr rsrttftaeq leelerafer |
| 241 | thypdiytre elaqraklte arvqvwfsnr rarwrkqaga nqlmafnhli pggfpptamp |
| 301 | tlptyqlset syqptsipqa vsdpsstvhr pqplppstvh qstipsnpds ssayclpstr |
| 361 | hgfssytdsf vppsgpsnpm nptignglsp qvpfiissqi slgfksf |
| Paired box protein Pax-3, isoform PAX3g NP_852126.1 | |
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkqvt tpdvekkiee |
| 121 | ykrenpgmfs weirdkllkd avcdrntvps vssisrilrs kfgkgeeeea dlerkeaees |
| 181 | ekkakhsidg ilserasapq sdegsdidse pdlplkrkqr rsrttftaeq leelerafer |
| 241 | thypdiytre elaqraklte arvqvwfsnr rarwrkqaga nqlmafnhli pggfpptamp |
| 301 | tlptyqlset syqptsipqa vsdpsstvhr pqplppstvh qstipsnpds ssayclpstr |
| 361 | hgfssytdsf vppsgpsnpm nptignglsp qvpfiissqi srk |
| Paired box protein Pax-5, isoform 1 NP_057953.1 | |
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp svssinriir tkvqqppnqp vpasshsivs tgsvtqvssv stdsagssys |
| 181 | isgilgitsp sadtnkrkrd egiqespvpn ghslpgrdfl rkqmrgdlft qqqlevldrv |
| 241 | ferqhysdif tttepikpeq tteysamasl agglddmkan lasptpadig ssvpgpqsyp |
| 301 | ivtgrdlast tlpgypphvp pagqgsysap tltgmvpgse fsgspyshpq yssyndswrf |
| 361 | pnpgllgspy yysaaargaa ppaaataydr h |
| Paired box protein Pax-5, isoform 2 NP_001267476.1 | |
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp svssinriir tkvqqppnqp vpasshsivs tgsvtqvssv stdsagssys |
| 181 | isgilgitsp sadtnkrkrd egiqespvpn ghslpgrdfl rkqmrgdlft qqqlevldrv |
| 241 | ferqhysdif tttepikpeq tteysamasl agglddmkan lasptpadig ssvpgpqsyp |
| 301 | ivtgsefsgs pyshpqyssy ndswrfpnpg llgspyyysa aargaappaa ataydrh |
| Paired box protein Pax-5, isoform 3 NP_001267477.1 | |
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp svssinriir tkvqqppnqp vpasshsivs tgsvtqvssv stdsagssys |
| 181 | isgilgitsp sadtnkrkrd egiqespvpn ghslpgrdfl rkqmrgdlft qqqlevldrv |
| 241 | ferqhysdif tttepikpeq tteysamasl agglddmkan lasptpadig ssvpgpqsyp |
| 301 | ivtgrdlast tlpgypphvp pagqgsysap tltgmvpgsp yyysaaarga appaaatayd |
| 361 | rh |
| Paired box protein Pax-5, isoform 4 NP_001267478.1 | |
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp svssinriir tkvqqppnqp vpasshsivs tgsvtqvssv stdsagssys |
| 181 | isgilgitsp sadtnkrkrd egiqespvpn ghslpgrdfl rkqmrgdlft qqqlevldrv |
| 241 | ferqhysdif tttepikpeq gvsfpgvpta tlsiprtttp ggsptrgcla pptiialppe |
| 301 | epphlqpplp mtvtdpwsqa gtkh |
| Paired box protein Pax-5, isoform 5 NP_001267479.1 | |
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp svssinriir tkvqqppnqp vpasshsivs tgsvtqvssv stdsagssys |
| 181 | isgilgitsp sadtnkrkrd egiqespvpn ghslpgrdfl rkqmrgdlft qqqlevldrv |
| 241 | ferqhysdif tttepikpeq apptiialpp eepphlqppl pmtvtdpwsq agtkh |
| Paired box protein Pax-5, isoform 6 NP_001267480.1 | |
| 1 | mfaweirdrl laervcdndt vpsvssinri irtkvqqppn qpvpasshsi vstgsvtqvs |
| 61 | svstdsagss ysisgilgit spsadtnkrk rdegiqespv pnghslpgrd flrkqmrgdl |
| 121 | ftqqqlevld rvferqhysd iftttepikp eqtteysama slagglddmk anlasptpad |
| 181 | igssvpgpqs ypivtgspyy ysaaargaap paaataydrh |
| Paired box protein Pax-5, isoform 7 NP_001267481.1 | |
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp svssinriir tkvqqppnqp vpasshsivs tgsvtqvssv stdsagssys |
| 181 | isgilgitsp sadtnkrkrd egiqespvpn ghslpgrdfl rkqmrgdlft qqqlevldrv |
| 241 | ferqhysdif tttepikpeq tteysamasl agglddmkan lasptpadig ssvpgpqsyp |
| 301 | ivtgspyyys aaargaappa aataydrh |
| Paired box protein Pax-5, isoform 8 NP_001267482.1 | |
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp svssinriir tkvqqppnqp vpasshsigi qespvpnghs lpgrdflrkg |
| 181 | mrgdlftqqq levldrvfer qhysdifttt epikpeqtte ysamaslagg lddmkanlas |
| 241 | ptpadigssv pgpqsypivt grdlasttlp gypphvppag qgsysaptlt gmvpgspyyy |
| 301 | saaargaapp aaataydrh |
| Paired box protein Pax-5, isoform 9 NP_001267483.1 | |
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp svssinriir tkvqqppnqp vpasshsigi qespvpnghs lpgrdflrkg |
| 181 | mrgdlftqqq levldrvfer qhysdifttt epikpeqtte ysamaslagg lddmkanlas |
| 241 | ptpadigssv pgpqsypivt grdlasttlp gypphvppag qgsysaptlt gmvpgsefsg |
| 301 | spyshpqyss yndswrfpnp gllgspyyys aaargaappa aataydrh |
| Paired box protein Pax-5, isoform 10 NP_001267484.1 | |
| 1 | mdleknyptp rtsrtghggv nqlggvfvng rplpdvvrqr ivelahqgvr pcdisrqlrv |
| 61 | shgcvskilg riirtkvqqp pnqpvpassh sivstgsvtq vssvstdsag ssysisgilg |
| 121 | itspsadtnk rkrdegiqes pvpnghslpg rdflrkqmrg dlftqqqlev ldrvferqhy |
| 181 | sdiftttepi kpeqtteysa maslaggldd mkanlasptp adigssvpgp qsypivtgse |
| 241 | fsgspyshpq yssyndswrf pnpgllgspy yysaaargaa ppaaataydr h |
| Paired box protein Pax-5, isoform 11 NP_001267485.1 | |
| 1 | mfaweirdrl laervcdndt vpsvssinri irtkvqqppn qpvpasshsi vstgsvtqvs |
| 61 | svstdsagss ysisgilgit spsadtnkrk rdegiqespv pnghslpgrd flrkqmrgdl |
| 121 | ftqqqlevld rvferqhysd iftttepikp eqtteysama slagglddmk anlasptpad |
| 181 | igssvpgpqs ypivtgrdla sttlpgypph vppagqgsys aptltgmvpg sefsgspysh |
| 241 | pqyssyndsw rfpnpgllgs pyyysaaarg aappaaatay drh |
| Platelet-derived growth factor receptor beta, isoform 1 NP_002600.1 | |
| 1 | mrlpgampal alkgelllls lllllepqis qglvvtppgp elvlnvsstf vltcsgsapv |
| 61 | vwermsqepp qemakaqdgt fssvltltnl tgldtgeyfc thndsrglet derkrlyifv |
| 121 | pdptvgflpn daeelfiflt eiteitipcr vtdpqlvvtl hekkgdvalp vpydhqrgfs |
| 181 | gifedrsyic kttigdrevd sdayyvyrlq vssinvsvna vqtvvrqgen itlmcivign |
| 241 | evvnfewtyp rkesgrlvep vtdflldmpy hirsilhips aeledsgtyt cnvtesvndh |
| 301 | qdekainitv vesgyvrllg evgtlqfael hrsrtlqvvf eayppptvlw fkdnrtlgds |
| 361 | sageialstr nvsetryvse ltlvrvkvae aghytmrafh edaevqlsfq lqinvpvrvl |
| 421 | elseshpdsg eqtvrcrgrg mpqpniiwsa crdlkrcpre lpptllgnss eeesqletnv |
| 481 | tyweeeqefe vvstlrlqhv drplsvrctl rnavgqdtqe vivvphslpf kvvvisaila |
| 541 | lvvltiisli ilimlwqkkp ryeirwkvie svssdgheyi yvdpmqlpyd stwelprdql |
| 601 | vlgrtlgsga fgqvveatah glshsqatmk vavkmlksta rssekqalms elkimshlgp |
| 661 | hlnvvnllga ctkggpiyii teycrygdlv dylhrnkhtf lqhhsdkrrp psaelysnal |
| 721 | pvglplpshv sltgesdggy mdmskdesvd yvpmldmkgd vkyadiessn ymapydnyvp |
| 781 | sapertcrat linespvlsy mdlvgfsyqv angmeflask ncvhrdlaar nvlicegklv |
| 841 | kicdfglard imrdsnyisk gstflplkwm apesifnsly ttlsdvwsfg illweiftlg |
| 901 | gtpypelpmn eqfynaikrg yrmaqpahas deiyeimqkc weekfeirpp fsqlvlller |
| 961 | llgegykkky qqvdeeflrs dhpailrsqa rlpgfhglrs pldtssvlyt avqpnegdnd |
| 1021 | yiiplpdpkp evadegpleg spslasstln evntsstisc dsplepqdep epepqlelqv |
| 1081 | epepeleqlp dsgcpaprae aedsfl |
| Platelet-derived growth factor receptor beta, isoform 2 NP_001341945.1 | |
| 1 | msqeppqema kaqdgtfssv ltltnltgld tgeyfcthnd srgletderk rlyifvpdpt |
| 61 | vgflpndaee lfiflteite itipcrvtdp qlvvtlhekk gdvalpvpyd hqrgfsgife |
| 121 | drsyicktti gdrevdsday yvyrlqvssi nvsvnavqtv vrqgenitlm civignevvn |
| 181 | fewtyprkes grlvepvtdf lldmpyhirs ilhipsaele dsgtytcnvt esvndhqdek |
| 241 | ainitvvesg yvrllgevgt lqfaelhrsr tlqvvfeayp pptvlwfkdn rtlgdssage |
| 301 | ialstrnvse tryvseltlv rvkvaeaghy tmrafhedae vqlsfqlqin vpvrvlelse |
| 361 | shpdsgeqtv rcrgrgmpqp niiwsacrdl krcprelppt llgnsseees qletnvtywe |
| 421 | eeqefevvst lrlqhvdrpl svrctlrnav gqdtqevivv phslpfkvvv isailalvvl |
| 481 | tiisliilim lwqkkpryei rwkviesvss dgheyiyvdp mqlpydstwe lprdqlvlgr |
| 541 | tlgsgafgqv veatahglsh sqatmkvavk mlkstarsse kqalmselki mshlgphlnv |
| 601 | vnllgactkg gpiyiiteyc rygdlvdylh rnkhtflqhh sdkrrppsae lysnalpvgl |
| 661 | plpshvsltg esdggymdms kdesvdyvpm ldmkgdvkya diessnymap ydnyvpsape |
| 721 | rtcratline spvlsymdlv gfsyqvangm eflaskncvh rdlaarnvli cegklvkicd |
| 781 | fglardimrd snyiskgstf lplkwmapes ifnslyttls dvwsfgillw eiftlggtpy |
| 841 | pelpmneqfy naikrgyrma qpahasdeiy eimqkcweek feirppfsql vlllerllge |
| 901 | gykkkyqqvd eeflrsdhpa ilrsqarlpg fhglrspldt ssvlytavqp negdndyiip |
| 961 | lpdpkpevad egplegspsl asstlnevnt sstiscdspl epqdepepep qlelqvepep |
| 1021 | eleqlpdsgc papraeaeds fl |
| Platelet-derived growth factor receptor beta, isoform 3 NP_001341946.1 | |
| 1 | mitnvaflvs lrteatsakp plgtgrwilm ptmstdsrvs plsglmlsrv ssinvsvnav |
| 61 | qtvvrqgeni tlmcivigne vvnfewtypr kesgrlvepv tdflldmpyh irsilhipsa |
| 121 | eledsgtytc nvtesvndhq dekainitvv esgyvrllge vgtlqfaelh rsrtlqvvfe |
| 181 | ayppptvlwf kdnrtlgdss ageialstrn vsetryvsel tlvrvkvaea ghytmrafhe |
| 241 | daevqlsfql qinvpvrvle lseshpdsge qtvrcrgrgm pqpniiwsac rdlkrcprel |
| 301 | pptllgnsse eesqletnvt yweeeqefev vstlrlqhvd rplsvrctlr navgqdtqev |
| 361 | ivvphslpfk vvvisailal vvltiislii limlwqkkpr yeirwkvies vssdgheyiy |
| 421 | vdpmqlpyds twelprdqlv lgrtlgsgaf gqvveatahg lshsqatmkv avkmlkstar |
| 481 | ssekqalmse lkimshlgph lnvvnllgac tkggpiyiit eycrygdlvd ylhrnkhtfl |
| 541 | qhhsdkrrpp saelysnalp vglplpshvs ltgesdggym dmskdesvdy vpmldmkgdv |
| 601 | kyadiessny mapydnyvps apertcratl inespvlsym dlvgfsyqva ngmeflaskn |
| 661 | cvhrdlaarn vlicegklvk icdfglardi mrdsnyiskg stflplkwma pesifnslyt |
| 721 | tlsdvwsfgi llweiftlgg tpypelpmne qfynaikrgy rmaqpahasd eiyeimqkcw |
| 781 | eekfeirppf sqlvlllerl lgegykkkyq qvdeeflrsd hpailrsqar lpgfhglrsp |
| 841 | ldtssvlyta vqpnegdndy iiplpdpkpe vadegplegs pslasstlne vntsstiscd |
| 901 | splepqdepe pepqlelqve pepeleqlpd sgcpapraea edsfl |
| Placenta-specific protein 1 precursor NP_001303816.1, NP_001303817.1, | |
| NP_001303818.1, NP_068568.1 | |
| 1 | mkvfkfiglm illtsafsag sgqspmtvlc sidwfmvtvh pfmlnndvcv hfhelhlglg |
| 61 | cppnhvqpha yqftyrvtec girakavsqd mviysteihy sskgtpskfv ipvscaapqk |
| 121 | spwltkpcsm rvasksrata qkdekcyevf slsqssqrpn cdcppcvfse eehtqvpchq |
| 181 | agaqeaqplq pshfldised wslhtddmig sm |
| Melanoma antigen preferentially expressed in tumors, isoform a | |
| NP_001278644.1, NP_001278645.1, NP_006106.1, NP_996836.1, NP_996837.1, | |
| NP_996838.1, NP_996839.1 | |
| 1 | merrrlwgsi qsryismsvw tsprrlvela gqsllkdeal aiaalellpr elfpplfmaa |
| 61 | fdgrhsqtlk amvqawpftc lplgvlmkgq hlhletfkav ldgldvllaq evrprrwklq |
| 121 | vldlrknshq dfwtvwsgnr aslysfpepe aaqpmtkkrk vdglsteaeq pfipvevlvd |
| 181 | lflkegacde lfsyliekvk rkknvlrlcc kklkifampm qdikmilkmv qldsiedlev |
| 241 | tctwklptla kfspylgqmi nlrrlllshi hassyispek eeqyiaqfts qflslqclqa |
| 301 | lyvdslfflr grldqllrhv mnpletlsit ncrlsegdvm hlsqspsvsq lsvlslsgvm |
| 361 | ltdvspeplq allerasatl qdlvfdecgi tddqllallp slshcsqltt lsfygnsisi |
| 421 | salqsllqhl iglsnlthvl ypvplesyed ihgtlhlerl aylharlrel lcelgrpsmv |
| 481 | wlsanpcphc gdrtfydpep ilcpcfmpn |
| Melanoma antigen preferentially expressed in tumors, isoform b | |
| NP_001278646.1, NP_001278648.1, NP_001305055.1, NP_001305056.1 | |
| 1 | msvwtsprrl velagqsllk dealaiaale llprelfppl fmaafdgrhs qtlkamvqaw |
| 61 | pftclplgvl mkgqhlhlet fkavldgldv llaqevrprr wklqvldlrk nshqdfwtvw |
| 121 | sgnraslysf pepeaaqpmt kkrkvdglst eaeqpfipve vlvdlflkeg acdelfsyli |
| 181 | ekvkrkknvl rlcckklkif ampmqdikmi lkmvqldsie dlevtctwkl ptlakfspyl |
| 241 | gqminlrrll lshihassyi spekeeqyia qftsqflslq clqalyvdsl fflrgrldql |
| 301 | lrhvmnplet lsitncrlse gdvmhlsgsp sysqlsvlsl sgvmltdvsp eplqallera |
| 361 | satlqdlvfd ecgitddqll allpslshcs qlttlsfygn sisisalqsl lqhliglsnl |
| 421 | thvlypvple syedihgtlh lerlaylhar lrellcelgr psmvwlsanp cphcgdrtfy |
| 481 | dpepilcpcf mpn |
| Phosphatidylinositol 3,4,5-triphosphate-dependent Rac exchanger 2 | |
| protein, isoform a NP_079146.2 | |
| 1 | msedsrgdsr aesakdlekq lrlrvcvlse lqkterdyvg tleflvsafl hrmnqcaask |
| 61 | vdknvteetv kmlfsniedi lavhkeflkv veeclhpepn aqqevgtcfl hfkdkfriyd |
| 121 | eycsnhekaq klllelnkir tirtfllncm llggrkntdv plegylvtpi qrickyplil |
| 181 | kellkrtprk hsdyaavmea lqamkavcsn ineakrqmek levleewqsh iegwegsnit |
| 241 | dtctemlmcg vllkissgni qervfflfdn llvyckrkhr rlknskastd ghrylfrgri |
| 301 | ntevmevenv ddgtadfhss ghivvngwki hntaknkwfv cmaktpeekh ewfeailker |
| 361 | errkglklgm eqdtwvmise qgeklykmmc rqgnlikdrk rklttfpkcf lgsefvswll |
| 421 | eigeihrpee gvhlgqalle ngiihhvtdk hqfkpeqmly rfryddgtfy prnemqdvis |
| 481 | kgvrlycrlh slftpvirdk dyhlrtyksv vmanklidwl iaqgdcrtre eamifgvglc |
| 541 | dngfmhhvle ksefkdepll frffsdeeme gsnmkhrlmk hdlkvvenvi akslliksne |
| 601 | gsygfgledk nkvpiiklve kgsnaemagm evgkkifain gdlvfmrpfn evdcflkscl |
| 661 | nsrkplrvlv stkpretvki pdsadglgfq irgfgpsvvh avgrgtvaaa aglhpgqcii |
| 721 | kvnginvske thasviahvt acrkyrrptk qdsiqwvyns iesaqedlqk shskppgdea |
| 781 | gdafdckvee vidkfntmai idgkkehvsl tvdnvhleyg vvyeydstag ikcnvvekmi |
| 841 | epkgffslta kilealaksd ehfvqnctsl nslneviptd lqskfsalcs eriehlcqri |
| 901 | ssykkfsrvl knrawptfkq akskisplhs sdfcptnchv nvmevsypkt stslgsafgv |
| 961 | qldsrkhnsh dkenksseqg klspmvyiqh tittmaapsg lslgqqdghg lryllkeedl |
| 1021 | etqdiyqkll gklqtalkev emcvcqiddl lssityspkl erktsegiip tdsdnekger |
| 1081 | nskrvcfnva gdeqedsghd tisnrdsysd cnsnrnsias ftsicssqcs syfhsdemds |
| 1141 | gdelplsvri shdkqdkihs clehlfsqvd sitnllkgqa vvrafdqtky ltpgrglqef |
| 1201 | qqemepklsc pkrlrlhikq dpwnlpssvr tlaqnirkfv eevkcrllla lleysdsetq |
| 1261 | lrrdmvfcqt lvatvcafse qlmaalnqmf dnskenemet weasrrwldq ianagvlfhf |
| 1321 | qsllspnltd eqamledtlv alfdlekvsf yfkpseeepl vanvpltyqa egsrgalkvy |
| 1381 | fyidsyhfeq lpqrlknggg fkihpvlfaq alesmegyyy rdnvsveefq aqinaaslek |
| 1441 | vkgynqklra fyldksnspp nstskaayvd klmrplnald elyrlvasfi rskrtaacan |
| 1501 | tacsasgvgl lsysselcnr lgachiimcs sgvhrctlsv tleqaiilar shglppryim |
| 1561 | qatdvmrkqg arvqntaknl gvrdrtpqsa prlyklcepp ppagee |
| Phosphatidylinositol 3,4,5-triphosphate-dependent Rac exchanger 2 | |
| protein, isoform b NP_079446.3 | |
| 1 | msedsrgdsr aesakdlekq lrlrvcvlse lqkterdyvg tleflvsafl hrmnqcaask |
| 61 | vdknvteetv kmlfsniedi lavhkeflkv veeclhpepn aqqevgtcfl hfkdkfriyd |
| 121 | eycsnhekaq klllelnkir tirtfllncm llggrkntdv plegylvtpi qrickyplil |
| 181 | kellkrtprk hsdyaavmea lqamkavcsn ineakrqmek levleewqsh iegwegsnit |
| 241 | dtctemlmcg vllkissgni qervfflfdn llvyckrkhr rlknskastd ghrylfrgri |
| 301 | ntevmevenv ddgtadfhss ghivvngwki hntaknkwfv cmaktpeekh ewfeailker |
| 361 | errkglklgm eqdtwvmise qgeklykmmc rqgnlikdrk rklttfpkcf lgsefvswll |
| 421 | eigeihrpee gvhlgqalle ngiihhvtdk hqfkpeqmly rfryddgtfy prnemqdvis |
| 481 | kgvrlycrlh slftpvirdk dyhlrtyksv vmanklidwl iaqgdcrtre eamifgvglc |
| 541 | dngfmhhvle ksefkdepll frffsdeeme gsnmkhrlmk hdlkvvenvi akslliksne |
| 601 | gsygfgledk nkvpiiklve kgsnaemagm evgkkifain gdlvfmrpfn evdcflkscl |
| 661 | nsrkplrvlv stkpretvki pdsadglgfq irgfgpsvvh avgrgtvaaa aglhpgqcii |
| 721 | kvnginvske thasviahvt acrkyrrptk qdsigwvyns iesaqedlqk shskppgdea |
| 781 | gdafdckvee vidkfntmai idgkkehvsl tvdnvhleyg vvyeydstag ikcnvvekmi |
| 841 | epkgffslta kilealaksd ehfvqnctsl nslneviptd lqskfsalcs eriehlcqri |
| 901 | ssykkvqase rfynftarha vwehsfdlhs vsstfpvpvt meflllpppl lgisqdgrqh |
| 961 | cipedlpsqe mllaerapv |
| Protamine-2, isoform 1 NP_002753.2 | |
| 1 | mvryrvrsls ershevyrqq lhgqeqghhg qeeqglspeh vevyerthgq shyrrrhcsr |
| 61 | rrlhrihrrq hrscrrrkrr scrhrrrhrr gcrtrkrtcr rh |
| Protamine-2, isoform 2 NP_001273285.1 | |
| 1 | mvryrvrsls ershevyrqq lhgqeqghhg qeeqglspeh vevyerthgq shyrrrhcsr |
| 61 | rrlhrihrrq hrscrrrkrr scrhrrrhrr eslgdplnqn flsqkaaepg rehaegtklp |
| 121 | gpltpswklr ksrpkhqvrp |
| Protamine-2, isoform 3 NP_001273286.1 | |
| 1 | mvryrvrsls ershevyrqq lhgqeqghhg qeeqglspeh vevyerthgq shyrrrhcsr |
| 61 | rrlhrihrrq hrscrrh |
| Protamine-2, isoform 4 NP_001273287.1 | |
| 1 | mvryrvrsls ershevyrqq lhgqeqghhg qeeqglspeh vevyerthgq shyrrrhcsr |
| 61 | rrlhrihrrq hrscrrrkrr scrhrrrhrr epgrehaegt klpgpltpsw klrksrpkhq |
| 121 | vrp |
| Protamine-2, isoform 5 NP_001273288.1 | |
| 1 | mvryrvrsls ershevyrqq lhgqeqghhg qeeqglspeh vevyerthgq shyrrrhcsr |
| 61 | rrlhrihrrq hrscrrrkrr scrhrrrhrr glpapppcpa cp |
| Progranulin NP_002078.1 | |
| 1 | mwtlvswval taglvagtrc pdgqfcpvac cldpggasys ccrplldkwp ttlsrhlggp |
| 61 | cqvdahcsag hsciftvsgt ssccpfpeav acgdghhccp rgfhcsadgr scfqrsgnns |
| 121 | vgaiqcpdsq fecpdfstcc vmvdgswgcc pmpqascced rvhccphgaf cdlvhtrcit |
| 181 | ptgthplakk lpaqrtnrav alsssvmcpd arsrcpdgst ccelpsgkyg ccpmpnatcc |
| 241 | sdhlhccpqd tvcdliqskc lskenattdl ltklpahtvg dvkcdmevsc pdgytccrlq |
| 301 | sgawgccpft qavccedhih ccpagftcdt qkgtceqgph qvpwmekapa hlslpdpgal |
| 361 | krdvpcdnvs scpssdtccq ltsgewgccp ipeavccsdh qhccpqgytc vaegqcqrgs |
| 421 | eivaglekmp arraslshpr digcdqhtsc pvgqtccpsl ggswaccqlp havccedrqh |
| 481 | ccpagytcnv karscekevv saqpatflar sphvgvkdve cgeghfchdn qtccrdnrqg |
| 541 | waccpyrqgv ccadrrhccp agfrcaargt kclrreaprw daplrdpalr qll |
| Myeloblastin precursor NP_002768.3 | |
| 1 | mahrppspal asvllallls gaaraaeivg gheaqphsrp ymaslqmrgn pgshfcggtl |
| 61 | ihpsfvltaa hclrdipqrl vnvvlgahnv rtqeptqqhf svaqvflnny daenklndvl |
| 121 | liqlsspanl sasvatvqlp qqdqpvphgt qclamgwgrv gandppaqvl qelnvtvvtf |
| 181 | fcrphnictf vprrkagicf gdsggplicd giiqgidsfv iwgcatrlfp dfftrvalyv |
| 241 | dwirstlrrv eakgrp |
| Prostate stem cell antigen preportein NP_005663.2 | |
| 1 | maglalqpgt allcysckaq vsnedclqve nctqlgeqcw tariravgll tviskgcsln |
| 61 | cvddsqdyyv gkknitccdt dlcnasgaha lqpaaailal lpalglllwg pgql |
| Ras-related C3 botulinum toxin substrate 1 isoform Rac1b NP_061485.1 | |
| 1 | mqaikcvvvg dgavgktcll isyttnafpg eyiptvfdny sanvmvdgkp vnlglwdtag |
| 61 | qedydrlrpl sypqtvgety gkditsrgkd kpiadvflic fslvspasfe nvrakwypev |
| 121 | rhhcpntpii lvgtkldlrd dkdtieklke kkltpitypq glamakeiga vkylecsalt |
| 181 | qrglktvfde airavlcppp vkkrkrkcll l |
| Regenerating islet-derived protein 3-alpha precursor NP_002571.1, | |
| NP_620354.1, NP_620355.1 | |
| 1 | mlppmalpsv swmllsclml lsqvqgeepq relpsarirc pkgskaygsh cyalflspks |
| 61 | wtdadlacqk rpsgnlvsvl sgaegsfvss lvksignsys yvwiglhdpt qgtepngegw |
| 121 | ewsssdvmny fawernpsti sspghcasls rstaflrwkd yncnvrlpyv ckftd |
| Regulator of G-protein signaling 5, isoform 1 NP_003608.1 | |
| 1 | mckglaalph sclerakeik iklgillqkp dsvgdlvipy nekpekpakt qktsldealq |
| 61 | wrdsldkllq nnyglasfks flksefseen lefwiacedy kkikspakma ekakqiyeef |
| 121 | iqteapkevn idhftkditm knlvepslss fdmaqkriha lmekdslprf vrsefyqeli |
| 181 | k |
| Regulator of G-protein signaling 5, isoform 2 NP_001182232.1, | |
| NP_001241677.1 | |
| 1 | maekakqiye efiqteapke vnidhftkdi tmknlvepsl ssfdmaqkri halmekdslp |
| 61 | rfvrsefyqe lik |
| Regulator of G-protein signaling 5, isoform 3 NP_001241678.1 | |
| 1 | mckglaalph sclerakeik iklgillqkp dsvgdlvipy nekpekpakt qktsldealq |
| 61 | wrdsldkllq nnyglasfks flksefseen lefwiacedy kkikspakma ekakqiyeef |
| 121 | iqteapkevg lwvnidhftk ditmknlvep slssfdmaqk rihalmekds lprfvrsefy |
| 181 | qelik |
| Rho-related GTP-binding protein RhoC precursor NP_001036143.1, | |
| NP_001036144.1, NP_786886.1 | |
| 1 | maairkklvi vgdgacgktc llivfskdqf pevyvptvfe nyiadievdg kqvelalwdt |
| 61 | agqedydrlr plsypdtdvi lmcfsidspd slenipekwt pevkhfcpnv piilvgnkkd |
| 121 | lrqdehtrre lakmkqepvr seegrdmanr isafgylecs aktkegvrev fematraglq |
| 181 | vrknkrrrgc pil |
| Sarcoma antigen 1 NP_061136.2 | |
| 1 | mqasplqtsq ptppeelhaa ayvftndgqq mrsdevnlva tghqskkkhs rkskrhsssk |
| 61 | rrksmsswld kqedaavths iceerinngq pvadnvlsta ppwpdatiah nireermeng |
| 121 | qsrtdkvlst appqlvhmaa agipsmstrd lhstvthnir eermengqpq pdnvlstgpt |
| 181 | glinmaatpi pamsardlya tvthnvceqk menvqpapdn vlltlrprri nmtdtgispm |
| 241 | strdpyatit ynvpeekmek gqpqpdnils tastglinva gagtpaistn glystvphnv |
| 301 | ceekmendqp qpnnvlstvq pviiyltatg ipgmntrdqy atithnvcee rvvnnqplps |
| 361 | nalstvlpgl aylatadmpa mstrdqhati ihnlreekkd nsqptpdnvl savtpelinl |
| 421 | agagippmst rdqyatvnhh vhearmengq rkqdnvlsnv lsglinmaga sipamssrdl |
| 481 | yatithsvre ekmesgkpqt dkvisndapq lghmaaggip smstkdlyat vtqnvheerm |
| 541 | ennqpqpsyd lstvlpglty ltvagipams trdqyatvth nvheekikng qaasdnvfst |
| 601 | vppafinmaa tgvssmstrd qyaavthnir eekinnsqpa pgnilstapp wlrhmaaagi |
| 661 | sstitrdlyv tathsvheek mtngqqapdn slstvppgci nlsgagiscr strdlyatvi |
| 721 | hdiqeeemen dqtppdgfls nsdspelinm tghcmppnal dsfshdftsl skdellykpd |
| 781 | snefavgtkn ysvsagdppv tvmslvetvp ntpqispama kkinddikyq lmkevrrfgq |
| 841 | nyerifille evqgsmkvkr qfveftikea arfkkvvliq qlekalkeid shchlrkvkh |
| 901 | mrkr |
| Squamous cell carcinoma antigen recognized by T-cells 3 NP_055521.1 | |
| 1 | mataaetsas epeaeskagp kadgeedevk aartrrkvls ravaaatykt mgpawdqqee |
| 61 | gvsesdgdey amassaessp geyeweydee eeknqleier leeqlsinvy dynchvdlir |
| 121 | llrlegeltk vrmarqkmse ifplteelwl ewlhdeisma qdgldrehvy dlfekavkdy |
| 181 | icpniwleyg qysvggigqk gglekvrsvf eralssvglh mtkglalwea yrefesaive |
| 241 | aarlekvhsl frrqlaiply dmeatfaeye ewsedpipes viqnynkalq qlekykpyee |
| 301 | allqaeaprl aeyqayidfe mkigdpariq liferalven clvpdlwiry sqyldrqlkv |
| 361 | kdlvlsvhnr airncpwtva lwsryllame rhgvdhqvis vtfekalnag fiqatdyvei |
| 421 | wqayldylrr rvdfkqdssk eleelraaft raleylkqev eerfnesgdp scvimqnwar |
| 481 | iearlcnnmq karelwdsim trgnakyanm wleyynlera hgdtqhcrka lhravqctsd |
| 541 | ypehvcevll tmertegsle dwdiavqkte trlarvneqr mkaaekeaal vqqeeekaeq |
| 601 | rkraraekka lkkkkkirgp ekrgadedde kewgddeeeq pskrrrvens ipaagetqnv |
| 661 | evaagpagkc aavdveppsk qkekaaslkr dmpkvlhdss kdsitvfvsn lpysmqepdt |
| 721 | klrplfeacg evvqirpifs nrgdfrgycy vefkeeksal qalemdrksv egrpmfvspc |
| 781 | vdksknpdfk vfrystslek hklfisglpf sctkeeleei ckahgtvkdl rlvtnragkp |
| 841 | kglayveyen esqasqavmk mdgmtikeni ikvaisnppq rkvpekpetr kapggpmllp |
| 901 | qtygargkgr tqlsllpral qrpsaaapqa engpaaapav aapaateapk msnadfaklf |
| 961 | lrk |
| Secretory leukocyte protein inhibitor NP_003055.1 | |
| 1 | mkssglfpfl vllalgtlap wavegsgksf kagvcppkks aqclrykkpe cqsdwqcpgk |
| 61 | krccpdtcgi kcldpvdtpn ptrrkpgkcp vtygqclmln ppnfcemdgq ckrdlkccmg |
| 121 | mcgkscvspv ka |
| Transcription factor SOX-10 NP_008872.1 | |
| 1 | maeeqdlsev elspvgseep rclspgsaps lgpdgggggs glraspgpge lgkvkkeqqd |
| 61 | geadddkfpv cireavsqvl sgydwtlvpm pvrvngasks kphvkrpmna fmvwaqaarr |
| 121 | kladqyphlh naelsktlgk lwrllnesdk rpfieeaerl rmqhkkdhpd ykyqprrrkn |
| 181 | gkaaqgeaec pggeaeqggt aaiqahyksa hldhrhpgeg spmsdgnpeh psgqshgppt |
| 241 | ppttpktelq sgkadpkrdg rsmgeggkph idfgnvdige ishevmsnme tfdvaeldqy |
| 301 | lppnghpghv ssysaagygl gsalavasgh sawiskppgv alptvsppgv dakaqvktet |
| 361 | agpqgpphyt dqpstsqiay tslslphygs afpsisrpqf dysdhqpsgp yyghsgqasg |
| 421 | lysafsymgp sqrplytais dpspsgpqsh spthweqpvy ttlsrp |
| Sperm surface protein Sp17 NP_059121.1 | |
| 1 | msipfsnthy ripqgfgnll egltreilre qpdnipafaa ayfesllekr ektnfdpaew |
| 61 | gskvedrfyn nhafeeqepp eksdpkqees qisgkeeets vtildsseed kekeevaavk |
| 121 | iqaafrghia reeakkmktn slqneekeen k |
| Protein SSX2, isoform a NP_003138.3 | |
| 1 | mngddafarr ptvgaqipek iqkafddiak yfskeewekm kasekifyvy mkrkyeamtk |
| 61 | lgfkatlppf mcnkraedfq gndldndpnr gnqverpqmt fgrlqgispk impkkpaeeg |
| 121 | ndseevpeas gpqndgkelc ppgkpttsek ihersgnrea qekeerrgta hrwssqnthn |
| 181 | igrfslstsm gavhgtpkti thnrdpkggn mpgptdcvre nsw |
| Protein SSX2, isoform b NP_783629.1 | |
| 1 | mngddafarr ptvgaqipek iqkafddiak yfskeewekm kasekifyvy mkrkyeamtk |
| 61 | lgfkatlppf mcnkraedfq gndldndpnr gnqverpqmt fgrlqgispk impkkpaeeg |
| 121 | ndseevpeas gpqndgkelc ppgkpttsek ihersgpkrg ehawthrlre rkqlviyeei |
| 181 | sdpeedde |
| Protein SSX2, isoform c NP_001265626.1 | |
| 1 | mngddafarr ptvgaqipek iqkafddiak yfskeewekm kasekifyvy mkrkyeamtk |
| 61 | lgfkatlppf mcnkraedfq gndldndpnr gnqverpqmt fgrlqgispk impkkpaeeg |
| 121 | ndseevpeas gpqndgkelc ppgkpttsek ihersgnrea qekeerrgta hrwssqnthn |
| 181 | igpkrgehaw thrlrerkql viyeeisdpe edde |
| Lactosylceramide alpha-2,3-sialyltransferase, isoform 1 NP_003887.3 | |
| 1 | mrtkaagcae rrplqprtea aaapagramp seytyvklrs dcsrpslqwy traqskmrrp |
| 61 | slllkdilkc tllvfgvwil yilklnytte ecdmkkmhyv dpdhvkraqk yaqqvlqkec |
| 121 | rpkfaktsma llfehrysvd llpfvqkapk dseaeskydp pfgfrkfssk vqtllellpe |
| 181 | hdlpehlkak tcrrcvvigs ggilhglelg htlnqfdvvi rlnsapvegy sehvgnktti |
| 241 | rmtypegapl sdleyysndl fvavlfksvd fnwlqamvkk etlpfwvrlf fwkqvaekip |
| 301 | lqpkhfriln pviiketafd ilqysepqsr fwgrdknvpt igviavvlat hlcdevslag |
| 361 | fgydlnqprt plhyfdsqcm aamnfqtmhn vttetkfllk lvkegvvkdl sggidref |
| Lactosylceramide alpha-2,3-sialyltransferase, isoform 2 NP_001035902.1 | |
| 1 | masvpmpsey tyvklrsdcs rpslqwytra qskmrrpsll lkdilkctll vfgvwilyil |
| 61 | klnytteecd mkkmhyvdpd hvkraqkyaq qvlqkecrpk faktsmallf ehrysvdllp |
| 121 | fvqkapkdse aeskydppfg frkfsskvqt llellpehdl pehlkaktcr rcvvigsggi |
| 181 | lhglelghtl nqfdvvirln sapvegyseh vgnkttirmt ypegaplsdl eyysndlfva |
| 241 | vlfksvdfnw lqamvkketl pfwvrlffwk qvaekiplqp khfrilnpvi iketafdilq |
| 301 | ysepqsrfwg rdknvptigv iavvlathlc devslagfgy dlnqprtplh yfdsqcmaam |
| 361 | nfqtmhnvtt etkfllklvk egvvkdlsgg idref |
| Lactosylceramide alpha-2,3-sialyltransferase, isoform 3 | |
| NP_001341152.1, NP_001341153.1, NP_001341155.1, NP_001341162.1, | |
| NP_001341163.1, NP_001341177.1 | |
| 1 | mallfehrys vdllpfvqka pkdseaesky dppfgfrkfs skvqtllell pehdlpehlk |
| 61 | aktcrrcvvi gsggilhgle lghtlnqfdv virinsapve gysehvgnkt tirmtypega |
| 121 | plsdleyysn dlfvavlfks vdfnwlqamv kketlpfwvr lffwkqvaek iplqpkhfri |
| 181 | lnpviiketa fdilqysepq srfwgrdknv ptigviavvl athlcdevsl agfgydlnqp |
| 241 | rtplhyfdsq cmaamnfqtm hnvttetkfl lklvkegvvk dlsggidref |
| Lactosylceramide alpha-2,3-sialyltransferase, isoform 4 | |
| NP_001341156.1, NP_001341158.1, NP_001341167.1 | |
| 1 | mpseytyvkl rsdcsrpslq wytraqskmr rpslllkdil kctllvfgvw ilyilklnyt |
| 61 | teecdmkkmh yvdpdhvkra qkyaqqvlqk ecrpkfakts mallfehrys vdllpfvqka |
| 121 | pkdseaesky dppfgfrkfs skvqtllell pehdlpehlk aktcrrcvvi gsggilhgle |
| 181 | lghtlnqfdv virlnsapve gysehvgnkt tirmtypega plsdleyysn dlfvavlfks |
| 241 | vdfnwlqamv kketlpfwvr lffwkqvaek iplqpkhfri lnpviiketa fdilqysepq |
| 301 | srfwgrdknv ptigviavvl athlcdevsl agfgydlnqp rtplhyfdsq cmaamnfqtm |
| 361 | hnvttetkfl lklvkegvvk dlsggidref |
| Lactosylceramide alpha-2,3-sialyltransferase, isoform 5 NP_001341176.1 | |
| 1 | mtypegapls dleyysndlf vavlfksvdf nwlqamvkke tlpfwvrlff wkqvaekipl |
| 61 | qpkhfrilnp viiketafdi lqysepqsrf wgrdknvpti gviavvlath lcdevslagf |
| 121 | gydlnqprtp lhyfdsqcma amnfqtmhnv ttetkfllkl vkegvvkdls ggidref |
| Alpha-N-acetylneuraminide alpha-2,8-sialyltransferase, isoform 1 | |
| NP_003025.1 | |
| 1 | mspcgrarrq tsrgamavla wkfprtrlpm gasalcvvvl cwlyifpvyr lpnekeivqg |
| 61 | vlqqgtawrr nqtaarafrk qmedccdpah lfamtkmnsp mgksmwydge flysftidns |
| 121 | tyslfpqatp fqlplkkcav vgnggilkks gcgrqidean fvmrcnlppl sseytkdvgs |
| 181 | ksqlvtanps iirgrfqnll wsrktfvdnm kiynhsyiym pafsmktgte pslrvyytls |
| 241 | dvganqtvlf anpnflrsig kfwksrgiha krlstglflv saalglceev aiygfwpfsv |
| 301 | nmheqpishh yydnvlpfsg fhampeeflq lwylhkigal rmqldpcedt slqpts |
| Alpha-N-acetylneuraminide alpha-2,8-sialyltransferase, isoform 2 | |
| NP_001291379.1 | |
| 1 | mtgsfythsp ltiqltlssh rcnlpplsse ytkdvgsksq lvtanpsiir qrfqnllwsr |
| 61 | ktfvdnmkiy nhsyiympaf smktgtepsl rvyytlsdvg anqtvlfanp nflrsigkfw |
| 121 | ksrgihakrl stglflvsaa lglceevaiy gfwpfsvnmh eqpishhyyd nvlpfsgfha |
| 181 | mpeeflqlwy lhkigalrmq ldpcedtslq pts |
| Survivin, isoform 1 NP_001159.2 | |
| 1 | mgaptlppaw qpflkdhris tfknwpfleg cactpermae agfihcpten epdlaqcffc |
| 61 | fkelegwepd ddpieehkkh ssgcaflsvk kqfeeltlge flkldrerak nkiaketnnk |
| 121 | kkefeetaek vrraieqlaa md |
| Survivin, isoform 2 NP_001012270.1 | |
| 1 | mgaptlppaw qpflkdhris tfknwpfleg cactpermae agfihcpten epdlaqcffc |
| 61 | fkelegwepd ddpmqrkpti rrknlrklrr kcavpssswl pwieasgrsc lvpewlhhfq |
| 121 | glfpgatslp vgplams |
| Survivin, isoform 3 NP_001012271.1 | |
| 1 | mgaptlppaw qpflkdhris tfknwpfleg cactpermae agfihcpten epdlaqcffc |
| 61 | fkelegwepd ddpigpgtva yacntstlgg rggritreeh kkhssgcafl svkkqfeelt |
| 121 | lgeflkldre raknkiaket nnkkkefeet aekvrraieq laamd |
| T-box 4, isoform 1 NP_001308049.1 | |
| 1 | mlqdkglses eeafrapgpa lgeasaanap epalaapgls gaalgsppgp gadvvaaaaa |
| 61 | eqtienikvg lhekelwkkf heagtemiit kagrrmfpsy kvkvtgmnpk tkyillidiv |
| 121 | paddhrykfc dnkwmvagka epampgrlyv hpdspatgah wmrqlvsfqk lkltnnhldp |
| 181 | fghiilnsmh kyqprlhivk adennafgsk ntafcthvfp etsfisvtsy qnhkitqlki |
| 241 | ennpfakgfr gsddsdlrva rlqskeypvi sksimrqrli spqlsatpdv gpllgthqal |
| 301 | qhyqhengah sqlaepqdlp lstfptqrds slfyhclkrr adgtrhldlp ckrsyleaps |
| 361 | svgedhyfrs pppydqqmls psycsevtpr eacmysgsgp eiagvsgvdd lpppplscnm |
| 421 | wtsvspytsy svqtmetvpy qpfpthftat tmmprlptls aqssqppgna hfsvynqlsq |
| 481 | sqvrergpsa sfprerglpq gcerkppsph lnaaneflys qtfslsress lqyhsgmgtv |
| 541 | enwtdg |
| T-box 4, isoform 2 NP_060958.2 | |
| 1 | mlqdkglses eeafrapgpa lgeasaanap epalaapgls gaalgsppgp gadvvaaaaa |
| 61 | eqtienikvg lhekelwkkf heagtemiit kagrrmfpsy kvkvtgmnpk tkyillidiv |
| 121 | paddhrykfc dnkwmvagka epampgrlyv hpdspatgah wmrqlvsfqk lkltnnhldp |
| 181 | fghiilnsmh kyqprlhivk adennafgsk ntafcthvfp etsfisvtsy qnhkitqlki |
| 241 | ennpfakgfr gsddsdlrva rlqskeypvi sksimrqrli spqlsatpdv gpllgthqal |
| 301 | qhyqhengah sqlaepqdlp lstfptqrds slfyhclkrr dgtrhldlpc krsyleapss |
| 361 | vgedhyfrsp ppydqqmlsp sycsevtpre acmysgsgpe iagvsgvddl pppplscnmw |
| 421 | tsvspytsys vqtmetvpyq pfpthftatt mmprlptlsa qssqppgnah fsvynqlsqs |
| 481 | qvrergpsas fprerglpqg cerkppsphl naaneflysq tfslsressl qyhsgmgtve |
| 541 | nwtdg |
| Angiopoietin-1 receptor, isoform 1 NP_000450.2 | |
| 1 | mdslaslvlc gvslllsgtv egamdlilin slplvsdaet sltciasgwr phepitigrd |
| 61 | fealmnqhqd plevtqdvtr ewakkvvwkr ekaskingay fcegrvrgea irirtmkmrq |
| 121 | qasflpatlt mtvdkgdnvn isfkkvlike edaviykngs fihsvprhev pdilevhlph |
| 181 | aqpqdagvys aryiggnlft saftrlivrr ceaqkwgpec nhlctacmnn gvchedtgec |
| 241 | icppgfmgrt cekacelhtf grtckercsg qegcksyvfc lpdpygcsca tgwkglqcne |
| 301 | achpgfygpd cklrcscnng emcdrfqgcl cspgwqglqc eregiprmtp kivdlpdhie |
| 361 | vnsgkfnpic kasgwplptn eemtlvkpdg tvlhpkdfnh tdhfsvaift ihrilppdsg |
| 421 | vwvcsvntva gmvekpfnis vkvlpkplna pnvidtghnf avinissepy fgdgpikskk |
| 481 | llykpvnhye awqhiqvtne ivtlnylepr teyelcvqlv rrgeggeghp gpvrrfttas |
| 541 | iglppprgln llpksqttln ltwqpifpss eddfyvever rsvqksdqqn ikvpgnltsv |
| 601 | llnnlhpreq yvvrarvntk aqgewsedlt awtlsdilpp qpenikisni thssaviswt |
| 661 | ildgysissi tirykvqgkn edqhvdvkik natitqyqlk glepetayqv difaennigs |
| 721 | snpafshelv tlpesqapad lgggkmllia ilgsagmtcl tvllafliil qlkranvqrr |
| 781 | maqafqnvre epavqfnsgt lalnrkvknn pdptiypvld wndikfqdvi gegnfgqvlk |
| 841 | arikkdglrm daaikrmkey askddhrdfa gelevlcklg hhpniinllg acehrgylyl |
| 901 | aieyaphgnl ldflrksrvl etdpafaian stastlssqq llhfaadvar gmdylsqkqf |
| 961 | ihrdlaarni lvgenyvaki adfglsrgqe vyvkktmgrl pvrwmaiesl nysvyttnsd |
| 1021 | vwsygvllwe ivslggtpyc gmtcaelyek lpqgyrlekp lncddevydl mrqcwrekpy |
| 1081 | erpsfaqilv slnrmleerk tyvnttlyek ftyagidcsa eeaa |
| Angiopoietin-1 receptor, isoform 2 NP_001277006.1 | |
| 1 | mdslaslvlc gvslllsgtv egamdlilin slplvsdaet sltciasgwr phepitigrd |
| 61 | fealmnqhqd plevtqdvtr ewakkvvwkr ekaskingay fcegrvrgea irirtmkmrq |
| 121 | qasflpatlt mtvdkgdnvn isfkkvlike edaviykngs fihsvprhev pdilevhlph |
| 181 | aqpqdagvys aryiggnlft saftrlivrr ceaqkwgpec nhlctacmnn gvchedtgec |
| 241 | icppgfmgrt cekacelhtf grtckercsg qegcksyvfc lpdpygcsca tgwkglqcne |
| 301 | giprmtpkiv dlpdhievns gkfnpickas gwplptneem tlvkpdgtvl hpkdfnhtdh |
| 361 | fsvaiftihr ilppdsgvwv csvntvagmv ekpfnisvkv lpkplnapnv idtghnfavi |
| 421 | nissepyfgd gpikskklly kpvnhyeawq hiqvtneivt lnyleprtey elcvqlvrrg |
| 481 | eggeghpgpv rrfttasigl ppprglnllp ksqttlnltw qpifpssedd fyveverrsv |
| 541 | qksdqqnikv pgnltsvlln nlhpreqyvv rarvntkaqg ewsedltawt lsdilppqpe |
| 601 | nikisniths saviswtild gysissitir ykvqgknedq hvdvkiknat itqyqlkgle |
| 661 | petayqvdif aennigssnp afshelvtlp esqapadlgg gkmlliailg sagmtcltvl |
| 721 | lafliilqlk ranvqrrmaq afqnvreepa vqfnsgtlal nrkvknnpdp tiypvldwnd |
| 781 | ikfqdvigeg nfgqvlkari kkdglrmdaa ikrmkeyask ddhrdfagel evlcklghhp |
| 841 | niinllgace hrgylylaie yaphgnlldf lrksrvletd pafaiansta stlssqqllh |
| 901 | faadvargmd ylsqkqfihr dlaarnilvg enyvakiadf glsrgqevyv kktmgrlpvr |
| 961 | wmaieslnys vyttnsdvws ygvllweivs lggtpycgmt caelyeklpq gyrlekplnc |
| 1021 | ddevydlmrq cwrekpyerp sfaqilvsln rmleerktyv nttlyekfty agidcsaeea |
| 1081 | a |
| Angiopoietin-1 receptor, isoform 3 NP_001277007.1 | |
| 1 | mdslaslvlc gvslllsasf lpatltmtvd kgdnvnisfk kvlikeedav iykngsfihs |
| 61 | vprhevpdil evhlphaqpq dagvysaryi ggnlftsaft rlivrrceaq kwgpecnhlc |
| 121 | tacmnngvch edtgecicpp gfmgrtceka celhtfgrtc kercsgqegc ksyvfclpdp |
| 181 | ygcscatgwk glqcnegipr mtpkivdlpd hievnsgkfn pickasgwpl ptneemtlvk |
| 241 | pdgtvlhpkd fnhtdhfsva iftihrilpp dsgvwvcsvn tvagmvekpf nisvkvlpkp |
| 301 | lnapnvidtg hnfaviniss epyfgdgpik skkllykpvn hyeawqhiqv tneivtlnyl |
| 361 | eprteyelcv qlvrrgegge ghpgpvrrft tasiglpppr glnllpksqt tlnltwqpif |
| 421 | psseddfyve verrsvqksd qqnikvpgnl tsvllnnlhp reqyvvrarv ntkaqgewse |
| 481 | dltawtlsdi lppqpeniki snithssavi swtildgysi ssitirykvq gknedqhvdv |
| 541 | kiknatitqy qlkglepeta yqvdifaenn igssnpafsh elvtlpesqa padlgggkml |
| 601 | liailgsagm tcltvllafl iilqlkranv qrrmaqafqn reepavqfns gtlalnrkvk |
| 661 | nnpdptiypv ldwndikfqd vigegnfgqv lkarikkdgl rmdaaikrmk eyaskddhrd |
| 721 | fagelevlck lghhpniinl lgacehrgyl ylaieyaphg nlldflrksr vletdpafai |
| 781 | anstastlss qqllhfaadv argmdylsqk qfihrdlaar nilvgenyva kiadfglsrg |
| 841 | qevyvkktmg rlpvrwmaie slnysvyttn sdvwsygvll weivslggtp ycgmtcaely |
| 901 | eklpqgyrle kplncddevy dlmrqcwrek pyerpsfaqi lvslnrmlee rktyvnttly |
| 961 | ekftyagidc saeeaa |
| Telomerase reverse transcriptase, isoform 1 NP_937983.2 | |
| 1 | mpraprcrav rsllrshyre vlplatfvrr lgpqgwrlvq rgdpaafral vagclvcvpw |
| 61 | darpppaaps frqvsclkel varvlqrlce rgaknvlafg falldgargg ppeafttsvr |
| 121 | sylpntvtda lrgsgawgll lrrvgddvlv hllarcalfv lvapscayqv cgpplyqlga |
| 181 | atqarpppha sgprrrlgce rawnhsvrea gvplglpapg arrrggsasr slplpkrprr |
| 241 | gaapepertp vgqgswahpg rtrgpsdrgf cvvsparpae eatslegals gtrhshpsvg |
| 301 | rqhhagppst srpprpwdtp cppvyaetkh flyssgdkeq lrpsfllssl rpsltgarrl |
| 361 | vetiflgsrp wmpgtprrlp rlpqrywqmr plflellgnh aqcpygvllk thcplraavt |
| 421 | paagvcarek pqgsvaapee edtdprrlvq llrqhsspwq vygfvraclr rlvppglwgs |
| 481 | rhnerrflrn tkkfislgkh aklslqeltw kmsvrdcawl rrspgvgcvp aaehrlreei |
| 541 | lakflhwlms vyvvellrsf fyvtettfqk nrlffyrksv wsklqsigir qhlkrvqlre |
| 601 | lseaevrqhr earpalltsr lrfipkpdgl rpivnmdyvv gartfrrekr aerltsrvka |
| 661 | lfsvlnyera rrpgllgasv lglddihraw rtfvlrvraq dpppelyfvk vdvtgaydti |
| 721 | pqdrltevia siikpqntyc vrryavvqka ahghvrkafk shvstltdlq pymrqfvahl |
| 781 | qetsplrdav vieqssslne assglfdvfl rfmchhavri rgksyvqcqg ipqgsilstl |
| 841 | lcslcygdme nklfagirrd glllrlvddf llvtphltha ktflrtlvrg vpeygcvvnl |
| 901 | rktvvnfpve dealggtafv qmpahglfpw cgllldtrtl evqsdyssya rtsirasltf |
| 961 | nrgfkagrnm rrklfgvlrl kchslfldlq vnslqtvctn iykilllqay rfhacvlqlp |
| 1021 | fhqqvwknpt fflrvisdta slcysilkak nagmslgakg aagplpseav qwlchqafll |
| 1081 | kltrhrvtyv pllgslrtaq tqlsrklpgt tltaleaaan palpsdfkti ld |
| Telomerase reverse transcriptase, isoform 2 NP_001180305.1 | |
| 1 | mpraprcrav rsllrshyre vlplatfvrr lgpqgwrlvq rgdpaafral vaqclvcvpw |
| 61 | darpppaaps frqvsclkel varvlqrlce rgaknvlafg falldgargg ppeafttsvr |
| 121 | sylpntvtda lrgsgawgll lrrvgddvlv hllarcalfv lvapscayqv cgpplyqlga |
| 181 | atqarpppha sgprrrlgce rawnhsvrea gvplglpapg arrrggsasr slplpkrprr |
| 241 | gaapepertp vgqgswahpg rtrgpsdrgf cvvsparpae eatslegals gtrhshpsvg |
| 301 | rqhhagppst srpprpwdtp cppvyaetkh flyssgdkeq lrpsfllssl rpsltgarrl |
| 361 | vetiflgsrp wmpgtprrlp rlpqrywqmr plflellgnh aqcpygvllk thcplraavt |
| 421 | paagvcarek pqgsvaapee edtdprrlvq llrqhsspwq vygfvraclr rlvppglwgs |
| 481 | rhnerrflrn tkkfislgkh aklslqeltw kmsvrdcawl rrspgvgcvp aaehrlreei |
| 541 | lakflhwlms vyvvellrsf fyvtettfqk nrlffyrksv wsklqsigir qhlkrvqlre |
| 601 | lseaevrqhr earpalltsr lrfipkpdgl rpivnmdyvv gartfrrekr aerltsrvka |
| 661 | lfsvlnyera rrpgllgasv lglddihraw rtfvlrvraq dpppelyfvk vdvtgaydti |
| 721 | pqdrltevia siikpqntyc vrryavvqka ahghvrkafk shvstltdlq pymrqfvahl |
| 781 | qetsplrdav vieqssslne assglfdvfl rfmchhavri rgksyvqcqg ipqgsilstl |
| 841 | lcslcygdme nklfagirrd glllrlvddf llvtphltha ktflsyarts irasltfnrg |
| 901 | fkagrnmrrk lfgvlrlkch slfldlqvns lqtvctniyk illlqayrfh acvlqlpfhq |
| 961 | qvwknptffl rvisdtaslc ysilkaknag mslgakgaag plpseavqwl chqafllklt |
| 1021 | rhrvtyvpll gslrtaqtql srklpgttlt aleaaanpal psdfktild |
| Cellular tumor antigen p53, isoform a NP_000537.3, NP_001119584.1 | |
| 1 | meepqsdpsv epplsqetfs dlwkllpenn vlsplpsqam ddlmlspddi eqwftedpgp |
| 61 | deaprmpeaa ppvapapaap tpaapapaps wplsssvpsq ktyqgsygfr lgflhsgtak |
| 121 | svtctyspal nkmfcqlakt cpvqlwvdst pppgtrvram aiykqsqhmt evvrrcphhe |
| 181 | rcsdsdglap pqhlirvegn lrveylddrn tfrhsvvvpy eppevgsdct tihynymcns |
| 241 | scmggmnrrp iltiitleds sgnllgrnsf evrvcacpgr drrteeenlr kkgephhelp |
| 301 | pgstkralpn ntssspqpkk kpldgeyftl qirgrerfem frelnealel kdaqagkepg |
| 361 | gsrahsshlk skkgqstsrh kklmfktegp dsd |
| Cellular tumor antigen p53, isoform b NP_001119586.1 | |
| 1 | meepqsdpsv epplsqetfs dlwkllpenn vlsplpsqam ddlmlspddi eqwftedpgp |
| 61 | deaprmpeaa ppvapapaap tpaapapaps wplsssvpsq ktyqgsygfr lgflhsgtak |
| 121 | svtctyspal nkmfcqlakt cpvqlwvdst pppgtrvram aiykqsqhmt evvrrcphhe |
| 181 | rcsdsdglap pqhlirvegn lrveylddrn tfrhsvvvpy eppevgsdct tihynymcns |
| 241 | scmggmnrrp iltiitleds sgnllgrnsf evrvcacpgr drrteeenlr kkgephhelp |
| 301 | pgstkralpn ntssspqpkk kpldgeyftl qdqtsfqken c |
| Cellular tumor antigen p53, isoform c NP_001119585.1 | |
| 1 | meepqsdpsv epplsqetfs dlwkllpenn vlsplpsqam ddlmlspddi eqwftedpgp |
| 61 | deaprmpeaa ppvapapaap tpaapapaps wplsssvpsq ktyqgsygfr lgflhsgtak |
| 121 | svtctyspal nkmfcqlakt cpvqlwvdst pppgtrvram aiykqsqhmt evvrrcphhe |
| 181 | rcsdsdglap pqhlirvegn lrveylddrn tfrhsvvvpy eppevgsdct tihynymcns |
| 241 | scmggmnrrp iltiitleds sgnllgrnsf evrvcacpgr drrteeenlr kkgephhelp |
| 301 | pgstkralpn ntssspqpkk kpldgeyftl qmlldlrwcy flinss |
| Cellular tumor antigen p53, isoform d NP_001119587.1 | |
| 1 | mfcqlaktcp vqlwvdstpp pgtrvramai ykqsqhmtev vrrcphherc sdsdglappq |
| 61 | hlirvegnlr veylddrntf rhsvvvpyep pevgsdctti hynymcnssc mggmnrrpil |
| 121 | tiitledssg nllgrnsfev rvcacpgrdr rteeenlrkk gephhelppg stkralpnnt |
| 181 | ssspqpkkkp ldgeyftlqi rgrerfemfr elnealelkd aqagkepggs rahsshlksk |
| 241 | kgqstsrhkk lmfktegpds d |
| Cellular tumor antigen p53, isoform e NP_001119588.1 | |
| 1 | mfcqlaktcp vqlwvdstpp pgtrvramai ykqsqhmtev vrrcphherc sdsdglappq |
| 61 | hlirvegnlr veylddrntf rhsvvvpyep pevgsdctti hynymcnssc mggmnrrpil |
| 121 | tiitledssg nllgrnsfev rvcacpgrdr rteeenlrkk gephhelppg stkralpnnt |
| 181 | ssspqpkkkp ldgeyftlqd qtsfqkenc |
| Cellular tumor antigen p53, isoform f NP_001119589.1 | |
| 1 | mfcqlaktcp vqlwvdstpp pgtrvramai ykqsqhmtev vrrcphherc sdsdglappq |
| 61 | hlirvegnlr veylddrntf rhsvvvpyep pevgsdctti hynymcnssc mggmnrrpil |
| 121 | tiitledssg nllgrnsfev rvcacpgrdr rteeenlrkk gephhelppg stkralpnnt |
| 181 | ssspqpkkkp ldgeyftlqm lldlrwcyfl inss |
| Cellular tumor antigen p53, isoform g NP_001119590.1, NP_001263689.1, | |
| NP_001263690.1 | |
| 1 | mddlmlspdd ieqwftedpg pdeaprmpea appvapapaa ptpaapapap swplsssvps |
| 61 | qktyqgsygf rlgflhsgta ksvtctyspa lnkmfcqlak tcpvqlwvds tpppgtrvra |
| 121 | maiykqsqhm tevvrrcphh ercsdsdgla ppqhlirveg nlrveylddr ntfrhsvvvp |
| 181 | yeppevgsdc ttihynymcn sscmggmnrr piltiitled ssgnllgrns fevrvcacpg |
| 241 | rdrrteeenl rkkgephhel ppgstkralp nntssspqpk kkpldgeyft lqirgrerfe |
| 301 | mfrelneale lkdaqagkep ggsrahsshl kskkgqstsr hkklmfkteg pdsd |
| Cellular tumor antigen p53, isoform h NP_001263624.1 | |
| 1 | mddlmlspdd ieqwftedpg pdeaprmpea appvapapaa ptpaapapap swplsssvps |
| 61 | qktyqgsygf rlgflhsgta ksvtctyspa lnkmfcqlak tcpvqlwvds tpppgtrvra |
| 121 | maiykqsqhm tevvrrcphh ercsdsdgla ppqhlirveg nlrveylddr ntfrhsvvvp |
| 181 | yeppevgsdc ttihynymcn sscmggmnrr piltiitled ssgnllgrns fevrvcacpg |
| 241 | rdrrteeenl rkkgephhel ppgstkralp nntssspqpk kkpldgeyft lqmlldlrwc |
| 301 | yflinss |
| Cellular tumor antigen p53, isoform i NP_001263625.1 | |
| 1 | mddlmlspdd ieqwftedpg pdeaprmpea appvapapaa ptpaapapap swplsssvps |
| 61 | qktyqgsygf rlgflhsgta ksvtctyspa lnkmfcqlak tcpvqlwvds tpppgtrvra |
| 121 | maiykqsqhm tevvrrcphh ercsdsdgla ppqhlirveg nlrveylddr ntfrhsvvvp |
| 181 | yeppevgsdc ttihynymcn sscmggmnrr piltiitled ssgnllgrns fevrvcacpg |
| 241 | rdrrteeenl rkkgephhel ppgstkralp nntssspqpk kkpldgeyft lqdqtsfqke |
| 301 | nc |
| Cellular tumor antigen p53, isoform j NP_001263626.1 | |
| 1 | maiykqsqhm tevvrrcphh ercsdsdgla ppqhlirveg nlrveylddr ntfrhsvvvp |
| 61 | yeppevgsdc ttihynymcn sscmggmnrr piltiitled ssgnllgrns fevrvcacpg |
| 121 | rdrrteeenl rkkgephhel ppgstkralp nntssspqpk kkpldgeyft lqirgrerfe |
| 181 | mfrelneale lkdaqagkep ggsrahsshl kskkgqstsr hkklmfkteg pdsd |
| Cellular tumor antigen p53, isoform k NP_001263627.1 | |
| 1 | maiykqsqhm tevvrrcphh ercsdsdgla ppqhlirveg nlrveylddr ntfrhsvvvp |
| 61 | yeppevgsdc ttihynymcn sscmggmnrr piltiitled ssgnllgrns fevrvcacpg |
| 121 | rdrrteeenl rkkgephhel ppgstkralp nntssspqpk kkpldgeyft lqdqtsfqke |
| 181 | nc |
| Cellular tumor antigen p53, isoform l NP_001263628.1 | |
| 1 | maiykqsqhm tevvrrcphh ercsdsdgla ppqhlirveg nlrveylddr ntfrhsvvvp |
| 61 | yeppevgsdc ttihynymcn sscmggmnrr piltiitled ssgnllgrns fevrvcacpg |
| 121 | rdrrteeenl rkkgephhel ppgstkralp nntssspqpk kkpldgeyft lqmlldlrwc |
| 181 | yflinss |
| Dopachrome tautomerase, isoform 1 NP_001913.2 | |
| 1 | msplwwgfll sclgckilpg aqgqfprvcm tvdslvnkec cprlgaesan vcgsqqgrgq |
| 61 | ctevradtrp wsgpyilrnq ddrelwprkf fhrtckctgn fagyncgdck fgwtgpncer |
| 121 | kkppvirqni hslspgereq flgaldlakk rvhpdyvitt qhwlgllgpn gtqpqfancs |
| 181 | vydffvwlhy ysvrdtllgp grpyraidfs hqgpafvtwh ryhllclerd lqrlignesf |
| 241 | alpywnfatg rnecdvctdq lfgaarpddp tlisrnsrfs swetvcdsld dynhlvtlcn |
| 301 | gtyegllrrn qmgrnsmklp tlkdirdcls lqkfdnppff qnstfsfrna legfdkadgt |
| 361 | ldsqvmslhn lvhsflngtn alphsaandp ifvvlhsftd aifdewmkrf nppadawpqe |
| 421 | lapighnrmy nmvpffppvt neelfltsdq lgysyaidlp vsveetpgwp ttllvvmgtl |
| 481 | valvglfvll aflqyrrlrk gytplmethl sskryteea |
| Dopachrome tautomerase, isoform 2 NP_001123361.1 | |
| 1 | msplwwgfll sclgckilpg aqgqfprvcm tvdslvnkec cprlgaesan vcgsqqgrgq |
| 61 | ctevradtrp wsgpyilrnq ddrelwprkf fhrtckctgn fagyncgdck fgwtgpncer |
| 121 | kkppvirqni hslspgereq flgaldlakk rvhpdyvitt qhwlgllgpn gtqpqfancs |
| 181 | vydffvwlhy ysvrdtllgp grpyraidfs hqgpafvtwh ryhllclerd lqrlignesf |
| 241 | alpywnfatg rnecdvctdq lfgaarpddp tlisrnsrfs swetvcdsld dynhlvtlcn |
| 301 | gtyegllrrn qmgrnsmklp tlkdirdcls lqkfdnppff qnstfsfrna legfdkadgt |
| 361 | ldsqvmslhn lvhsflngtn alphsaandp ifvvisnrll ynattnileh vrkekatkel |
| 421 | pslhvlvlhs ftdaifdewm krfnppadaw pqelapighn rmynmvpffp pvtneelflt |
| 481 | sdqlgysyai dlpvsveetp gwpttllvvm gtlvalvglf vllaflqyrr lrkgytplme |
| 541 | thlsskryte ea |
| Dopachrome tautomerase, isoform 3 NP_001309111.1, NP_001309112.1, | |
| NP_001309113.1, NP_001309114.1 | |
| 1 | mgrnsmklpt lkdirdclsl qkfdnppffq nstfsfrnal egfdkadgtl dsqvmslhnl |
| 61 | vhsflngtna lphsaandpi fvvlhsftda ifdewmkrfn ppadawpqel apighnrmyn |
| 121 | mvpffppvtn eelfltsdql gysyaidlpv sveetpgwpt tllvvmgtlv alvglfvlla |
| 181 | flqyrrlrkg ytplmethls skryteea |
| Dopachrome tautomerase, isoform 4, NP_001309115.1 | |
| 1 | mllgiqrqmk crlrsdvtkr leedehvnth spmrrgnfag yncgdckfgw tgpncerkkp |
| 61 | pvirqnihsl spqereqflg aldlakkrvh pdyvittqhw lgllgpngtq pqfancsvyd |
| 121 | ffvwlhyysv rdtllgpgrp yraidfshqg pafvtwhryh llclerdlqr lignesfalp |
| 181 | ywnfatgrne cdvctdqlfg aarpddptli srnsrfsswe tvcdslddyn hlvtlcngty |
| 241 | egllrrnqmg rnsmklptlk dirdclslqk fdnppffqns tfsfrnaleg fdkadgtlds |
| 301 | qvmslhnlvh sflngtnalp hsaandpifv vlhsftdaif dewmkrfnpp adawpqelap |
| 361 | ighnrmynmv pffppvtnee lfltsdqlgy syaidlpvsv eetpgwpttl lvvmgtlval |
| 421 | vglfvllafl qyrrlrkgyt plmethlssk ryteea |
| Transformation/transcription domain associated protein, isoform 1 | |
| NP_001231509.1 | |
| 1 | mafvatqgat vvdqttlmkk ylqfvaaltd vntpdetklk mmqevsenfe nvtsspqyst |
| 61 | flehiiprfl tflqdgevqf lqekpaqqlr klvleiihri ptnehlrpht knvlsvmfrf |
| 121 | leteneenvl iclriiielh kqfrppitqe ihhfldfvkq iykelpkvvn ryfenpqvip |
| 181 | entvpppemv gmittiavkv nperedsetr thsiiprgsl slkvlaelpi ivvlmyqlyk |
| 241 | lnihnvvaef vplimntiai qvsaqarqhk lynkelyadf iaaqiktlsf layiiriyqe |
| 301 | lvtkysqqmv kgmlqllsnc paetahlrke lliaakhilt telrnqfipc mdklfdesil |
| 361 | igsgytaret lrplaystla dlvhhvrqhl plsdlslavq lfakniddes lpssiqtmsc |
| 421 | klllnlvdci rskseqesgn grdvlmrmle vfvlkfhtia ryqlsaifkk ckpqselgav |
| 481 | eaalpgvpta paapgpapsp apvpappppp pppppatpvt papvppfekq gekdkedkqt |
| 541 | fqvtdcrslv ktlvcgvkti twgitsckap geaqfipnkq lqpketqiyi klvkyamqal |
| 601 | diyqvqiagn gqtyirvanc qtvrmkeeke vlehfagvft mmnpltfkei fqttvpymve |
| 661 | risknyalqi vansflanpt tsalfatilv eylldrlpem gsnvelsnly lklfklvfgs |
| 721 | vslfaaeneq mlkphlhkiv nssmelaqta kepynyflll ralfrsiggg shdllygefl |
| 781 | pllpnllqgl nmlqsglhkq hmkdlfvelc ltvpvrlssl lpylpmlmdp lvsalngsqt |
| 841 | lvsqglrtle lcvdnlqpdf lydhiqpvra elmqalwrtl rnpadsishv ayrvlgkfgg |
| 901 | snrkmlkesq klhyvvtevq gpsitvefsd ckaslqlpme kaietaldcl ksantepyyr |
| 961 | rqawevikcf lvammsledn khalyqllah pnftektipn viishrykaq dtparktfeq |
| 1021 | altgafmsav ikdlrpsalp fvaslirhyt mvavaqqcgp fllpcyqvgs qpstamfhse |
| 1081 | engskgmdpl vlidaiaicm ayeekelcki gevalavifd vasiilgske racqlplfsy |
| 1141 | iverlcaccy eqawyaklgg vvsikflmer lpltwvlqnq qtflkallfv mmdltgevsn |
| 1201 | gavamakttl eqllmrcatp lkdeeraeei vaaqeksfhh vthdlvrevt spnstvrkqa |
| 1261 | mhslqvlaqv tgksvtvime phkevlqdmv ppkkhllrhq panaqiglme gntfcttlqp |
| 1321 | rlftmdlnvv ehkvfytell nlceaedsal tklpcykslp slvplriaal nalaacnylp |
| 1381 | qsrekiiaal fkalnstnse lqeageacmr kflegatiev dqihthmrpl lmmlgdyrsl |
| 1441 | tlnvvnrlts vtrlfpnsfn dkfcdqmmqh lrkwmevvvi thkggqrsdg nesisecgrc |
| 1501 | plspfcqfee mkicsaiinl fhlipaapqt lvkpllevvm kteramliea gspfreplik |
| 1561 | fltrhpsqtv elfmmeatln dpqwsrmfms flkhkdarpl rdvlaanpnr fitlllpgga |
| 1621 | qtavrpgsps tstmrldlqf qaikiisiiv knddswlasq hslvsqlrrv wvsenfqerh |
| 1681 | rkenmaatnw kepkllaycl lnyckrnygd iellfqllra ftgrflcnmt flkeymeeei |
| 1741 | pknysiaqkr alffrfvdfn dpnfgdelka kvlqhilnpa flysfekgeg eqllgppnpe |
| 1801 | gdnpesitsv fitkvldpek qadmldslri yllqyatllv ehaphhihdn nknrnsklrr |
| 1861 | lmtfawpcll skacvdpack ysghlllahi iakfaihkki vlqvfhsllk ahamearaiv |
| 1921 | rqamailtpa vparmedghq mlthwtrkii veeghtvpql vhilhlivqh fkvyypvrhh |
| 1981 | lvqhmvsamq rlgftpsvti eqrrlavdls evvikwelqr ikdqqpdsdm dpnssgegvn |
| 2041 | svsssikrgl svdsaqevkr frtatgaisa vfgrsqslpg adsllakpid kqhtdtvvnf |
| 2101 | lirvacqvnd ntntagspge vlsrrcvnll ktalrpdmwp kselklqwfd kllmtveqpn |
| 2161 | qvnygnictg levlsflltv lqspailssf kplqrgiaac mtcgntkvlr avhsllsrlm |
| 2221 | sifptepsts svaskyeele clyaavgkvi yegltnyeka tnanpsqlfg tlmilksacs |
| 2281 | nnpsyidrli svfmrslqkm vrehlnpqaa sgsteatsgt selvmlslel vktrlavmsm |
| 2341 | emrknfiqai ltsliekspd akilravvki veewvknnsp maanqtptlr eksillvkmm |
| 2401 | tyiekrfped lelnaqfldl vnyvyrdetl sgseltakle paflsglrca qplirakffe |
| 2461 | vfdnsmkrrv yerllyvtcs qnweamgnhf wikqcielll avcekstpig tscqgamlps |
| 2521 | itnvinlads hdraafamvt hvkqeprere nseskeedve idielapgdq tstpktkels |
| 2581 | ekdignqlhm ltnrhdkfld tlrevktgal lsafvqlchi sttlaektwv qlfprlwkil |
| 2641 | sdrqqhalag eispflcsgs hqvqrdcqps alncfveams qcvppipirp cvlkylgkth |
| 2701 | nlwfrstlml ehqafekgls lqikpkqtte fyeqesitpp qqeildslae lysllqeedm |
| 2761 | waglwqkrck ysetataiay eqhgffeqaq esyekamdka kkehersnas paifpeyqlw |
| 2821 | edhwircske lnqwealtey gqskghinpy lvlecawrvs nwtamkealv qvevscpkem |
| 2881 | awkvnmyrgy laichpeeqq lsfierlvem asslairewr rlphvvshvh tpllqaaqqi |
| 2941 | ielqeaaqin aglqptnlgr nnslhdmktv vktwrnrlpi vsddlshwss ifmwrqhhyq |
| 3001 | gkptwsgmhs ssivtayens sqhdpssnna mlgvhasasa iiqygkiark qglvnvaldi |
| 3061 | lsrihtiptv pivdcfqkir qqvkcylqla gvmgknecmq gleviestnl kyftkemtae |
| 3121 | fyalkgmfla qinkseeank afsaavqmhd vlvkawamwg dylenifvke rqlhlgvsai |
| 3181 | tcylhacrhq nesksrkyla kvlwllsfdd dkntladavd kycigvppiq wlawipqllt |
| 3241 | clvgsegkll lnlisqvgrv ypqavyfpir tlyltlkieq reryksdpgp iratapmwrc |
| 3301 | srimhmqrel hptllssleg ivdqmvwfre nwheevlrql qqglakcysv afeksgavsd |
| 3361 | akitphtlnf vkklvstfgv glenvsnvst mfssaasesl arraqataqd pvfqklkgqf |
| 3421 | ttdfdfsvpg smklhnlisk lkkwikilea ktkqlpkffl ieekcrflsn fsaqtaevei |
| 3481 | pgeflmpkpt hyyikiarfm prveivqkhn taarrlyirg hngkiypylv mndacltesr |
| 3541 | reervlqllr llnpclekrk ettkrhlfft vprvvayspq mrlvednpss lslveiykqr |
| 3601 | cakkgiehdn pisryydrla tvqargtqas hqvlrdilke vqsnmvprsm lkewalhtfp |
| 3661 | natdywtfrk mftiqlalig faefvlhlnr lnpemlqiaq dtgklnvayf rfdindatgd |
| 3721 | ldanrpvpfr ltpniseflt tigvsgplta smiavarcfa qpnfkvdgil ktvlrdeiia |
| 3781 | whkktqedts splsaagqpe nmdsqqlvsl vqkavtaimt rlhnlaqfeg geskvntlva |
| 3841 | aansldnlcr mdpawhpwl |
| Transformation/transcription domain associated protein, isoform 2 | |
| NP_003487.1 | |
| 1 | mafvatqgat vvdqttlmkk ylqfvaaltd vntpdetklk mmqevsenfe nvtsspqyst |
| 61 | flehiiprfl tflqdgevqf lqekpaqqlr klvleiihri ptnehlrpht knvlsvmfrf |
| 121 | leteneenvl iclriiielh kqfrppitqe ihhfldfvkq iykelpkvvn ryfenpqvip |
| 181 | entvpppemv gmittiavkv nperedsetr thsiiprgsl slkvlaelpi ivvlmyqlyk |
| 241 | lnihnvvaef vplimntiai qvsaqarqhk lynkelyadf iaaqiktlsf layiiriyqe |
| 301 | lvtkysqqmv kgmlqllsnc paetahlrke lliaakhilt telrnqfipc mdklfdesil |
| 361 | igsgytaret lrplaystla dlvhhvrqhl plsdlslavq lfakniddes lpssiqtmsc |
| 421 | klllnlvdci rskseqesgn grdvlmrmle vfvlkfhtia ryqlsaifkk ckpqselgav |
| 481 | eaalpgvpta paapgpapsp apvpappppp pppppatpvt papvppfekq gekdkedkqt |
| 541 | fqvtdcrslv ktlvcgvkti twgitsckap geaqfipnkq lqpketqiyi klvkyamqal |
| 601 | diyqvqiagn gqtyirvanc qtvrmkeeke vlehfagvft mmnpltfkei fqttvpymve |
| 661 | risknyalqi vansflanpt tsalfatilv eylldrlpem gsnvelsnly lklfklvfgs |
| 721 | vslfaaeneq mlkphlhkiv nssmelaqta kepynyflll ralfrsiggg shdllyqefl |
| 781 | pllpnllqgl nmlqsglhkq hmkdlfvelc ltvpvrlssl lpylpmlmdp lvsalngsqt |
| 841 | lvsqglrtle lcvdnlqpdf lydhiqpvra elmqalwrtl rnpadsishv ayrvlgkfgg |
| 901 | snrkmlkesq klhyvvtevq gpsitvefsd ckaslqlpme kaietaldcl ksantepyyr |
| 961 | rqawevikcf lvammsledn khalyqllah pnftektipn viishrykaq dtparktfeq |
| 1021 | altgafmsav ikdlrpsalp fvaslirhyt mvavaqqcgp fllpcyqvgs qpstamfhse |
| 1081 | engskgmdpl vlidaiaicm ayeekelcki gevalavifd vasiilgske racqlplfsy |
| 1141 | iverlcaccy eqawyaklgg vvsikflmer lpltwvlqnq qtflkallfv mmdltgevsn |
| 1201 | gavamakttl eqllmrcatp lkdeeraeei vaaqeksfhh vthdlvrevt spnstvrkqa |
| 1261 | mhslqvlaqv tgksvtvime phkevlqdmv ppkkhllrhq panaqiglme gntfcttlqp |
| 1321 | rlftmdlnvv ehkvfytell nlceaedsal tklpcykslp slvplriaal nalaacnylp |
| 1381 | qsrekiiaal fkalnstnse lqeageacmr kflegatiev dqihthmrpl lmmlgdyrsl |
| 1441 | tlnvvnrlts vtrlfpnsfn dkfcdqmmqh lrkwmevvvi thkggqrsdg nemkicsaii |
| 1501 | nlfhlipaap qtlvkpllev vmkteramli eagspfrepl ikfltrhpsq tvelfmmeat |
| 1561 | lndpqwsrmf msflkhkdar plrdvlaanp nrfitlllpg gaqtavrpgs pststmrldl |
| 1621 | qfqaikiisi ivknddswla sqhslvsqlr rvwvsenfqe rhrkenmaat nwkepkllay |
| 1681 | cllnyckrny gdiellfqll raftgrflcn mtflkeymee eipknysiaq kralffrfvd |
| 1741 | fndpnfgdel kakvlqhiln paflysfekg egeqllgppn pegdnpesit svfitkvldp |
| 1801 | ekqadmldsl riyllqyatl lvehaphhih dnnknrnskl rrlmtfawpc llskacvdpa |
| 1861 | ckysghllla hiiakfaihk kivlqvfhsl lkahameara ivrqamailt pavparmedg |
| 1921 | hqmlthwtrk iiveeghtvp qlvhilhliv qhfkvyypvr hhlvqhmvsa mqrlgftpsv |
| 1981 | tieqrrlavd lsevvikwel qrikdqqpds dmdpnssgeg vnsvsssikr glsvdsaqev |
| 2041 | krfrtatgai savfgrsqsl pgadsllakp idkqhtdtvv nflirvacqv ndntntagsp |
| 2101 | gevlsrrcvn llktalrpdm wpkselklqw fdkllmtveq pnqvnygnic tglevlsfll |
| 2161 | tvlqspails sfkplqrgia acmtcgntkv lravhsllsr lmsifpteps tssvaskyee |
| 2221 | leclyaavgk viyegltnye katnanpsql fgtlmilksa csnnpsyidr lisvfmrslq |
| 2281 | kmvrehlnpq aasgsteats gtselvmlsl elvktrlavm smemrknfiq ailtslieks |
| 2341 | pdakilravv kiveewvknn spmaanqtpt lreksillvk mmtyiekrfp edlelnaqfl |
| 2401 | dlvnyvyrde tlsgseltak lepaflsglr caqplirakf fevfdnsmkr rvyerllyvt |
| 2461 | csqnweamgn hfwikqciel llavcekstp igtscqgaml psitnvinla dshdraafam |
| 2521 | vthvkqepre renseskeed veidielapg dqtstpktke lsekdignql hmltnrhdkf |
| 2581 | ldtlrevktg allsafvqlc histtlaekt wvqlfprlwk ilsdrqqhal ageispflcs |
| 2641 | gshqvqrdcq psalncfvea msqcvppipi rpcvlkylgk thnlwfrstl mlehqafekg |
| 2701 | lslqikpkqt tefyeqesit ppqqeildsl aelysllqee dmwaglwqkr ckysetatai |
| 2761 | ayeqhgffeq aqesyekamd kakkehersn aspaifpeyq lwedhwircs kelnqwealt |
| 2821 | eygqskghin pylvlecawr vsnwtamkea lvqvevscpk emawkvnmyr gylaichpee |
| 2881 | qqlsfierlv emasslaire wrrlphvvsh vhtpllqaaq qiielqeaaq inaglqptnl |
| 2941 | grnnslhdmk tvvktwrnrl pivsddlshw ssifmwrqhh yqaivtayen ssqhdpssnn |
| 3001 | amlgvhasas aiiqygkiar kqglvnvald ilsrihtipt vpivdcfqki rqqvkcylql |
| 3061 | agvmgknecm qgleviestn lkyftkemta efyalkgmfl aqinkseean kafsaavqmh |
| 3121 | dvlvkawamw gdylenifvk erqlhlgvsa itcylhacrh qnesksrkyl akvlwllsfd |
| 3181 | ddkntladav dkycigvppi qwlawipgll tclvgsegkl llnlisqvgr vypqavyfpi |
| 3241 | rtlyltlkie qreryksdpg piratapmwr csrimhmqre lhptllssle givdqmvwfr |
| 3301 | enwheevlrq lqqglakcys vafeksgavs dakitphtln fvkklvstfg vglenvsnvs |
| 3361 | tmfssaases larraqataq dpvfqklkgq fttdfdfsvp gsmklhnlis klkkwikile |
| 3421 | aktkqlpkff lieekcrfls nfsaqtaeve ipgeflmpkp thyyikiarf mprveivqkh |
| 3481 | ntaarrlyir ghngkiypyl vmndacltes rreervlqll rllnpclekr kettkrhlff |
| 3541 | tvprvvavsp qmrlvednps slslveiykq rcakkgiehd npisryydrl atvqargtqa |
| 3601 | shqvlrdilk evqsnmvprs mlkewalhtf pnatdywtfr kmftiqlali gfaefvlhln |
| 3661 | rlnpemlqia qdtgklnvay frfdindatg dldanrpvpf rltpnisefl ttigvsgplt |
| 3721 | asmiavarcf aqpnfkvdgi lktvlrdeii awhkktqedt ssplsaagqp enmdsqqlvs |
| 3781 | lvqkavtaim trlhnlaqfe ggeskvntlv aaansldnlc rmdpawhpwl |
| Tyrosinase precursor NP_000363.1 | |
| 1 | mllavlycll wsfqtsaghf pracvssknl mekeccppws gdrspcgqls grgscqnill |
| 61 | snaplgpqfp ftgvddresw psvfynrtcq csgnfmgfnc gnckfgfwgp ncterrllvr |
| 121 | rnifdlsape kdkffayltl akhtissdyv ipigtygqmk ngstpmfndi niydlfvwmh |
| 181 | yyvsmdallg gseiwrdidf aheapaflpw hrlfllrweq eiqkltgden ftipywdwrd |
| 241 | aekcdictde ymggqhptnp nllspasffs swqivcsrle eynshqslcn gtpegplrrn |
| 301 | pgnhdksrtp rlpssadvef clsltqyesg smdkaanfsf rntlegfasp ltgiadasqs |
| 361 | smhnalhiym ngtmsqvqgs andpifllhh afvdsifeqw lrrhrplqev ypeanapigh |
| 421 | nresymvpfi plyrngdffi sskdlgydys ylqdsdpdsf qdyiksyleq asriwswllg |
| 481 | aamvgavlta llaglvsllc rhkrkqlpee kqpllmeked yhslyqshl |
| Vascular endothelial growth factor A, isoform a NP_001020537.2 | |
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe kksvrgkgkg qkrkrkksry kswsvyvgar cclmpwslpg |
| 361 | phpcgpcser rkhlfvqdpq tckcsckntd srckarqlel nertcrcdkp rr |
| Vascular endothelial growth factor A, isoform b NP_003367.4 | |
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe kksvrgkgkg qkrkrkksry kswsvpcgpc serrkhlfvq |
| 361 | dpqtckcsck ntdsrckarq lelnertcrc dkprr |
| Vascular endothelial growth factor A, isoform c NP_001020538.2 | |
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe kksvrgkgkg qkrkrkksrp cgpcserrkh lfvqdpqtck |
| 361 | csckntdsrc karqlelner tcrcdkprr |
| Vascular endothelial growth factor A, isoform d NP_001020539.2 | |
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe npcgpcserr khlfvqdpqt ckcsckntds rckarqleln |
| 361 | ertcrcdkpr r |
| Vascular endothelial growth factor A, isoform e NP_001020540.2 | |
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe npcgpcserr khlfvqdpqt ckcsckntds rckm |
| Vascular endothelial growth factor A, isoform f NP_001020541.2 | |
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe kcdkprr |
| Vascular endothelial growth factor A, isoform g NP_001028928.1 | |
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe npcgpcserr khlfvqdpqt ckcsckntds rckarqleln |
| 361 | ertcrsltrk d |
| Vascular endothelial growth factor A, isoform h NP_001165093.1 | |
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rcdkprr |
| Vascular endothelial growth factor A, isoform i NP_001165094.1 | |
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe kksvrgkgkg qkrkrkksry kswsvyvgar cclmpwslpg |
| 181 | phpcgpcser rkhlfvqdpq tckcsckntd srckarqlel nertcrcdkp rr |
| Vascular endothelial growth factor A, isoform j NP_001165095.1 | |
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe kksvrgkgkg qkrkrkksry kswsvpcgpc serrkhlfvq |
| 181 | dpqtckcsck ntdsrckarq lelnertcrc dkprr |
| Vascular endothelial growth factor A, isoform k NP_001165096.1 | |
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe kksvrgkgkg qkrkrkksrp cgpcserrkh lfvqdpqtck |
| 181 | csckntdsrc karqlelner tcrcdkprr |
| Vascular endothelial growth factor A, isoform l NP_001165097.1 | |
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe npcgpcserr khlfvqdpqt ckcsckntds rckarqleln |
| 181 | ertcrcdkpr r |
| Vascular endothelial growth factor A, isoform m NP_001165098.1 | |
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe npcgpcserr khlfvqdpqt ckcsckntds rckm |
| Vascular endothelial growth factor A, isoform n NP_001165099.1 | |
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe kcdkprr |
| Vascular endothelial growth factor A, isoform o NP_001165100.1 | |
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe npcgpcserr khlfvqdpqt ckcsckntds rckarqleln |
| 181 | ertcrsltrk d |
| Vascular endothelial growth factor A, isoform p NP_001165101.1 | |
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rcdkprr |
| Vascular endothelial growth factor A, isoform q NP_001191313.1 | |
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe kksvrgkgkg qkrkrkksry kswsvcdkpr r |
| Vascular endothelial growth factor A, isoform r NP_001191314.1 | |
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe kksvrgkgkg qkrkrkksry kswsvcdkpr r |
| Vascular endothelial growth factor A, isoform s NP_001273973.1 | |
| 1 | maegggqnhh evvkfmdvyq rsychpietl vdifqeypde ieyifkpscv plmrcggccn |
| 61 | deglecvpte esnitmqimr ikphqgqhig emsflqhnkc ecrpkkdrar qenpcgpcse |
| 121 | rrkhlfvqdp qtckcscknt dsrckarqle lnertcrcdk prr |
| Vascular endothelial growth factor A, isoform VEGF-Ax precursor | |
| NP_001303939.1 | |
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe npcgpcserr khlfvqdpqt ckcsckntds rckarqleln |
| 181 | ertcrcdkpr rsagqeegas lrvsgtrslt rkd |
| WD repeat-containing protein 46, isoform 1 NP_005443.3 | |
| 1 | metapkpgkd vppkkdklqt krkkprrywe eetvpttaga spgpprnkkn relrpqrpkn |
| 61 | ayilkksris kkpqvpkkpr ewknpesqrg lsgtqdpfpg papvpvevvq kfcridksrk |
| 121 | lphskaktrs rlevaeaeee etsikaarse lllaeepgfl egedgedtak icqadiveav |
| 181 | diasaakhfd lnlrqfgpyr lnysrtgrhl afggrrghva aldwvtkklm ceinvmeavr |
| 241 | dirflhseal lavaqnrwlh iydnqgielh cirrcdrvtr leflpfhfll atasetgflt |
| 301 | yldvsvgkiv aalnaragrl dvmsqnpyna vihlghsngt vslwspamke plakilchrg |
| 361 | gvravavdst gtymatsgld hqlkifdlrg tyqplstrtl phgaghlafs qrgllvagmg |
| 421 | dvvniwagqg kasppsleqp ylthrlsgpv hglqfcpfed vlgvghtggi tsmlvpgage |
| 481 | pnfdglesnp yrsrkqrqew evkallekvp aelicldpra laevdvisle qgkkeqierl |
| 541 | gydpqakapf qpkpkqkgrs staslvkrkr kvmdeehrdk vrqslqqqhh keakakptga |
| 601 | rpsaldrfvr |
| WD repeat-containing protein 46, isoform 2 NP_001157739.1 | |
| 1 | metapkpgkd vppkkdklqt krkkprewkn pesqrglsgt qdpfpgpapv pvevvqkfcr |
| 61 | idksrklphs kaktrsrlev aeaeeeetsi kaarsellla eepgfleged gedtakicqa |
| 121 | diveavdias aakhfdlnlr qfgpyrlnys rtgrhlafgg rrghvaaldw vtkklmcein |
| 181 | vmeavrdirf lhseallava qnrwlhiydn qgielhcirr cdrvtrlefl pfhfllatas |
| 241 | etgfltyldv svgkivaaln aragrldvms qnpynavihl ghsngtvslw spamkeplak |
| 301 | ilchrggvra vavdstgtym atsgldhqlk ifdlrgtyqp lstrtlphga ghlafsqrgl |
| 361 | lvagmgdvvn iwagqgkasp psleqpylth rlsgpvhglq fcpfedvlgv ghtggitsml |
| 421 | vpgagepnfd glesnpyrsr kqrqewevka llekvpaeli cldpralaev dvisleqgkk |
| 481 | eqierlgydp qakapfqpkp kqkgrsstas lvkrkrkvmd eehrdkvrqs lqqqhhkeak |
| 541 | akptgarpsa ldrfvr |
| Wilms tumor protein, isoform A NP_000369.4 | |
| 1 | mdflllqdpa stcvpepasq htlrsgpgcl qqpeqqgvrd pggiwaklga aeasaerlqg |
| 61 | rrsrgasgse pqqmgsdvrd lnallpavps lgggggcalp vsgaaqwapv ldfappgasa |
| 121 | ygslggpapp papppppppp phsfikqeps wggaepheeq clsaftvhfs gqftgtagac |
| 181 | rygpfgpppp sgassgqarm fpnapylpsc lesqpairnq gystvtfdgt psyghtpshh |
| 241 | aaqfpnhsfk hedpmgqqgs lgeqqysvpp pvygchtptd sctgsqalll rtpyssdnly |
| 301 | qmtsqlecmt wnqmnlgatl kghstgyesd nhttpilcga qyrihthgvf rgiqdvrrvp |
| 361 | gvaptlvrsa setsekrpfm caypgcnkry fklshlqmhs rkhtgekpyq cdfkdcerrf |
| 421 | srsdqlkrhq rrhtgvkpfq cktcqrkfsr sdhlkthtrt htgekpfscr wpscqkkfar |
| 481 | sdelvrhhnm hqrnmtklql al |
| Wilms tumor protein, isoform B NP_077742.3 | |
| 1 | mdflllqdpa stcvpepasq htlrsgpgcl qqpeqqgvrd pggiwaklga aeasaerlqg |
| 61 | rrsrgasgse pqqmgsdvrd lnallpavps lgggggcalp vsgaaqwapv ldfappgasa |
| 121 | ygslggpapp papppppppp phsfikqeps wggaepheeq clsaftvhfs gqftgtagac |
| 181 | rygpfgpppp sqassgqarm fpnapylpsc lesqpairnq gystvtfdgt psyghtpshh |
| 241 | aaqfpnhsfk hedpmgqqgs lgeqqysvpp pvygchtptd sctgsqalll rtpyssdnly |
| 301 | qmtsqlecmt wnqmnlgatl kgvaagssss vkwtegqsnh stgyesdnht tpilcgagyr |
| 361 | ihthgvfrgi qdvrrvpgva ptlvrsaset sekrpfmcay pgcnkryfkl shlqmhsrkh |
| 421 | tgekpyqcdf kdcerrfsrs dqlkrhqrrh tgvkpfqckt cqrkfsrsdh lkthtrthtg |
| 481 | ekpfscrwps cqkkfarsde lvrhhnmhqr nmtklqlal |
| Wilms tumor protein, isoform D NP_077744.4 | |
| 1 | mdflllqdpa stcvpepasq htlrsgpgcl qqpeqqgvrd pggiwaklga aeasaerlqg |
| 61 | rrsrgasgse pqqmgsdvrd lnallpavps lgggggcalp vsgaaqwapv ldfappgasa |
| 121 | ygslggpapp papppppppp phsfikqeps wggaepheeq clsaftvhfs gqftgtagac |
| 181 | rygpfgpppp sqassgqarm fpnapylpsc lesqpairnq gystvtfdgt psyghtpshh |
| 241 | aaqfpnhsfk hedpmgqqgs lgeqqysvpp pvygchtptd sctgsqalll rtpyssdnly |
| 301 | qmtsqlecmt wnqmnlgatl kgvaagssss vkwtegqsnh stgyesdnht tpilcgaqyr |
| 361 | ihthgvfrgi qdvrrvpgva ptlvrsaset sekrpfmcay pgcnkryfkl shlqmhsrkh |
| 421 | tgekpyqcdf kdcerrfsrs dqlkrhqrrh tgvkpfqckt cqrkfsrsdh lkthtrthtg |
| 481 | ktsekpfscr wpscqkkfar sdelvrhhnm hqrnmtklql al |
| Wilms tumor protein, isoform E NP_001185480.1 | |
| 1 | mekgystvtf dgtpsyghtp shhaaqfpnh sfkhedpmgq qgslgeqqys vpppvygcht |
| 61 | ptdsctgsqa lllrtpyssd nlyqmtsqle cmtwnqmnlg atlkgvaags sssvkwtegq |
| 121 | snhstgyesd nhttpilcga qyrihthgvf rgiqdvrrvp gvaptlvrsa setsekrpfm |
| 181 | caypgcnkry fklshlqmhs rkhtgekpyq cdfkdcerrf srsdqlkrhq rrhtgvkpfq |
| 241 | cktcqrkfsr sdhlkthtrt htgekpfscr wpscqkkfar sdelvrhhnm hqrnmtklql |
| 301 | al |
| Wilms tumor protein, isoform F NP_001185481.1 | |
| 1 | mekgystvtf dgtpsyghtp shhaaqfpnh sfkhedpmgq qgslgeqqys vpppvygcht |
| 61 | ptdsctgsqa lllrtpyssd nlyqmtsqle cmtwnqmnlg atlkghstgy esdnhttpil |
| 121 | cgaqyrihth gvfrgiqdvr rvpgvaptlv rsasetsekr pfmcaypgcn kryfklshlq |
| 181 | mhsrkhtgek pyqcdfkdce rrfsrsdqlk rhqrrhtgvk pfqcktcqrk fsrsdhlkth |
| 241 | trthtgktse kpfscrwpsc qkkfarsdel vrhhnmhqrn mtklqlal |
| X antigen family member 1, isoform a NP_001091063.2 | |
| 1 | mespkkknqq lkvgilhlgs rqkkiriqlr sqcatwkvic kscisqtpgi nldlgsgvkv |
| 61 | kiipkeehck mpeageeqpq v |
| X antigen family member 1, isoform d NP_001091065.1 | |
| 1 | mespkkknqq lkvgilhlgs rqkkiriqlr sqvlgremrd megdlqelhq sntgdksgfg |
| 61 | frrqgednt |
| X-linked inhibitor of apoptosis NP_001158.2, NP_001191330.1 | |
| 1 | mtfnsfegsk tcvpadinke eefveefnrl ktfanfpsgs pvsastlara gflytgegdt |
| 61 | vrcfschaav drwqygdsav grhrkvspnc rfingfylen satqstnsgi qngqykveny |
| 121 | lgsrdhfald rpsethadyl lrtgqvvdis dtiyprnpam yseearlksf qnwpdyahlt |
| 181 | prelasagly ytgigdqvqc fccggklknw epcdrawseh rrhfpncffv lgrnlnirse |
| 241 | sdayssdrnf pnstnlprnp smadyearif tfgtwiysvn keqlaragfy algegdkvkc |
| 301 | fhcgggltdw kpsedpweqh akwypgckyl leqkgqeyin nihlthslee clvrttektp |
| 361 | sltrriddti fqnpmvqeai rmgfsfkdik kimeekiqis gsnykslevl vadlvnaqkd |
| 421 | smqdessqts lqkeisteeq lrrlqeeklc kicmdrniai vfvpcghlvt ckqcaeavdk |
| 481 | cpmcytvitf kqkifms |

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims:

We claim:

1. A method of inducing an immune response in a subject, comprising:
administering to the subject (i) at least one inhibitory antigen and (ii) an effective amount of an agent or a combination of agents, thereby inducing an immune response in the subject,
wherein administration of the inhibitory antigen to the subject, without an effective amount of the agent or the combination of agents, induces an immune response that impairs or reduces immune control of a tumor or cancer cell in the subject.

2. The method of claim 1, wherein the administering step induces an immune response that enhances immune control of the tumor or cancer.

3. The method of claim 1 or 2, wherein administration of the effective amount of the agent or combination of agents redirects an immune response to the inhibitory antigen.

4. The method of claim 3, wherein the immune response to the inhibitory antigen is redirected from an immune response that impairs or reduces immune control of the tumor or cancer to an immune response that enhances immune control of the tumor or cancer.

5. The method of any one of claims 1-4, wherein the agent or combination of agents comprises an adjuvant.

6. The method of claim 5, wherein the adjuvant or combination of adjuvants comprises one or more of a TLR agonist, an inflammasome activator, a NOD2 agonist, a RIG1 helicase inhibitor, or a STING agonist.

7. The method of claim 6, wherein the adjuvant or combination of adjuvants comprises QS-21. or a synthetic variant.

8. The method of claim 6, wherein the adjuvant or combination of adjuvants comprises a TLR4 agonist, a TLR9 agonist, or a TLR4 agonist and a TLR9 agonist.

9. The method of claim 8, wherein the adjuvant or combination of adjuvants comprises 3D-PHAD, CpG, or 3D-PHAD and CpG.

10. The method of claim 6, wherein the adjuvant or combination of adjuvants comprises a TLR4 agonist, a TLR9 agonist, and an inflammasome activator.

11. The method of claim 10, wherein the adjuvant or combination of adjuvants comprises 3D-PHAD, CpG, and QS-21.

12. The method of any one of claims 1-4, wherein the agent or combination of agents comprises a checkpoint inhibitor (e.g., a PD-1 inhibitor, a PD-L1 inhibitor, or a CTLA-4 inhibitor).

13. The method of any one of claims 1-12, wherein the combination of agents comprises a checkpoint inhibitor and an adjuvant.

14. The method of any one of claims 1-4, wherein the agent or combination of agents comprises a viral vector, a bacterial vector, an exosome, a liposome, DNA, mRNA, or saRNA.

15. The method of any one of claims 1-4, wherein the agent or combination of agents comprises a chemotherapeutic agent or an IDO inhibitor.

16. The method of any one of claims 1-15, wherein the inhibitory antigen is a tumor antigen (e.g., tumor specific antigen [TSA or neoantigen], tumor associated antigen [TAA], or cancer/testis antigen [CTA]).

17. The method of any one of claims 1-16, wherein the immune response comprises a T cell-mediated immune response.

18. The method of any one of claims 1-16, wherein the immune response comprises an antigen presenting cell (APC)-mediated immune response.

19. The method of any one of claims 1-16, wherein the immune response comprises a B cell-mediated immune response.

20. The method of any one of claims 1-16, wherein the immune response comprises a response mediated by one or more cells of the innate immune system (e.g., an NK cell, an NKT cell, or a monocyte).

21. The method of any one of claims 1-20, wherein an immune response that impairs or reduces immune control of a tumor or cancer cell comprises a deleterious or non-beneficial lymphocyte response.

22. The method of claim 21, wherein the deleterious or non-beneficial lymphocyte response comprises a decrease or no measurable change, relative to a control, in the level of one or more immune co-stimulatory molecules or signals, one or more immune cytokines or cytokine signals, or one or more MHC molecules.

23. The method of claim 21 or 22, wherein the deleterious or non-beneficial lymphocyte response comprises a decrease or no measurable change, relative to a control, in storage or secretion of immune lytic molecules (e.g., granzyme, or perforin), or other immune effector molecules.

24. The method of any one of claims 21-23, wherein the deleterious or non-beneficial lymphocyte response comprises a decrease or no measurable change, relative to a control, in cytotoxic CD8⁺ T cell and/or CD4⁺ Th1 activity.

25. The method of any one of claims 21-24, wherein the deleterious or non-beneficial lymphocyte response comprises a decrease or no measurable change, relative to a control, in recruitment of beneficial immune cell types.

26. The method of claims 21-25, wherein the deleterious or non-beneficial lymphocyte of any one response comprises a reduction, relative to a control, in a level of an anti-tumor antibody.

27. The method of any one of claims 21-26, wherein the deleterious or non-beneficial lymphocyte response comprises a reduction, relative to a control, in a level of antibody-dependent cell-mediated toxicity (ADCC) against a tumor.

28. The method of any one of claims 21-27, wherein the deleterious or non-beneficial lymphocyte response comprises a reduction, relative to a control, in a level of an antibody that binds the inhibitory antigen expressed by, or present on a surface of, the tumor.

29. The method of any one of claims 1-28, wherein an immune response that enhances immune control of a tumor or cancer cell comprises a beneficial lymphocyte response.

30. The method of claim 29, wherein the beneficial lymphocyte response comprises an increase, relative to a control, in the level of one or more immune co-stimulatory molecules or signals, one or more immune cytokines or cytokine signals, or one or more MHC molecules.

31. The method of claim 29 or 30, wherein the beneficial lymphocyte response comprises an increase, relative to a control, in storage or secretion of immune lytic molecules (e.g., granzyme, or perforin), or other immune effector molecules.

32. The method of any one of claims 29-31, wherein the beneficial lymphocyte response comprises an increase, relative to a control, in cytotoxic CD8⁺ T cell and/or CD4⁺ Th1 activity.

33. The method of any one of claims 29-32, wherein the beneficial lymphocyte response comprises an increase, relative to a control, in recruitment of beneficial immune cell types.

34. The method of any one of claims 29-33, wherein the beneficial lymphocyte response comprises an increase, relative to a control, in a level of an anti-tumor antibody.

35. The method of any one of claims 29-34, wherein the beneficial lymphocyte response comprises an increase, relative to a control, in a level of antibody-dependent cell-mediated toxicity (ADCC) against a tumor.

36. The method of any one of claims 29-35, wherein the beneficial lymphocyte response comprises an increase, relative to a control, in a level of an antibody that binds the inhibitory antigen expressed by, or present on a surface of, the tumor.

37. The method of any one of claims 1-36, wherein the inhibitory antigen and the agent or combination of agents are co-administered.

38. The method of claim 37, wherein the inhibitory antigen and the agent or combination of agents are co-administered as a single composition.

39. The method of claim 37, wherein the inhibitory antigen and the agent or combination of agents are co-administered as separate compositions.

40. The method of any one of claims 1-36, wherein the inhibitory antigen is administered prior to the agent or combination of agents.

41. The method of any one of claims 1-36, wherein the inhibitory antigen is administered after the agent or combination of agents.

42. The method of any one of claims 1-41, wherein an immune response that enhances immune control of the tumor or cancer comprises one or more beneficial clinical responses.

43. The method of any one of claims 1-42, wherein an immune response that enhances immune control of the tumor or cancer comprises clearance, regression, or stabilization of the tumor or cancer, e.g., a level of one or more clinical measures associated with clearance, regression, or stabilization of a cancer.

44. The method of any one of claims 1-42, wherein an immune response that enhances immune control of the tumor or cancer comprises an absence of relapse, recurrence, and/or metastasis of a cancer, e.g., over a defined period of time (e.g., at least 1, 2, 3, 4, 5, 6, 7, 8, 9, 10, 11, 12 weeks, or at least 1, 2, 3, 4, 5, 6, 7, 8, 9, 10, 11, 12 months, or at least 1, 2, 3, 4, 5, 6, 7, 8, 9, 10, 11, 12 years).

45. The method of any one of claims 1-42, wherein an immune response that enhances immune control of the tumor or cancer comprises a positive cancer prognosis.

46. The method of any one of claims 1-42, wherein an immune response that enhances immune control of the tumor or cancer comprises an absence or reduction of one or more toxic responses and/or side effects (e.g., one or more measurable toxic responses and/or side effects) to a cancer therapy or combination of therapies.

47. The method of any one of claims 1-46, further comprising administering to the subject a cancer therapy or combination of therapies.

48. An immunogenic composition comprising (i) at least one inhibitory antigen (e.g., an inhibitory antigen described herein), and (ii) an effective amount of an agent or a combination of agents described herein.
